(19) United States
(12) Patent Application Publication
(10) Pub. No.: US 2025/0137051 A1
(43) Pub. Date: May 1, 2025

(54) AN IN VITRO METHOD FOR DIAGNOSIS FOR ISCHEMIC VASCULAR DISEASES AND RELATED COMPLICATIONS

(71) Applicant: PROSTEM CARDIOLOGY B.V., Maastricht (NL)

(72) Inventors: Conradus Ghosal GHO, Maastricht (NL); Eric DUCKERS, Maastricht (NL); Benedictus Constantinus Ghosal GHO, Maastricht (NL)

(21) Appl. No.: 18/689,460

(22) PCT Filed: Sep. 6, 2022
(86) PCT No.: PCT/EP2022/074728

§ 371 Date: Mar. 6, 2024

(30) Foreign Application Priority Data
Sep. 6, 2021 (EP) 21195112.4

(51) Int. Cl. C12Q 1/6883

(52) U.S. Cl. CPC C12Q 1/6883; C12Q 2600/158

(57) ABSTRACT

An in vitro method for diagnosis of occurrence and/or assessing severity of ischemic vascular disease (IVD) and/or ischemic vascular disease-related complication (IVDC) in a female subject.

### FIELD OF THE INVENTION

The invention relates to an in vitro method for diagnosis of occurrence and/or assessing severity of ischemic vascular disease (IVD) and/or ischemic vascular disease-related complication (IVDC) in subjects.

### BACKGROUND OF THE INVENTION

One of the manifestations of ischemic vascular diseases is coronary artery disease (also referred to as atherosclerotic heart disease, atherosclerotic cardiovascular disease, and coronary heart disease). Coronary artery disease is a disease of the coronary arteries that leads to, or is associated with, the occurrence of several life-threating conditions or complications including myocardial ischemia, angina pectoris, myocardial infarction (also referred to as heart attack), sudden coronary death, coronary death, heart arrhythmia, cardiogenic shock, and others.

Coronary artery disease and related complications are associated or develop as a result of a limitation of the blood flow to the heart as a result of, for instance, a partial or complete blockage of the coronary arteries (e.g. stenosis and/or occlusion of coronary vessels). A common underlying cause for narrowing arteries is atherosclerosis, which is the long-term accumulation of cholesterol-rich plaques in the coronary arteries. Factors which have been associated with atherosclerosis and promote the atherosclerotic plaque development in coronary arteries include (but are not limited to) smoking, high blood pressure, high cholesterol, diabetes or insulin-resistance, aging, sedentary lifestyle, genetic predisposition and others.

Limitation of the blood flow to the heart due to partial or complete blockage of the coronary arteries can cause myocardial ischemia, which is a situation resulting from a lack of oxygen supply to the heart. Depending on the severity of the ischemic event, the lack of blood supply and oxygen may cause a range of manifesting symptoms ranging from relatively mild symptoms, such as chest pain, chest discomfort and shortness of breath (also known as angina pectoris), to relatively more severe complications, such as unstable angina pectoris (chest pain in rest) or myocardial infarction (e.g. heart attack) when the blockage of a coronary artery is severe or occurs suddenly. This leads to loss of cardiomyocytes. Over time, IVD and related complications may cause irreversible damage to the heart such as heart muscle damage accompanied with myocardial scarring without heart muscle regrowth and adverse cardiac remodeling. Under these conditions, the heart can no longer pump blood sufficiently, leading to heart failure with, for instance, symptoms like fluid retention in the lungs, liver, and legs of a patient.

IVD and related complications are the leading cause of death in Western Society and a major cause of hospital admissions. Therefore, IVD and related complication represent a major health concern, which have triggered significant efforts towards finding ways to reduce the mortality and morbidity associated with IVD and related complications. Particularly, efforts have been devoted to devise tests or assays, which help physicians in diagnosing ongoing IVD and related complications or predicting a patient's risk of developing IVD and related complications as early and as reliably as possible.

For the purpose of diagnosing IVD and related complications, physicians largely rely on standard clinical tests, which involve performing a review of a patient's medical history and submitting the patient to standard of care tests and procedures. For the diagnosis of ischemic coronary artery disease these tests and procedures include electrocardiogram (ECG), (stress) echocardiograms, physical stress test, nuclear perfusion scintigraphy scans, coronary angiography, cardiac CT scans, cardiac magnetic resonance (perfusion) imaging or using Holter monitoring, and the like. These tests and procedures suffer from the following drawbacks. First, because some of these tests involve percutaneous measures, they are necessarily invasive and associated with considerable risks and discomfort for the patient. Second, these tests are in general laborious and require specific infrastructure and expertise for analyzing the test results. Therefore, for each patient, the diagnosis of IVD and related complications is complex, time-consuming and cost-inefficient. More importantly, these procedures lack sensitivity and specificity for diagnosing IVD and related complications in general and particularly for diagnosing myocardial or (cerebrovascular and peripheral vascular) vascular ischemia. That is because early ischemic conditions, such as myocardial ischemia can be quiescent or transient and associated with no or little tissue damage. Further, ischemic conditions may also not present with cholesterol-rich plaques or other obstructions. For these reasons, myocardial ischemia may escape detection by these conventional diagnostic methods (e.g. ECG, bicycle stress exercise test, and others). For ischemic vascular disease there are no adequate (or quantitative) detection methods.

Other types of diagnostic blood tests that have been developed over the last decade require the occurrence of irreversible tissue damage (and loss of cells) due to the ischemia leading to the release of intracellular proteins (or substances) into the circulation. One example of such tests consists of diagnostic tests or assays relying on the use of a single pathophysiological marker or blood biomarker (protein or substances circulating in the blood, e.g. in serum, plasma or buffy coat), such as the troponin assay. Troponin is a complex of three regulatory proteins (troponin C, troponin I, and troponin T) that is integral to muscle contraction in skeletal muscle and cardiac muscle, but not in smooth muscle. Troponin has been used as a biomarker of heart diseases, specifically when irreversible (myocardial) tissue damage occurs, for instance during myocardial infarction. This causes the intracellular troponin to be released from the damaged muscle cells into the circulation. Thus, increased levels of circulating troponin have served as an indicator of the extent cardiac muscle cell death/loss. However, this test suffers from a lack of sensitivity and specificity, and often give false-positive results and fails to detect (early) IVD without any significant myocardial damage (such as in for example angina pectoris or silent angina). Hence, the troponin assay can only diagnose patients with a myocardial infarction where extensive ischemia occurred and caused irreversible tissue damage.

Other examples of diagnostic blood tests or assays for detection of ischemic coronary artery disease relying on the use of a single serum biomarker are tests measuring blood levels of heart-type fatty acid-binding protein (H-FABP) or peptidoglycan recognition protein (PGLYRP1) or interleukin-1 receptor-associated kinase 3 (IRAK3) or vanin 3 (VNN3) or creatine kinase-MB isoenzyme (CK-MB) or myoglobin or others. These assays also suffer from the same limitations as the troponin assay, i.e. they can detect tissue necrosis caused by extensive and persistent ischemia but they cannot detect IVD that occurred without necrosis or tissue damage (for instance angina pectoris or silent angina).

Another category of diagnostic blood tests consists of tests relying on the use of a single protein-gene biomarker or a combination of biomarkers. Examples of biomarkers used in such tests include MEF2A, LDLR, ApoB-100, PCSK9, CYP7A1, ARH, LP8, ALOX5AP, PED4D, LTA4, LGALS2, and others (for instance see Table 1 of Robin et al in Journal of the American College of Cardiology, Vol. 50, No. 8 (2007), pp. 727-737 or see Table 1 of Gibbons et al in Circulation (2004) Suppl. IV: IV-47-IV-58)). For example, LDLR, APOB, and PCSK are associated with hypercholesterolemia and cholesterol-rich plaque formation and may indirectly be linked to a higher chance of IVD, which, however, may not form or give a late sign of vascular disease.

The biomarkers used in these types of diagnostic tests typically derive from the results of genome wide analysis or linkage analysis, as part of studies investigating the genetic underpinnings of vascular diseases in general. These tests may be used to determine a subject's (predisposed) risk of developing atherosclerotic lesions, but not to identify/diagnose, stratify patients suffering from IVD (i.e. with insufficient blood flow to the heart) or monitor their response to therapy. Moreover, these biomarkers suffer from the same drawbacks mentioned above, i.e. they lack specificity and sensitivity for diagnosing (progression and monitoring of treatment response) IVD and related complications, particularly (but not limited to) myocardial ischemia, cerebrovascular ischemia (such as cerebrovascular ischemic events (CVA, TIA)) and peripheral artery disease, among others. The clinical utility of these tests has proven to be rather limited as these tests failed to translate into intervention programs or treatment plans that improve outcome of patients who tested positive. Furthermore, due to the genetic heterogeneity inherent to different populations and because of sex differences, the results of these tests do not replicate across populations, i.e. the tests do not identify individuals at risk or already suffering from IVD and related complications in all populations.

Another major drawback associated with the diagnostic tests discussed above is the lack of sex-specific diagnostic tests for IVD and related complications. It is well-recognized that major differences exist in the presentation of symptoms, diagnosis, management and outcomes between men and women with IVD and related complications such as myocardial ischemia. IVD and related complications, particularly myocardial ischemia, are frequently under-diagnosed in women compared to men because the current diagnostic tests or assays are not sensitive or reliable enough to detect sex-related differences. Overall, the atypical clinical manifestation and underdiagnosis of IVD in women calls for more objective and tailored diagnostic tests for both men and women.

Therefore, there is a need for alternative diagnostic tests, which are more reliable and more sensitive in diagnosing IVD and related complications, particularly ongoing IVD, such as myocardial ischemia, particularly during the early stage of myocardial ischemia where no significant tissue damage or loss has occurred. Further, there is a need for alternative diagnostic tests, which rely on the use of biomarkers, which are more proximate to the underlying cause(s) of IVD and related complications or ongoing physical conditions associated with IVD and related complications, and which can serve as surrogate biomarkers or surrogate endpoint markers. Further, there is a need for alternative tests or assays which can be used for diagnosing, detecting the occurrence of, assessing severity of or progression of ischemic vascular diseases (IVD) and related complications (e.g. myocardial ischemia) and/or monitoring IVD and related complications (e.g. myocardial ischemia) over time or in response to treatment in female subjects and male subjects, in a more sensitive and reliable manner as well as in a sex-specific manner.

### OBJECTS OF THE INVENTION

It is an object of the present invention to provide to an in vitro method for diagnosis of occurrence and/or assessing severity of ischemic vascular disease (IVD) and/or ischemic vascular disease-related complication (IVDC) in subjects.

It is a further object of the invention to provide to an in vitro method for diagnosis of occurrence and/or assessing severity of ischemic vascular disease (IVD) and/or ischemic vascular disease-related complication (IVDC) in specific for female subjects.

It is a further object of the invention to provide to an in vitro method for diagnosis of occurrence and/or assessing severity of ischemic vascular disease (IVD) and/or ischemic vascular disease-related complication (IVDC) in specific for male subjects.

### SUMMARY OF THE INVENTION

An in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) from a test sample collected from said female subject, and b. comparing said first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) collected from said female subject with a control expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level. An in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and b. comparing said first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level.

The present inventor surprisingly found that a specific sub-group of the RNA biomarkers expressed in or on CD45-positive cells, isolated from a blood sample taken from a subject can be used as reliable and specific RNA biomarkers for diagnosing, detecting ongoing IVD and/or IVDCs (e.g. myocardial ischemia, particularly in the early stage of myocardial ischemia when tissue damage or loss is minimal, absent or not-detectable using current diagnostic methods), determining the damage caused by or severity of IVD and/or IVDCs and/or monitoring the progression of IVD and/or IVDCs over time in either a female subject or a male subject.

The present inventor found that the RNA biomarkers as taught herein can be reliably detected in a sample from a subject comprising total polynucleotide isolated from CD45+ MNC cells using hybridization probes specifically designed to hybridize to a nucleic acid sequence segment of the RNA biomarkers as taught herein, said probe sets having nucleic acid sequences selected from the nucleic acid sequences of tables 1 to 6.

The present inventor found that that detecting the expression level of at least two, preferably at least five, preferably at least ten, more preferably at least fourteen, of the RNA biomarkers as taught herein, could be used in a sample comprising total polynucleotide isolated from CD45+ MNC cells or biofluids obtained from an subject suffering from IVD and/or IVDCs (i.e. for instance myocardial ischemia) compared to the expression of the same RNA biomarkers in a sample comprising total polynucleotide isolated from CD45+ MNC cells or biofluids from an subject not suffering from IVD and/or IVDCs (i.e. myocardial ischemia) could be used as a reliable and sensitive method for diagnosing, detecting ongoing IVD and/or IVDCs (e.g. myocardial ischemia), determining induced stress on cells or tissue (such as for example hypoxic stress), potential damage caused by or severity of IVD and/or IVDCs and/or monitoring the progression of IVD and/or IVDCs over time and/or after treatment in female subjects. The present inventor found that detecting the expression level of at least two, preferably at least ten, preferably at least fourteen, more preferably at least sixteen, more preferably at least eighteen or more of the RNA biomarkers as taught herein of the RNA biomarkers as taught herein, in a sample comprising total polynucleotide isolated from CD45+ MNC cells or biofluids obtained from an subject suffering from IVD and/or IVDCs (i.e. for instance myocardial ischemia) compared to the expression of the same RNA biomarkers in a sample comprising total polynucleotide isolated from CD45+ MNC cells or biofluids from an subject not suffering from IVD and/or IVDCs (i.e. myocardial ischemia) could be used as a reliable and sensitive method for diagnosing, detecting ongoing IVD and/or IVDCs (e.g. myocardial ischemia), determining induced stress on cells or tissue (such as for example hypoxic stress), potential damage caused by or severity of IVD and/or IVDCs and/or monitoring the progression of IVD and/or IVDCs over time and/or after treatment in male subjects. The present inventor found that compared to traditional diagnostic method and RNA biomarkers, the RNA biomarkers as taught herein represent more reliable and sensitive set of RNA biomarkers for the purposes mentioned herein.

Further, the present inventor has found that the RNA biomarkers as taught herein can be divided into two distinct (i.e. non-overlapping) groups of RNA biomarkers, which are referred to herein as ‘sex-specific RNA biomarkers’. The sex-specific RNA biomarkers as taught herein can be used for diagnosing, detecting ongoing IVD and/or IVDCs (e.g. myocardial ischemia), determining induced stress on cells or tissue (such as for example hypoxic stress or mechanical stress), potential damage caused by or severity of IVD and/or IVDC, and/or monitoring the progression of IVD and/or IVDC over time and/or after treatment in each sex (female and male), separately.

The female-specific RNA biomarkers can be detected in a sample, under stringent hybridization conditions, using hybridization probes having a nucleic acid sequence selected from the nucleic acid sequences of tables 1, 2 and 3 while the male-specific RNA biomarkers can be detected in a sample, under stringent hybridization conditions known in the art and indicated by manufacturers (such as for example Affymetrix Human Gene 2.1 ST array), using hybridization probes having a nucleic acid sequence selected from the nucleic acid sequences of tables 4, 5 and 6.

Without being bound to any theories, the RNA biomarkers and methods of the present inventions are believed to be more reliable and more sensitive than conventional RNA biomarkers used in methods for diagnosing, detecting ongoing IVD and/or IVDCs (e.g. myocardial ischemia), determining induced stress on cells or tissue (such as for example hypoxic stress), potential damage caused by or severity of IVD and/or IVDC, and/or monitoring the progression of IVD and/or IVDC over time and/or after treatment in each sex separately because of the nature of the cells from which the RNA biomarkers as taught herein derive, i.e. CD45+ MNC cells. Since CD45+ MNC cells are involved in angiogenesis (formation of new blood vessels from existing ones) and since compensatory angiogenesis is known to occur in tissue undergoing ischemia, the RNA biomarkers of the invention are believed to be more proximate to the underlying cause(s) of IVD and/or IVDCs or ongoing physical conditions associated with IVD and/or IVDCs. RNA biomarkers can be detected in CD45+ MNC cells, as well as in samples that contain CD45+ MNC cells or in biofluids in which the relevant RNA biomarkers have been secreted (from the CD45+ MNC cells).

For instance, the present inventor found that the RNA biomarkers of the invention were more reliable and more sensitive for diagnosing, detecting ongoing IVD and/or IVDCs (particularly the early stage of myocardial ischemia, where tissue damage or loss is minimal or absent), determining induced stress on cells or tissue (such as for example hypoxic stress), potential damage caused by or severity of IVD and/or IVDCs (particularly myocardial ischemia), and/or monitoring the progression of IVD and/or IVDCs (particularly myocardial ischemia) over time and/or after treatment in each sex (female and male) separately, than conventional diagnostic tests and RNA biomarkers used for the same purposes.

### BRIEF DESCRIPTION OF THE DRAWINGS

FIG. 1: Misclassification scores for the classification models depending on the number of optimized male exons (Number of selected variables) of identified RNA biomarkers and prediction models/methods used (DLDA, FDA, LDA, PLSDA, PLSRF, QDA and RF). The Linear Discriminant Analysis (LDA) gave an optimal RNA biomarker set of 16 RNA biomarkers.

FIG. 2: Misclassification scores for the classification models depending on the number of optimized female exons (Number of selected variables) of identified RNA biomarkers and prediction models/methods used (DLDA, FDA, LDA, PLSDA, PLSRF, QDA and RF). The Linear Discriminant Analysis (LDA) gave an optimal RNA biomarker set of 14 RNA biomarkers.

### DETAILED DESCRIPTION OF THE INVENTION

### Definitions

The term ‘ischemic vascular diseases’ (abbreviated as IVD) as used herein refers to a disease of the coronary, cerebrovascular or peripheral arteries, where the arteries become damaged, obstructed, vasospastic or dysfunctional. Damaged or diseased coronary arteries can cause (an absolute or relative) reduced blood flow to the organ (for instance (but not limited to) the heart or brain, due to a partial or complete blockage of the arteries (for instance atherosclerotic stenosis and/or occlusion of coronary vessels). A common underlying cause for blocked arteries is atherosclerosis, which is the long-term accumulation of cholesterol-rich plaques in the arteries. Progression of artherosclerosis is accelerated in patients by factors such as smoking, high blood pressure, high cholesterol, diabetes or insulin resistance, sedentary lifestyle, genetic predisposition and others. But in 30% of men and 40-60% of women “non-obstructive” coronary artery disease is found, which does not present with cholesterol-rich plaques or other obstructions. For instance, limitation of the blood flow to the heart or partial or complete blockage of the coronary arteries may cause myocardial ischemia, which is a situation where myocardial cells lack sufficient supply of oxygen by the coronary arteries. Ischemic vascular diseases comprise, but is not limited to, ‘coronary artery disease’, ‘atherosclerotic heart disease’, ‘atherosclerotic vascular disease’, ‘coronary heart disease’, cerebrovascular ischemic disease, peripheral artery disease, vascular nephropathy, diabetic vasculopathy, Buergers disease, to name a few.

The term ‘ischemic vascular disease-related complications’ (abbreviated as IVDCs) as used herein refers to complications which develop as a consequence or in association with IVD. Depending on the severity of IVD, the lack of blood supply and oxygen may cause a host of IVDCs for instance relatively mild symptoms and complications, such as myocardial ischemia (angina pectoris) and a transient ischemic cerebrovascular attack (TIA) to relatively more severe complications such irreversible tissue damage due to the acute occlusion of vessels (like for instance myocardial infarction, e.g. also known as ‘heart attack’, or a cerebrovascular accident (CVA)). For instance, myocardial ischemia ((angina pectoris) may manifest itself as or causes ‘chest pain’, however may also show no apparent chest pain (silent myocardial ischemia). Other manifestations of angina pectoris may typically include shortness of breath, chest pain associated with shoulder, arm, back, neck, or jaw pain and sensation of heartburn, heart arrhythmia, cardiogenic shock, cardiac arrest, and others. Cerebrovascular transient ischemic events may range from a temporary loss of neurological function with loss of vision or speech, whereas CVA may manifest from persistent speech impairment to hemiplegia. Peripheral artery disease may present itself as claudication intermittent (muscle cramps upon exercise and fatigue). Over time, IVD and IVDCs may cause irreversible damage to the organ that they perfuse for instance the heart which may result in progressive heart muscle loss, myocardial scarring without heart muscle regrowth, adverse tissue remodeling and heart failure. Under these conditions, the heart can no longer pump blood sufficiently.

The term ‘ischemia’, as used herein, refers to an absolute or relative shortage of the blood supply or an inadequate flow of blood to an organ (e.g. heart, brain), body part or tissue. Relative shortage refers to the discrepancy between blood supply (oxygen delivery) and blood request/demand (oxygen consumption by tissue). The restriction in blood supply, generally due to factors in the blood vessels, is most often, but not exclusively, caused by constriction or blockage of the blood vessels by thromboembolism (blood clots) or atherosclerosis (lipid-laden plaques obstructing the lumen of arteries). But in 30% of men and 40-60% of women “non-obstructive” coronary artery disease is found, which does not present with cholesterol-rich plaques or other obstructions. Ischemia result in damage or dysfunction of tissue (e.g. IVD and IVDCs, myocardial ischemia, stroke, transient ischemic attack (TIA) and the like).

The term ‘gene’ as used herein refers to a sequence of nucleotides in DNA or RNA that encodes the synthesis of a gene product, either RNA or protein.

The term ‘exon’ as used herein refers to any part of a gene that will encode a part of the final mature RNA or non-coding RNA transcripts produced by that gene after introns have been removed by RNA splicing.

The term ‘RNA biomarkers’ as used herein refers to a measurable indicator of some biological state or condition or diseases or complications relating to a disease. RNA biomarkers can be any RNA molecule (such as microRNA (miRNA) and long non coding RNA (lncRNA), messenger RNA (mRNA)) circulating in the blood, present in the biofluid, cells or tissue. In an embodiment of the present invention, the RNA biomarkers are present in or on CD45+ circulating mononuclear cells (MNC) isolated from a sample from a subject, or present in blood or any biofluids (for example urine, serum, plasma, buffy coat, stool, saliva, tissue specimen). The RNA biomarkers of the invention may be detected using hybridization probes from a hybridization probe set for each RNA biomarker, having nucleic acid sequences selected from the nucleic acid sequences detailed in tables 1 to 6. These tables contain the complementary DNA sequences that hybridize to the designated claimed RNA biomarkers, which may be in the form of DNA or RNA depending on the test method used to detect the RNA biomarkers. Alternatively, the RNA biomarker or its gene product can be detected using antibodies raised against said RNA biomarker or its gene product.

The terms ‘hybridization probe’ and ‘hybridization probe set’ may also be referred to as ‘probe’ and ‘probe set’, respectively.

The term ‘stringent hybridization conditions’ as used herein refers to a process used to identify polynucleotide sequences, which are substantially identical to a given nucleic acid sequence of a probe. The stringency of the hybridization conditions is sequence dependent and will be different in different circumstances. Method for establishing hybridization conditions are known to the person skilled in the art. Further, hybridization conditions are indicated by manufactures, such as for example Affymetrix Exon Microarray Human Gene 2.1 ST. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridizes to a perfectly matched probe. Typically, stringent conditions will be chosen in which the salt (NaCl) concentration is about 0.02 molar at pH 7 and the temperature is at least 60° C. Lowering the salt concentration and/or increasing the temperature increases stringency. Stringent conditions for RNA-DNA hybridizations (Northern blots using a probe) are for example those which include at least one wash in 0.2×SSC at 63° C. for 20 min, or equivalent conditions. Stringent conditions for DNA-DNA hybridization (Southern blots using a probe) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

In an embodiment, ‘high stringency’ conditions can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrrolidone, 2% Bovine Serum Albumin), 0.5% sodium dodecyl sulphate (SDS), and 20 μg/ml denatured carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.1-0.2×SSC, 0.1% SDS.

In an embodiment, ‘moderate stringency’ refers to conditions equivalent to hybridization in the above-described solution but at about 60-62° C. In that case the final wash is performed at the hybridization temperature in 1×SSC, 0.1% SDS.

In an embodiment, ‘low stringency’ refers to conditions equivalent to hybridization in the above-described solution at about 50-52° C. In that case, the final wash is performed at the hybridization temperature in 2×SSC, 0.1% SDS. See also Sambrook et al. (1989) and Sambrook and Russell (2001).

The term ‘probe’ or ‘hybridization probe’ (which can be sex-specific, female or male specific) as used herein refers to a fragment of DNA of variable length (usually 15-1000 bases long), which may be labelled (e.g. with radioactivity or fluorescent label or others). Probes are typically used in samples, comprising total polynucleotides (DNA and/or RNA) to detect the presence of polynucleotide sequences that are complementary to the nucleic acid sequence in the probe. The probe thereby hybridizes to a single-stranded nucleic acid (DNA or RNA) base sequence via probe-target base pairing due to complementarity between the probe and target. The labelled probe may be hybridized to the target ssDNA (Southern blotting) or RNA (Northern blotting) immobilized on a membrane or in situ. Female-specific probe sets, probes and RNA biomarkers are listed in tables 1, 2 and 3 while male-specific probe sets, probes and RNA biomarkers are listed in tables 4, 5 and 6.

To detect hybridization of the probe to its polynucleotide sequence, the probe can be tagged (or “labelled”) with a molecular marker of either radioactive or fluorescent molecules; commonly used markers are 32P (a radioactive isotope of phosphorus incorporated into the phosphodiester bond in the probe DNA) or biotin-labeling or Digoxigenin, which is a non-radioactive, antibody-based marker, or others. DNA or RNA sequences that have moderate to high sequence similarity to the probe can then be detected by visualizing the hybridized probe via autoradiography or other imaging techniques. Normally, either X-ray pictures are taken of the filter, or the filter is placed under UV light. Detection of sequences with moderate or high similarity depends on how stringent the hybridization conditions were applied—high stringency, such as high hybridization temperature and low salt in hybridization buffers, permits only hybridization between nucleic acid sequences that are highly similar, whereas low stringency, such as lower temperature and high salt, allows hybridization when the sequences are less similar. Hybridization probes used in DNA microarrays refer to DNA covalently attached to an inert surface, such as coated glass slides or gene chips, to which a mobile cDNA target is hybridized. The RNA samples here were detected using a microarray analysis (photolithography—Affymetrix Exon Microarray Human Gene 2.1 ST, Affymetrix Santa Clara, CA, USA). Alternative methods of detecting and (semi) quantitative analysis of RNA may include (but is not limited to quantitative PCR reaction, Luminex detection). Any future methodology to detect the RNA (or DNA) sequences (and products derived thereof, such as non-coding RNAs and proteins) will be covered by the patent.

The term ‘differentially expressed’ in samples comprising total polynucleotide isolated from cells, such as for example CD45+ MNC cells, or differentially expressed in or on cells, such as for example CD45+ MNC cells, isolated from a blood or tissue sample from a subject refers to differences in levels or quantity or frequency (incidence of occurrence) of two or more RNA biomarker as taught herein present in said sample taken from a subject as compared to a control subject or a control population (e.g. reference biobank or database). RNA biomarkers may also be detected in any biofluid (for example stool, saliva, urine, serum, blood, plasma, buffy coat or a tissue specimen) or tissue sample. For example, the level of expression of two or more RNA biomarkers as taught herein, detected from a test sample can be found to be elevated/up-regulated or decreased/down-regulated compared to the levels of the same RNA biomarker detected from a control sample taken from control subject. In the present invention, a RNA biomarker which is differentially expressed in a test sample relative to a control sample is a RNA biomarker that is detected at a substantially higher or lower level or in substantially higher or lower amount or quantity in the test sample relative to the control sample. Preferably, the level of expression of the two or more RNA biomarkers (or products thereof) detected in a sample taken from a subject are statistically higher or lower than the levels of the same two or more RNA biomarkers detected in a sample taken from a control subject. It is understood that the subject and the control subject can either be a female subject or a male subject. Preferably, the subject and the control subject are sex-matched.

The term ‘subject’ or ‘patient’ as used herein refers to a subject or a patient, suspected or known to have IVD or IVDCs, respectively. The term ‘control subject’ as used herein refers to a subject assumed to be without IVD or IVDCs or a control population assumed to be without IVD or IVDCs.

The term ‘increased expression level’ and ‘decreased expression level’ of two or more RNA biomarkers (and products derived thereof, such as non-coding RNAs and proteins) as used herein refers to a substantially or significantly increased expression level or substantially or significantly decreased expression level of two or more RNA biomarkers (and products derived thereof, such as non-coding RNAs and proteins) as taught herein. For instance, a level of expression in a test sample is increased or decreased when it is at least 5%, such as 10%, 15%, 20%, 25%, 30%, 35%, 40%, 45%, 50% higher or lower, than the corresponding expression level in a control sample.

The term ‘test sample’ as used herein refers to a sample (e.g. blood sample, any biofluid sample, or cell sample or tissue sample) taken from a subject or a patient who is at risk of suffering or is suffering from a disease or a related condition (e.g. IVD and/or IVDCs). Typically, a test sample derives from an unhealthy subject or patient.

The term ‘control sample’ as used herein, refers to a sample (e.g. blood sample, biofluids sample or cell sample or tissue sample) taken from a control subject not suffering or who is not at risk of suffering from a disease or a related condition (e.g. IVD and/or IVDCs). Typically, a control sample derives from a healthy subject.

The term ‘determining the damage or severity of IVD and/or IVDCs’ as used herein refers to assessing the degree of ischemia, damage or induced stress on cells or tissue (such as for example hypoxic stress) by IVD and/or IVDCs or assessing the severity of IVD and/or IVDCs in a female or male subject by detecting, in a sample (for instance in or on CD45+ MNC cells isolated from a blood, biofluids or tissue samples from a subject, or in a sample comprising total polynucleotides isolated from CD45+ MNC cells (or biofluids/blood (e.g. buffy coat, serum or plasma) that previously contained CD45+ MNC cells), the expression levels of the female-specific RNA biomarkers or male-specific RNA biomarkers as taught herein. Quantitation of the levels of RNA biomarkers as taught herein can be expressed or reported either in absolute amount (e.g. μg/ml) or a relative amount (e.g., relative intensity of signals or fold changes, etc.) to a control sample. The levels of expression of the RNA biomarkers as taught herein can be determined by any suitable methods such as northern blotting, nuclease protection assay, in situ hybridization, DNA microarray, exon DNA microarray or quantitative RT-PCR, biochemical assays, Luminex technology, chromatographic assays, mass spectrometric assays or hybridization assays using labelled hybridization probes such as those as taught herein), RNA/DNA microarray assay, RNA/DNA Chip assays or immunoassays, ELISA assays. For instance, in an embodiment of the invention, the damage, induced stress on cells or tissue (such as for example hypoxic stress) or ischemia caused by, or the severity of IVD and/or IVDCs may be determined by detecting how divergent the expression level of two or more RNA biomarkers in a test sample is from the expression level of a control value or the expression level of the same RNA biomarker(s) in a control sample (or control population).

The term “angiogenesis” as used herein refers to the formation of blood vessels from existing ones. Vasculogenesis and angiogenesis both occur in adulthood and embryonic development.

The term ‘about’, as used herein indicates a range of normal tolerance in the art, for example within 2 standard deviations of the mean. The term ‘about’ can be understood as encompassing values that deviate at most 10%, 9%, 8%, 7%, 6%, 5%, 4%, 3%, 2%, 1%, 0.5%, 0.1%, 0.05%, or 0.01% of the indicated value.

The term ‘comprising’ or ‘to comprise’ and their conjugations, as used herein, refer to a situation wherein said terms are used in their non-limiting sense to mean that items following the word are included, but items not specifically mentioned are not excluded. It also encompasses the more limiting verb ‘to consist essentially of’ and ‘to consist of’.

Reference to an element by the indefinite article ‘a’ or ‘an’ does not exclude the possibility that more than one of the elements is present, unless the context clearly requires that there be one and only one of the elements. The indefinite article ‘a’ or ‘an’ thus usually means ‘at least one’.

Circulating Mononuclear Cells (MNC cells) have been shown to be involved in vessel formation and maintenance in development and adult mammals (angiogenesis and vascular homeostasis). MNC cells can be isolated from tissue (for instance, the bone marrow, myocardial tissue) or from a blood or biofluid sample obtained from a subject (e.g. for instance from the mononuclear cell fraction of blood, plasma, saliva, urine, stool sample) using standards cell isolation protocol including, but not limited to Ficoll isolation, immune separation using anti-MNC antibodies. In adulthood, MNC cells are detected in the blood of healthy individuals. However, the amount of MNC cells substantially increases in individuals undergoing or having vascular injury or other diseases or conditions causing vasculogenesis (i.e. formation of new blood vessels) and angiogenesis (i.e. formation of blood vessels from existing ones). For example, CD45+ and/or CD14+ MNC cells have been shown to contribute to new vessel formation (Bastiaansen et al 2014 and Bot et al 2020).

Samples can be taken preferably from CD45+ MNC cells isolated from tissue/blood or biofluid samples, but can also be derived from samples without these relevant cells (since the relevant RNA biomarkers have been secreted (from the cells) into the sample).

CD45+ MNC cells are involved in vasculogenesis and vascular homeostasis (maintenance of existing blood vessels) during embryogenesis and adulthood. CD45+ MNC cells can be isolated from tissue (for instance, the bone marrow, myocardial tissue or tumor) or from a blood or biofluid sample obtained from a subject (e.g. for instance from the mononuclear cell fraction of blood, plasma, buffy coat, saliva, urine, serum, blood, stool sample or a tissue specimen) using a standards cell isolation protocol including, but not limited to, immune separation using anti-CD45 and/or anti-CD14 antibodies. In adulthood, the amount of CD45+ MNC cells substantially increases in individuals undergoing or having vascular injury or other diseases or conditions causing angiogenesis (i.e. formation of blood vessels from existing ones in adult subjects).

In a first aspect the invention provides an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) from a test sample collected from said female subject, and b. comparing said first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) collected from said female subject with a control expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level.

Tables 1 2, and 3 are shown below. Table 1 shows the names of the RNA biomarkers identified using the probes in the probe sets (Probe Set ID), further the chromosomes and strands these are found on, for the female subjects. Table 2 details for the corresponding Probe Set IDs, the Exon_ID, the start and end of the exon and the sequences of the probes within each probe set for the female subjects are shown. Table 3 details the corresponding Probe Set IDs, the Exon_ID, the start and end of the exon and the sequences of the probes within the optimal 14 probe set for the female subjects are shown.

TABLE 1
RNA biomarkers, which expression is decreased or increased in a sample comprising
total polynucleotides isolated from CD45-positive cells (or biofluids) from
a female subject, and corresponding chromosomes, strands and probe set (Probe
Set ID) used to identify said female specific RNA biomarkers.

| | | | PROBE |
|---|---|---|---|
| IDENTIFIED RNA BIOMARKER | CHROMOSOME | STRAND | SET ID |
| AK125911 // MMP11 /// | chr22 | + | 16928030 |
| ENST00000465385 // MMP11 /// | | | |
| ENST00000465730 // MMP11 /// | | | |
| ENST00000428253 // MMP11 | | | |
| AK299689 // NKAIN3 /// | chr8 | + | 17069388 |
| ENST00000523367 // NKAIN3 /// | | | |
| ENST00000545532 // NKAIN3 | | | |
| BC039373 // LPAR6 | chr13 | − | 16779021 |
| ENST00000468156 // TBCB | chr19 | + | 16861376 |
| ENST00000493991 // COL2A1 /// | chr12 | − | 16763818 |
| AL834148 // COL2A1 | | | |
| ENST00000531175 // PTS | chr11 | + | 16731259 |
| NM_001102398 // HNRNPR /// | chr1 | − | 16683280 |
| NM_005826 // HNRNPR /// | | | |
| NM_001102399 // HNRNPR /// | | | |
| NM_001102397 // HNRNPR /// | | | |
| ENST00000374616 // HNRNPR /// | | | |
| ENST00000374612 // HNRNPR /// | | | |
| ENST00000302271 // HNRNPR /// | | | |
| AF000364 // HNRNPR /// | | | |
| BC001449 // HNRNPR /// | | | |
| DQ351905 // HNRNPR /// | | | |
| AK297382 // HNRNPR /// | | | |
| AK297405 // HNRNPR /// | | | |
| AK300029 // HNRNPR /// | | | |
| BX647784 // HNRNPR /// | | | |
| ENST00000478691 // HNRNPR /// | | | |
| ENST00000427764 // HNRNPR /// | | | |
| ENST00000426846 // HNRNPR /// | | | |
| ENST00000463552 // HNRNPR /// | | | |
| ENST00000470941 // HNRNPR /// | | | |
| ENST00000476451 // HNRNPR /// | | | |
| ENST00000491459 // HNRNPR | | | |
| NM_001113490 // AMOT /// | chrX | − | 17113427 |
| NM_133265 // AMOT /// | | | |
| ENST00000304758 // AMOT /// | | | |
| ENST00000371959 // AMOT /// | | | |
| ENST00000371962 // AMOT /// | | | |
| ENST00000524145 // AMOT /// | | | |
| AF286598 // AMOT /// | | | |
| ENST00000432214 // AMOT /// | | | |
| ENST00000371958 // AMOT | | | |
| NM_001265577 // KIF18B /// | chr17 | − | 16845809 |
| NM_001264573 // KIF18B /// | | | |
| BC144271 // KIF18B /// | | | |
| ENST00000438933 // KIF18B /// | | | |
| ENST00000339151 // KIF18B /// | | | |
| ENST00000376982 // KIF18B | | | |
| NM_015080 // NRXN2 /// | chr11 | − | 16739924 |
| NM_138732 // NRXN2 /// | | | |
| ENST00000377551 // NRXN2 /// | | | |
| ENST00000377559 // NRXN2 /// | | | |
| ENST00000265459 // NRXN2 /// | | | |
| AB035266 // NRXN2 /// | | | |
| ENST00000345863 // NRXN2 | | | |
| NM_015690 // STK36 /// | chr2 | + | 16891029 |
| NM_001243313 // STK36 /// | | | |
| ENST00000295709 // STK36 /// | | | |
| ENST00000392105 // STK36 /// | | | |
| ENST00000440309 // STK36 /// | | | |
| AF200815 // STK36 /// BC026158 // | | | |
| STK36 /// ENST00000392106 // | | | |
| STK36 /// ENST00000486644 // | | | |
| STK36 /// ENST00000473681 // | | | |
| STK36 /// ENST00000431040 // | | | |
| STK36 | | | |
| NM_016328 // GTF2IRD1 /// | chr7 | + | 17047164 |
| NM_005685 // GTF2IRD1 /// | | | |
| NM_001199207 // GTF2IRD1 /// | | | |
| ENST00000265755 // GTF2IRD1 /// | | | |
| ENST00000424337 // GTF2IRD1 /// | | | |
| ENST00000574416 // GTF2IRD1 /// | | | |
| ENST00000573543 // GTF2IRD1 /// | | | |
| ENST00000455841 // GTF2IRD1 /// | | | |
| ENST00000573381 // GTF2IRD1 /// | | | |
| AF151354 // GTF2IRD1 /// | | | |
| AF104923 // GTF2IRD1 /// | | | |
| AF156489 // GTF2IRD1 /// | | | |
| AY648295 // GTF2IRD1 /// | | | |
| AF118270 // GTF2IRD1 /// | | | |
| AF089107 // GTF2IRD1 /// | | | |
| BC018136 // GTF2IRD1 /// | | | |
| AB209389 // GTF2IRD1 /// | | | |
| ENST00000476977 // GTF2IRD1 /// | | | |
| ENST00000470715 // GTF2IRD1 /// | | | |
| ENST00000573869 // GTF2IRD1 /// | | | |
| ENST00000572452 // GTF2IRD1 | | | |
| NM_144639 // UROC1 /// | chr3 | − | 16958674 |
| NM_001165974 // UROC1 /// | | | |
| ENST00000290868 // UROC1 /// | | | |
| ENST00000383579 // UROC1 /// | | | |
| BC115405 // UROC1 | | | |
| NM_144973 // DENND5B /// | chr12 | − | 16762944 |
| ENST00000389082 // DENND5B /// | | | |
| AK125323 // DENND5B /// | | | |
| ENST00000306833 // DENND5B /// | | | |
| ENST00000536562 // DENND5B | | | |
| NM_198082 // CCDC57 /// | chr17 | − | 16850156 |
| ENST00000389641 // CCDC57 /// | | | |
| ENST00000392347 // CCDC57 /// | | | |
| BC110997 // CCDC57 | | | |
| NM_213596 // FOXN4 /// | chr12 | − | 16769889 |
| ENST00000299162 // FOXN4 /// | | | |
| BC146825 // FOXN4 /// | | | |
| ENST00000355216 // FOXN4 /// | | | |
| ENST00000266856 // FOXN4 /// | | | |
| ENST00000423960 // FOXN4 | | | |
| TCONS_00015170-XLOC_007214 | chr8 | − | 17081037 |
| // chr8 // 100 // 1 // 1 // 0 | | | |
| AK094845 // chr12 // 100 // 1 // 1 // 0 | chr12 | − | 16770033 |
| ENST00000257359 // ADAMTS8 /// | chr11 | − | 16746216 |
| NM_007037 // ADAMTS8 /// | | | |
| AF060153 // ADAMTS8 /// | | | |
| ENST00000414575 // ADAMTS8 | | | |
| ENST00000504429 // NBLA00301 | chr4 | + | 16972455 |
| NM_001142553 // DCHS2 /// | chr4 | − | 16980805 |
| ENST00000443500 // DCHS2 /// | | | |
| NM_001142552 // DCHS2 /// | | | |
| ENST00000339452 // DCHS2 /// | | | |
| ENST00000544161 // DCHS2 | | | |
| NM_003211 // TDG /// | chr12 | + | 16756127 |
| ENST00000392872 // TDG /// | | | |
| AK303631 // TDG /// AK295387 // | | | |
| TDG /// BC037557 // TDG /// | | | |
| ENST00000542926 // TDG /// | | | |
| ENST00000540956 // TDG /// | | | |
| ENST00000542036 // TDG /// | | | |
| AK309792 // TDG /// | | | |
| ENST00000266775 // TDG /// | | | |
| ENST00000544861 // TDG | | | |
| NM_024638 // QTRTD1 /// | chr3 | + | 16944179 |
| NM_001256835 // QTRTD1 /// | | | |
| ENST00000281273 // QTRTD1 /// | | | |
| ENST00000485050 // QTRTD1 /// | | | |
| CR457296 // QTRTD1 /// | | | |
| BC034559 // QTRTD1 /// | | | |
| BC039265 // QTRTD1 /// | | | |
| ENST00000466050 // QTRTD1 /// | | | |
| ENST00000490183 // QTRTD1 /// | | | |
| ENST00000466870 // QTRTD1 /// | | | |
| ENST00000482307 // QTRTD1 /// | | | |
| ENST00000472599 // QTRTD1 | | | |
| NM_033637 // BTRC /// | chr10 | + | 16708482 |
| NM_003939 // BTRC /// | | | |
| NM_001256856 // BTRC /// | | | |
| ENST00000370187 // BTRC /// | | | |
| ENST00000393441 // BTRC /// | | | |
| ENST00000408038 // BTRC /// | | | |
| GU727631 // BTRC /// BC027994 // | | | |
| BTRC /// AF129530 // BTRC /// | | | |
| AK295870 // BTRC /// | | | |
| ENST00000493877 // BTRC | | | |
| NM_033655 // CNTNAP3 /// | chr9 | − | 17094394 |
| NM_001201380 // CNTNAP3B /// | | | |
| ENST00000297668 // CNTNAP3 /// | | | |
| ENST00000377564 // CNTNAP3B | | | |
| /// ENST00000377564 // CNTNAP3 | | | |
| /// AF333769 // CNTNAP3 /// | | | |
| ENST00000477002 // CNTNAP3 /// | | | |
| ENST00000484254 // CNTNAP3B | | | |
| /// ENST00000484254 // CNTNAP3 | | | |
| /// ENST00000467854 // | | | |
| CNTNAP3B /// ENST00000467854 | | | |
| // CNTNAP3 | | | |
| NM_152891 // PRSS33 /// | chr16 | − | 16823203 |
| ENST00000293851 // PRSS33 /// | | | |
| ENST00000570702 // PRSS33 /// | | | |
| BC036846 // PRSS33 | | | |

TABLE 2
Probe sets and probes that identifies specific exons in the RNA biomarker, which
are used for detecting that expression is decreased or increased of RNA biomarkers
in a sample comprising total polynucleotides isolated from CD45-positive cells (or biofluids)
from a female subject, corresponding Probe Set IDs the Exon_ID, the start and
end of the exon and the nucleic acid sequences of the probes (Nucleic Acid Sequences
of Probes) within each probe set for the female subjects.

| PROBE | | | | NUCLEIC ACID |
|---|---|---|---|---|
| SET ID | EXON_ID | START | STOP | SEQUENCES OF PROBES |
| 16928030 | 5187159 | 24121613 | 24121639 | SEQ ID NO: 1, SEQ ID NO: 2, |
| | | | | SEQ ID NO: 3, SEQ ID NO: 4, |
| | | | | SEQ ID NO: 5, SEQ ID NO: 6, |
| | | | | SEQ ID NO: 7, SEQ ID NO: 8, |
| | | | | SEQ ID NO: 9, SEQ ID NO: 10, |
| | | | | SEQ ID NO: 11, SEQ ID NO: 12, |
| | | | | SEQ ID NO: 13, SEQ ID NO: 14, |
| | | | | SEQ ID NO: 15, SEQ ID NO: 16, |
| | | | | SEQ ID NO: 17, SEQ ID NO: 18, |
| | | | | SEQ ID NO: 19, SEQ ID NO: 20, |
| | | | | SEQ ID NO: 21, SEQ ID NO: 22, |
| | | | | SEQ ID NO: 23, SEQ ID NO: 24, |
| | | | | SEQ ID NO: 25, SEQ ID NO: 26, |
| | | | | SEQ ID NO: 27, SEQ ID NO: 28, |
| | | | | SEQ ID NO: 29, SEQ ID NO: 30, |
| | | | | SEQ ID NO: 31, SEQ ID NO: 32 |
| 17069388 | 5294444 | 63161464 | 63161488 | SEQ ID NO: 33, SEQ ID NO: 34, |
| | | | | SEQ ID NO: 35, SEQ ID NO: 36, |
| | | | | SEQ ID NO: 37, SEQ ID NO: 38, |
| | | | | SEQ ID NO: 39, SEQ ID NO: 40, |
| | | | | SEQ ID NO: 41, SEQ ID NO: 42, |
| | | | | SEQ ID NO: 43, SEQ ID NO: 44, |
| | | | | SEQ ID NO: 45, SEQ ID NO: 46, |
| | | | | SEQ ID NO: 47, SEQ ID NO: 48, |
| | | | | SEQ ID NO: 49, SEQ ID NO: 50, |
| | | | | SEQ ID NO: 51 |
| 16779021 | 5069104 | 49000747 | 49000882 | SEQ ID NO: 52, SEQ ID NO: 53, |
| | | | | SEQ ID NO: 54, SEQ ID NO: 55, |
| | | | | SEQ ID NO: 56, SEQ ID NO: 57, |
| | | | | SEQ ID NO: 58, SEQ ID NO: 59, |
| | | | | SEQ ID NO: 60, SEQ ID NO: 61, |
| | | | | SEQ ID NO: 62, SEQ ID NO: 63, |
| | | | | SEQ ID NO: 64, SEQ ID NO: 65, |
| | | | | SEQ ID NO: 66, SEQ ID NO: 67, |
| | | | | SEQ ID NO: 68, SEQ ID NO: 69, |
| | | | | SEQ ID NO: 70, SEQ ID NO: 71 |
| 16861376 | 5134829 | 36612868 | 36613067 | SEQ ID NO: 72, SEQ ID NO: 73, |
| | | | | SEQ ID NO: 74, SEQ ID NO: 75, |
| | | | | SEQ ID NO: 76, SEQ ID NO: 77, |
| | | | | SEQ ID NO: 78, SEQ ID NO: 79, |
| | | | | SEQ ID NO: 80, SEQ ID NO: 81, |
| | | | | SEQ ID NO: 82, SEQ ID NO: 83, |
| | | | | SEQ ID NO: 84, SEQ ID NO: 85, |
| | | | | SEQ ID NO: 86, SEQ ID NO: 87, |
| | | | | SEQ ID NO: 88, SEQ ID NO: 89, |
| | | | | SEQ ID NO: 90, SEQ ID NO: 91, |
| | | | | SEQ ID NO: 92, SEQ ID NO: 93, |
| | | | | SEQ ID NO: 94, SEQ ID NO: 95, |
| | | | | SEQ ID NO: 96, SEQ ID NO: 97, |
| | | | | SEQ ID NO: 98, SEQ ID NO: 99, |
| | | | | SEQ ID NO: 100, SEQ ID NO: |
| | | | | 101, SEQ ID NO: 102 |
| 16763818 | 5057504 | 48382221 | 48382245 | SEQ ID NO: 103, SEQ ID NO: |
| | | | | 104, SEQ ID NO: 105, SEQ ID |
| | | | | NO: 106, SEQ ID NO: 107, SEQ |
| | | | | ID NO: 108, SEQ ID NO: 109, |
| | | | | SEQ ID NO: 110, SEQ ID NO: |
| | | | | 111, SEQ ID NO: 112, SEQ ID |
| | | | | NO: 113, SEQ ID NO: 114, SEQ |
| | | | | ID NO: 115, SEQ ID NO: 116, |
| | | | | SEQ ID NO: 117, SEQ ID NO: |
| | | | | 118, SEQ ID NO: 119, SEQ ID |
| | | | | NO: 120, SEQ ID NO: 121, SEQ |
| | | | | ID NO: 122, SEQ ID NO: 123, |
| | | | | SEQ ID NO: 124, SEQ ID NO: |
| | | | | 125, SEQ ID NO: 126, SEQ ID |
| | | | | NO: 127, SEQ ID NO: 128, SEQ |
| | | | | ID NO: 129, SEQ ID NO: 130 |
| 16731259 | 5033908 | 112100845 | 112100894 | SEQ ID NO: 131, SEQ ID NO: |
| | | | | 132, SEQ ID NO: 133, SEQ ID |
| | | | | NO: 134, SEQ ID NO: 135, SEQ |
| | | | | ID NO: 136, SEQ ID NO: 137, |
| | | | | SEQ ID NO: 138, SEQ ID NO: |
| | | | | 139, SEQ ID NO: 140, SEQ ID |
| | | | | NO: 141, SEQ ID NO: 142, SEQ |
| | | | | ID NO: 143, SEQ ID NO: 144, |
| | | | | SEQ ID NO: 145, SEQ ID NO: |
| | | | | 146, SEQ ID NO: 147, SEQ ID |
| | | | | NO: 148, SEQ ID NO: 149, SEQ |
| | | | | ID NO: 150, SEQ ID NO: 151, |
| | | | | SEQ ID NO: 152, SEQ ID NO: |
| | | | | 153, SEQ ID NO: 154 |
| 16683280 | 4997115 | 23645050 | 23645174 | SEQ ID NO: 155, SEQ ID NO: |
| | | | | 156, SEQ ID NO: 157, SEQ ID |
| | | | | NO: 158, SEQ ID NO: 159, SEQ |
| | | | | ID NO: 160, SEQ ID NO: 161, |
| | | | | SEQ ID NO: 162, SEQ ID NO: |
| | | | | 163, SEQ ID NO: 164, SEQ ID |
| | | | | NO: 165, SEQ ID NO: 166, SEQ |
| | | | | ID NO: 167, SEQ ID NO: 168, |
| | | | | SEQ ID NO: 169, SEQ ID NO: |
| | | | | 170, SEQ ID NO: 171, SEQ ID |
| | | | | NO: 172, SEQ ID NO: 173, SEQ |
| | | | | ID NO: 174, SEQ ID NO: 175, |
| | | | | SEQ ID NO: 176, SEQ ID NO: |
| | | | | 177, SEQ ID NO: 178, SEQ ID |
| | | | | NO: 179, SEQ ID NO: 180, SEQ |
| | | | | ID NO: 181, SEQ ID NO: 182, |
| | | | | SEQ ID NO: 183, SEQ ID NO: |
| | | | | 184, SEQ ID NO: 185, SEQ ID |
| | | | | NO: 186 |
| 17113427 | 5328651 | 112025776 | 112025874 | SEQ ID NO: 187, SEQ ID NO: |
| | | | | 188, SEQ ID NO: 189, SEQ ID |
| | | | | NO: 190, SEQ ID NO: 191, SEQ |
| | | | | ID NO: 192, SEQ ID NO: 193, |
| | | | | SEQ ID NO: 194, SEQ ID NO: |
| | | | | 195, SEQ ID NO: 196, SEQ ID |
| | | | | NO: 197, SEQ ID NO: 198, SEQ |
| | | | | ID NO: 199, SEQ ID NO: 200, |
| | | | | SEQ ID NO: 201, SEQ ID NO: |
| | | | | 202, SEQ ID NO: 203, SEQ ID |
| | | | | NO: 204, SEQ ID NO: 205, SEQ |
| | | | | ID NO: 206, SEQ ID NO: 207, |
| | | | | SEQ ID NO: 208, SEQ ID NO: |
| | | | | 209, SEQ ID NO: 210, SEQ ID |
| | | | | NO: 211, SEQ ID NO: 212, SEQ |
| | | | | ID NO: 213, SEQ ID NO: 214, |
| | | | | SEQ ID NO: 215, SEQ ID NO: |
| | | | | 216, SEQ ID NO: 217, SEQ ID |
| | | | | NO: 218, SEQ ID NO: 219 |
| 16845809 | 5121729 | 43012694 | 43012718 | SEQ ID NO: 220, SEQ ID NO: |
| | | | | 221, SEQ ID NO: 222, SEQ ID |
| | | | | NO: 223, SEQ ID NO: 224, SEQ |
| | | | | ID NO: 225, SEQ ID NO: 226, |
| | | | | SEQ ID NO: 227, SEQ ID NO: |
| | | | | 228, SEQ ID NO: 229, SEQ ID |
| | | | | NO: 230, SEQ ID NO: 231, SEQ |
| | | | | ID NO: 232, SEQ ID NO: 233, |
| | | | | SEQ ID NO: 234, SEQ ID NO: |
| | | | | 235, SEQ ID NO: 236, SEQ ID |
| | | | | NO: 237, SEQ ID NO: 238 |
| 16739924 | 5039969 | 64415691 | 64415715 | SEQ ID NO: 239, SEQ ID NO: |
| | | | | 240, SEQ ID NO: 241, SEQ ID |
| | | | | NO: 242, SEQ ID NO: 243, SEQ |
| | | | | ID NO: 244, SEQ ID NO: 245, |
| | | | | SEQ ID NO: 246, SEQ ID NO: |
| | | | | 247, SEQ ID NO: 248, SEQ ID |
| | | | | NO: 249, SEQ ID NO: 250, SEQ |
| | | | | ID NO: 251, SEQ ID NO: 252, |
| | | | | SEQ ID NO: 253, SEQ ID NO: |
| | | | | 254, SEQ ID NO: 255 |
| 16891029 | 5158213 | 219559290 | 219559314 | SEQ ID NO: 256, SEQ ID NO: |
| | | | | 257, SEQ ID NO: 258, SEQ ID |
| | | | | NO: 259, SEQ ID NO: 260, SEQ |
| | | | | ID NO: 261, SEQ ID NO: 262, |
| | | | | SEQ ID NO: 263, SEQ ID NO: |
| | | | | 264, SEQ ID NO: 265, SEQ ID |
| | | | | NO: 266, SEQ ID NO: 267, SEQ |
| | | | | ID NO: 268, SEQ ID NO: 269, |
| | | | | SEQ ID NO: 270, SEQ ID NO: |
| | | | | 271, SEQ ID NO: 272, SEQ ID |
| | | | | NO: 273, SEQ ID NO: 274, SEQ |
| | | | | ID NO: 275, SEQ ID NO: 276, |
| | | | | SEQ ID NO: 277, SEQ ID NO: |
| | | | | 278, SEQ ID NO: 279, SEQ ID |
| | | | | NO: 280, SEQ ID NO: 281, SEQ |
| | | | | ID NO: 282, SEQ ID NO: 283, |
| | | | | SEQ ID NO: 284, SEQ ID NO: |
| | | | | 285, SEQ ID NO: 286, SEQ ID |
| | | | | NO: 287, SEQ ID NO: 288, SEQ |
| | | | | ID NO: 289 |
| 17047164 | 5277350 | 74009348 | 74009372 | SEQ ID NO: 290, SEQ ID NO: |
| | | | | 291, SEQ ID NO: 292, SEQ ID |
| | | | | NO: 293, SEQ ID NO: 294, SEQ |
| | | | | ID NO: 295, SEQ ID NO: 296, |
| | | | | SEQ ID NO: 297, SEQ ID NO: |
| | | | | 298, SEQ ID NO: 299, SEQ ID |
| | | | | NO: 300, SEQ ID NO: 301, SEQ |
| | | | | ID NO: 302, SEQ ID NO: 303, |
| | | | | SEQ ID NO: 304, SEQ ID NO: |
| | | | | 305, SEQ ID NO: 306, SEQ ID |
| | | | | NO: 307, SEQ ID NO: 308, SEQ |
| | | | | ID NO: 309, SEQ ID NO: 310, |
| | | | | SEQ ID NO: 311 |
| 16958674 | 5210076 | 126214905 | 126214951 | SEQ ID NO: 312, SEQ ID NO: |
| | | | | 313, SEQ ID NO: 314, SEQ ID |
| | | | | NO: 315, SEQ ID NO: 316, SEQ |
| | | | | ID NO: 317, SEQ ID NO: 318, |
| | | | | SEQ ID NO: 319, SEQ ID NO: |
| | | | | 320, SEQ ID NO: 321, SEQ ID |
| | | | | NO: 322, SEQ ID NO: 323, SEQ |
| | | | | ID NO: 324, SEQ ID NO: 325, |
| | | | | SEQ ID NO: 326, SEQ ID NO: |
| | | | | 327, SEQ ID NO: 328, SEQ ID |
| | | | | NO: 329, SEQ ID NO: 330, SEQ |
| | | | | ID NO: 331, SEQ ID NO: 332, |
| | | | | SEQ ID NO: 333, SEQ ID NO: |
| | | | | 334, SEQ ID NO: 335, SEQ ID |
| | | | | NO: 336, SEQ ID NO: 337, SEQ |
| | | | | ID NO: 338, SEQ ID NO: 339, |
| | | | | SEQ ID NO: 340, SEQ ID NO: |
| | | | | 341, SEQ ID NO: 342, SEQ ID |
| | | | | NO: 343, SEQ ID NO: 344, SEQ |
| | | | | ID NO: 345 |
| 16762944 | 5056811 | 31542322 | 31542346 | SEQ ID NO: 346, SEQ ID NO: |
| | | | | 347, SEQ ID NO: 348, SEQ ID |
| | | | | NO: 349, SEQ ID NO: 350, SEQ |
| | | | | ID NO: 351, SEQ ID NO: 352, |
| | | | | SEQ ID NO: 353, SEQ ID NO: |
| | | | | 354, SEQ ID NO: 355, SEQ ID |
| | | | | NO: 356, SEQ ID NO: 357, SEQ |
| | | | | ID NO: 358, SEQ ID NO: 359, |
| | | | | SEQ ID NO: 360, SEQ ID NO: |
| | | | | 361, SEQ ID NO: 362, SEQ ID |
| | | | | NO: 363, SEQ ID NO: 364, SEQ |
| | | | | ID NO: 365, SEQ ID NO: 366, |
| | | | | SEQ ID NO: 367, SEQ ID NO: |
| | | | | 368, SEQ ID NO: 369 |
| 16850156 | 5125247 | 80059559 | 80059583 | SEQ ID NO: 370, SEQ ID NO: |
| | | | | 371, SEQ ID NO: 372, SEQ ID |
| | | | | NO: 373, SEQ ID NO: 374, SEQ |
| | | | | ID NO: 375, SEQ ID NO: 376, |
| | | | | SEQ ID NO: 377, SEQ ID NO: |
| | | | | 378, SEQ ID NO: 379, SEQ ID |
| | | | | NO: 380, SEQ ID NO: 381, SEQ |
| | | | | ID NO: 382, SEQ ID NO: 383, |
| | | | | SEQ ID NO: 384, SEQ ID NO: |
| | | | | 385, SEQ ID NO: 386, SEQ ID |
| | | | | NO: 387, SEQ ID NO: 388, SEQ |
| | | | | ID NO: 389, SEQ ID NO: 390, |
| | | | | SEQ ID NO: 391 |
| 16769889 | 5061882 | 109724524 | 109724548 | SEQ ID NO: 392, SEQ ID NO: |
| | | | | 393, SEQ ID NO: 394, SEQ ID |
| | | | | NO: 395, SEQ ID NO: 396, SEQ |
| | | | | ID NO: 397, SEQ ID NO: 398, |
| | | | | SEQ ID NO: 399, SEQ ID NO: |
| | | | | 400, SEQ ID NO: 401, SEQ ID |
| | | | | NO: 402, SEQ ID NO: 403, SEQ |
| | | | | ID NO: 404, SEQ ID NO: 405, |
| | | | | SEQ ID NO: 406, SEQ ID NO: |
| | | | | 407, SEQ ID NO: 408 |
| 17081037 | 5303509 | 128200292 | 128200316 | SEQ ID NO: 409, SEQ ID NO: |
| | | | | 410, SEQ ID NO: 411, SEQ ID |
| | | | | NO: 412, SEQ ID NO: 413, SEQ |
| | | | | ID NO: 414, SEQ ID NO: 415, |
| | | | | SEQ ID NO: 416, SEQ ID NO: |
| | | | | 417, SEQ ID NO: 418, SEQ ID |
| | | | | NO: 419, SEQ ID NO: 420, SEQ |
| | | | | ID NO: 421, SEQ ID NO: 422, |
| | | | | SEQ ID NO: 423, SEQ ID NO: |
| | | | | 424, SEQ ID NO: 425, SEQ ID |
| | | | | NO: 426, SEQ ID NO: 427, SEQ |
| | | | | ID NO: 428, SEQ ID NO: 429, |
| | | | | SEQ ID NO: 430, SEQ ID NO: |
| | | | | 431, SEQ ID NO: 432, SEQ ID |
| | | | | NO: 433 |
| 16770033 | 5061983 | 110488818 | 110488842 | SEQ ID NO: 434, SEQ ID NO: |
| | | | | 435, SEQ ID NO: 436, SEQ ID |
| | | | | NO: 437, SEQ ID NO: 438, SEQ |
| | | | | ID NO: 439, SEQ ID NO: 440, |
| | | | | SEQ ID NO: 441, SEQ ID NO: |
| | | | | 442, SEQ ID NO: 443, SEQ ID |
| | | | | NO: 444, SEQ ID NO: 445, SEQ |
| | | | | ID NO: 446, SEQ ID NO: 447, |
| | | | | SEQ ID NO: 448, SEQ ID NO: |
| | | | | 449, SEQ ID NO: 450, SEQ ID |
| | | | | NO: 451, SEQ ID NO: 452, SEQ |
| | | | | ID NO: 453, SEQ ID NO: 454, |
| | | | | SEQ ID NO: 455, SEQ ID NO: |
| | | | | 456, SEQ ID NO: 457, SEQ ID |
| | | | | NO: 458, SEQ ID NO: 459, SEQ |
| | | | | ID NO: 460, SEQ ID NO: 461, |
| | | | | SEQ ID NO: 462, SEQ ID NO: |
| | | | | 463, SEQ ID NO: 464, SEQ ID |
| | | | | NO: 465, SEQ ID NO: 466, SEQ |
| | | | | ID NO: 467, SEQ ID NO: 468 |
| 16746216 | 5044431 | 130298287 | 130298880 | SEQ ID NO: 469, SEQ ID NO: |
| | | | | 470, SEQ ID NO: 471, SEQ ID |
| | | | | NO: 472, SEQ ID NO: 473, SEQ |
| | | | | ID NO: 474, SEQ ID NO: 475, |
| | | | | SEQ ID NO: 476, SEQ ID NO: |
| | | | | 477, SEQ ID NO: 478, SEQ ID |
| | | | | NO: 479, SEQ ID NO: 480, SEQ |
| | | | | ID NO: 481, SEQ ID NO: 482, |
| | | | | SEQ ID NO: 483, SEQ ID NO: |
| | | | | 484, SEQ ID NO: 485, SEQ ID |
| | | | | NO: 486, SEQ ID NO: 487, SEQ |
| | | | | ID NO: 488, SEQ ID NO: 489 |
| 16972455 | 5221002 | 174452893 | 174452917 | SEQ ID NO: 490, SEQ ID NO: |
| | | | | 491, SEQ ID NO: 492, SEQ ID |
| | | | | NO: 493, SEQ ID NO: 494, SEQ |
| | | | | ID NO: 495, SEQ ID NO: 496, |
| | | | | SEQ ID NO: 497, SEQ ID NO: |
| | | | | 498, SEQ ID NO: 499, SEQ ID |
| | | | | NO: 500, SEQ ID NO: 501, SEQ |
| | | | | ID NO: 502, SEQ ID NO: 503, |
| | | | | SEQ ID NO: 504, SEQ ID NO: |
| | | | | 505, SEQ ID NO: 506, SEQ ID |
| | | | | NO: 507, SEQ ID NO: 508, SEQ |
| | | | | ID NO: 509, SEQ ID NO: 510, |
| | | | | SEQ ID NO: 511, SEQ ID NO: |
| | | | | 512, SEQ ID NO: 513, SEQ ID |
| | | | | NO: 514, SEQ ID NO: 515, SEQ |
| | | | | ID NO: 516, SEQ ID NO: 517, |
| | | | | SEQ ID NO: 518 |
| 16980805 | 5227735 | 155412547 | 155412910 | SEQ ID NO: 519, SEQ ID NO: |
| | | | | 520, SEQ ID NO: 521, SEQ ID |
| | | | | NO: 522, SEQ ID NO: 523, SEQ |
| | | | | ID NO: 524, SEQ ID NO: 525, |
| | | | | SEQ ID NO: 526, SEQ ID NO: |
| | | | | 527, SEQ ID NO: 528, SEQ ID |
| | | | | NO: 529, SEQ ID NO: 530, SEQ |
| | | | | ID NO: 531, SEQ ID NO: 532, |
| | | | | SEQ ID NO: 533, SEQ ID NO: |
| | | | | 534, SEQ ID NO: 535, SEQ ID |
| | | | | NO: 536, SEQ ID NO: 537, SEQ |
| | | | | ID NO: 538, SEQ ID NO: 539, |
| | | | | SEQ ID NO: 540, SEQ ID NO: |
| | | | | 541, SEQ ID NO: 542, SEQ ID |
| | | | | NO: 543, SEQ ID NO: 544, SEQ |
| | | | | ID NO: 545, SEQ ID NO: 546, |
| | | | | SEQ ID NO: 547, SEQ ID NO: |
| | | | | 548, SEQ ID NO: 549, SEQ ID |
| | | | | NO: 550, SEQ ID NO: 551, SEQ |
| | | | | ID NO: 552, SEQ ID NO: 553, |
| | | | | SEQ ID NO: 554, SEQ ID NO: |
| | | | | 555, SEQ ID NO: 556, SEQ ID |
| | | | | NO: 557 |
| 16756127 | 5051713 | 104379448 | 104379506 | SEQ ID NO: 558, SEQ ID NO: |
| | | | | 559, SEQ ID NO: 560, SEQ ID |
| | | | | NO: 561, SEQ ID NO: 562, SEQ |
| | | | | ID NO: 563, SEQ ID NO: 564, |
| | | | | SEQ ID NO: 565, SEQ ID NO: |
| | | | | 566, SEQ ID NO: 567, SEQ ID |
| | | | | NO: 568, SEQ ID NO: 569, SEQ |
| | | | | ID NO: 570, SEQ ID NO: 571, |
| | | | | SEQ ID NO: 572, SEQ ID NO: |
| | | | | 573, SEQ ID NO: 574, SEQ ID |
| | | | | NO: 575, SEQ ID NO: 576, SEQ |
| | | | | ID NO: 577, SEQ ID NO: 578, |
| | | | | SEQ ID NO: 579, SEQ ID NO: |
| | | | | 580, SEQ ID NO: 581 |
| 16944179 | 5199144 | 113784250 | 113784274 | SEQ ID NO: 582, SEQ ID NO: |
| | | | | 583, SEQ ID NO: 584, SEQ ID |
| | | | | NO: 585, SEQ ID NO: 586, SEQ |
| | | | | ID NO: 587, SEQ ID NO: 588, |
| | | | | SEQ ID NO: 589, SEQ ID NO: |
| | | | | 590, SEQ ID NO: 591, SEQ ID |
| | | | | NO: 592, SEQ ID NO: 593, SEQ |
| | | | | ID NO: 594, SEQ ID NO: 595, |
| | | | | SEQ ID NO: 596, SEQ ID NO: |
| | | | | 597, SEQ ID NO: 598, SEQ ID |
| | | | | NO: 599, SEQ ID NO: 600, SEQ |
| | | | | ID NO: 601, SEQ ID NO: 602, |
| | | | | SEQ ID NO: 603, SEQ ID NO: 604 |
| 16708482 | 5016644 | 103295201 | 103295225 | SEQ ID NO: 605, SEQ ID NO: |
| | | | | 606, SEQ ID NO: 607, SEQ ID |
| | | | | NO: 608, SEQ ID NO: 609, SEQ |
| | | | | ID NO: 610, SEQ ID NO: 611, |
| | | | | SEQ ID NO: 612, SEQ ID NO: |
| | | | | 613, SEQ ID NO: 614, SEQ ID |
| | | | | NO: 615, SEQ ID NO: 616, SEQ |
| | | | | ID NO: 617, SEQ ID NO: 618, |
| | | | | SEQ ID NO: 619, SEQ ID NO: |
| | | | | 620, SEQ ID NO: 621, SEQ ID |
| | | | | NO: 622, SEQ ID NO: 623, SEQ |
| | | | | ID NO: 624, SEQ ID NO: 625, |
| | | | | SEQ ID NO: 626, SEQ ID NO: |
| | | | | 627, SEQ ID NO: 628, SEQ ID |
| | | | | NO: 629, SEQ ID NO: 630, SEQ |
| | | | | ID NO: 631, SEQ ID NO: 632, |
| | | | | SEQ ID NO: 633, SEQ ID NO: |
| | | | | 634, SEQ ID NO: 635 |
| 17094394 | 5313941 | 47304476 | 47304508 | SEQ ID NO: 636, SEQ ID NO: |
| | | | | 637, SEQ ID NO: 638, SEQ ID |
| | | | | NO: 639, SEQ ID NO: 640, SEQ |
| | | | | ID NO: 641, SEQ ID NO: 642, |
| | | | | SEQ ID NO: 643, SEQ ID NO: |
| | | | | 644, SEQ ID NO: 645, SEQ ID |
| | | | | NO: 646, SEQ ID NO: 647, SEQ |
| | | | | ID NO: 648, SEQ ID NO: 649, |
| | | | | SEQ ID NO: 650, SEQ ID NO: |
| | | | | 651, SEQ ID NO: 652, SEQ ID |
| | | | | NO: 653, SEQ ID NO: 654, SEQ |
| | | | | ID NO: 655, SEQ ID NO: 656, |
| | | | | SEQ ID NO: 657, SEQ ID NO: |
| | | | | 658, SEQ ID NO: 659, SEQ ID |
| | | | | NO: 660, SEQ ID NO: 661, SEQ |
| | | | | ID NO: 662, SEQ ID NO: 663, |
| | | | | SEQ ID NO: 664 |
| 16823203 | 5103477 | 2834647 | 2834792 | SEQ ID NO: 665, SEQ ID NO: |
| | | | | 666, SEQ ID NO: 667, SEQ ID |
| | | | | NO: 668, SEQ ID NO: 669, SEQ |
| | | | | ID NO: 670, SEQ ID NO: 671, |
| | | | | SEQ ID NO: 672, SEQ ID NO: |
| | | | | 673, SEQ ID NO: 674, SEQ ID |
| | | | | NO: 675, SEQ ID NO: 676, SEQ |
| | | | | ID NO: 677, SEQ ID NO: 678, |
| | | | | SEQ ID NO: 679, SEQ ID NO: |
| | | | | 680, SEQ ID NO: 681, SEQ ID |
| | | | | NO: 682, SEQ ID NO: 683, SEQ |
| | | | | ID NO: 684, SEQ ID NO: 685 |

TABLE 3
Optimal Set of 14 probe sets that identifies specific exons in the RNA biomarker,
which are used for detecting that expression is decreased or increased of RNA biomarkers
in a sample comprising total polynucleotides isolated from CD45-positive cells (or biofluids)
from a female subject, corresponding Probe Set IDs the Exon_ID, the start and
end of the exon and the nucleic acid sequences of the probes (Nucleic Acid Sequences
of Probes) within each probe set for the female subjects.

| PROBE | | | | NUCLEIC ACID |
|---|---|---|---|---|
| SET ID | EXON_ID | START | STOP | SEQUENCES OF PROBES |
| 16944179 | 5199144 | 113784250 | 113784274 | SEQ ID NO: 582, SEQ ID NO: 583, |
| | | | | SEQ ID NO: 584, SEQ ID NO: 585, |
| | | | | SEQ ID NO: 586, SEQ ID NO: 587, |
| | | | | SEQ ID NO: 588, SEQ ID NO: 589, |
| | | | | SEQ ID NO: 590, SEQ ID NO: 591, |
| | | | | SEQ ID NO: 592, SEQ ID NO: 593, |
| | | | | SEQ ID NO: 594, SEQ ID NO: 595, |
| | | | | SEQ ID NO: 596, SEQ ID NO: 597, |
| | | | | SEQ ID NO: 598, SEQ ID NO: 599, |
| | | | | SEQ ID NO: 600, SEQ ID NO: 601, |
| | | | | SEQ ID NO: 602, SEQ ID NO: 603, |
| | | | | SEQ ID NO: 604 |
| 17113427 | 5328651 | 112025776 | 112025874 | SEQ ID NO: 187, SEQ ID NO: 188, |
| | | | | SEQ ID NO: 189, SEQ ID NO: 190, |
| | | | | SEQ ID NO: 191, SEQ ID NO: 192, |
| | | | | SEQ ID NO: 193, SEQ ID NO: 194, |
| | | | | SEQ ID NO: 195, SEQ ID NO: 196, |
| | | | | SEQ ID NO: 197, SEQ ID NO: 198, |
| | | | | SEQ ID NO: 199, SEQ ID NO: 200, |
| | | | | SEQ ID NO: 201, SEQ ID NO: 202, |
| | | | | SEQ ID NO: 203, SEQ ID NO: 204, |
| | | | | SEQ ID NO: 205, SEQ ID NO: 206, |
| | | | | SEQ ID NO: 207, SEQ ID NO: 208, |
| | | | | SEQ ID NO: 209, SEQ ID NO: 210, |
| | | | | SEQ ID NO: 211, SEQ ID NO: 212, |
| | | | | SEQ ID NO: 213, SEQ ID NO: 214, |
| | | | | SEQ ID NO: 215, SEQ ID NO: 216, |
| | | | | SEQ ID NO: 217, SEQ ID NO: 218, |
| | | | | SEQ ID NO: 219 |
| 16762944 | 5056811 | 31542322 | 31542346 | SEQ ID NO: 346, SEQ ID NO: 347, |
| | | | | SEQ ID NO: 348, SEQ ID NO: 349, |
| | | | | SEQ ID NO: 350, SEQ ID NO: 351, |
| | | | | SEQ ID NO: 352, SEQ ID NO: 353, |
| | | | | SEQ ID NO: 354, SEQ ID NO: 355, |
| | | | | SEQ ID NO: 356, SEQ ID NO: 357, |
| | | | | SEQ ID NO: 358, SEQ ID NO: 359, |
| | | | | SEQ ID NO: 360, SEQ ID NO: 361, |
| | | | | SEQ ID NO: 362, SEQ ID NO: 363, |
| | | | | SEQ ID NO: 364, SEQ ID NO: 365, |
| | | | | SEQ ID NO: 366, SEQ ID NO: 367, |
| | | | | SEQ ID NO: 368, SEQ ID NO: 369 |
| 16972455 | 5221002 | 174452893 | 174452917 | SEQ ID NO: 490, SEQ ID NO: 491, |
| | | | | SEQ ID NO: 492, SEQ ID NO: 493, |
| | | | | SEQ ID NO: 494, SEQ ID NO: 495, |
| | | | | SEQ ID NO: 496, SEQ ID NO: 497, |
| | | | | SEQ ID NO: 498, SEQ ID NO: 499, |
| | | | | SEQ ID NO: 500, SEQ ID NO: 501, |
| | | | | SEQ ID NO: 502, SEQ ID NO: 503, |
| | | | | SEQ ID NO: 504, SEQ ID NO: 505, |
| | | | | SEQ ID NO: 506, SEQ ID NO: 507, |
| | | | | SEQ ID NO: 508, SEQ ID NO: 509, |
| | | | | SEQ ID NO: 510, SEQ ID NO: 511, |
| | | | | SEQ ID NO: 512, SEQ ID NO: 513, |
| | | | | SEQ ID NO: 514, SEQ ID NO: 515, |
| | | | | SEQ ID NO: 516, SEQ ID NO: 517, |
| | | | | SEQ ID NO: 518 |
| 16756127 | 5051713 | 104379448 | 104379506 | SEQ ID NO: 558, SEQ ID NO: 559, |
| | | | | SEQ ID NO: 560, SEQ ID NO: 561, |
| | | | | SEQ ID NO: 562, SEQ ID NO: 563, |
| | | | | SEQ ID NO: 564, SEQ ID NO: 565, |
| | | | | SEQ ID NO: 566, SEQ ID NO: 567, |
| | | | | SEQ ID NO: 568, SEQ ID NO: 569, |
| | | | | SEQ ID NO: 570, SEQ ID NO: 571, |
| | | | | SEQ ID NO: 572, SEQ ID NO: 573, |
| | | | | SEQ ID NO: 574, SEQ ID NO: 575, |
| | | | | SEQ ID NO: 576, SEQ ID NO: 577, |
| | | | | SEQ ID NO: 578, SEQ ID NO: 579, |
| | | | | SEQ ID NO: 580, SEQ ID NO: 581 |
| 16763818 | 5057504 | 48382221 | 48382245 | SEQ ID NO: 103, SEQ ID NO: 104, |
| | | | | SEQ ID NO: 105, SEQ ID NO: 106, |
| | | | | SEQ ID NO: 107, SEQ ID NO: 108, |
| | | | | SEQ ID NO: 109, SEQ ID NO: 110, |
| | | | | SEQ ID NO: 111, SEQ ID NO: 112, |
| | | | | SEQ ID NO: 113, SEQ ID NO: 114, |
| | | | | SEQ ID NO: 115, SEQ ID NO: 116, |
| | | | | SEQ ID NO: 117, SEQ ID NO: 118, |
| | | | | SEQ ID NO: 119, SEQ ID NO: 120, |
| | | | | SEQ ID NO: 121, SEQ ID NO: 122, |
| | | | | SEQ ID NO: 123, SEQ ID NO: 124, |
| | | | | SEQ ID NO: 125, SEQ ID NO: 126, |
| | | | | SEQ ID NO: 127, SEQ ID NO: 128, |
| | | | | SEQ ID NO: 129, SEQ ID NO: 130 |
| 16823203 | 5103477 | 2834647 | 2834792 | SEQ ID NO: 665, SEQ ID NO: 666, |
| | | | | SEQ ID NO: 667, SEQ ID NO: 668, |
| | | | | SEQ ID NO: 669, SEQ ID NO: 670, |
| | | | | SEQ ID NO: 671, SEQ ID NO: 672, |
| | | | | SEQ ID NO: 673, SEQ ID NO: 674, |
| | | | | SEQ ID NO: 675, SEQ ID NO: 676, |
| | | | | SEQ ID NO: 677, SEQ ID NO: 678, |
| | | | | SEQ ID NO: 679, SEQ ID NO: 680, |
| | | | | SEQ ID NO: 681, SEQ ID NO: 682, |
| | | | | SEQ ID NO: 683, SEQ ID NO: 684, |
| | | | | SEQ ID NO: 685 |
| 16708482 | 5016644 | 103295201 | 103295225 | SEQ ID NO: 605, SEQ ID NO: 606, |
| | | | | SEQ ID NO: 607, SEQ ID NO: 608, |
| | | | | SEQ ID NO: 609, SEQ ID NO: 610, |
| | | | | SEQ ID NO: 611, SEQ ID NO: 612, |
| | | | | SEQ ID NO: 613, SEQ ID NO: 614, |
| | | | | SEQ ID NO: 615, SEQ ID NO: 616, |
| | | | | SEQ ID NO: 617, SEQ ID NO: 618, |
| | | | | SEQ ID NO: 619, SEQ ID NO: 620, |
| | | | | SEQ ID NO: 621, SEQ ID NO: 622, |
| | | | | SEQ ID NO: 623, SEQ ID NO: 624, |
| | | | | SEQ ID NO: 625, SEQ ID NO: 626, |
| | | | | SEQ ID NO: 627, SEQ ID NO: 628, |
| | | | | SEQ ID NO: 629, SEQ ID NO: 630, |
| | | | | SEQ ID NO: 631, SEQ ID NO: 632, |
| | | | | SEQ ID NO: 633, SEQ ID NO: 634, |
| | | | | SEQ ID NO: 635 |
| 16980805 | 5227735 | 155412547 | 155412910 | SEQ ID NO: 519, SEQ ID NO: 520, |
| | | | | SEQ ID NO: 521, SEQ ID NO: 522, |
| | | | | SEQ ID NO: 523, SEQ ID NO: 524, |
| | | | | SEQ ID NO: 525, SEQ ID NO: 526, |
| | | | | SEQ ID NO: 527, SEQ ID NO: 528, |
| | | | | SEQ ID NO: 529, SEQ ID NO: 530, |
| | | | | SEQ ID NO: 531, SEQ ID NO: 532, |
| | | | | SEQ ID NO: 533, SEQ ID NO: 534, |
| | | | | SEQ ID NO: 535, SEQ ID NO: 536, |
| | | | | SEQ ID NO: 537, SEQ ID NO: 538, |
| | | | | SEQ ID NO: 539, SEQ ID NO: 540, |
| | | | | SEQ ID NO: 541, SEQ ID NO: 542, |
| | | | | SEQ ID NO: 543, SEQ ID NO: 544, |
| | | | | SEQ ID NO: 545, SEQ ID NO: 546, |
| | | | | SEQ ID NO: 547, SEQ ID NO: 548, |
| | | | | SEQ ID NO: 549, SEQ ID NO: 550, |
| | | | | SEQ ID NO: 551, SEQ ID NO: 552, |
| | | | | SEQ ID NO: 553, SEQ ID NO: 554, |
| | | | | SEQ ID NO: 555, SEQ ID NO: 556, |
| | | | | SEQ ID NO: 557 |
| 16779021 | 5069104 | 49000747 | 49000882 | SEQ ID NO: 52, SEQ ID NO: 53, |
| | | | | SEQ ID NO: 54, SEQ ID NO: 55, |
| | | | | SEQ ID NO: 56, SEQ ID NO: 57, |
| | | | | SEQ ID NO: 58, SEQ ID NO: 59, |
| | | | | SEQ ID NO: 60, SEQ ID NO: 61, |
| | | | | SEQ ID NO: 62, SEQ ID NO: 63, |
| | | | | SEQ ID NO: 64, SEQ ID NO: 65, |
| | | | | SEQ ID NO: 66, SEQ ID NO: 67, |
| | | | | SEQ ID NO: 68, SEQ ID NO: 69, |
| | | | | SEQ ID NO: 70, SEQ ID NO: 71 |
| 16770033 | 5061983 | 110488818 | 110488842 | SEQ ID NO: 434, SEQ ID NO: 435, |
| | | | | SEQ ID NO: 436, SEQ ID NO: 437, |
| | | | | SEQ ID NO: 438, SEQ ID NO: 439, |
| | | | | SEQ ID NO: 440, SEQ ID NO: 441, |
| | | | | SEQ ID NO: 442, SEQ ID NO: 443, |
| | | | | SEQ ID NO: 444, SEQ ID NO: 445, |
| | | | | SEQ ID NO: 446, SEQ ID NO: 447, |
| | | | | SEQ ID NO: 448, SEQ ID NO: 449, |
| | | | | SEQ ID NO: 450, SEQ ID NO: 451, |
| | | | | SEQ ID NO: 452, SEQ ID NO: 453, |
| | | | | SEQ ID NO: 454, SEQ ID NO: 455, |
| | | | | SEQ ID NO: 456, SEQ ID NO: 457, |
| | | | | SEQ ID NO: 458, SEQ ID NO: 459, |
| | | | | SEQ ID NO: 460, SEQ ID NO: 461, |
| | | | | SEQ ID NO: 462, SEQ ID NO: 463, |
| | | | | SEQ ID NO: 464, SEQ ID NO: 465, |
| | | | | SEQ ID NO: 466, SEQ ID NO: 467, |
| | | | | SEQ ID NO: 468 |
| 17094394 | 5313941 | 47304476 | 47304508 | SEQ ID NO: 636, SEQ ID NO: 637, |
| | | | | SEQ ID NO: 638, SEQ ID NO: 639, |
| | | | | SEQ ID NO: 640, SEQ ID NO: 641, |
| | | | | SEQ ID NO: 642, SEQ ID NO: 643, |
| | | | | SEQ ID NO: 644, SEQ ID NO: 645, |
| | | | | SEQ ID NO: 646, SEQ ID NO: 647, |
| | | | | SEQ ID NO: 648, SEQ ID NO: 649, |
| | | | | SEQ ID NO: 650, SEQ ID NO: 651, |
| | | | | SEQ ID NO: 652, SEQ ID NO: 653, |
| | | | | SEQ ID NO: 654, SEQ ID NO: 655, |
| | | | | SEQ ID NO: 656, SEQ ID NO: 657, |
| | | | | SEQ ID NO: 658, SEQ ID NO: 659, |
| | | | | SEQ ID NO: 660, SEQ ID NO: 661, |
| | | | | SEQ ID NO: 662, SEQ ID NO: 663, |
| | | | | SEQ ID NO: 664 |
| 16746216 | 5044431 | 130298287 | 130298880 | SEQ ID NO: 469, SEQ ID NO: 470, |
| | | | | SEQ ID NO: 471, SEQ ID NO: 472, |
| | | | | SEQ ID NO: 473, SEQ ID NO: 474, |
| | | | | SEQ ID NO: 475, SEQ ID NO: 476, |
| | | | | SEQ ID NO: 477, SEQ ID NO: 478, |
| | | | | SEQ ID NO: 479, SEQ ID NO: 480, |
| | | | | SEQ ID NO: 481, SEQ ID NO: 482, |
| | | | | SEQ ID NO: 483, SEQ ID NO: 484, |
| | | | | SEQ ID NO: 485, SEQ ID NO: 486, |
| | | | | SEQ ID NO: 487, SEQ ID NO: 488, |
| | | | | SEQ ID NO: 489 |
| 16928030 | 5187159 | 24121613 | 24121639 | SEQ ID NO: 1, SEQ ID NO: 2, |
| | | | | SEQ ID NO: 3, SEQ ID NO: 4, |
| | | | | SEQ ID NO: 5, SEQ ID NO: 6, |
| | | | | SEQ ID NO: 7, SEQ ID NO: 8, |
| | | | | SEQ ID NO: 9, SEQ ID NO: 10, |
| | | | | SEQ ID NO: 11, SEQ ID NO: 12, |
| | | | | SEQ ID NO: 13, SEQ ID NO: 14, |
| | | | | SEQ ID NO: 15, SEQ ID NO: 16, |
| | | | | SEQ ID NO: 17, SEQ ID NO: 18, |
| | | | | SEQ ID NO: 19, SEQ ID NO: 20, |
| | | | | SEQ ID NO: 21, SEQ ID NO: 22, |
| | | | | SEQ ID NO: 23, SEQ ID NO: 24, |
| | | | | SEQ ID NO: 25, SEQ ID NO: 26, |
| | | | | SEQ ID NO: 27, SEQ ID NO: 28, |
| | | | | SEQ ID NO: 29, SEQ ID NO: 30, |
| | | | | SEQ ID NO: 31, SEQ ID NO: 32 |

In a specific aspect the invention provides an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) from a test sample collected from said female subject, and b. comparing said first expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) collected from said female subject with a control expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level.

In a further specific aspect the invention provides an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG, in CD45+ mononuclear cells (MNC) from a test sample collected from said female subject, and b. comparing said first expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG, in CD45+ mononuclear cells (MNC) collected from said female subject with a control expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level.

In a second aspect the invention provides an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and b. comparing said first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level.

Tables 4, 5 and 6 are shown below. Table 4 shows the names of the RNA biomarkers identified using the probes in the probe sets (Probe Set ID), further the chromosomes and strands these are found on, for the male subjects. Table 5 details for the corresponding Probe Set IDs the Exon_ID, the start and end of the exon and the sequences of the probes within each probe set for the male subjects are shown. Table 6 details the corresponding Probe Set IDs, the Exon_ID, the start and end of the exon and the sequences of the probes within the optimal 16 probe set for the male subjects are shown.

TABLE 4
RNA biomarkers, which expression is decreased or increased in a sample comprising
total polynucleotides isolated from CD45-positive cells (or biofluids)
from a male subject, and corresponding chromosomes, strands and probe set
(Probe Set ID) used to identify said male specific RNA biomarkers.

| | | | PROBE |
|---|---|---|---|
| IDENTIFIED RNA BIOMARKER | CHROMOSOME | STRAND | SET ID |
| ENST00000440123 // chr10 // 100 | chr10 | + | 16708531 |
| // 12 // 12 // 0 | | | |
| TCONS_00025321-XLOC_012127 // | chr17 | + | 16831561 |
| chr17 // 100 // 12 // 11 // 0 | | | |
| ENST00000488290 // MTPAP /// | chr10 | − | 16712992 |
| AK125739 // LOC729668 | | | |
| ENST00000430988 // chr2 // 100 | chr2 | − | 16895114 |
| // 9 // 9 // 0 | | | |
| ENST00000440657 // USP12-AS1 | chr13 | + | 16773468 |
| ProbSetID_17118935 | chrU | + | 17118935 |
| NM_007117 // TRH /// ENST00000302649 | chr3 | + | 16945454 |
| // TRH /// BC110515 // TRH | | | |
| XR_109422 // LOC100507049 | chr17 | − | 16848054 |
| ENST00000456944 // LOC100507009 /// | chr9 | − | 17095583 |
| XR_108972 // LOC100507009 | | | |
| NM_080717 // TBX5 /// | chr12 | + | 16757527 |
| ENST00000349716 // TBX5 /// | | | |
| ENST00000310346 // TBX5 /// | | | |
| NR_038440 // TBX5-AS1 /// | | | |
| ENST00000531202 // TBX5-AS1 /// | | | |
| ENST00000532697 // TBX5-AS1 /// | | | |
| ENST00000531024 // TBX5-AS1 | | | |
| TCONS_I2_00026656-XLOC_I2_013873 // | chr7 | − | 17058221 |
| chr7 // 100 // 3 // 3 // 0 | | | |
| ExonID_5262586 | chr6 | − | 17025653 |
| AK293404 // RGL3 /// ENST00000453604 | chr19 | − | 16868954 |
| // RGL3 | | | |
| ENST00000338315 // MFSD2B /// | chr2 | + | 16877665 |
| ENST00000469562 // MFSD2B | | | |
| ENST00000432646 // CALD1 /// | chr7 | + | 17051908 |
| ENST00000443197 // CALD1 | | | |
| ENST00000518932 // chr8 // 100 // | chr8 | − | 17080053 |
| 1 // 1 // 0 | | | |
| NM_001033551 // TOM1L2 /// | chr17 | − | 16842003 |
| NM_001082968 // TOM1L2 /// | | | |
| ENST00000379504 // TOM1L2 /// | | | |
| ENST00000318094 // TOM1L2 /// | | | |
| AK303305 // TOM1L2 /// AK295111 // | | | |
| TOM1L2 /// AK299872 // TOM1L2 /// | | | |
| AK301944 // TOM1L2 /// AK294846 // | | | |
| TOM1L2 /// BC051650 // TOM1L2 /// | | | |
| ENST00000395739 // TOM1L2 /// | | | |
| ENST00000535933 // TOM1L2 /// | | | |
| ENST00000540946 // TOM1L2 /// | | | |
| ENST00000542206 // TOM1L2 /// | | | |
| ENST00000537091 // TOM1L2 | | | |
| NM_001105206 // LAMA4 /// NM_002290 | chr6 | − | 17022799 |
| // LAMA4 /// NM_001105207 // LAMA4 /// | | | |
| ENST00000230538 // LAMA4 /// | | | |
| ENST00000522006 // LAMA4 /// | | | |
| ENST00000389463 // LAMA4 /// | | | |
| ENST00000424408 // LAMA4 /// | | | |
| BC066552 // LAMA4 /// | | | |
| ENST00000454881 // LAMA4 /// | | | |
| ENST00000521398 // LAMA4 /// | | | |
| ENST00000542588 // LAMA4 /// | | | |
| ENST00000524032 // LAMA4 /// | | | |
| ENST00000423735 // LAMA4 /// | | | |
| ENST00000368639 // LAMA4 | | | |
| NM_182480 // COQ6 /// | chr14 | + | 16786369 |
| ENST00000394026 // COQ6 /// AK296040 | | | |
| // COQ6 /// ENST00000554341 // COQ6 /// | | | |
| ENST00000238709 // COQ6 | | | |
| ENST00000481363 // SALL4 | chr20 | − | 16920377 |
| NM_138375 // CABLES1 /// | chr18 | + | 16851430 |
| ENST00000420687 // CABLES1 /// | | | |
| BC037218 // CABLES1 | | | |
| NM_005035 // POLRMT /// | chr19 | − | 16866470 |
| ENST00000215591 // POLRMT /// | | | |
| BC098387 // POLRMT | | | |
| ENST00000503919 // SLC23A1 | chr5 | − | 17000583 |
| NM_024807 // TREML2 /// | chr6 | − | 17019044 |
| ENST00000483722 // TREML2 /// | | | |
| BC125078 // TREML2 | | | |
| NR_003697 // SNHG15 /// AK096179 // | chr7 | − | 17057414 |
| SNHG15 | | | |
| NM_000786 // CYP51A1 /// | chr7 | − | 17059640 |
| NM_001146152 // CYP51A1 /// | | | |
| ENST00000003100 // CYP51A1 /// | | | |
| ENST00000496998 // CYP51A1 /// | | | |
| ENST00000450723 // CYP51A1 /// | | | |
| U23942 // CYP51A1 /// | | | |
| ENST00000422722 // LRRD1 | | | |
| ExonID_5299845 | chr8 | − | 17076434 |
| TCONS_00017554-XLOC_008249 // chrX | chrX | − | 17114699 |
| // 100 // 2 // 2 // 0 | | | |

TABLE 5
Probe sets and probes that identifies specific exons in the RNA biomarker, which
are used for detecting that expression is decreased or increased of RNA biomarkers
in a sample comprising total polynucleotides isolated from CD45-positive cells (or biofluids)
from a male subject, corresponding Probe Set IDs the Exon_ID, the start and
end of the exon and the nucleic acid sequences of the probes (Nucleic Acid Sequences
of Probes) within each probe set for the male subjects.

| PROBE | | | | NUCLEIC ACID |
|---|---|---|---|---|
| SET ID | EXON_ID | START | STOP | SEQUENCES OF PROBES |
| 16708531 | 5016680 | 103880410 | 103880730 | SEQ ID NO: 686, SEQ ID NO: |
| | | | | 687, SEQ ID NO: 688, SEQ ID |
| | | | | NO: 689, SEQ ID NO: 690, SEQ |
| | | | | ID NO: 691, SEQ ID NO: 692, |
| | | | | SEQ ID NO: 693, SEQ ID NO: |
| | | | | 694, SEQ ID NO: 695, SEQ ID |
| | | | | NO: 696, SEQ ID NO: 697, SEQ |
| | | | | ID NO: 698, SEQ ID NO: 699, |
| | | | | SEQ ID NO: 700, SEQ ID NO: |
| | | | | 701, SEQ ID NO: 702, SEQ ID |
| | | | | NO: 703 |
| 16831561 | 5110382 | 16926501 | 16926546 | SEQ ID NO: 704, SEQ ID NO: |
| | | | | 705, SEQ ID NO: 706, SEQ ID |
| | | | | NO: 707, SEQ ID NO: 708, SEQ |
| | | | | ID NO: 709, SEQ ID NO: 710, |
| | | | | SEQ ID NO: 711, SEQ ID NO: |
| | | | | 712, SEQ ID NO: 713, SEQ ID |
| | | | | NO: 714, SEQ ID NO: 715, SEQ |
| | | | | ID NO: 716, SEQ ID NO: 717, |
| | | | | SEQ ID NO: 718, SEQ ID NO: |
| | | | | 719, SEQ ID NO: 720, SEQ ID |
| | | | | NO: 721, SEQ ID NO: 722 |
| 16712992 | 5020340 | 30656698 | 30656743 | SEQ ID NO: 723, SEQ ID NO: |
| | | | | 724, SEQ ID NO: 725, SEQ ID |
| | | | | NO: 726, SEQ ID NO: 727, SEQ |
| | | | | ID NO: 728, SEQ ID NO: 729, |
| | | | | SEQ ID NO: 730, SEQ ID NO: |
| | | | | 731, SEQ ID NO: 732 |
| 16895114 | 5161268 | 23746962 | 23747184 | SEQ ID NO: 733, SEQ ID NO: |
| | | | | 734, SEQ ID NO: 735, SEQ ID |
| | | | | NO: 736, SEQ ID NO: 737, SEQ |
| | | | | ID NO: 738, SEQ ID NO: 739, |
| | | | | SEQ ID NO: 740, SEQ ID NO: |
| | | | | 741, SEQ ID NO: 742, SEQ ID |
| | | | | NO: 743, SEQ ID NO: 744, SEQ |
| | | | | ID NO: 745, SEQ ID NO: 746, |
| | | | | SEQ ID NO: 747, SEQ ID NO: |
| | | | | 748, SEQ ID NO: 749, SEQ ID |
| | | | | NO: 750, SEQ ID NO: 751, SEQ |
| | | | | ID NO: 752, SEQ ID NO: 753, |
| | | | | SEQ ID NO: 754, SEQ ID NO: |
| | | | | 755, SEQ ID NO: 756, SEQ ID |
| | | | | NO: 757 |
| 16773468 | 5064532 | 27737001 | 27737060 | SEQ ID NO: 758, SEQ ID NO: |
| | | | | 759, SEQ ID NO: 760, SEQ ID |
| | | | | NO: 761, SEQ ID NO: 762, SEQ |
| | | | | ID NO: 763, SEQ ID NO: 764, |
| | | | | SEQ ID NO: 765, SEQ ID NO: |
| | | | | 766, SEQ ID NO: 767, SEQ ID |
| | | | | NO: 768, SEQ ID NO: 769, SEQ |
| | | | | ID NO: 770, SEQ ID NO: 771, |
| | | | | SEQ ID NO: 772, SEQ ID NO: |
| | | | | 773, SEQ ID NO: 774 |
| 17118935 | 0 | 17 | 47 | SEQ ID NO: 775, SEQ ID NO: |
| | | | | 776, SEQ ID NO: 777, SEQ ID |
| | | | | NO: 778, SEQ ID NO: 779, SEQ |
| | | | | ID NO: 780, SEQ ID NO: 781 |
| 16945454 | 5200110 | 129695566 | 129696022 | SEQ ID NO: 782, SEQ ID NO: |
| | | | | 783, SEQ ID NO: 784, SEQ ID |
| | | | | NO: 785, SEQ ID NO: 786, SEQ |
| | | | | ID NO: 787, SEQ ID NO: 788, |
| | | | | SEQ ID NO: 789, SEQ ID NO: |
| | | | | 790, SEQ ID NO: 791, SEQ ID |
| | | | | NO: 792, SEQ ID NO: 793, SEQ |
| | | | | ID NO: 794, SEQ ID NO: 795, |
| | | | | SEQ ID NO: 796, SEQ ID NO: |
| | | | | 797, SEQ ID NO: 798, SEQ ID |
| | | | | NO: 799, SEQ ID NO: 800, SEQ |
| | | | | ID NO: 801, SEQ ID NO: 802, |
| | | | | SEQ ID NO: 803 |
| 16848054 | 5123540 | 65713622 | 65713732 | SEQ ID NO: 804, SEQ ID NO: |
| | | | | 805, SEQ ID NO: 806, SEQ ID |
| | | | | NO: 807, SEQ ID NO: 808, SEQ |
| | | | | ID NO: 809, SEQ ID NO: 810, |
| | | | | SEQ ID NO: 811, SEQ ID NO: |
| | | | | 812, SEQ ID NO: 813, SEQ ID |
| | | | | NO: 814, SEQ ID NO: 815, SEQ |
| | | | | ID NO: 816, SEQ ID NO: 817, |
| | | | | SEQ ID NO: 818, SEQ ID NO: |
| | | | | 819, SEQ ID NO: 820, SEQ ID |
| | | | | NO: 821, SEQ ID NO: 822, SEQ |
| | | | | ID NO: 823 |
| 17095583 | 5314892 | 91703547 | 91703806 | SEQ ID NO: 824, SEQ ID NO: |
| | | | | 825, SEQ ID NO: 826, SEQ ID |
| | | | | NO: 827, SEQ ID NO: 828, SEQ |
| | | | | ID NO: 829, SEQ ID NO: 830, |
| | | | | SEQ ID NO: 831, SEQ ID NO: |
| | | | | 832, SEQ ID NO: 833, SEQ ID |
| | | | | NO: 834, SEQ ID NO: 835, SEQ |
| | | | | ID NO: 836, SEQ ID NO: 837, |
| | | | | SEQ ID NO: 838, SEQ ID NO: |
| | | | | 839, SEQ ID NO: 840, SEQ ID |
| | | | | NO: 841 |
| 16757527 | 5052776 | 114845996 | 114846089 | SEQ ID NO: 842, SEQ ID NO: |
| | | | | 843, SEQ ID NO: 844, SEQ ID |
| | | | | NO: 845, SEQ ID NO: 846, SEQ |
| | | | | ID NO: 847, SEQ ID NO: 848, |
| | | | | SEQ ID NO: 849, SEQ ID NO: |
| | | | | 850, SEQ ID NO: 851, SEQ ID |
| | | | | NO: 852, SEQ ID NO: 853, SEQ |
| | | | | ID NO: 854, SEQ ID NO: 855, |
| | | | | SEQ ID NO: 856, SEQ ID NO: |
| | | | | 857, SEQ ID NO: 858, SEQ ID |
| | | | | NO: 859, SEQ ID NO: 860, SEQ |
| | | | | ID NO: 861, SEQ ID NO: 862, |
| | | | | SEQ ID NO: 863, SEQ ID NO: |
| | | | | 864, SEQ ID NO: 865, SEQ ID |
| | | | | NO: 866, SEQ ID NO: 867 |
| 17058221 | 5285817 | 65957267 | 65957293 | SEQ ID NO: 868, SEQ ID NO: |
| | | | | 869, SEQ ID NO: 870, SEQ ID |
| | | | | NO: 871, SEQ ID NO: 872, SEQ |
| | | | | ID NO: 873, SEQ ID NO: 874, |
| | | | | SEQ ID NO: 875, SEQ ID NO: |
| | | | | 876, SEQ ID NO: 877, SEQ ID |
| | | | | NO: 878, SEQ ID NO: 879 |
| 17025653 | 5262586 | 167271642 | 167271666 | SEQ ID NO: 880, SEQ ID NO: |
| | | | | 881, SEQ ID NO: 882, SEQ ID |
| | | | | NO: 883, SEQ ID NO: 884, SEQ |
| | | | | ID NO: 885, SEQ ID NO: 886, |
| | | | | SEQ ID NO: 887, SEQ ID NO: |
| | | | | 888, SEQ ID NO: 889, SEQ ID |
| | | | | NO: 890, SEQ ID NO: 891, SEQ |
| | | | | ID NO: 892, SEQ ID NO: 893, |
| | | | | SEQ ID NO: 894, SEQ ID NO: |
| | | | | 895, SEQ ID NO: 896, SEQ ID |
| | | | | NO: 897, SEQ ID NO: 898, SEQ |
| | | | | ID NO: 899, SEQ ID NO: 900, |
| | | | | SEQ ID NO: 901, SEQ ID NO: |
| | | | | 902, SEQ ID NO: 903, SEQ ID |
| | | | | NO: 904 |
| 16868954 | 5140844 | 11513296 | 11513320 | SEQ ID NO: 905, SEQ ID NO: |
| | | | | 906, SEQ ID NO: 907, SEQ ID |
| | | | | NO: 908, SEQ ID NO: 909, SEQ |
| | | | | ID NO: 910, SEQ ID NO: 911, |
| | | | | SEQ ID NO: 912, SEQ ID NO: |
| | | | | 913, SEQ ID NO: 914, SEQ ID |
| | | | | NO: 915, SEQ ID NO: 916, SEQ |
| | | | | ID NO: 917, SEQ ID NO: 918, |
| | | | | SEQ ID NO: 919, SEQ ID NO: 920 |
| 16877665 | 5147827 | 24248302 | 24248326 | SEQ ID NO: 921, SEQ ID NO: |
| | | | | 922, SEQ ID NO: 923, SEQ ID |
| | | | | NO: 924, SEQ ID NO: 925, SEQ |
| | | | | ID NO: 926, SEQ ID NO: 927, |
| | | | | SEQ ID NO: 928, SEQ ID NO: |
| | | | | 929, SEQ ID NO: 930, SEQ ID |
| | | | | NO: 931, SEQ ID NO: 932, SEQ |
| | | | | ID NO: 933, SEQ ID NO: 934, |
| | | | | SEQ ID NO: 935, SEQ ID NO: |
| | | | | 936, SEQ ID NO: 937, SEQ ID |
| | | | | NO: 938, SEQ ID NO: 939, SEQ |
| | | | | ID NO: 940, SEQ ID NO: 941, |
| | | | | SEQ ID NO: 942 |
| 17051908 | 5280941 | 134647642 | 134647666 | SEQ ID NO: 943, SEQ ID NO: |
| | | | | 944, SEQ ID NO: 945, SEQ ID |
| | | | | NO: 946, SEQ ID NO: 947, SEQ |
| | | | | ID NO: 948, SEQ ID NO: 949, |
| | | | | SEQ ID NO: 950, SEQ ID NO: |
| | | | | 951, SEQ ID NO: 952, SEQ ID |
| | | | | NO: 953, SEQ ID NO: 954, SEQ |
| | | | | ID NO: 955, SEQ ID NO: 956, |
| | | | | SEQ ID NO: 957, SEQ ID NO: |
| | | | | 958, SEQ ID NO: 959, SEQ ID |
| | | | | NO: 960, SEQ ID NO: 961, SEQ |
| | | | | ID NO: 962, SEQ ID NO: 963, |
| | | | | SEQ ID NO: 964, SEQ ID NO: |
| | | | | 965, SEQ ID NO: 966, SEQ ID |
| | | | | NO: 967, SEQ ID NO: 968, SEQ |
| | | | | ID NO: 969, SEQ ID NO: 970, |
| | | | | SEQ ID NO: 971, SEQ ID NO: |
| | | | | 972, SEQ ID NO: 973, SEQ ID |
| | | | | NO: 974, SEQ ID NO: 975, SEQ |
| | | | | ID NO: 976 |
| 17080053 | 5302714 | 106924048 | 106924072 | SEQ ID NO: 977, SEQ ID NO: |
| | | | | 978, SEQ ID NO: 979, SEQ ID |
| | | | | NO: 980, SEQ ID NO: 981, SEQ |
| | | | | ID NO: 982, SEQ ID NO: 983, |
| | | | | SEQ ID NO: 984, SEQ ID NO: |
| | | | | 985, SEQ ID NO: 986, SEQ ID |
| | | | | NO: 987, SEQ ID NO: 988, SEQ |
| | | | | ID NO: 989, SEQ ID NO: 990, |
| | | | | SEQ ID NO: 991, SEQ ID NO: |
| | | | | 992, SEQ ID NO: 993, SEQ ID |
| | | | | NO: 994, SEQ ID NO: 995, SEQ |
| | | | | ID NO: 996, SEQ ID NO: 997, |
| | | | | SEQ ID NO: 998, SEQ ID NO: |
| | | | | 999, SEQ ID NO: 1,000, SEQ ID |
| | | | | NO: 1,001, SEQ ID NO: 1,002 |
| 16842003 | 5118807 | 17875577 | 17875601 | SEQ ID NO: 1,003, SEQ ID NO: |
| | | | | 1,004, SEQ ID NO: 1,005, SEQ ID |
| | | | | NO: 1,006, SEQ ID NO: 1,007, |
| | | | | SEQ ID NO: 1,008, SEQ ID NO: |
| | | | | 1,009, SEQ ID NO: 1,010, SEQ ID |
| | | | | NO: 1,011, SEQ ID NO: 1,012, |
| | | | | SEQ ID NO: 1,013, SEQ ID NO: |
| | | | | 1,014, SEQ ID NO: 1,015, SEQ ID |
| | | | | NO: 1,016, SEQ ID NO: 1,017, |
| | | | | SEQ ID NO: 1,018, SEQ ID NO: |
| | | | | 1,019, SEQ ID NO: 1,020, SEQ ID |
| | | | | NO: 1,021, SEQ ID NO: 1,022, |
| | | | | SEQ ID NO: 1,023, SEQ ID NO: |
| | | | | 1,024, SEQ ID NO: 1,025, SEQ ID |
| | | | | NO: 1,026, SEQ ID NO: 1,027, |
| | | | | SEQ ID NO: 1,028, SEQ ID NO: |
| | | | | 1,029, SEQ ID NO: 1,030, SEQ ID |
| | | | | NO: 1,031, SEQ ID NO: 1,032, |
| | | | | SEQ ID NO: 1,033, SEQ ID NO: |
| | | | | 1,034, SEQ ID NO: 1,035, SEQ ID |
| | | | | NO: 1,036, SEQ ID NO: 1,037, |
| | | | | SEQ ID NO: 1,038 |
| 17022799 | 5260293 | 112522836 | 112522860 | SEQ ID NO: 1,039, SEQ ID NO: |
| | | | | 1,040, SEQ ID NO: 1,041, SEQ ID |
| | | | | NO: 1,042, SEQ ID NO: 1,043, |
| | | | | SEQ ID NO: 1,044, SEQ ID NO: |
| | | | | 1,045, SEQ ID NO: 1,046, SEQ ID |
| | | | | NO: 1,047, SEQ ID NO: 1,048, |
| | | | | SEQ ID NO: 1,049, SEQ ID NO: |
| | | | | 1,050, SEQ ID NO: 1,051, SEQ ID |
| | | | | NO: 1,052, SEQ ID NO: 1,053, |
| | | | | SEQ ID NO: 1,054, SEQ ID NO: |
| | | | | 1,055, SEQ ID NO: 1,056, SEQ ID |
| | | | | NO: 1,057, SEQ ID NO: 1,058, |
| | | | | SEQ ID NO: 1,059, SEQ ID NO: |
| | | | | 1,060, SEQ ID NO: 1,061, SEQ ID |
| | | | | NO: 1,062, SEQ ID NO: 1,063, |
| | | | | SEQ ID NO: 1,064, SEQ ID NO: |
| | | | | 1,065, SEQ ID NO: 1,066, SEQ ID |
| | | | | NO: 1,067, SEQ ID NO: 1,068, |
| | | | | SEQ ID NO: 1,069, SEQ ID NO: |
| | | | | 1,070, SEQ ID NO: 1,071, SEQ ID |
| | | | | NO: 1,072, SEQ ID NO: 1,073, |
| | | | | SEQ ID NO: 1,074, SEQ ID NO: |
| | | | | 1,075, SEQ ID NO: 1,076, SEQ ID |
| | | | | NO: 1,077, SEQ ID NO: 1,078, |
| | | | | SEQ ID NO: 1,079, SEQ ID NO: |
| | | | | 1,080, SEQ ID NO: 1,081, SEQ ID |
| | | | | NO: 1,082, SEQ ID NO: 1,083, |
| | | | | SEQ ID NO: 1,084, SEQ ID NO: |
| | | | | 1,085, SEQ ID NO: 1,086, SEQ ID |
| | | | | NO: 1,087, SEQ ID NO: 1,088, |
| | | | | SEQ ID NO: 1,089, SEQ ID NO: |
| | | | | 1,090, SEQ ID NO: 1,091, SEQ ID |
| | | | | NO: 1,092, SEQ ID NO: 1,093, |
| | | | | SEQ ID NO: 1,094, SEQ ID NO: |
| | | | | 1,095, SEQ ID NO: 1,096, SEQ ID |
| | | | | NO: 1,097, SEQ ID NO: 1,098, |
| | | | | SEQ ID NO: 1,099, SEQ ID NO: |
| | | | | 1,100, SEQ ID NO: 1,101 |
| 16786369 | 5074755 | 74416815 | 74416839 | SEQ ID NO: 1,102, SEQ ID NO: |
| | | | | 1,103, SEQ ID NO: 1,104, SEQ ID |
| | | | | NO: 1,105, SEQ ID NO: 1,106, |
| | | | | SEQ ID NO: 1,107, SEQ ID NO: |
| | | | | 1,108, SEQ ID NO: 1,109, SEQ ID |
| | | | | NO: 1,110, SEQ ID NO: 1,111, |
| | | | | SEQ ID NO: 1,112, SEQ ID NO: |
| | | | | 1,113, SEQ ID NO: 1,114, SEQ ID |
| | | | | NO: 1,115, SEQ ID NO: 1,116, |
| | | | | SEQ ID NO: 1,117, SEQ ID NO: |
| | | | | 1,118, SEQ ID NO: 1,119, SEQ ID |
| | | | | NO: 1,120, SEQ ID NO: 1,121, |
| | | | | SEQ ID NO: 1,122, SEQ ID NO: |
| | | | | 1,123, SEQ ID NO: 1,124, SEQ ID |
| | | | | NO: 1,125, SEQ ID NO: 1,126, |
| | | | | SEQ ID NO: 1,127, SEQ ID NO: |
| | | | | 1,128, SEQ ID NO: 1,129, SEQ ID |
| | | | | NO: 1,130 |
| 16920377 | 5181208 | 50409490 | 50409566 | SEQ ID NO: 1,131, SEQ ID NO: |
| | | | | 1,132, SEQ ID NO: 1,133, SEQ ID |
| | | | | NO: 1,134, SEQ ID NO: 1,135, |
| | | | | SEQ ID NO: 1,136, SEQ ID NO: |
| | | | | 1,137, SEQ ID NO: 1,138, SEQ ID |
| | | | | NO: 1,139, SEQ ID NO: 1,140, |
| | | | | SEQ ID NO: 1,141, SEQ ID NO: |
| | | | | 1,142, SEQ ID NO: 1,143, SEQ ID |
| | | | | NO: 1,144, SEQ ID NO: 1,145, |
| | | | | SEQ ID NO: 1,146, SEQ ID NO: |
| | | | | 1,147, SEQ ID NO: 1,148, SEQ ID |
| | | | | NO: 1,149, SEQ ID NO: 1,150, |
| | | | | SEQ ID NO: 1,151, SEQ ID NO: |
| | | | | 1,152, SEQ ID NO: 1,153, SEQ ID |
| | | | | NO: 1,154, SEQ ID NO: 1,155, |
| | | | | SEQ ID NO: 1,156, SEQ ID NO: |
| | | | | 1,157, SEQ ID NO: 1,158 |
| 16851430 | 5126344 | 20735797 | 20735851 | SEQ ID NO: 1,159, SEQ ID NO: |
| | | | | 1,160, SEQ ID NO: 1,161, SEQ ID |
| | | | | NO: 1,162, SEQ ID NO: 1,163, |
| | | | | SEQ ID NO: 1,164, SEQ ID NO: |
| | | | | 1,165, SEQ ID NO: 1,166, SEQ ID |
| | | | | NO: 1,167, SEQ ID NO: 1,168, |
| | | | | SEQ ID NO: 1,169, SEQ ID NO: |
| | | | | 1,170, SEQ ID NO: 1,171, SEQ ID |
| | | | | NO: 1,172, SEQ ID NO: 1,173, |
| | | | | SEQ ID NO: 1,174, SEQ ID NO: |
| | | | | 1,175, SEQ ID NO: 1,176, SEQ ID |
| | | | | NO: 1,177, SEQ ID NO: 1,178, |
| | | | | SEQ ID NO: 1,179, SEQ ID NO: |
| | | | | 1,180, SEQ ID NO: 1,181, SEQ ID |
| | | | | NO: 1,182, SEQ ID NO: 1,183 |
| 16866470 | 5138794 | 622162 | 622229 | SEQ ID NO: 1,184, SEQ ID NO: |
| | | | | 1,185, SEQ ID NO: 1,186, SEQ ID |
| | | | | NO: 1,187, SEQ ID NO: 1,188, |
| | | | | SEQ ID NO: 1,189, SEQ ID NO: |
| | | | | 1,190, SEQ ID NO: 1,191, SEQ ID |
| | | | | NO: 1,192, SEQ ID NO: 1,193, |
| | | | | SEQ ID NO: 1,194, SEQ ID NO: |
| | | | | 1,195, SEQ ID NO: 1,196, SEQ ID |
| | | | | NO: 1,197, SEQ ID NO: 1,198, |
| | | | | SEQ ID NO: 1,199, SEQ ID NO: |
| | | | | 1,200, SEQ ID NO: 1,201, SEQ ID |
| | | | | NO: 1,202, SEQ ID NO: 1,203, |
| | | | | SEQ ID NO: 1,204, SEQ ID NO: |
| | | | | 1,205, SEQ ID NO: 1,206, SEQ ID |
| | | | | NO: 1,207, SEQ ID NO: 1,208, |
| | | | | SEQ ID NO: 1,209, SEQ ID NO: |
| | | | | 1,210 |
| 17000583 | 5243330 | 138716329 | 138716372 | SEQ ID NO: 1,211, SEQ ID NO: |
| | | | | 1,212, SEQ ID NO: 1,213, SEQ ID |
| | | | | NO: 1,214, SEQ ID NO: 1,215, |
| | | | | SEQ ID NO: 1,216, SEQ ID NO: |
| | | | | 1,217, SEQ ID NO: 1,218, SEQ ID |
| | | | | NO: 1,219, SEQ ID NO: 1,220, |
| | | | | SEQ ID NO: 1,221, SEQ ID NO: |
| | | | | 1,222, SEQ ID NO: 1,223, SEQ ID |
| | | | | NO: 1,224, SEQ ID NO: 1,225, |
| | | | | SEQ ID NO: 1,226, SEQ ID NO: |
| | | | | 1,227, SEQ ID NO: 1,228, SEQ ID |
| | | | | NO: 1,229, SEQ ID NO: 1,230, |
| | | | | SEQ ID NO: 1,231, SEQ ID NO: |
| | | | | 1,232, SEQ ID NO: 1,233, SEQ ID |
| | | | | NO: 1,234, SEQ ID NO: 1,235, |
| | | | | SEQ ID NO: 1,236, SEQ ID NO: |
| | | | | 1,237, SEQ ID NO: 1,238, SEQ ID |
| | | | | NO: 1,239, SEQ ID NO: 1,240 |
| 17019044 | 5257313 | 41162239 | 41162438 | SEQ ID NO: 1,241, SEQ ID NO: |
| | | | | 1,242, SEQ ID NO: 1,243, SEQ ID |
| | | | | NO: 1,244, SEQ ID NO: 1,245, |
| | | | | SEQ ID NO: 1,246, SEQ ID NO: |
| | | | | 1,247, SEQ ID NO: 1,248, SEQ ID |
| | | | | NO: 1,249, SEQ ID NO: 1,250, |
| | | | | SEQ ID NO: 1,251, SEQ ID NO: |
| | | | | 1,252, SEQ ID NO: 1,253, SEQ ID |
| | | | | NO: 1,254, SEQ ID NO: 1,255, |
| | | | | SEQ ID NO: 1,256 |
| 17057414 | 5285139 | 45022748 | 45022834 | SEQ ID NO: 1,257, SEQ ID NO: |
| | | | | 1,258, SEQ ID NO: 1,259, SEQ ID |
| | | | | NO: 1,260, SEQ ID NO: 1,261, |
| | | | | SEQ ID NO: 1,262, SEQ ID NO: |
| | | | | 1,263, SEQ ID NO: 1,264, SEQ ID |
| | | | | NO: 1,265, SEQ ID NO: 1,266, |
| | | | | SEQ ID NO: 1,267, SEQ ID NO: |
| | | | | 1,268, SEQ ID NO: 1,269, SEQ ID |
| | | | | NO: 1,270, SEQ ID NO: 1,271, |
| | | | | SEQ ID NO: 1,272, SEQ ID NO: |
| | | | | 1,273, SEQ ID NO: 1,274, SEQ ID |
| | | | | NO: 1,275, SEQ ID NO: 1,276 |
| 17059640 | 5286964 | 91758297 | 91758322 | SEQ ID NO: 1,277, SEQ ID NO: |
| | | | | 1,278, SEQ ID NO: 1,279, SEQ ID |
| | | | | NO: 1,280, SEQ ID NO: 1,281, |
| | | | | SEQ ID NO: 1,282, SEQ ID NO: |
| | | | | 1,283, SEQ ID NO: 1,284, SEQ ID |
| | | | | NO: 1,285, SEQ ID NO: 1,286, |
| | | | | SEQ ID NO: 1,287, SEQ ID NO: |
| | | | | 1,288, SEQ ID NO: 1,289, SEQ ID |
| | | | | NO: 1,290, SEQ ID NO: 1,291, |
| | | | | SEQ ID NO: 1,292, SEQ ID NO: |
| | | | | 1,293, SEQ ID NO: 1,294, SEQ ID |
| | | | | NO: 1,295, SEQ ID NO: 1,296, |
| | | | | SEQ ID NO: 1,297, SEQ ID NO: |
| | | | | 1,298, SEQ ID NO: 1,299 |
| 17076434 | 5299845 | 38280311 | 38280335 | SEQ ID NO: 1,300, SEQ ID NO: |
| | | | | 1,301, SEQ ID NO: 1,302, SEQ ID |
| | | | | NO: 1,303, SEQ ID NO: 1,304, |
| | | | | SEQ ID NO: 1,305, SEQ ID NO: |
| | | | | 1,306, SEQ ID NO: 1,307, SEQ ID |
| | | | | NO: 1,308, SEQ ID NO: 1,309, |
| | | | | SEQ ID NO: 1,310, SEQ ID NO: |
| | | | | 1,311, SEQ ID NO: 1,312, SEQ ID |
| | | | | NO: 1,313, SEQ ID NO: 1,314, |
| | | | | SEQ ID NO: 1,315, SEQ ID NO: |
| | | | | 1,316, SEQ ID NO: 1,317, SEQ ID |
| | | | | NO: 1,318, SEQ ID NO: 1,319, |
| | | | | SEQ ID NO: 1,320, SEQ ID NO: |
| | | | | 1,321, SEQ ID NO: 1,322, SEQ ID |
| | | | | NO: 1,323, SEQ ID NO: 1,324, |
| | | | | SEQ ID NO: 1,325, SEQ ID NO: |
| | | | | 1,326, SEQ ID NO: 1,327, SEQ ID |
| | | | | NO: 1,328, SEQ ID NO: 1,329, |
| | | | | SEQ ID NO: 1,330, SEQ ID NO: |
| | | | | 1,331, SEQ ID NO: 1,332, SEQ ID |
| | | | | NO: 1,333, SEQ ID NO: 1,334, |
| | | | | SEQ ID NO: 1,335 |
| 17114699 | 5329708 | 139805916 | 139805949 | SEQ ID NO: 1,336, SEQ ID NO: |
| | | | | 1,337, SEQ ID NO: 1,338, SEQ ID |
| | | | | NO: 1,339, SEQ ID NO: 1,340, |
| | | | | SEQ ID NO: 1,341, SEQ ID NO: |
| | | | | 1,342, SEQ ID NO: 1,343, SEQ ID |
| | | | | NO: 1,344, SEQ ID NO: 1,345, |
| | | | | SEQ ID NO: 1,346, SEQ ID NO: |
| | | | | 1,347, SEQ ID NO: 1,348 |

TABLE 6
Optimal Set of 16 probe sets that identifies specific exons in the RNA
biomarker, which are used for detecting that expression is decreased
or increased of RNA biomarkers in a sample comprising total polynucleotides
isolated from CD45-positive cells (or biofluids) from a male subject, corresponding
Probe Set IDs the Exon_ID, the start and end of the exon and the
nucleic acid sequences of the probes (Nucleic Acid Sequences of Probes)
within each probe set for the male subjects.

| PROBE | | | | NUCLEIC ACID |
|---|---|---|---|---|
| SET ID | EXON_ID | START | STOP | SEQUENCES OF PROBES |
| 17059640 | 5286964 | 91758297 | 91758322 | SEQ ID NO: 1,277, SEQ ID |
| | | | | NO: 1,278, SEQ ID NO: |
| | | | | 1,279, SEQ ID NO: 1,280, |
| | | | | SEQ ID NO: 1,281, SEQ ID |
| | | | | NO: 1,282, SEQ ID NO: |
| | | | | 1,283, SEQ ID NO: 1,284, |
| | | | | SEQ ID NO: 1,285, SEQ ID |
| | | | | NO: 1,286, SEQ ID NO: |
| | | | | 1,287, SEQ ID NO: 1,288, |
| | | | | SEQ ID NO: 1,289, SEQ ID |
| | | | | NO: 1,290, SEQ ID NO: |
| | | | | 1,291, SEQ ID NO: 1,292, |
| | | | | SEQ ID NO: 1,293, SEQ ID |
| | | | | NO: 1,294, SEQ ID NO: |
| | | | | 1,295, SEQ ID NO: 1,296, |
| | | | | SEQ ID NO: 1,297, SEQ ID |
| | | | | NO: 1,298, SEQ ID NO: |
| | | | | 1,299 |
| 17019044 | 5257313 | 41162239 | 41162438 | SEQ ID NO: 1,241, SEQ ID |
| | | | | NO: 1,242, SEQ ID NO: |
| | | | | 1,243, SEQ ID NO: 1,244, |
| | | | | SEQ ID NO: 1,245, SEQ ID |
| | | | | NO: 1,246, SEQ ID NO: |
| | | | | 1,247, SEQ ID NO: 1,248, |
| | | | | SEQ ID NO: 1,249, SEQ ID |
| | | | | NO: 1,250, SEQ ID NO: |
| | | | | 1,251, SEQ ID NO: 1,252, |
| | | | | SEQ ID NO: 1,253, SEQ ID |
| | | | | NO: 1,254, SEQ ID NO: |
| | | | | 1,255, SEQ ID NO: 1,256 |
| 17076434 | 5299845 | 38280311 | 38280335 | SEQ ID NO: 1,300, SEQ ID |
| | | | | NO: 1,301, SEQ ID NO: |
| | | | | 1,302, SEQ ID NO: 1,303, |
| | | | | SEQ ID NO: 1,304, SEQ ID |
| | | | | NO: 1,305, SEQ ID NO: |
| | | | | 1,306, SEQ ID NO: 1,307, |
| | | | | SEQ ID NO: 1,308, SEQ ID |
| | | | | NO: 1,309, SEQ ID NO: |
| | | | | 1,310, SEQ ID NO: 1,311, |
| | | | | SEQ ID NO: 1,312, SEQ ID |
| | | | | NO: 1,313, SEQ ID NO: |
| | | | | 1,314, SEQ ID NO: 1,315, |
| | | | | SEQ ID NO: 1,316, SEQ ID |
| | | | | NO: 1,317, SEQ ID NO: |
| | | | | 1,318, SEQ ID NO: 1,319, |
| | | | | SEQ ID NO: 1,320, SEQ ID |
| | | | | NO: 1,321, SEQ ID NO: |
| | | | | 1,322, SEQ ID NO: 1,323, |
| | | | | SEQ ID NO: 1,324, SEQ ID |
| | | | | NO: 1,325, SEQ ID NO: |
| | | | | 1,326, SEQ ID NO: 1,327, |
| | | | | SEQ ID NO: 1,328, SEQ ID |
| | | | | NO: 1,329, SEQ ID NO: |
| | | | | 1,330, SEQ ID NO: 1,331, |
| | | | | SEQ ID NO: 1,332, SEQ ID |
| | | | | NO: 1,333, SEQ ID NO: |
| | | | | 1,334, SEQ ID NO: 1,335 |
| 16945454 | 5200110 | 129695566 | 129696022 | SEQ ID NO: 782, SEQ ID |
| | | | | NO: 783, SEQ ID NO: 784, |
| | | | | SEQ ID NO: 785, SEQ ID |
| | | | | NO: 786, SEQ ID NO: 787, |
| | | | | SEQ ID NO: 788, SEQ ID |
| | | | | NO: 789, SEQ ID NO: 790, |
| | | | | SEQ ID NO: 791, SEQ ID |
| | | | | NO: 792, SEQ ID NO: 793, |
| | | | | SEQ ID NO: 794, SEQ ID |
| | | | | NO: 795, SEQ ID NO: 796, |
| | | | | SEQ ID NO: 797, SEQ ID |
| | | | | NO: 798, SEQ ID NO: 799, |
| | | | | SEQ ID NO: 800, SEQ ID |
| | | | | NO: 801, SEQ ID NO: 802, |
| | | | | SEQ ID NO: 803 |
| 17058221 | 5285817 | 65957267 | 65957293 | SEQ ID NO: 868, SEQ ID |
| | | | | NO: 869, SEQ ID NO: 870, |
| | | | | SEQ ID NO: 871, SEQ ID |
| | | | | NO: 872, SEQ ID NO: 873, |
| | | | | SEQ ID NO: 874, SEQ ID |
| | | | | NO: 875, SEQ ID NO: 876, |
| | | | | SEQ ID NO: 877, SEQ ID |
| | | | | NO: 878, SEQ ID NO: 879 |
| 17000583 | 5243330 | 138716329 | 138716372 | SEQ ID NO: 1,211, SEQ ID |
| | | | | NO: 1,212, SEQ ID NO: |
| | | | | 1,213, SEQ ID NO: 1,214, |
| | | | | SEQ ID NO: 1,215, SEQ ID |
| | | | | NO: 1,216, SEQ ID NO: |
| | | | | 1,217, SEQ ID NO: 1,218, |
| | | | | SEQ ID NO: 1,219, SEQ ID |
| | | | | NO: 1,220, SEQ ID NO: |
| | | | | 1,221, SEQ ID NO: 1,222, |
| | | | | SEQ ID NO: 1,223, SEQ ID |
| | | | | NO: 1,224, SEQ ID NO: |
| | | | | 1,225, SEQ ID NO: 1,226, |
| | | | | SEQ ID NO: 1,227, SEQ ID |
| | | | | NO: 1,228, SEQ ID NO: |
| | | | | 1,229, SEQ ID NO: 1,230, |
| | | | | SEQ ID NO: 1,231, SEQ ID |
| | | | | NO: 1,232, SEQ ID NO: |
| | | | | 1,233, SEQ ID NO: 1,234, |
| | | | | SEQ ID NO: 1,235, SEQ ID |
| | | | | NO: 1,236, SEQ ID NO: |
| | | | | 1,237, SEQ ID NO: 1,238, |
| | | | | SEQ ID NO: 1,239, SEQ ID |
| | | | | NO: 1,240 |
| 17114699 | 5329708 | 139805916 | 139805949 | SEQ ID NO: 1,336, SEQ ID |
| | | | | NO: 1,337, SEQ ID NO: |
| | | | | 1,338, SEQ ID NO: 1,339, |
| | | | | SEQ ID NO: 1,340, SEQ ID |
| | | | | NO: 1,341, SEQ ID NO: |
| | | | | 1,342, SEQ ID NO: 1,343, |
| | | | | SEQ ID NO: 1,344, SEQ ID |
| | | | | NO: 1,345, SEQ ID NO: |
| | | | | 1,346, SEQ ID NO: 1,347, |
| | | | | SEQ ID NO: 1,348 |
| 16866470 | 5138794 | 622162 | 622229 | SEQ ID NO: 1,184, SEQ ID |
| | | | | NO: 1,185, SEQ ID NO: |
| | | | | 1,186, SEQ ID NO: 1,187, |
| | | | | SEQ ID NO: 1,188, SEQ ID |
| | | | | NO: 1,189, SEQ ID NO: |
| | | | | 1,190, SEQ ID NO: 1,191, |
| | | | | SEQ ID NO: 1,192, SEQ ID |
| | | | | NO: 1,193, SEQ ID NO: |
| | | | | 1,194, SEQ ID NO: 1,195, |
| | | | | SEQ ID NO: 1,196, SEQ ID |
| | | | | NO: 1,197, SEQ ID NO: |
| | | | | 1,198, SEQ ID NO: 1,199, |
| | | | | SEQ ID NO: 1,200, SEQ ID |
| | | | | NO: 1,201, SEQ ID NO: |
| | | | | 1,202, SEQ ID NO: 1,203, |
| | | | | SEQ ID NO: 1,204, SEQ ID |
| | | | | NO: 1,205, SEQ ID NO: |
| | | | | 1,206, SEQ ID NO: 1,207, |
| | | | | SEQ ID NO: 1,208, SEQ ID |
| | | | | NO: 1,209, SEQ ID NO: |
| | | | | 1,210 |
| 17080053 | 5302714 | 106924048 | 106924072 | SEQ ID NO: 977, SEQ ID |
| | | | | NO: 978, SEQ ID NO: 979, |
| | | | | SEQ ID NO: 980, SEQ ID |
| | | | | NO: 981, SEQ ID NO: 982, |
| | | | | SEQ ID NO: 983, SEQ ID |
| | | | | NO: 984, SEQ ID NO: 985, |
| | | | | SEQ ID NO: 986, SEQ ID |
| | | | | NO: 987, SEQ ID NO: 988, |
| | | | | SEQ ID NO: 989, SEQ ID |
| | | | | NO: 990, SEQ ID NO: 991, |
| | | | | SEQ ID NO: 992, SEQ ID |
| | | | | NO: 993, SEQ ID NO: 994, |
| | | | | SEQ ID NO: 995, SEQ ID |
| | | | | NO: 996, SEQ ID NO: 997, |
| | | | | SEQ ID NO: 998, SEQ ID |
| | | | | NO: 999, SEQ ID NO: |
| | | | | 1,000, SEQ ID NO: 1,001, |
| | | | | SEQ ID NO: 1,002 |
| 16786369 | 5074755 | 74416815 | 74416839 | SEQ ID NO: 1,102, SEQ ID |
| | | | | NO: 1,103, SEQ ID NO: |
| | | | | 1,104, SEQ ID NO: 1,105, |
| | | | | SEQ ID NO: 1,106, SEQ ID |
| | | | | NO: 1,107, SEQ ID NO: |
| | | | | 1,108, SEQ ID NO: 1,109, |
| | | | | SEQ ID NO: 1,110, SEQ ID |
| | | | | NO: 1,111, SEQ ID NO: |
| | | | | 1,112, SEQ ID NO: 1,113, |
| | | | | SEQ ID NO: 1,114, SEQ ID |
| | | | | NO: 1,115, SEQ ID NO: |
| | | | | 1,116, SEQ ID NO: 1,117, |
| | | | | SEQ ID NO: 1,118, SEQ ID |
| | | | | NO: 1,119, SEQ ID NO: |
| | | | | 1,120, SEQ ID NO: 1,121, |
| | | | | SEQ ID NO: 1,122, SEQ ID |
| | | | | NO: 1,123, SEQ ID NO: |
| | | | | 1,124, SEQ ID NO: 1,125, |
| | | | | SEQ ID NO: 1,126, SEQ ID |
| | | | | NO: 1,127, SEQ ID NO: |
| | | | | 1,128, SEQ ID NO: 1,129, |
| | | | | SEQ ID NO: 1,130 |
| 17118935 | 0 | 17 | 47 | SEQ ID NO: 775, SEQ ID |
| | | | | NO: 776, SEQ ID NO: 777, |
| | | | | SEQ ID NO: 778, SEQ ID |
| | | | | NO: 779, SEQ ID NO: 780, |
| | | | | SEQ ID NO: 781 |
| 16877665 | 5147827 | 24248302 | 24248326 | SEQ ID NO: 921, SEQ ID |
| | | | | NO: 922, SEQ ID NO: 923, |
| | | | | SEQ ID NO: 924, SEQ ID |
| | | | | NO: 925, SEQ ID NO: 926, |
| | | | | SEQ ID NO: 927, SEQ ID |
| | | | | NO: 928, SEQ ID NO: 929, |
| | | | | SEQ ID NO: 930, SEQ ID |
| | | | | NO: 931, SEQ ID NO: 932, |
| | | | | SEQ ID NO: 933, SEQ ID |
| | | | | NO: 934, SEQ ID NO: 935, |
| | | | | SEQ ID NO: 936, SEQ ID |
| | | | | NO: 937, SEQ ID NO: 938, |
| | | | | SEQ ID NO: 939, SEQ ID |
| | | | | NO: 940, SEQ ID NO: 941, |
| | | | | SEQ ID NO: 942 |
| 17057414 | 5285139 | 45022748 | 45022834 | SEQ ID NO: 1,257, SEQ ID |
| | | | | NO: 1,258, SEQ ID NO: |
| | | | | 1,259, SEQ ID NO: 1,260, |
| | | | | SEQ ID NO: 1,261, SEQ ID |
| | | | | NO: 1,262, SEQ ID NO: |
| | | | | 1,263, SEQ ID NO: 1,264, |
| | | | | SEQ ID NO: 1,265, SEQ ID |
| | | | | NO: 1,266, SEQ ID NO: |
| | | | | 1,267, SEQ ID NO: 1,268, |
| | | | | SEQ ID NO: 1,269, SEQ ID |
| | | | | NO: 1,270, SEQ ID NO: |
| | | | | 1,271, SEQ ID NO: 1,272, |
| | | | | SEQ ID NO: 1,273, SEQ ID |
| | | | | NO: 1,274, SEQ ID NO: |
| | | | | 1,275, SEQ ID NO: 1,276 |
| 16851430 | 5126344 | 20735797 | 20735851 | SEQ ID NO: 1,159, SEQ ID |
| | | | | NO: 1,160, SEQ ID NO: |
| | | | | 1,161, SEQ ID NO: 1,162, |
| | | | | SEQ ID NO: 1,163, SEQ ID |
| | | | | NO: 1,164, SEQ ID NO: |
| | | | | 1,165, SEQ ID NO: 1,166, |
| | | | | SEQ ID NO: 1,167, SEQ ID |
| | | | | NO: 1,168, SEQ ID NO: |
| | | | | 1,169, SEQ ID NO: 1,170, |
| | | | | SEQ ID NO: 1,171, SEQ ID |
| | | | | NO: 1,172, SEQ ID NO: |
| | | | | 1,173, SEQ ID NO: 1,174, |
| | | | | SEQ ID NO: 1,175, SEQ ID |
| | | | | NO: 1,176, SEQ ID NO: |
| | | | | 1,177, SEQ ID NO: 1,178, |
| | | | | SEQ ID NO: 1,179, SEQ ID |
| | | | | NO: 1,180, SEQ ID NO: |
| | | | | 1,181, SEQ ID NO: 1,182, |
| | | | | SEQ ID NO: 1,183 |
| 16868954 | 5140844 | 11513296 | 11513320 | SEQ ID NO: 905, SEQ ID |
| | | | | NO: 906, SEQ ID NO: 907, |
| | | | | SEQ ID NO: 908, SEQ ID |
| | | | | NO: 909, SEQ ID NO: 910, |
| | | | | SEQ ID NO: 911, SEQ ID |
| | | | | NO: 912, SEQ ID NO: 913, |
| | | | | SEQ ID NO: 914, SEQ ID |
| | | | | NO: 915, SEQ ID NO: 916, |
| | | | | SEQ ID NO: 917, SEQ ID |
| | | | | NO: 918, SEQ ID NO: 919, |
| | | | | SEQ ID NO: 920 |
| 16831561 | 5110382 | 16926501 | 16926546 | SEQ ID NO: 704, SEQ ID |
| | | | | NO: 705, SEQ ID NO: 706, |
| | | | | SEQ ID NO: 707, SEQ ID |
| | | | | NO: 708, SEQ ID NO: 709, |
| | | | | SEQ ID NO: 710, SEQ ID |
| | | | | NO: 711, SEQ ID NO: 712, |
| | | | | SEQ ID NO: 713, SEQ ID |
| | | | | NO: 714, SEQ ID NO: 715, |
| | | | | SEQ ID NO: 716, SEQ ID |
| | | | | NO: 717, SEQ ID NO: 718, |
| | | | | SEQ ID NO: 719, SEQ ID |
| | | | | NO: 720, SEQ ID NO: 721, |
| | | | | SEQ ID NO: 722 |

In a specific aspect the invention provides an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM1L2, LAMA4 and SALL4, in CD45+ mononuclear cells (MNC) from a test sample collected from said male subject, and b. comparing said first expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM1L2, LAMA4 and SALL4, in CD45+ mononuclear cells (MNC) collected from said male subject with a control expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM1L2, LAMA4 and SALL4, from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level.

In a further specific aspect the invention provides an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and b. comparing said first expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level.

In a preferred embodiment, CD45+ MNC cells may be isolated from a blood sample or tissue or biofluids sample taken from a female or male subject using conventional methods, for instance CD45+ MNC cells may be isolated from the blood sample using conventional blood cell isolation techniques and immunohistochemistry techniques (e.g. using antibodies which are specific for capturing or binding CD45+ MNC cells, such as CD32 Monoclonal Antibodies by Invitrogen (6C4 (CD32)), Functional Grade, eBioscience). Alternatively, the specific RNA biomarker panel may be detected in unsorted cells from blood samples (as long as (1) CD45+ MNC cells are present, or (2) have been present, or (3) have been able to secrete the RNA biomarker into the biofluids). The skilled person is well-acquainted with CD45+ MNC cells and is aware of methods for isolating them from the blood. In a subsequent step, total polynucleotides may be isolated from said CD45+ MNC cells or biofluids, which in turn may be used for detecting the expression levels of the RNA biomarkers of the invention, using the probes as taught herein (or conventional antibody detection methods).

In the methods of the invention (i.e. for both female-specific and male-specific methods), the term ‘making a diagnosis’ or ‘diagnosing’ as used herein refers to the act of determining which disease(s) or condition(s) explains a person's symptoms and signs. The information required for diagnosis is typically collected from a history and physical examination of the person (female or male) seeking medical care. Often, two or more diagnostic procedures, such as diagnostic tests, are also done during the process.

Diagnostic tests allow physicians to make informed decisions about treatment and establish a prognosis. The act of ‘make a diagnosis’ or ‘diagnosing’ (as well as the opinion reached thereby) does not necessarily involve elucidation of the aetiology of the diseases or conditions of interest. Additionally, for follow up/monitoring of patients (the natural progression of disease).

In an embodiment the IVDC is selected from the group consisting of myocardial ischemia, myocardial infarction and angina pectoris. In a preferred embodiment, the IVDC is myocardial ischemia, preferably early onset myocardial ischemia/silent ischemia, as discussed above.

In a preferred embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) from a test sample collected from said female subject, and b. comparing said first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) collected from said female subject with a control expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. determining a second expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) from a further test sample collected from said subject at a later time point than the first test sample, e. comparing the first expression level of each RNA biomarker with the second expression level of each RNA biomarker, f. diagnosing whether the progression of IVD and/or IVDC over time in said female subject has occurred based on the difference of at least two RNA biomarkers from said group between the first expression level and the second expression level in said female subject.

In a further preferred embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) from a test sample collected from said female subject, and b. comparing said first expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) collected from said female subject with a control expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. determining a second expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) from a further test sample collected from said subject at a later time point than the first test sample, e. comparing the first expression level of each RNA biomarker with the second expression level of each RNA biomarker, f. diagnosing whether the progression of IVD and/or IVDC over time in said female subject has occurred based on the difference of at least two RNA biomarkers from said group between the first expression level and the second expression level in said female subject.

In an even further preferred embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG, in CD45+ mononuclear cells (MNC) from a test sample collected from said female subject, and b. comparing said first expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG, in CD45+ mononuclear cells (MNC) collected from said female subject with a control expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. determining a second expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG, in CD45+ mononuclear cells (MNC) from a further test sample collected from said subject at a later time point than the first test sample, e. comparing the first expression level of each RNA biomarker with the second expression level of each RNA biomarker, f. diagnosing whether the progression of IVD and/or IVDC over time in said female subject has occurred based on the difference of at least two RNA biomarkers from said group between the first expression level and the second expression level in said female subject.

In a preferred embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) from a test sample collected from said female subject, and b. comparing said first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) collected from said female subject with a control expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. monitoring a treatment for IVD and/or IVDC after step c., e. determining a second expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) from a further test sample collected from said subject at a later time point than the first test sample, f. comparing the first expression level of each RNA biomarker with the second expression level of each RNA biomarker, g. determining whether the treatment for IVD and/or IVDC has improved the occurrence and/or severity of IVD and/or IVDC in said female subject based on the difference of at least two RNA biomarkers from said group between the first expression level and the second expression level in said female subject.

In a further embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) from a test sample collected from said female subject, and b. comparing said first expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) collected from said female subject with a control expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. monitoring a treatment for IVD and/or IVDC after step c., e. determining a second expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) from a further test sample collected from said subject at a later time point than the first test sample, f. comparing the first expression level of each RNA biomarker with the second expression level of each RNA biomarker, g. determining whether the treatment for IVD and/or IVDC has improved the occurrence and/or severity of IVD and/or IVDC in said female subject based on the difference of at least two RNA biomarkers from said group between the first expression level and the second expression level in said female subject.

In an even further preferred embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG, in CD45+ mononuclear cells (MNC) from a test sample collected from said female subject, and b. comparing said first expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG, in CD45+ mononuclear cells (MNC) collected from said female subject with a control expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. monitoring a treatment for IVD and/or IVDC after step c., e. determining a second expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG, in CD45+ mononuclear cells (MNC) from a further test sample collected from said subject at a later time point than the first test sample, f. comparing the first expression level of each RNA biomarker with the second expression level of each RNA biomarker, g. determining whether the treatment for IVD and/or IVDC has improved the occurrence and/or severity of IVD and/or IVDC in said female subject based on the difference of at least two RNA biomarkers from said group between the first expression level and the second expression level in said female subject.

In a preferred embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) from a test sample collected from said female subject, and b. comparing said first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) collected from said female subject with a control expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least five, preferably at least ten RNA biomarkers from said group are different between the first expression level and the control expression level.

In a further preferred embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) from a test sample collected from said female subject, and b. comparing said first expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) collected from said female subject with a control expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least five, preferably at least ten RNA biomarkers from said group are different between the first expression level and the control expression level.

In an even further preferred embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG, in CD45+ mononuclear cells (MNC) from a test sample collected from said female subject, and b. comparing said first expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG, in CD45+ mononuclear cells (MNC) collected from said female subject with a control expression level of each RNA biomarker from the group consisting of ADAMTS8, AK094845, AMOT, BTRC, CNTNAP3, COL2A1, DCHS2, DENND5B, LPAR6, NBLA00301, MMP11, PRSS33, QTRTD1 and TDG, from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least five, preferably at least ten RNA biomarkers from said group are different between the first expression level and the control expression level.

In a preferred embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) from a test sample collected from said female subject, and b. comparing said first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) collected from said female subject with a control expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least fourteen RNA biomarkers from said group are different between the first expression level and the control expression level.

In a preferred embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) from a test sample collected from said female subject, and b. comparing said first expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1, in CD45+ mononuclear cells (MNC) collected from said female subject with a control expression level of each RNA biomarker from the group consisting of CCDC57, FOXN4, GTF21RD1, HNRNPR, KIF18B, NKAIN3, NRXN2, PTS, STK36, TBCB, TCONS_00015170-XLOC_007214 and UROC1 from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least twelve RNA biomarkers from said group are different between the first expression level and the control expression level.

In a preferred embodiment the method of the invention concerns an in vitro method for diagnosis of occurrence and/or assessing severity of ischemic vascular disease (IVD) and/or ischemic vascular disease-related complication (IVDC) in a female subject, said method comprising the steps of a. determining a first expression level of each RNA biomarker from the group consisting of ADAMTS8, AK094845, AMOT, BTRC, CNTNAP3, COL2A1, DCHS2, DENND5B, LPAR6, MMP11, NBLA00301, PRSS33, QTRTD1 and TDG, in CD45+ mononuclear cells (MNC) from a test sample collected from said female subject, and b. comparing said first expression level of each RNA biomarker from the group consisting of ADAMTS8, AK094845, AMOT, BTRC, CNTNAP3, COL2A1, DCHS2, DENND5B, LPAR6, MMP11, NBLA00301, PRSS33, QTRTD1 and TDG, in CD45+ mononuclear cells (MNC) collected from said female subject with a control expression level of each RNA biomarker from the group consisting of ADAMTS8, AK094845, AMOT, BTRC, CNTNAP3, COL2A1, DCHS2, DENND5B, LPAR6, MMP11, NBLA00301, PRSS33, QTRTD1 and TDG from a control sample collected from control female subject(s), c. diagnosing occurrence and/or assessing severity of IVD and/or IVDC based on the differences between the first expression level and the control expression level, wherein it is determined that IVD and/or IVDC occurred or is severe if at least fourteen RNA biomarkers from said group are different between the first expression level and the control expression level.

In a preferred embodiment the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and b. comparing said first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. determining a second expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) from a further test sample collected from said subject at a later time point than the first test sample, e. comparing the first expression level of each RNA biomarker with the second expression level of each RNA biomarker, f. diagnosing whether the progression of IVD and/or IVDC over time in said male subject has occurred based on the difference of at least two RNA biomarkers from said group between the first expression level and the second expression level in said male subject.

In a further preferred embodiment, the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM1L2, LAMA4 and SALL4 in CD45+ mononuclear cells (MNC) from a test sample collected from said male subject, and b. comparing said first expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM11L2, LAMA4 and SALL4, in CD45+ mononuclear cells (MNC) collected from said male subject with a control expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM11L2, LAMA4 and SALL4 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. determining a second expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM1 L2, LAMA4 and SALL4, in CD45+ mononuclear cells (MNC) from a further test sample collected from said subject at a later time point than the first test sample, e. comparing the first expression level of each RNA biomarker with the second expression level of each RNA biomarker, f. diagnosing whether the progression of IVD and/or IVDC over time in said male subject has occurred based on the difference of at least two RNA biomarkers from said group between the first expression level and the second expression level in said male subject.

In an even further preferred embodiment the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and b. comparing said first expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. determining a second expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) from a further test sample collected from said subject at a later time point than the first test sample, e. comparing the first expression level of each RNA biomarker with the second expression level of each RNA biomarker, f. diagnosing whether the progression of IVD and/or IVDC over time in said male subject has occurred based on the difference of at least two RNA biomarkers from said group between the first expression level and the second expression level in said male subject.

In a preferred embodiment the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and b. comparing said first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. monitoring a treatment for IVD and/or IVDC after step c., e. determining a second expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) from a further test sample collected from said subject at a later time point than the first test sample, g. comparing the first expression level of each RNA biomarker with the second expression level of each RNA biomarker, h. determining whether the treatment for IVD and/or IVDC has improved the occurrence and/or severity of IVD and/or IVDC in said male subject based on the difference of at least two RNA biomarkers from said group between the first expression level and the second expression level in said male subject.

In a further preferred embodiment the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM1 L2, LAMA4 and SALL4, in CD45+ mononuclear cells (MNC) from a test sample collected from said male subject, and b. comparing said first expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM11L2, LAMA4 and SALL4, in CD45+ mononuclear cells (MNC) collected from said male subject with a control expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM11L2, LAMA4 and SALL4 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. monitoring a treatment for IVD and/or IVDC after step c., e. determining a second expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM11L2, LAMA4 and SALL4, in CD45+ mononuclear cells (MNC) from a further test sample collected from said subject at a later time point than the first test sample, g. comparing the first expression level of each RNA biomarker with the second expression level of each RNA biomarker, h. determining whether the treatment for IVD and/or IVDC has improved the occurrence and/or severity of IVD and/or IVDC in said male subject based on the difference of at least two RNA biomarkers from said group between the first expression level and the second expression level in said male subject.

In an even further preferred embodiment the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and b. comparing said first expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least two RNA biomarkers from said group are different between the first expression level and the control expression level, d. monitoring a treatment for IVD and/or IVDC after step c., e. determining a second expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) from a further test sample collected from said subject at a later time point than the first test sample, g. comparing the first expression level of each RNA biomarker with the second expression level of each RNA biomarker, h. determining whether the treatment for IVD and/or IVDC has improved the occurrence and/or severity of IVD and/or IVDC in said male subject based on the difference of at least two RNA biomarkers from said group between the first expression level and the second expression level in said male subject.

In a preferred embodiment the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and b. comparing said first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least ten, preferably at least fourteen, RNA biomarkers from said group are different between the first expression level and the control expression level.

In a further preferred embodiment the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM1 L2, LAMA4 and SALL4, in CD45+ mononuclear cells (MNC) from a test sample collected from said male subject, and b. comparing said first expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM11L2, LAMA4 and SALL4, in CD45+ mononuclear cells (MNC) collected from said male subject with a control expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM11L2, LAMA4 and SALL4 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least ten, preferably at least fourteen, RNA biomarkers from said group are different between the first expression level and the control expression level.

In an even further preferred embodiment, the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and b. comparing said first expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least ten, preferably at least fourteen, RNA biomarkers from said group are different between the first expression level and the control expression level.

In a preferred embodiment the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and b. comparing said first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249 from a control sample collected from control male subject(s), c. diagnosing occurrence and/or assessing severity of IVD and/or IVDC based on the differences between the first expression level and the control expression level, wherein it is determined that IVD and/or IVDC occurred or is severe at least sixteen, preferably at least eighteen RNA biomarkers from said group are different between the first expression level and the control expression level.

In a further preferred embodiment the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM1 L2, LAMA4 and SALL4, in CD45+ mononuclear cells (MNC) from a test sample collected from said male subject, and b. comparing said first expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM11L2, LAMA4 and SALL4, in CD45+ mononuclear cells (MNC) collected from said male subject with a control expression level of each RNA biomarker from the group consisting of ENST00000440123, MTPAP, ENST00000430988, USP12-AS1, XR_109422//LOC100507049, ENST00000456944, TBX5, ExonID_5262586, CALD1, TOM11L2, LAMA4 and SALL4 from a control sample collected from control male subject(s), c. diagnosing occurrence and/or assessing severity of IVD and/or IVDC based on the differences between the first expression level and the control expression level, wherein it is determined that IVD and/or IVDC occurred or is severe at least twelve RNA biomarkers from said group are different between the first expression level and the control expression level.

In an even further preferred embodiment, the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and b. comparing said first expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of TCONS_00025321-XLOC_012127, ProbSetlD_17118935, TRH, TCONS_I2_00026656-XLOC_I2_013873, RGL3, MFSD2B, ENST00000518932, COQ6, CABLES1, POLRMT, SLC23A1, TREML2, SNHG15, CYP51A1, ExonID_5299845 and TCONS_00017554-XLOC_008249 from a control sample collected from control male subject(s), c. diagnosing occurrence and/or assessing severity of IVD and/or IVDC based on the differences between the first expression level and the control expression level, wherein it is determined that IVD and/or IVDC occurred or is severe at least sixteen RNA biomarkers from said group are different between the first expression level and the control expression level.

In a preferred embodiment the method of the invention concerns an in vitro method for diagnosis of occurrence and/or assessing severity of ischemic vascular disease (IVD) and/or ischemic vascular disease-related complication (IVDC) in a male subject, said method comprising the steps of a. determining a first expression level of each RNA biomarker from the group consisting of CYP51A1, TREML2, ProbSetlD_17118935, TRH, TCONS_I2_00026656-XLOC_I2_013873, SLC23A1, TCONS_00017554-XLOC_008249, POLRMT, ENST00000518932, COQ6, ExonID_5299845, MFSD2B, SNHG15, CABLES1, RGL3 and TCONS_00025321-XLOC_012127, in CD45+ mononuclear cells (MNC) from a test sample collected from said male subject, and b. comparing said first expression level of each RNA biomarker from the group consisting of CYP51A1, TREML2, ProbSetlD_17118935, TRH, TCONS_I2_00026656-XLOC_I2_013873, SLC23A1, TCONS_00017554-XLOC_008249, POLRMT ENST00000518932, COQ6, ExonID_5299845, MFSD2B, SNHG15, CABLES1, RGL3 and TCONS_00025321-XLOC_012127, in CD45+ mononuclear cells (MNC) collected from said male subject with a control expression level of each RNA biomarker from the group consisting of CYP51A1, TREML2, ProbSetlD_17118935, TRH, TCONS_I2_00026656-XLOC_I2_013873, SLC23A1, TCONS_00017554-XLOC_008249, POLRMT, ENST00000518932, COQ6, ExonID_5299845, MFSD2B, SNHG15, CABLES1, RGL3 and TCONS_00025321-XLOC_012127 from a control sample collected from control male subject(s), c. diagnosing occurrence and/or assessing severity of IVD and/or IVDC based on the differences between the first expression level and the control expression level, wherein it is determined that IVD and/or IVDC occurred or is severe if at least sixteen RNA biomarkers from said group are different between the first expression level and the control expression level.

### EXAMPLES

### Example 1

### Female-Specific Study

A study was performed to assess the pattern of expression of the total polynucleotides expressed in or on CD45+ MNC cells obtained from female patients (n=31) suffering from myocardial ischemia vs female control subjects in which myocardial ischemia was excluded (n=91). the goal of the study was, to first, determine whether any differential pattern of expression of the total polynucleotides expressed in or on CD45+ MNC cells exist between female patients suffering from myocardial ischemia as compared to control subjects, and second, to use the results (i.e. the differentially expressed polynucleotides) to identify which differentially expressed polynucleotides may be used as a RNA biomarker for diagnosing, detecting the occurrence of, progression of, quantifying myocardial ischemia and/or monitoring myocardial ischemia over time or in response to treatment in female subject or patient.

Patients suffering from myocardial ischemia and control subjects were selected as follows. Female subjects were referred by the primary physician to be examined for chest pain complaints (e.g. myocardial ischemia or angina pectoris) at the cardiology outpatient clinic. Chest pain and/or chest discomfort may be caused by myocardial ischemia, but may also be caused by unrelated diseases, including anxiety, gastrointestinal complaints (for instance gastric reflux, stomach ulcer), or muscle ache. Therefore, to rule out unrelated conditions and to select patients suffering from myocardial ischemia, patients were screened using standard protocols for anamnesis, ECG, blood analysis, exercise testing (ergometry) and multi-slice CT scan. In case of ambiguous results, the patients were subjected to nuclear myocardial perfusion imaging (MIBI SPECT analysis) to rule out any myocardial ischemia. Patients suffering from myocardial ischemia, which were selected based on the battery of tests described therein, provided blood samples for further analysis (e.g. isolation of total polynucleotides from immune-isolated CD45+ cells and subsequent polynucleotide expression profiling using a commercial Affymetrix Human Gene 2.1 ST array). Blood samples were also obtained from healthy subjects that also have been referred for further diagnosis of chest discomfort, however myocardial ischemia was ruled out using standard of care diagnosis (using ergometry, MSCT and MIBI SPECT). These patients also received a telephone follow-up at 3 months after this diagnosis to ensure the absence of any cardiac event during the 3 months following the diagnosis (including any admission or intervention due to angina pectoris). The samples from the healthy subjects served as a control group.

### Example 2

### Male-Specific Study

The experimental procedure of example 1 was repeated for male patients (n=98) suffering from myocardial ischemia and male control subjects not suffering from myocardial ischemia (n=84).

### Example 3

Isolation of CD45+ MNC Cells from Blood Samples

The protocol below was applied to blood samples obtained from all patients (both sexes, with and without manifest myocardial ischemia).

The following buffers and mediums were prepared according to standard procedures:
Buffer 1: consists of a PBS buffer with 0.4% EDTA.
Buffer 2: consists of a PBS buffer with 0.2% EDTA and 2% FCS.
Freeze medium: consists of a DMEM medium with 10% FCS and 10% DMSO.

The blood samples were processed as follows:
step 1: A total of 6 tubes of whole blood for each patient were collected and labelled accordingly.
step 2: an amount of 20 ml of whole blood was transferred in one 50 cc tube and 28 ml of with buffer was added.
step 3: A total of 12 tubes containing 9 ml of Ficoll (used to separate blood to its components) were prepared per patient.
step 4: An amount of 12 ml of dilated blood (obtained from step 2) was added to each tube of step 3.
step 5: The tubes of step 4 were centrifuged at a speed of 1400 rpm, for 40 minutes.
step 6: The buffy coat present at the surface of the tubes of step 5 was gently pipetted and transferred to a new tube. Specifically, an amount corresponding to the buffy coats taken from 4 tubes (step 5) was transferred into one tube, after which the volume in the tube was completed to 50 ml with buffer 2.
step 7: The tubes of step 6 were centrifuged at a speed of 1400 rpm, for 10 min.
step 8: The pellet (containing the CD45+ MNC cells) formed in each tube (step 7) was re-dissolved in 10 ml of buffer 2.
step 9: An amount of 5 ml of CD45+ cell solution (from step 8) was pipetted into 2 new tubes.
step 10: The tubes of step 9 were centrifuged at a speed of 1400 rpm, for 10 min.
step 11: The supernatant (from the tube of step 10) was decanted and allowed to dry.
step 12: The pellet formed in the tubes of step 11, was put in a new tube (1 pellet/tube) on ice and re-dissolved in 3 ml of freeze medium and stored at −80° C.
step 13: After approximately 1 week, the samples of step 12 were thawed and dissolved in buffer 2 (1 pellet in 100 ul buffer 2).
step 14: the CD45+ MNC cells were recovered using a CD32 antibody according to standard immunohistochemistry procedures.

### Example 4

Detecting the Expression Level of Total Polynucleotides Isolated from CD45-Positive Cells

The protocol below was applied to both blood samples obtained from female and male patients suffering from myocardial ischemia and female and male control subjects not suffering from myocardial ischemia

Total RNA was isolated from CD45+ MNC cells, which themselves were isolated from blood sample from subjects (i.e. female or male suffering from myocardial ischemia) and control subjects (i.e. female or male subjects not suffering from myocardial ischemia) according to standard protocols.

A genome wide polynucleotide expression exon microarray analysis was performed according to the manufacturer protocol (Affymetrix Human Gene 2.1 ST Exon Microarray, Affymetrix Santa Clara, CA, USA) on the total RNA that was isolated from CD45+ MNC cells obtained from each experimental group and for each sex so as to identify potential RNA biomarkers that are differentially expressed in CD45+ circulating cells derived from sample obtained from female and male patients suffering from myocardial ischemia (i.e. angina pectoris) vs. control female and male subjects in which myocardial ischemia was ruled out. The polynucleotide expression exon microarray analysis was performed according to the manufacturer's instructions. The relative polynucleotide expression profile between female patients suffering from myocardial ischemia and female control subjects was established. The same was performed for the male patients suffering from myocardial ischemia and male control subjects. An in depth bioinformatical analysis was performed to determine the probe sets (thereby RNA biomarkers) being indicative of IVD or IVDC in respectively female and male subjects (see Example 5).

### Example 5

### Statistical Analysis of Affymetrix Output for Female and Male Specific RNA Biomarkers and Number of RNA Biomarkers

Affymetrix software was used to extract, normalize, and summarize intensity data from the Affymetrix Exon Microarray Human Gene 2.1 ST.

684 probe sets from the Affymetrix exon microarray based upon ANOVA significance were evaluated for RNA biomarker signature for male subjects for differentiating IVD or IVDCs subjects from control subjects without IVD or IVDCs. The resulting RNA biomarker signature was composed of an optimal 16 probe set for male subjects using Linear Discriminant Analysis (LDA) in Limma statistical package in Bioconductor. The inclusion criterion was statistical significance using a false discovery rate (FDR) of <0.02.

451 probe sets from the Affymetrix exon microarray based upon ANOVA significance were evaluated for RNA biomarker signature for female subjects for differentiating IVD or IVDCs subjects from control subjects without IVD or IVDCs. The resulting RNA biomarker signature was composed of an optimal 14 probe set for male subjects using Linear Discriminant Analysis (LDA) in Limma statistical package in Bioconductor. The inclusion criterion was statistical significance using a p value of <0.001. The MisClassification LDA for the optimal 14 probe set was p<0.001.

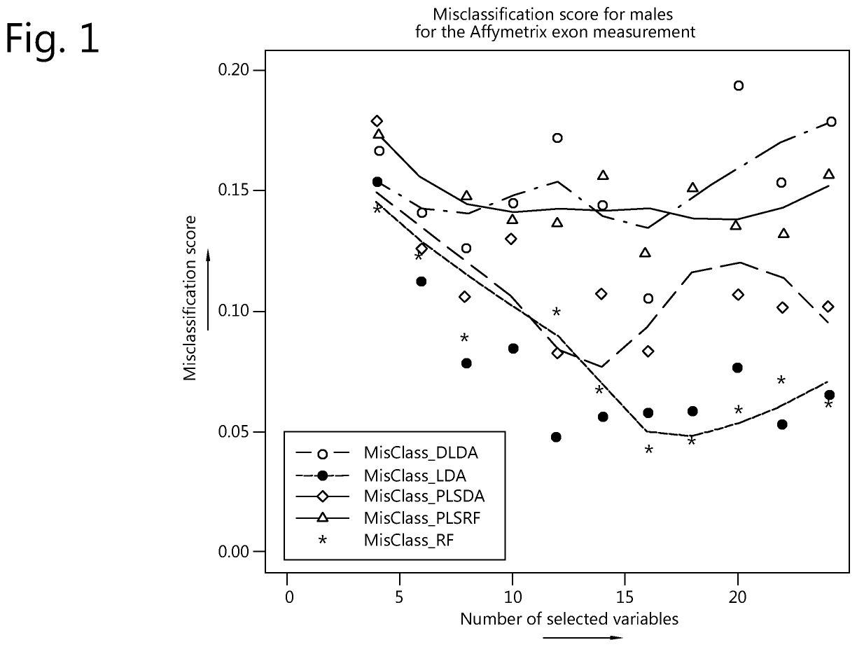

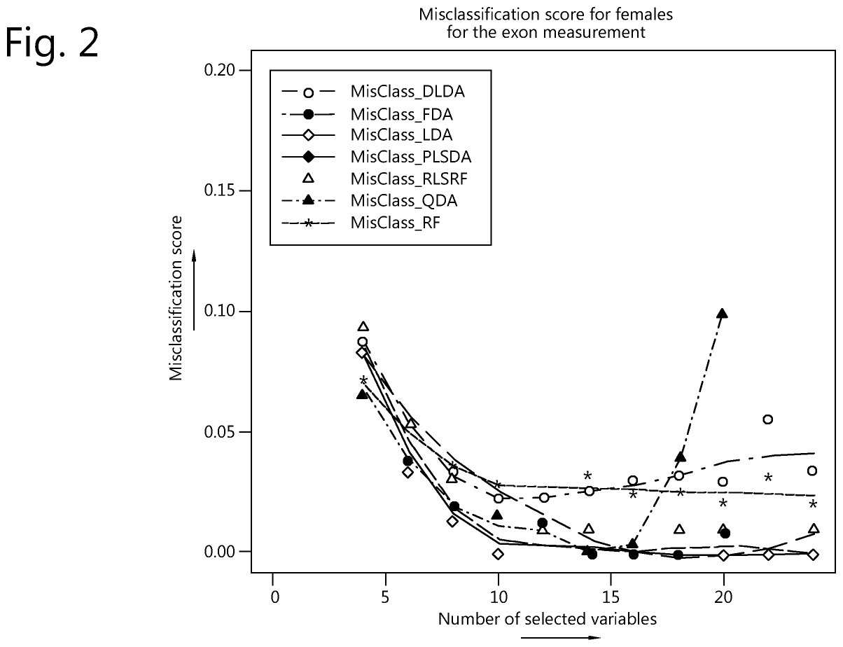

FIGS. 1 and 2 show misclassification scores for the male and female exons probe set for identifying the RNA biomarkers, respectively, depending on the number of optimized exons and for different prediction methods. Methods of data analysis comprised the Diagonal Linear Discriminant Analysis (DLDA), (regular) Linear Discriminant Analysis (LDA), Fisher Discriminant Analysis (FDA), Quadratic Discriminant Analysis (QDA), Partial Least Squares followed by Linear Discriminant Analysis (PLSLDA), Partial Least Squares followed by Random Forrest (PLSRF) or Random Forest (RF) for the classification of the microarray data.

For both the female and male exon probe set data showed that the Linear Discriminant Analysis (LDA) gave the best performance due to its sensitivity to outliers and robustness compared to the other methods. FIGS. 1 and 2 show that the best performance, the preferred number of optimized exons is 16 and 14 for male and female exon probe sets (for identifying the RNA biomarkers), respectively. The optimal probe sets and RNA biomarkers for female subjects and male subjects were identified and are presented in tables 3 and 6, respectively.

### REFERENCES

Bastiaansen et al. TLR4 accessory molecule RP105 (CD180) regulates monocyte-driven arteriogenesis in a murine hind limb ischemia model. PLoS One. 2014 Jun. 19; 9(6).
Bot et al. Local Mast Cell Activation Promotes Neovascularization. Cells. 2020 Mar. 12; 9(3):701.
Gibbons et al. in Circulation (2004) Suppl. IV: IV-47-IV-58.
Robin et al. in Journal of the American College of Cardiology, Vol. 50, No. 8 (2007), pp. 727-737.
Sambrook et al. (1989).
Sambrook and Russell (2001).
Smyth, G. K. Limma: linear models for microarray data. Bioinformatics and Computational Biology Solutions using R and Bioconductor. Springer, 2005, 397-420.

What is claimed is:

1. An in vitro method for diagnosis of occurrence and/or assessing severity of ischemic vascular disease (IVD) and/or ischemic vascular disease-related complication (IVDC) in a female subject, said method comprising the steps of:
a. determining a first expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) from a test sample collected from said female subject, and
b. comparing said first expression level of each RNA biomarker from the group consisting of ADAMTS8, AK094845, AMOT, BTRC, CCDC57, CNTNAP3, COL2A1, DCHS2, DENND5B, FOXN4, GTF2IRD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) collected from said female subject with a control expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC 007214, TDG and UROC1 from a control sample collected from control female subject(s),
c. diagnosing occurrence and/or assessing severity of IVD and/or IVDC based on the differences between the first expression level and the control expression level, wherein it is determined that IVD and/or IVDC occurred or is severe if at least two, preferably at least five, preferably at least ten, more preferably at least fourteen, RNA biomarkers from said group are different between the first expression level and the control expression level.

2. The method according to claim 1, comprising the steps of:
d. determining a second expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) from a further test sample collected from said subject at a later time point than the first test sample,
e. comparing the first expression level of each RNA biomarker with the second expression level of each RNA biomarker,
f. diagnosing whether the progression of IVD and/or IVDC over time in said female subject has occurred based on the difference of at least two, preferably at least five, preferably at least ten, more preferably at least fourteen, RNA biomarkers from said group between the first expression level and the second expression level in said female subject.

3. The method according to claim 1, comprising the steps of:
d. monitoring a treatment for IVD and/or IVDC after step c.,
e. determining a second expression level of each RNA biomarker from the group consisting of ADAMTS8, AK094845, AMOT, BTRC, CCDC57, CNTNAP3, COL2A1, DCHS2, DENND5B, FOXN4, GTF21RD1, HNRNPR, KIF18B, LPAR6, NBLA00301, NKAIN3, NRXN2, MMP11, PRSS33, PTS, QTRTD1, STK36, TBCB, TCONS_00015170-XLOC_007214, TDG and UROC1, in CD45+ mononuclear cells (MNC) from a further test sample collected from said subject at a later time point than the first test sample,
f. comparing the first expression level of each RNA biomarker with the second expression level of each RNA biomarker,
g. determining whether the treatment for IVD and/or IVDC has improved the occurrence and/or severity of IVD and/or IVDC in said female subject based on the difference of at least two, preferably at least five, preferably at least ten, more preferably at least fourteen, RNA biomarkers from said group between the first expression level and the second expression level in said female subject.

4. An in vitro method for diagnosis of occurrence and/or assessing severity of ischemic vascular disease (IVD) and/or ischemic vascular disease-related complication (IVDC) in a male subject, said method comprising the steps of:
a. determining a first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) from a test sample collected from said male subject, and
b. comparing said first expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) collected from said male subject with a control expression level of each RNA biomarker from the group consisting of ENST00000440123, TCONS_00025321-XLOC_012127, MTPAP, ENST00000430988, USP12-AS1, ProbSetID_17118935, TRH, XR_109422/1 LOC100507049, ENST00000456944, TBX5, TCONS 12_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID 5299845 and TCONS 00017554-XLOC_008249 from a control sample collected from control male subject(s),
c. diagnosing occurrence and/or assessing severity of IVD and/or IVDC based on the differences between the first expression level and the control expression level, wherein it is determined that IVD and/or IVDC occurred or is severe if at least two, preferably at least ten, more preferably at least fourteen, more preferably at least sixteen, more preferably at least eighteen, RNA biomarkers from said group are different between the first expression level and the control expression level.

5. The method according to claim 4, comprising the steps of:
d. determining a second expression level of each RNA biomarker from the group consisting of ENST00000440123, TCONS_00025321-XLOC_012127, MTPAP, ENST00000430988, USP12-AS1, ProbSetlD_17118935, TRH, XR_109422 I/LOC100507049, ENST00000456944, TBX5, TCONS_I2_00026656-XLOC_I2_013873, ExonID_5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) from a further test sample collected from said subject at a later time point than the first test sample,
e. comparing the first expression level of each RNA biomarker with the second expression level of each RNA biomarker,
f. diagnosing whether the progression of IVD and/or IVDC over time in said male subject has occurred based on the difference of at least two, preferably at least ten, more preferably at least fourteen, more preferably at least sixteen, more preferably at least eighteen, RNA biomarkers from said group between the first expression level and the second expression level in said male subject.

6. The method according to claim 4, comprising the steps of:
d. monitoring a treatment for IVD and/or IVDC after step c.,
e. determining a second expression level of each RNA biomarker from the group consisting of ENST00000440123, TCONS_00025321-XLOC_012127, MTPAP, ENST00000430988, USP12-AS1, ProbSetlD_17118935, TRH, XR_109422//LOC100507049, ENST00000456944, TBX5, TCONS_I2_00026656-XLOC_I2_013873, ExonID-5262586, RGL3, MFSD2B, CALD1, ENST00000518932, TOM1L2, LAMA4, COQ6, SALL4, CABLES1, POLRMT, SLC23A1, TREML2, SNHG15, CYP51A1, ExonID_5299845 and TCONS_00017554-XLOC_008249, in CD45+ mononuclear cells (MNC) from a further test sample collected from said subject at a later time point than the first test sample,
f. comparing the first expression level of each RNA biomarker with the second expression level of each RNA biomarker,
g. determining whether the treatment for IVD and/or IVDC has improved the occurrence and/or severity of IVD and/or IVDC in said male subject based on the difference of at least two, preferably at least ten, more preferably at least fourteen, more preferably at least sixteen, more preferably at least eighteen, RNA biomarkers from said group between the first expression level and the second expression level in said male subject.

7. The method according to claim 1, wherein IVD and/or IVDC is early onset of IVD and/or early onset of IVDC in said subject.

8. The method according to claim 1, wherein the test sample and the control sample are a biological sample selected from the group consisting of urine, serum, plasma, buffy coat, stool, saliva, tissue specimen, blood, blood cells or preferably mononuclear cells.

9. The method according to claim 1, wherein the expression level of each RNA biomarker is determined by northern blotting, nuclease protection assay, in situ hybridization, DNA microarray, exon DNA microarray or quantitative RT-PCR, biochemical assays, Luminex technology, chromatographic assays, mass spectrometric assays or hybridization assays using labelled hybridization probes such as those as taught herein), RNA/DNA microarray assay, RNA/DNA Chip assays or immunoassays, ELISA assays.

10. The method according to claim 1, wherein at least fourteen RNA biomarkers from the group consisting of ADAMTS8, AK094845, AMOT, BTRC, CNTNAP3, COL2A1, DCHS2, DENND5B, LPAR6, MMP11, NBLA00301, PRSS33, QTRTD1 and TDG expression levels are different between the first test sample and the further or control sample.

11. The method according to claim 4, wherein at least sixteen RNA biomarkers from the group consisting of CYP51A1, TREML2, ProbSetlD_17118935, TRH, TCONS_I2_00026656-XLOC 12_013873, SLC23A1, TCONS_00017554-XLOC_008249, POLRMT, ENST00000518932, COQ6, ExonID_5299845, MFSD2B, SNHG15, CABLES1, RGL3 and TCONS_00025321-XLOC_012127 expression levels are different between the first test sample and the further or control sample.


SEQUENCE LISTING (1348 sequences)

SEQ ID NO: 1 - DNA, 25 bp, synthetic construct
  misc_feature 1..25: note = >Female | AK125911 | 16928030
  source 1..25: mol_type = other DNA
cattttggccaacagtcgaagacgg

SEQ ID NO: 2 - DNA, 25 bp, synthetic construct
  misc_feature 1..25: note = >Female | AK125911 | 16928030
  source 1..25: mol_type = other DNA
cacagcttattcaagaagactccac

SEQ ID NO: 3 - DNA, 25 bp, synthetic construct
  misc_feature 1..25: note = >Female | AK125911 | 16928030
  source 1..25: mol_type = other DNA
tgtttcacagcttattcaagaagac

SEQ ID NO: 4 - DNA, 25 bp, synthetic construct
  misc_feature 1..25: note = >Female | AK125911 | 16928030
  source 1..25: mol_type = other DNA
tgcagagccacgatccaggcagtgg

SEQ ID NO: 5 - DNA, 25 bp, synthetic construct
  misc_feature 1..25: note = >Female | AK125911 | 16928030
  source 1..25: mol_type = other DNA
tctcactgggttgtgggttctggcc

SEQ ID NO: 6 - DNA, 25 bp, synthetic construct
  misc_feature 1..25: note = >Female | AK125911 | 16928030
  source 1..25: mol_type = other DNA
tggcacagcgcagtctcactgggtt

SEQ ID NO: 7 - DNA, 25 bp, synthetic construct
  misc_feature 1..25: note = >Female | AK125911 | 16928030
  source 1..25: mol_type = other DNA
ctcttctgtcggttgcgggcactca

SEQ ID NO: 8 - DNA, 25 bp, synthetic construct
  misc_feature 1..25: note = >Female | AK125911 | 16928030
  source 1..25: mol_type = other DNA
aggtgaggtccgtcttctcccagcg

SEQ ID NO: 9 - DNA, 25 bp, synthetic construct
  misc_feature 1..25: note = >Female | AK125911 | 16928030
  source 1..25: mol_type = other DNA
tgtaggtgaggtccgtcttctccca

SEQ ID NO: 10 - DNA, 25 bp, synthetic construct
  misc_feature 1..25: note = >Female | AK125911 | 16928030
  source 1..25: mol_type = other DNA
gtggcatcctagtgtcctgctccca

SEQ ID NO: 11 - DNA, 25 bp, synthetic construct
  misc_feature 1..25: note = >Female | AK125911 | 16928030
  source 1..25: mol_type = other DNA
aggtggcatcctagtgtcctgctcc

SEQ ID NO: 12 - DNA, 25 bp, synthetic construct
  misc_feature 1..25: note = >Female | AK125911 | 16928030
  source 1..25: mol_type = other DNA
tctttgcatgatgacggaggctctg

SEQ ID NO: 13 - DNA, 25 bp, synthetic construct
  misc_feature 1..25: note = >Female | AK125911 | 16928030
  source 1..25: mol_type = other DNA
taaaggctgcctgagtgactgaaca

SEQ ID NO: 14 - DNA, 25 bp, synthetic construct
  misc_feature 1..25: note = >Female | AK125911 | 16928030
  source 1..25: mol_type = other DNA
caactgccatgggaaccgaaggatc

SEQ ID NO: 15 - DNA, 25 bp, synthetic construct
  misc_feature 1..25: note = >Female | AK125911 | 16928030
  source 1..25: mol_type = other DNA
cctcagtaaaggtgagtggcgtcac

SEQ ID NO: 16 - DNA, 25 bp, synthetic construct
  misc_feature 1..25: note = >Female | AK125911 | 16928030
  source 1..25: mol_type = other DNA
tcatcatagtcgaagtggacatccc

SEQ ID NO: 17 - DNA, 25 bp, synthetic construct
  misc_feature 1..25: note = >Female | AK125911 | 16928030
  source 1..25: mol_type = other DNA
tggtcatccccgatagtccaggtct

SEQ ID NO: 18 - DNA, 25 bp, synthetic construct
  misc_feature 1..25: note = >Female | AK125911 | 16928030
  source 1..25: mol_type = other DNA
catataggtgttgaacgcccctgca

SEQ ID NO: 19 - DNA, 25 bp, synthetic construct
  misc_feature 1..25: note = >Female | AK125911 | 16928030
  source 1..25: mol_type = other DNA
tcattggtgtctatcccagcctggg

SEQ ID NO: 20 - DNA, 25 bp, synthetic construct
  misc_feature 1..25: note = >Female | AK125911 | 16928030
  source 1..25: mol_type = other DNA
catatgctgcagtcacagtggccag

SEQ ID NO: 21 - DNA, 25 bp, synthetic construct
  misc_feature 1..25: note = >Female | AK125911 | 16928030
  source 1..25: mol_type = other DNA
gacacatgctgagggcatatgctgc

SEQ ID NO: 22 - DNA, 25 bp, synthetic construct
  misc_feature 1..25: note = >Female | AK125911 | 16928030
  source 1..25: mol_type = other DNA
tttgaagaaaaagagctcgcctcgg

SEQ ID NO: 23 - DNA, 25 bp, synthetic construct
  misc_feature 1..25: note = >Female | AK125911 | 16928030
  source 1..25: mol_type = other DNA
cgagaacctcctacctgtgcctgag

SEQ ID NO: 24 - DNA, 25 bp, synthetic construct
  misc_feature 1..25: note = >Female | AK125911 | 16928030
  source 1..25: mol_type = other DNA
gagggttgaacgcaccaacacgagg

SEQ ID NO: 25 - DNA, 25 bp, synthetic construct
  misc_feature 1..25: note = >Female | AK125911 | 16928030
  source 1..25: mol_type = other DNA
tctgccagttgaggatggaaatccc

SEQ ID NO: 26 - DNA, 25 bp, synthetic construct
  misc_feature 1..25: note = >Female | AK125911 | 16928030
  source 1..25: mol_type = other DNA
tgtgaagtccctggcatctgcaatg

SEQ ID NO: 27 - DNA, 25 bp, synthetic construct
  misc_feature 1..25: note = >Female | AK125911 | 16928030
  source 1..25: mol_type = other DNA
tctgtaaactagtgcacgggtgagg

SEQ ID NO: 28 - DNA, 25 bp, synthetic construct
  misc_feature 1..25: note = >Female | AK125911 | 16928030
  source 1..25: mol_type = other DNA
caggactggcttttcaccgtcgtac

SEQ ID NO: 29 - DNA, 25 bp, synthetic construct
  misc_feature 1..25: note = >Female | AK125911 | 16928030
  source 1..25: mol_type = other DNA
agaccaaggcagcatggaccgggaa

SEQ ID NO: 30 - DNA, 25 bp, synthetic construct
  misc_feature 1..25: note = >Female | AK125911 | 16928030
  source 1..25: mol_type = other DNA
agagggcacccctctccagtcagtg

SEQ ID NO: 31 - DNA, 25 bp, synthetic construct
  misc_feature 1..25: note = >Female | AK125911 | 16928030
  source 1..25: mol_type = other DNA
aagagccagacttcctgtggcaggc

SEQ ID NO: 32 - DNA, 25 bp, synthetic construct
  misc_feature 1..25: note = >Female | AK125911 | 16928030
  source 1..25: mol_type = other DNA
gagccttcaccttcacagggtcaaa

SEQ ID NO: 33 - DNA, 25 bp, synthetic construct
  misc_feature 1..25: note = >Female | AK299689 | 17069388
  source 1..25: mol_type = other DNA
gaggatcggcctgtctagttaagag

SEQ ID NO: 34 - DNA, 25 bp, synthetic construct
  misc_feature 1..25: note = >Female | AK299689 | 17069388
  source 1..25: mol_type = other DNA
tgcgtcccactgtgcaggtctgttt

SEQ ID NO: 35 - DNA, 25 bp, synthetic construct
  misc_feature 1..25: note = >Female | AK299689 | 17069388
  source 1..25: mol_type = other DNA
gctcgcgcccgcctttgttagtttc

SEQ ID NO: 36 - DNA, 25 bp, synthetic construct
  misc_feature 1..25: note = >Female | AK299689 | 17069388
  source 1..25: mol_type = other DNA
ggcgctgactgccagagatcctcgt

SEQ ID NO: 37 - DNA, 25 bp, synthetic construct
  misc_feature 1..25: note = >Female | AK299689 | 17069388
  source 1..25: mol_type = other DNA
agaggcagatgagcgagcagcgtcc

SEQ ID NO: 38 - DNA, 25 bp, synthetic construct
  misc_feature 1..25: note = >Female | AK299689 | 17069388
  source 1..25: mol_type = other DNA
cgaggtctgtactgaatggtcccaa

SEQ ID NO: 39 - DNA, 25 bp, synthetic construct
  misc_feature 1..25: note = >Female | AK299689 | 17069388
  source 1..25: mol_type = other DNA
gagagtccacctacttccaaataaa

SEQ ID NO: 40 - DNA, 25 bp, synthetic construct
  misc_feature 1..25: note = >Female | AK299689 | 17069388
  source 1..25: mol_type = other DNA
tgtaatcgtccatcatgccatgggc

SEQ ID NO: 41 - DNA, 25 bp, synthetic construct
  misc_feature 1..25: note = >Female | AK299689 | 17069388
  source 1..25: mol_type = other DNA
tactggaagtcaacgatgcagcctg

SEQ ID NO: 42 - DNA, 25 bp, synthetic construct
  misc_feature 1..25: note = >Female | AK299689 | 17069388
  source 1..25: mol_type = other DNA
ggtctgcagtgagtacggctccaca

SEQ ID NO: 43 - DNA, 25 bp, synthetic construct
  misc_feature 1..25: note = >Female | AK299689 | 17069388
  source 1..25: mol_type = other DNA
taacaggcatacacaaaacccacca

SEQ ID NO: 44 - DNA, 25 bp, synthetic construct
  misc_feature 1..25: note = >Female | AK299689 | 17069388
  source 1..25: mol_type = other DNA
ggaaatactgatcacataacaggca

SEQ ID NO: 45 - DNA, 25 bp, synthetic construct
  misc_feature 1..25: note = >Female | AK299689 | 17069388
  source 1..25: mol_type = other DNA
gagtgtgtatcaagtccacctatga

SEQ ID NO: 46 - DNA, 25 bp, synthetic construct
  misc_feature 1..25: note = >Female | AK299689 | 17069388
  source 1..25: mol_type = other DNA
ggctttaactcaacatgatcctggt

SEQ ID NO: 47 - DNA, 25 bp, synthetic construct
  misc_feature 1..25: note = >Female | AK299689 | 17069388
  source 1..25: mol_type = other DNA
gaagcacagtctagcctctggaagg

SEQ ID NO: 48 - DNA, 25 bp, synthetic construct
  misc_feature 1..25: note = >Female | AK299689 | 17069388
  source 1..25: mol_type = other DNA
caaaggctgtgaccaaaatgctcca

SEQ ID NO: 49 - DNA, 25 bp, synthetic construct
  misc_feature 1..25: note = >Female | AK299689 | 17069388
  source 1..25: mol_type = other DNA
tattcaataatttgcagcatctgga

SEQ ID NO: 50 - DNA, 25 bp, synthetic construct
  misc_feature 1..25: note = >Female | AK299689 | 17069388
  source 1..25: mol_type = other DNA
gtctgtttggtgtaaggcgagctcc

SEQ ID NO: 51 - DNA, 25 bp, synthetic construct
  misc_feature 1..25: note = >Female | AK299689 | 17069388
  source 1..25: mol_type = other DNA
gatgccctccccacagaaatgagtg

SEQ ID NO: 52 - DNA, 25 bp, synthetic construct
  misc_feature 1..25: note = >Female | BC039373 | 16779021
  source 1..25: mol_type = other DNA
gtagatccagattccttttgttttc

SEQ ID NO: 53 - DNA, 25 bp, synthetic construct
  misc_feature 1..25: note = >Female | BC039373 | 16779021
  source 1..25: mol_type = other DNA
gctagggtttcctgatggtgccaaa

SEQ ID NO: 54 - DNA, 25 bp, synthetic construct
  misc_feature 1..25: note = >Female | BC039373 | 16779021
  source 1..25: mol_type = other DNA
gtaaaccgagacttctgtaacagac

SEQ ID NO: 55 - DNA, 25 bp, synthetic construct
  misc_feature 1..25: note = >Female | BC039373 | 16779021
  source 1..25: mol_type = other DNA
cacttaacgtggtcttaacatggca

SEQ ID NO: 56 - DNA, 25 bp, synthetic construct
  misc_feature 1..25: note = >Female | BC039373 | 16779021
  source 1..25: mol_type = other DNA
taagtacgtgtaagatgttagccct

SEQ ID NO: 57 - DNA, 25 bp, synthetic construct
  misc_feature 1..25: note = >Female | BC039373 | 16779021
  source 1..25: mol_type = other DNA
caaagacattacagattggcaccac

SEQ ID NO: 58 - DNA, 25 bp, synthetic construct
  misc_feature 1..25: note = >Female | BC039373 | 16779021
  source 1..25: mol_type = other DNA
tgaaagttctgtcccagtgagtcct

SEQ ID NO: 59 - DNA, 25 bp, synthetic construct
  misc_feature 1..25: note = >Female | BC039373 | 16779021
  source 1..25: mol_type = other DNA
tagactgaacaaaaacggcgggtgc

SEQ ID NO: 60 - DNA, 25 bp, synthetic construct
  misc_feature 1..25: note = >Female | BC039373 | 16779021
  source 1..25: mol_type = other DNA
gatcacagttaaccacacgccagtg

SEQ ID NO: 61 - DNA, 25 bp, synthetic construct
  misc_feature 1..25: note = >Female | BC039373 | 16779021
  source 1..25: mol_type = other DNA
catttcggactttgaggacgcagat

SEQ ID NO: 62 - DNA, 25 bp, synthetic construct
  misc_feature 1..25: note = >Female | BC039373 | 16779021
  source 1..25: mol_type = other DNA
tattaacccaagcacaaacaccatg

SEQ ID NO: 63 - DNA, 25 bp, synthetic construct
  misc_feature 1..25: note = >Female | BC039373 | 16779021
  source 1..25: mol_type = other DNA
gaaaagctgtgatctgtgaccagaa

SEQ ID NO: 64 - DNA, 25 bp, synthetic construct
  misc_feature 1..25: note = >Female | BC039373 | 16779021
  source 1..25: mol_type = other DNA
gaaatgccttctgcggtgctggcaa

SEQ ID NO: 65 - DNA, 25 bp, synthetic construct
  misc_feature 1..25: note = >Female | BC039373 | 16779021
  source 1..25: mol_type = other DNA
gatgattctcacatgtttgctgatg

SEQ ID NO: 66 - DNA, 25 bp, synthetic construct
  misc_feature 1..25: note = >Female | BC039373 | 16779021
  source 1..25: mol_type = other DNA
gaatgtgccacgaggaacgatgcat

SEQ ID NO: 67 - DNA, 25 bp, synthetic construct
  misc_feature 1..25: note = >Female | BC039373 | 16779021
  source 1..25: mol_type = other DNA
tttgccatggtgaaatgtgacttcc

SEQ ID NO: 68 - DNA, 25 bp, synthetic construct
  misc_feature 1..25: note = >Female | BC039373 | 16779021
  source 1..25: mol_type = other DNA
gtatatagtttagctctccttccag

SEQ ID NO: 69 - DNA, 25 bp, synthetic construct
  misc_feature 1..25: note = >Female | BC039373 | 16779021
  source 1..25: mol_type = other DNA
aaatgttctcgtagtaacaggtagc

SEQ ID NO: 70 - DNA, 25 bp, synthetic construct
  misc_feature 1..25: note = >Female | BC039373 | 16779021
  source 1..25: mol_type = other DNA
tcactttagcccaaatgctgggaaa

SEQ ID NO: 71 - DNA, 25 bp, synthetic construct
  misc_feature 1..25: note = >Female | BC039373 | 16779021
  source 1..25: mol_type = other DNA
tagtgttccagaaaataggctttgg

SEQ ID NO: 72 - DNA, 25 bp, synthetic construct
  misc_feature 1..25: note = >Female | ENST00000468156 | 16861376
  source 1..25: mol_type = other DNA
aagccatgaaacctctgcgcggaga

SEQ ID NO: 73 - DNA, 25 bp, synthetic construct
  misc_feature 1..25: note = >Female | ENST00000468156 | 16861376
  source 1..25: mol_type = other DNA
agctcgcccagtccgcgaagggtaa

SEQ ID NO: 74 - DNA, 25 bp, synthetic construct
  misc_feature 1..25: note = >Female | ENST00000468156 | 16861376
  source 1..25: mol_type = other DNA
tagttcacggttcttccaatagcgg

SEQ ID NO: 75 - DNA, 25 bp, synthetic construct
  misc_feature 1..25: note = >Female | ENST00000468156 | 16861376
  source 1..25: mol_type = other DNA
gatgaaaacggtcaccgtgggtgcc

SEQ ID NO: 76 - DNA, 25 bp, synthetic construct
  misc_feature 1..25: note = >Female | ENST00000468156 | 16861376
  source 1..25: mol_type = other DNA
ggctgtatcgcttctcggagcggaa

SEQ ID NO: 77 - DNA, 25 bp, synthetic construct
  misc_feature 1..25: note = >Female | ENST00000468156 | 16861376
  source 1..25: mol_type = other DNA
tccagttccatgcaggaagcagggc

SEQ ID NO: 78 - DNA, 25 bp, synthetic construct
  misc_feature 1..25: note = >Female | ENST00000468156 | 16861376
  source 1..25: mol_type = other DNA
cagcttgctgtagaacttgtcgtca

SEQ ID NO: 79 - DNA, 25 bp, synthetic construct
  misc_feature 1..25: note = >Female | ENST00000468156 | 16861376
  source 1..25: mol_type = other DNA
ggcagccgtcatctacagggtagga

SEQ ID NO: 80 - DNA, 25 bp, synthetic construct
  misc_feature 1..25: note = >Female | ENST00000468156 | 16861376
  source 1..25: mol_type = other DNA
gagtgtcccagatagagagtcctca

SEQ ID NO: 81 - DNA, 25 bp, synthetic construct
  misc_feature 1..25: note = >Female | ENST00000468156 | 16861376
  source 1..25: mol_type = other DNA
tatctcgggtatgactgagggcaaa

SEQ ID NO: 82 - DNA, 25 bp, synthetic construct
  misc_feature 1..25: note = >Female | ENST00000468156 | 16861376
  source 1..25: mol_type = other DNA
taatgatgggtctgtctctcgtcag

SEQ ID NO: 83 - DNA, 25 bp, synthetic construct
  misc_feature 1..25: note = >Female | ENST00000468156 | 16861376
  source 1..25: mol_type = other DNA
gtcctcatactcaccaaggcgggcg

SEQ ID NO: 84 - DNA, 25 bp, synthetic construct
  misc_feature 1..25: note = >Female | ENST00000468156 | 16861376
  source 1..25: mol_type = other DNA
cctctggtcgtaggcttcttgtgag

SEQ ID NO: 85 - DNA, 25 bp, synthetic construct
  misc_feature 1..25: note = >Female | ENST00000468156 | 16861376
  source 1..25: mol_type = other DNA
cgcttcaggaaagagcggaccgtgt

SEQ ID NO: 86 - DNA, 25 bp, synthetic construct
  misc_feature 1..25: note = >Female | ENST00000468156 | 16861376
  source 1..25: mol_type = other DNA
gttgtaccggccgagcttgctgcgc

SEQ ID NO: 87 - DNA, 25 bp, synthetic construct
  misc_feature 1..25: note = >Female | ENST00000468156 | 16861376
  source 1..25: mol_type = other DNA
ctacatacatgacggtgccccggcg

SEQ ID NO: 88 - DNA, 25 bp, synthetic construct
  misc_feature 1..25: note = >Female | ENST00000468156 | 16861376
  source 1..25: mol_type = other DNA
cgtggtctccgaggctccactgtgc

SEQ ID NO: 89 - DNA, 25 bp, synthetic construct
  misc_feature 1..25: note = >Female | ENST00000468156 | 16861376
  source 1..25: mol_type = other DNA
agagcgtggtctccgaggctccact

SEQ ID NO: 90 - DNA, 25 bp, synthetic construct
  misc_feature 1..25: note = >Female | ENST00000468156 | 16861376
  source 1..25: mol_type = other DNA
tccagggactgagatcgtttcgagt

SEQ ID NO: 91 - DNA, 25 bp, synthetic construct
  misc_feature 1..25: note = >Female | ENST00000468156 | 16861376
  source 1..25: mol_type = other DNA
cctccagggactgagatcgtttcga

SEQ ID NO: 92 - DNA, 25 bp, synthetic construct
  misc_feature 1..25: note = >Female | ENST00000468156 | 16861376
  source 1..25: mol_type = other DNA
ttcgaagcgcacaggccgcgtgcct

SEQ ID NO: 93 - DNA, 25 bp, synthetic construct
  misc_feature 1..25: note = >Female | ENST00000468156 | 16861376
  source 1..25: mol_type = other DNA
gctactccttctggcgggaactctt

SEQ ID NO: 94 - DNA, 25 bp, synthetic construct
  misc_feature 1..25: note = >Female | ENST00000468156 | 16861376
  source 1..25: mol_type = other DNA
tagccaggcttgaaatctgtgagac

SEQ ID NO: 95 - DNA, 25 bp, synthetic construct
  misc_feature 1..25: note = >Female | ENST00000468156 | 16861376
  source 1..25: mol_type = other DNA
gacaccaatccagtagccaggcttg

SEQ ID NO: 96 - DNA, 25 bp, synthetic construct
  misc_feature 1..25: note = >Female | ENST00000468156 | 16861376
  source 1..25: mol_type = other DNA
tttccccagtggctcatcatagcgg

SEQ ID NO: 97 - DNA, 25 bp, synthetic construct
  misc_feature 1..25: note = >Female | ENST00000468156 | 16861376
  source 1..25: mol_type = other DNA
ggcattcgaagtagcgtttcccatt

SEQ ID NO: 98 - DNA, 25 bp, synthetic construct
  misc_feature 1..25: note = >Female | ENST00000468156 | 16861376
  source 1..25: mol_type = other DNA
ttggcctggcattcgaagtagcgtt

SEQ ID NO: 99 - DNA, 25 bp, synthetic construct
  misc_feature 1..25: note = >Female | ENST00000468156 | 16861376
  source 1..25: mol_type = other DNA
tgctggcttgacaaaggcgccatac

SEQ ID NO: 100 - DNA, 25 bp, synthetic construct
  misc_feature 1..25: note = >Female | ENST00000468156 | 16861376
  source 1..25: mol_type = other DNA
taatgcatgagtctcaaaaatcaat

SEQ ID NO: 101 - DNA, 25 bp, synthetic construct
  misc_feature 1..25: note = >Female | ENST00000468156 | 16861376
  source 1..25: mol_type = other DNA
tagtgaatttaatgcatgagtctca

SEQ ID NO: 102 - DNA, 25 bp, synthetic construct
  misc_feature 1..25: note = >Female | ENST00000468156 | 16861376
  source 1..25: mol_type = other DNA
ctagtgaatttaatgcatgagtctc

SEQ ID NO: 103 - DNA, 25 bp, synthetic construct
  misc_feature 1..25: note = >Female | ENST00000493991 | 16763818
  source 1..25: mol_type = other DNA
tcaggtcaggtcagccattcagtgc

SEQ ID NO: 104 - DNA, 25 bp, synthetic construct
  misc_feature 1..25: note = >Female | ENST00000493991 | 16763818
  source 1..25: mol_type = other DNA
tgtgaccggtactcgataacagtct

SEQ ID NO: 105 - DNA, 25 bp, synthetic construct
  misc_feature 1..25: note = >Female | ENST00000493991 | 16763818
  source 1..25: mol_type = other DNA
cagaaaaccttcatggcgtccaagg

SEQ ID NO: 106 - DNA, 25 bp, synthetic construct
  misc_feature 1..25: note = >Female | ENST00000493991 | 16763818
  source 1..25: mol_type = other DNA
gcaaaggcggacatgtcgatgccag

SEQ ID NO: 107 - DNA, 25 bp, synthetic construct
  misc_feature 1..25: note = >Female | ENST00000493991 | 16763818
  source 1..25: mol_type = other DNA
cattagcaccatctttgccagaggg

SEQ ID NO: 108 - DNA, 25 bp, synthetic construct
  misc_feature 1..25: note = >Female | ENST00000493991 | 16763818
  source 1..25: mol_type = other DNA
tgcagaccagtgaagccacggtgtc

SEQ ID NO: 109 - DNA, 25 bp, synthetic construct
  misc_feature 1..25: note = >Female | ENST00000493991 | 16763818
  source 1..25: mol_type = other DNA
tccagctggtcctgagggtcccatg

SEQ ID NO: 110 - DNA, 25 bp, synthetic construct
  misc_feature 1..25: note = >Female | ENST00000493991 | 16763818
  source 1..25: mol_type = other DNA
tccttgcttgccagttggaccagcg

SEQ ID NO: 111 - DNA, 25 bp, synthetic construct
  misc_feature 1..25: note = >Female | ENST00000493991 | 16763818
  source 1..25: mol_type = other DNA
gctgtctcctcgagcacctttggga

SEQ ID NO: 112 - DNA, 25 bp, synthetic construct
  misc_feature 1..25: note = >Female | ENST00000493991 | 16763818
  source 1..25: mol_type = other DNA
taggaccagtcactccagtaggacc

SEQ ID NO: 113 - DNA, 25 bp, synthetic construct
  misc_feature 1..25: note = >Female | ENST00000493991 | 16763818
  source 1..25: mol_type = other DNA
ttctcgccattagcaccagctgggc

SEQ ID NO: 114 - DNA, 25 bp, synthetic construct
  misc_feature 1..25: note = >Female | ENST00000493991 | 16763818
  source 1..25: mol_type = other DNA
gctaggatcctaatgcccagcagtc

SEQ ID NO: 115 - DNA, 25 bp, synthetic construct
  misc_feature 1..25: note = >Female | ENST00000493991 | 16763818
  source 1..25: mol_type = other DNA
tttgggcccagcgataccagctgct

SEQ ID NO: 116 - DNA, 25 bp, synthetic construct
  misc_feature 1..25: note = >Female | ENST00000493991 | 16763818
  source 1..25: mol_type = other DNA
ggagagccacgttcacctgggaaac

SEQ ID NO: 117 - DNA, 25 bp, synthetic construct
  misc_feature 1..25: note = >Female | ENST00000493991 | 16763818
  source 1..25: mol_type = other DNA
tgagctgagccagtgttccagagac

SEQ ID NO: 118 - DNA, 25 bp, synthetic construct
  misc_feature 1..25: note = >Female | ENST00000493991 | 16763818
  source 1..25: mol_type = other DNA
tcttgacctgggaaaccgcggttgc

SEQ ID NO: 119 - DNA, 25 bp, synthetic construct
  misc_feature 1..25: note = >Female | ENST00000493991 | 16763818
  source 1..25: mol_type = other DNA
ctttgaagccagcaataccaggttc

SEQ ID NO: 120 - DNA, 25 bp, synthetic construct
  misc_feature 1..25: note = >Female | ENST00000493991 | 16763818
  source 1..25: mol_type = other DNA
cagagcctcctcaggtttcttctca

SEQ ID NO: 121 - DNA, 25 bp, synthetic construct
  misc_feature 1..25: note = >Female | ENST00000493991 | 16763818
  source 1..25: mol_type = other DNA
tggctgaccatcgttgcctcgggca

SEQ ID NO: 122 - DNA, 25 bp, synthetic construct
  misc_feature 1..25: note = >Female | ENST00000493991 | 16763818
  source 1..25: mol_type = other DNA
agcagggccagtccgtcctctttca

SEQ ID NO: 123 - DNA, 25 bp, synthetic construct
  misc_feature 1..25: note = >Female | ENST00000493991 | 16763818
  source 1..25: mol_type = other DNA
cattgggcccggagatccgttctca

SEQ ID NO: 124 - DNA, 25 bp, synthetic construct
  misc_feature 1..25: note = >Female | ENST00000493991 | 16763818
  source 1..25: mol_type = other DNA
tagcaccgtccaggcctggataacc

SEQ ID NO: 125 - DNA, 25 bp, synthetic construct
  misc_feature 1..25: note = >Female | ENST00000493991 | 16763818
  source 1..25: mol_type = other DNA
cacaatcaaggaaggcggcagcttc

SEQ ID NO: 126 - DNA, 25 bp, synthetic construct
  misc_feature 1..25: note = >Female | ENST00000493991 | 16763818
  source 1..25: mol_type = other DNA
gaaggacccctctagtgtctcaggc

SEQ ID NO: 127 - DNA, 25 bp, synthetic construct
  misc_feature 1..25: note = >Female | ENST00000493991 | 16763818
  source 1..25: mol_type = other DNA
gaggcagtctttcacgtcttcacag

SEQ ID NO: 128 - DNA, 25 bp, synthetic construct
  misc_feature 1..25: note = >Female | ENST00000493991 | 16763818
  source 1..25: mol_type = other DNA
gaatagcagggactagtctcctcca

SEQ ID NO: 129 - DNA, 25 bp, synthetic construct
  misc_feature 1..25: note = >Female | ENST00000493991 | 16763818
  source 1..25: mol_type = other DNA
tatccctggaaacagcctggtggca

SEQ ID NO: 130 - DNA, 25 bp, synthetic construct
  misc_feature 1..25: note = >Female | ENST00000493991 | 16763818
  source 1..25: mol_type = other DNA
tgacaccgaaggacagcggcgacga

SEQ ID NO: 131 - DNA, 25 bp, synthetic construct
  misc_feature 1..25: note = >Female | ENST00000531175 | 16731259
  source 1..25: mol_type = other DNA
cgggacacttgtgcctggcagcgac

SEQ ID NO: 132 - DNA, 25 bp, synthetic construct
  misc_feature 1..25: note = >Female | ENST00000531175 | 16731259
  source 1..25: mol_type = other DNA
ctgtacaatcggtggctcgcgctga

SEQ ID NO: 133 - DNA, 25 bp, synthetic construct
  misc_feature 1..25: note = >Female | ENST00000531175 | 16731259
  source 1..25: mol_type = other DNA
gggccttccacttttcaattcaaca

SEQ ID NO: 134 - DNA, 25 bp, synthetic construct
  misc_feature 1..25: note = >Female | ENST00000531175 | 16731259
  source 1..25: mol_type = other DNA
caactgatctgctcatgggccttcc

SEQ ID NO: 135 - DNA, 25 bp, synthetic construct
  misc_feature 1..25: note = >Female | ENST00000531175 | 16731259
  source 1..25: mol_type = other DNA
tataattgtgcccatggccatttgg

SEQ ID NO: 136 - DNA, 25 bp, synthetic construct
  misc_feature 1..25: note = >Female | ENST00000531175 | 16731259
  source 1..25: mol_type = other DNA
tactttcttcgaacactgctggcaa

SEQ ID NO: 137 - DNA, 25 bp, synthetic construct
  misc_feature 1..25: note = >Female | ENST00000531175 | 16731259
  source 1..25: mol_type = other DNA
cacaagttagcaacatacaagcaca

SEQ ID NO: 138 - DNA, 25 bp, synthetic construct
  misc_feature 1..25: note = >Female | ENST00000531175 | 16731259
  source 1..25: mol_type = other DNA
gactttcaacagatcaacatccaag

SEQ ID NO: 139 - DNA, 25 bp, synthetic construct
  misc_feature 1..25: note = >Female | ENST00000531175 | 16731259
  source 1..25: mol_type = other DNA
tgcacatttgccttcatcccagaga

SEQ ID NO: 140 - DNA, 25 bp, synthetic construct
  misc_feature 1..25: note = >Female | ENST00000531175 | 16731259
  source 1..25: mol_type = other DNA
tgataggctctccatattaaacggc

SEQ ID NO: 141 - DNA, 25 bp, synthetic construct
  misc_feature 1..25: note = >Female | ENST00000531175 | 16731259
  source 1..25: mol_type = other DNA
tgagatcagccagattcataaccat

SEQ ID NO: 142 - DNA, 25 bp, synthetic construct
  misc_feature 1..25: note = >Female | ENST00000531175 | 16731259
  source 1..25: mol_type = other DNA
ttgagatcagccagattcataacca

SEQ ID NO: 143 - DNA, 25 bp, synthetic construct
  misc_feature 1..25: note = >Female | ENST00000531175 | 16731259
  source 1..25: mol_type = other DNA
catctgcaaagtatggcacatccat

SEQ ID NO: 144 - DNA, 25 bp, synthetic construct
  misc_feature 1..25: note = >Female | ENST00000531175 | 16731259
  source 1..25: mol_type = other DNA
cataaggcaagatacagtgccaccc

SEQ ID NO: 145 - DNA, 25 bp, synthetic construct
  misc_feature 1..25: note = >Female | ENST00000531175 | 16731259
  source 1..25: mol_type = other DNA
tcagaaatcacacaacaatcaaagt

SEQ ID NO: 146 - DNA, 25 bp, synthetic construct
  misc_feature 1..25: note = >Female | ENST00000531175 | 16731259
  source 1..25: mol_type = other DNA
tataaacagctacattttcagtcgt

SEQ ID NO: 147 - DNA, 25 bp, synthetic construct
  misc_feature 1..25: note = >Female | ENST00000531175 | 16731259
  source 1..25: mol_type = other DNA
gaagaactttctggaggttgtccca

SEQ ID NO: 148 - DNA, 25 bp, synthetic construct
  misc_feature 1..25: note = >Female | ENST00000531175 | 16731259
  source 1..25: mol_type = other DNA
taagattcacctactcgaagtcaca

SEQ ID NO: 149 - DNA, 25 bp, synthetic construct
  misc_feature 1..25: note = >Female | ENST00000531175 | 16731259
  source 1..25: mol_type = other DNA
tcagtggtgggaacaaggtttcctc

SEQ ID NO: 150 - DNA, 25 bp, synthetic construct
  misc_feature 1..25: note = >Female | ENST00000531175 | 16731259
  source 1..25: mol_type = other DNA
gatgctctgggtaaacactggaagg

SEQ ID NO: 151 - DNA, 25 bp, synthetic construct
  misc_feature 1..25: note = >Female | ENST00000531175 | 16731259
  source 1..25: mol_type = other DNA
caggccgtcatgttgggagatgctc

SEQ ID NO: 152 - DNA, 25 bp, synthetic construct
  misc_feature 1..25: note = >Female | ENST00000531175 | 16731259
  source 1..25: mol_type = other DNA
tgctcactctgctgaagcaggccgt

SEQ ID NO: 153 - DNA, 25 bp, synthetic construct
  misc_feature 1..25: note = >Female | ENST00000531175 | 16731259
  source 1..25: mol_type = other DNA
caccccttggatgtgtacacgacat

SEQ ID NO: 154 - DNA, 25 bp, synthetic construct
  misc_feature 1..25: note = >Female | ENST00000531175 | 16731259
  source 1..25: mol_type = other DNA
cacagatcacgagatatatggcaga

SEQ ID NO: 155 - DNA, 25 bp, synthetic construct
  misc_feature 1..25: note = >Female | NM_001102398 | 16683280
  source 1..25: mol_type = other DNA
taatggcactgattacagaggaatc

SEQ ID NO: 156 - DNA, 25 bp, synthetic construct
  misc_feature 1..25: note = >Female | NM_001102398 | 16683280
  source 1..25: mol_type = other DNA
gactggtgagaaacgaagtacaccc

SEQ ID NO: 157 - DNA, 25 bp, synthetic construct
  misc_feature 1..25: note = >Female | NM_001102398 | 16683280
  source 1..25: mol_type = other DNA
ggaaaaggtcttgcaaatgtcccta

SEQ ID NO: 158 - DNA, 25 bp, synthetic construct
  misc_feature 1..25: note = >Female | NM_001102398 | 16683280
  source 1..25: mol_type = other DNA
tacaaggctgacataattgcccact

SEQ ID NO: 159 - DNA, 25 bp, synthetic construct
  misc_feature 1..25: note = >Female | NM_001102398 | 16683280
  source 1..25: mol_type = other DNA
catgacatgtcaggtcagggcagcc

SEQ ID NO: 160 - DNA, 25 bp, synthetic construct
  misc_feature 1..25: note = >Female | NM_001102398 | 16683280
  source 1..25: mol_type = other DNA
gcatatttgtatcggtctcaccagg

SEQ ID NO: 161 - DNA, 25 bp, synthetic construct
  misc_feature 1..25: note = >Female | NM_001102398 | 16683280
  source 1..25: mol_type = other DNA
tcatgatttaacagtgtgcccagct

SEQ ID NO: 162 - DNA, 25 bp, synthetic construct
  misc_feature 1..25: note = >Female | NM_001102398 | 16683280
  source 1..25: mol_type = other DNA
tgcaagcccaataatgtccaagagc

SEQ ID NO: 163 - DNA, 25 bp, synthetic construct
  misc_feature 1..25: note = >Female | NM_001102398 | 16683280
  source 1..25: mol_type = other DNA
gaaccacggcctctctgctgttgag

SEQ ID NO: 164 - DNA, 25 bp, synthetic construct
  misc_feature 1..25: note = >Female | NM_001102398 | 16683280
  source 1..25: mol_type = other DNA
tcatagccgtagtaatctggagggt

SEQ ID NO: 165 - DNA, 25 bp, synthetic construct
  misc_feature 1..25: note = >Female | NM_001102398 | 16683280
  source 1..25: mol_type = other DNA
ggtggcttggctaagactatttcaa

SEQ ID NO: 166 - DNA, 25 bp, synthetic construct
  misc_feature 1..25: note = >Female | NM_001102398 | 16683280
  source 1..25: mol_type = other DNA
tacgagccagacaaagattaacttc

SEQ ID NO: 167 - DNA, 25 bp, synthetic construct
  misc_feature 1..25: note = >Female | NM_001102398 | 16683280
  source 1..25: mol_type = other DNA
tatttcttctgtcaccgtagtagcc

SEQ ID NO: 168 - DNA, 25 bp, synthetic construct
  misc_feature 1..25: note = >Female | NM_001102398 | 16683280
  source 1..25: mol_type = other DNA
tctgtcaccgtagtagccaagtttc

SEQ ID NO: 169 - DNA, 25 bp, synthetic construct
  misc_feature 1..25: note = >Female | NM_001102398 | 16683280
  source 1..25: mol_type = other DNA
ttacttttccactcatcagccggcg

SEQ ID NO: 170 - DNA, 25 bp, synthetic construct
  misc_feature 1..25: note = >Female | NM_001102398 | 16683280
  source 1..25: mol_type = other DNA
ttgatgatagagaataacgtccacc

SEQ ID NO: 171 - DNA, 25 bp, synthetic construct
  misc_feature 1..25: note = >Female | NM_001102398 | 16683280
  source 1..25: mol_type = other DNA
tcggaatggatccaacaaaaagtct

SEQ ID NO: 172 - DNA, 25 bp, synthetic construct
  misc_feature 1..25: note = >Female | NM_001102398 | 16683280
  source 1..25: mol_type = other DNA
cactccaaggtgtttaccagggcga

SEQ ID NO: 173 - DNA, 25 bp, synthetic construct
  misc_feature 1..25: note = >Female | NM_001102398 | 16683280
  source 1..25: mol_type = other DNA
aatgcataccctctattctgaccgg

SEQ ID NO: 174 - DNA, 25 bp, synthetic construct
  misc_feature 1..25: note = >Female | NM_001102398 | 16683280
  source 1..25: mol_type = other DNA
taagacgtagatcccaaatgggtcc

SEQ ID NO: 175 - DNA, 25 bp, synthetic construct
  misc_feature 1..25: note = >Female | NM_001102398 | 16683280
  source 1..25: mol_type = other DNA
caattccaggttgcacgccagagta

SEQ ID NO: 176 - DNA, 25 bp, synthetic construct
  misc_feature 1..25: note = >Female | NM_001102398 | 16683280
  source 1..25: mol_type = other DNA
cgcttcatcaggtccctttgtggac

SEQ ID NO: 177 - DNA, 25 bp, synthetic construct
  misc_feature 1..25: note = >Female | NM_001102398 | 16683280
  source 1..25: mol_type = other DNA
tttgtggactcttgcaccttgctcc

SEQ ID NO: 178 - DNA, 25 bp, synthetic construct
  misc_feature 1..25: note = >Female | NM_001102398 | 16683280
  source 1..25: mol_type = other DNA
ccacttgttgatatgtgactgtatc

SEQ ID NO: 179 - DNA, 25 bp, synthetic construct
  misc_feature 1..25: note = >Female | NM_001102398 | 16683280
  source 1..25: mol_type = other DNA
gtgactgtatctagactgtctttgc

SEQ ID NO: 180 - DNA, 25 bp, synthetic construct
  misc_feature 1..25: note = >Female | NM_001102398 | 16683280
  source 1..25: mol_type = other DNA
ttctgaaactctgccacaagcagtt

SEQ ID NO: 181 - DNA, 25 bp, synthetic construct
  misc_feature 1..25: note = >Female | NM_001102398 | 16683280
  source 1..25: mol_type = other DNA
agacatttctgagtaacgaccttac

SEQ ID NO: 182 - DNA, 25 bp, synthetic construct
  misc_feature 1..25: note = >Female | NM_001102398 | 16683280
  source 1..25: mol_type = other DNA
gtagtacagacagagctccttcttc

SEQ ID NO: 183 - DNA, 25 bp, synthetic construct
  misc_feature 1..25: note = >Female | NM_001102398 | 16683280
  source 1..25: mol_type = other DNA
tcatcaagatcgacataagctacca

SEQ ID NO: 184 - DNA, 25 bp, synthetic construct
  misc_feature 1..25: note = >Female | NM_001102398 | 16683280
  source 1..25: mol_type = other DNA
gaggcctgcctctatcagtgtcttg

SEQ ID NO: 185 - DNA, 25 bp, synthetic construct
  misc_feature 1..25: note = >Female | NM_001102398 | 16683280
  source 1..25: mol_type = other DNA
tgtaccgcattaccattcacctgat

SEQ ID NO: 186 - DNA, 25 bp, synthetic construct
  misc_feature 1..25: note = >Female | NM_001102398 | 16683280
  source 1..25: mol_type = other DNA
aaaacgactagaaatggcgcgcgcg

SEQ ID NO: 187 - DNA, 25 bp, synthetic construct
  misc_feature 1..25: note = >Female | NM_001113490 | 17113427
  source 1..25: mol_type = other DNA
taaagcctctgtcgggtaaacacca

SEQ ID NO: 188 - DNA, 25 bp, synthetic construct
  misc_feature 1..25: note = >Female | NM_001113490 | 17113427
  source 1..25: mol_type = other DNA
ccattaatcacaagggaggccagaa

SEQ ID NO: 189 - DNA, 25 bp, synthetic construct
  misc_feature 1..25: note = >Female | NM_001113490 | 17113427
  source 1..25: mol_type = other DNA
gagtattggagtggaacacaggccc

SEQ ID NO: 190 - DNA, 25 bp, synthetic construct
  misc_feature 1..25: note = >Female | NM_001113490 | 17113427
  source 1..25: mol_type = other DNA
caaacgatgtggtccaggaccggta

SEQ ID NO: 191 - DNA, 25 bp, synthetic construct
  misc_feature 1..25: note = >Female | NM_001113490 | 17113427
  source 1..25: mol_type = other DNA
cacagccggagctgaagttggtgcc

SEQ ID NO: 192 - DNA, 25 bp, synthetic construct
  misc_feature 1..25: note = >Female | NM_001113490 | 17113427
  source 1..25: mol_type = other DNA
tcagtttgggtactgcagtctcggc

SEQ ID NO: 193 - DNA, 25 bp, synthetic construct
  misc_feature 1..25: note = >Female | NM_001113490 | 17113427
  source 1..25: mol_type = other DNA
gacatattcagcacggtagtctcca

SEQ ID NO: 194 - DNA, 25 bp, synthetic construct
  misc_feature 1..25: note = >Female | NM_001113490 | 17113427
  source 1..25: mol_type = other DNA
catgggtgcttatctcagggctctc

SEQ ID NO: 195 - DNA, 25 bp, synthetic construct
  misc_feature 1..25: note = >Female | NM_001113490 | 17113427
  source 1..25: mol_type = other DNA
gaaatggacatcagagacttcgctg

SEQ ID NO: 196 - DNA, 25 bp, synthetic construct
  misc_feature 1..25: note = >Female | NM_001113490 | 17113427
  source 1..25: mol_type = other DNA
ggcatccttctcaataatctgggca

SEQ ID NO: 197 - DNA, 25 bp, synthetic construct
  misc_feature 1..25: note = >Female | NM_001113490 | 17113427
  source 1..25: mol_type = other DNA
gcatccttctcaataatctgggcat

SEQ ID NO: 198 - DNA, 25 bp, synthetic construct
  misc_feature 1..25: note = >Female | NM_001113490 | 17113427
  source 1..25: mol_type = other DNA
catgtcaaggcaacgcttattggcc

SEQ ID NO: 199 - DNA, 25 bp, synthetic construct
  misc_feature 1..25: note = >Female | NM_001113490 | 17113427
  source 1..25: mol_type = other DNA
catagctggtgttaggagagtgact

SEQ ID NO: 200 - DNA, 25 bp, synthetic construct
  misc_feature 1..25: note = >Female | NM_001113490 | 17113427
  source 1..25: mol_type = other DNA
tattctgaaacgttggtgggctgac

SEQ ID NO: 201 - DNA, 25 bp, synthetic construct
  misc_feature 1..25: note = >Female | NM_001113490 | 17113427
  source 1..25: mol_type = other DNA
cagtcgtgtccggagacggtgctct

SEQ ID NO: 202 - DNA, 25 bp, synthetic construct
  misc_feature 1..25: note = >Female | NM_001113490 | 17113427
  source 1..25: mol_type = other DNA
actcagggcctgatctcggatttcg

SEQ ID NO: 203 - DNA, 25 bp, synthetic construct
  misc_feature 1..25: note = >Female | NM_001113490 | 17113427
  source 1..25: mol_type = other DNA
ctcattggtagaacgggcagtggcc

SEQ ID NO: 204 - DNA, 25 bp, synthetic construct
  misc_feature 1..25: note = >Female | NM_001113490 | 17113427
  source 1..25: mol_type = other DNA
caaagagctgcgagatggtttttct

SEQ ID NO: 205 - DNA, 25 bp, synthetic construct
  misc_feature 1..25: note = >Female | NM_001113490 | 17113427
  source 1..25: mol_type = other DNA
gaaatcatgcatcctccgaatctcg

SEQ ID NO: 206 - DNA, 25 bp, synthetic construct
  misc_feature 1..25: note = >Female | NM_001113490 | 17113427
  source 1..25: mol_type = other DNA
gaggttctcatatgcctccgagacg

SEQ ID NO: 207 - DNA, 25 bp, synthetic construct
  misc_feature 1..25: note = >Female | NM_001113490 | 17113427
  source 1..25: mol_type = other DNA
tctggaaacaatggcaaaagggtct

SEQ ID NO: 208 - DNA, 25 bp, synthetic construct
  misc_feature 1..25: note = >Female | NM_001113490 | 17113427
  source 1..25: mol_type = other DNA
agattgtagcaagggcaaggacccc

SEQ ID NO: 209 - DNA, 25 bp, synthetic construct
  misc_feature 1..25: note = >Female | NM_001113490 | 17113427
  source 1..25: mol_type = other DNA
gatgctcactatagaagtgccctgg

SEQ ID NO: 210 - DNA, 25 bp, synthetic construct
  misc_feature 1..25: note = >Female | NM_001113490 | 17113427
  source 1..25: mol_type = other DNA
taacaggtggatgggccttaactcc

SEQ ID NO: 211 - DNA, 25 bp, synthetic construct
  misc_feature 1..25: note = >Female | NM_001113490 | 17113427
  source 1..25: mol_type = other DNA
tagaaggcagctccaacactggcat

SEQ ID NO: 212 - DNA, 25 bp, synthetic construct
  misc_feature 1..25: note = >Female | NM_001113490 | 17113427
  source 1..25: mol_type = other DNA
gagagagagaaattgggcacctggg

SEQ ID NO: 213 - DNA, 25 bp, synthetic construct
  misc_feature 1..25: note = >Female | NM_001113490 | 17113427
  source 1..25: mol_type = other DNA
gagagagaaattgggcacctgggct

SEQ ID NO: 214 - DNA, 25 bp, synthetic construct
  misc_feature 1..25: note = >Female | NM_001113490 | 17113427
  source 1..25: mol_type = other DNA
aattcttgcttatgagcagctccag

SEQ ID NO: 215 - DNA, 25 bp, synthetic construct
  misc_feature 1..25: note = >Female | NM_001113490 | 17113427
  source 1..25: mol_type = other DNA
gccaagagtccaaccactggtcact

SEQ ID NO: 216 - DNA, 25 bp, synthetic construct
  misc_feature 1..25: note = >Female | NM_001113490 | 17113427
  source 1..25: mol_type = other DNA
tctcagccttccttctaaggagaca

SEQ ID NO: 217 - DNA, 25 bp, synthetic construct
  misc_feature 1..25: note = >Female | NM_001113490 | 17113427
  source 1..25: mol_type = other DNA
ggagacaaaagctctttcactctgc

SEQ ID NO: 218 - DNA, 25 bp, synthetic construct
  misc_feature 1..25: note = >Female | NM_001113490 | 17113427
  source 1..25: mol_type = other DNA
gaagacaaggtgacgactctctgct

SEQ ID NO: 219 - DNA, 25 bp, synthetic construct
  misc_feature 1..25: note = >Female | NM_001113490 | 17113427
  source 1..25: mol_type = other DNA
gggtctaccagtttaggcagccaga

SEQ ID NO: 220 - DNA, 25 bp, synthetic construct
  misc_feature 1..25: note = >Female | NM_001265577 | 16845809
  source 1..25: mol_type = other DNA
tcaggacattctggcggtcaggaca

SEQ ID NO: 221 - DNA, 25 bp, synthetic construct
  misc_feature 1..25: note = >Female | NM_001265577 | 16845809
  source 1..25: mol_type = other DNA
cattcctccggttgctagggcacag

SEQ ID NO: 222 - DNA, 25 bp, synthetic construct
  misc_feature 1..25: note = >Female | NM_001265577 | 16845809
  source 1..25: mol_type = other DNA
gaaggaaagtgacgctctgtccccg

SEQ ID NO: 223 - DNA, 25 bp, synthetic construct
  misc_feature 1..25: note = >Female | NM_001265577 | 16845809
  source 1..25: mol_type = other DNA
tgagatgcttggcaataccctaggc

SEQ ID NO: 224 - DNA, 25 bp, synthetic construct
  misc_feature 1..25: note = >Female | NM_001265577 | 16845809
  source 1..25: mol_type = other DNA
ctcgcaacgcgcttcttcttgcagg

SEQ ID NO: 225 - DNA, 25 bp, synthetic construct
  misc_feature 1..25: note = >Female | NM_001265577 | 16845809
  source 1..25: mol_type = other DNA
aaactgggctttgagggaggctcct

SEQ ID NO: 226 - DNA, 25 bp, synthetic construct
  misc_feature 1..25: note = >Female | NM_001265577 | 16845809
  source 1..25: mol_type = other DNA
atagtccgggtcacagggccagtgt

SEQ ID NO: 227 - DNA, 25 bp, synthetic construct
  misc_feature 1..25: note = >Female | NM_001265577 | 16845809
  source 1..25: mol_type = other DNA
caaactctgtgatcatgtcgggcgt

SEQ ID NO: 228 - DNA, 25 bp, synthetic construct
  misc_feature 1..25: note = >Female | NM_001265577 | 16845809
  source 1..25: mol_type = other DNA
tgcttagaacggtccccatccagtt

SEQ ID NO: 229 - DNA, 25 bp, synthetic construct
  misc_feature 1..25: note = >Female | NM_001265577 | 16845809
  source 1..25: mol_type = other DNA
tgagaagtggcccacgactggcttg

SEQ ID NO: 230 - DNA, 25 bp, synthetic construct
  misc_feature 1..25: note = >Female | NM_001265577 | 16845809
  source 1..25: mol_type = other DNA
gaagagttcccttccatggccctct

SEQ ID NO: 231 - DNA, 25 bp, synthetic construct
  misc_feature 1..25: note = >Female | NM_001265577 | 16845809
  source 1..25: mol_type = other DNA
tggtggtcccgacttgggagatcct

SEQ ID NO: 232 - DNA, 25 bp, synthetic construct
  misc_feature 1..25: note = >Female | NM_001265577 | 16845809
  source 1..25: mol_type = other DNA
gcatactggctgatgtgacagtcca

SEQ ID NO: 233 - DNA, 25 bp, synthetic construct
  misc_feature 1..25: note = >Female | NM_001265577 | 16845809
  source 1..25: mol_type = other DNA
ttcgcatgggtgctggatgcccgct

SEQ ID NO: 234 - DNA, 25 bp, synthetic construct
  misc_feature 1..25: note = >Female | NM_001265577 | 16845809
  source 1..25: mol_type = other DNA
gtgctgcgtgcggttacggttcccc

SEQ ID NO: 235 - DNA, 25 bp, synthetic construct
  misc_feature 1..25: note = >Female | NM_001265577 | 16845809
  source 1..25: mol_type = other DNA
tccacggtggtcaggtacatgatgc

SEQ ID NO: 236 - DNA, 25 bp, synthetic construct
  misc_feature 1..25: note = >Female | NM_001265577 | 16845809
  source 1..25: mol_type = other DNA
tgccaccccatttcaggccagggaa

SEQ ID NO: 237 - DNA, 25 bp, synthetic construct
  misc_feature 1..25: note = >Female | NM_001265577 | 16845809
  source 1..25: mol_type = other DNA
ccatcgggctcctcagggttaaaca

SEQ ID NO: 238 - DNA, 25 bp, synthetic construct
  misc_feature 1..25: note = >Female | NM_001265577 | 16845809
  source 1..25: mol_type = other DNA
tttcgtcggttctccgccgctggcg

SEQ ID NO: 239 - DNA, 25 bp, synthetic construct
  misc_feature 1..25: note = >Female | NM_015080 | 16739924
  source 1..25: mol_type = other DNA
caggctgctgctcgtggaatctaga

SEQ ID NO: 240 - DNA, 25 bp, synthetic construct
  misc_feature 1..25: note = >Female | NM_015080 | 16739924
  source 1..25: mol_type = other DNA
aaggagtcctccgtgataatgggca

SEQ ID NO: 241 - DNA, 25 bp, synthetic construct
  misc_feature 1..25: note = >Female | NM_015080 | 16739924
  source 1..25: mol_type = other DNA
cccattgtagtagaggccggacacc

SEQ ID NO: 242 - DNA, 25 bp, synthetic construct
  misc_feature 1..25: note = >Female | NM_015080 | 16739924
  source 1..25: mol_type = other DNA
taaagggaccttgcttcgctcactg

SEQ ID NO: 243 - DNA, 25 bp, synthetic construct
  misc_feature 1..25: note = >Female | NM_015080 | 16739924
  source 1..25: mol_type = other DNA
cttactatggcgttgggctcgtcga

SEQ ID NO: 244 - DNA, 25 bp, synthetic construct
  misc_feature 1..25: note = >Female | NM_015080 | 16739924
  source 1..25: mol_type = other DNA
ccacgtgtaggtgatgagcgctccc

SEQ ID NO: 245 - DNA, 25 bp, synthetic construct
  misc_feature 1..25: note = >Female | NM_015080 | 16739924
  source 1..25: mol_type = other DNA
ggtccggagccaacaaaatcaactg

SEQ ID NO: 246 - DNA, 25 bp, synthetic construct
  misc_feature 1..25: note = >Female | NM_015080 | 16739924
  source 1..25: mol_type = other DNA
gatcattgcagacagggcctccata

SEQ ID NO: 247 - DNA, 25 bp, synthetic construct
  misc_feature 1..25: note = >Female | NM_015080 | 16739924
  source 1..25: mol_type = other DNA
gaacatattcttgctcagaccgcca

SEQ ID NO: 248 - DNA, 25 bp, synthetic construct
  misc_feature 1..25: note = >Female | NM_015080 | 16739924
  source 1..25: mol_type = other DNA
tgagttccccttcatcaaggacggg

SEQ ID NO: 249 - DNA, 25 bp, synthetic construct
  misc_feature 1..25: note = >Female | NM_015080 | 16739924
  source 1..25: mol_type = other DNA
aatgaagtcattgccgttgcccgag

SEQ ID NO: 250 - DNA, 25 bp, synthetic construct
  misc_feature 1..25: note = >Female | NM_015080 | 16739924
  source 1..25: mol_type = other DNA
caagaacggcgtgctgcgactattc

SEQ ID NO: 251 - DNA, 25 bp, synthetic construct
  misc_feature 1..25: note = >Female | NM_015080 | 16739924
  source 1..25: mol_type = other DNA
taataggcgatgactaaccggctgc

SEQ ID NO: 252 - DNA, 25 bp, synthetic construct
  misc_feature 1..25: note = >Female | NM_015080 | 16739924
  source 1..25: mol_type = other DNA
taggttgatgaccagccagacagcc

SEQ ID NO: 253 - DNA, 25 bp, synthetic construct
  misc_feature 1..25: note = >Female | NM_015080 | 16739924
  source 1..25: mol_type = other DNA
ttttgttggctggtgcacatcgccg

SEQ ID NO: 254 - DNA, 25 bp, synthetic construct
  misc_feature 1..25: note = >Female | NM_015080 | 16739924
  source 1..25: mol_type = other DNA
gctgtttaacgtcaggtgagccgga

SEQ ID NO: 255 - DNA, 25 bp, synthetic construct
  misc_feature 1..25: note = >Female | NM_015080 | 16739924
  source 1..25: mol_type = other DNA
tacttgacggtgctcagcgtaagcg

SEQ ID NO: 256 - DNA, 25 bp, synthetic construct
  misc_feature 1..25: note = >Female | NM_015690 | 16891029
  source 1..25: mol_type = other DNA
tctgcggttgcctagaaacgacgct

SEQ ID NO: 257 - DNA, 25 bp, synthetic construct
  misc_feature 1..25: note = >Female | NM_015690 | 16891029
  source 1..25: mol_type = other DNA
gctcctcaggccaagggacggattc

SEQ ID NO: 258 - DNA, 25 bp, synthetic construct
  misc_feature 1..25: note = >Female | NM_015690 | 16891029
  source 1..25: mol_type = other DNA
tctccaatcatctccaacacgtggt

SEQ ID NO: 259 - DNA, 25 bp, synthetic construct
  misc_feature 1..25: note = >Female | NM_015690 | 16891029
  source 1..25: mol_type = other DNA
aaaggtatccctctttgtgcaacac

SEQ ID NO: 260 - DNA, 25 bp, synthetic construct
  misc_feature 1..25: note = >Female | NM_015690 | 16891029
  source 1..25: mol_type = other DNA
tccataagaagtatgagggagtcag

SEQ ID NO: 261 - DNA, 25 bp, synthetic construct
  misc_feature 1..25: note = >Female | NM_015690 | 16891029
  source 1..25: mol_type = other DNA
caattttgggatgaacttcagggcc

SEQ ID NO: 262 - DNA, 25 bp, synthetic construct
  misc_feature 1..25: note = >Female | NM_015690 | 16891029
  source 1..25: mol_type = other DNA
aatctctcgttgcaaattcctcagc

SEQ ID NO: 263 - DNA, 25 bp, synthetic construct
  misc_feature 1..25: note = >Female | NM_015690 | 16891029
  source 1..25: mol_type = other DNA
cacaatgttgggatgccgcagaccc

SEQ ID NO: 264 - DNA, 25 bp, synthetic construct
  misc_feature 1..25: note = >Female | NM_015690 | 16891029
  source 1..25: mol_type = other DNA
tgaaagagctctccctcagcatagt

SEQ ID NO: 265 - DNA, 25 bp, synthetic construct
  misc_feature 1..25: note = >Female | NM_015690 | 16891029
  source 1..25: mol_type = other DNA
gtacagggctgacaccaactgggca

SEQ ID NO: 266 - DNA, 25 bp, synthetic construct
  misc_feature 1..25: note = >Female | NM_015690 | 16891029
  source 1..25: mol_type = other DNA
tattggtgctcatagcccgggcaaa

SEQ ID NO: 267 - DNA, 25 bp, synthetic construct
  misc_feature 1..25: note = >Female | NM_015690 | 16891029
  source 1..25: mol_type = other DNA
tcaattctgaggctaagatcccggc

SEQ ID NO: 268 - DNA, 25 bp, synthetic construct
  misc_feature 1..25: note = >Female | NM_015690 | 16891029
  source 1..25: mol_type = other DNA
tgggaatgctcgttcacaatctggg

SEQ ID NO: 269 - DNA, 25 bp, synthetic construct
  misc_feature 1..25: note = >Female | NM_015690 | 16891029
  source 1..25: mol_type = other DNA
gatacctagaaagacgtccgctgca

SEQ ID NO: 270 - DNA, 25 bp, synthetic construct
  misc_feature 1..25: note = >Female | NM_015690 | 16891029
  source 1..25: mol_type = other DNA
gaacaccagtgaccaacggataggg

SEQ ID NO: 271 - DNA, 25 bp, synthetic construct
  misc_feature 1..25: note = >Female | NM_015690 | 16891029
  source 1..25: mol_type = other DNA
tcccggcagaaggaatacaaggcaa

SEQ ID NO: 272 - DNA, 25 bp, synthetic construct
  misc_feature 1..25: note = >Female | NM_015690 | 16891029
  source 1..25: mol_type = other DNA
gtaagaaggtcccataccaagattg

SEQ ID NO: 273 - DNA, 25 bp, synthetic construct
  misc_feature 1..25: note = >Female | NM_015690 | 16891029
  source 1..25: mol_type = other DNA
tccagattgaaggtacaggcaaagt

SEQ ID NO: 274 - DNA, 25 bp, synthetic construct
  misc_feature 1..25: note = >Female | NM_015690 | 16891029
  source 1..25: mol_type = other DNA
taaaaggctttggagatggcccggc

SEQ ID NO: 275 - DNA, 25 bp, synthetic construct
  misc_feature 1..25: note = >Female | NM_015690 | 16891029
  source 1..25: mol_type = other DNA
ggcagtcccacaggagcagtgcata

SEQ ID NO: 276 - DNA, 25 bp, synthetic construct
  misc_feature 1..25: note = >Female | NM_015690 | 16891029
  source 1..25: mol_type = other DNA
ggctgctgtcttcagttagctgatt

SEQ ID NO: 277 - DNA, 25 bp, synthetic construct
  misc_feature 1..25: note = >Female | NM_015690 | 16891029
  source 1..25: mol_type = other DNA
cataaacagggattccaaggccagg

SEQ ID NO: 278 - DNA, 25 bp, synthetic construct
  misc_feature 1..25: note = >Female | NM_015690 | 16891029
  source 1..25: mol_type = other DNA
cacaaggcaggttttggagccgaaa

SEQ ID NO: 279 - DNA, 25 bp, synthetic construct
  misc_feature 1..25: note = >Female | NM_015690 | 16891029
  source 1..25: mol_type = other DNA
taaagagagtagcaacgtcactgcc

SEQ ID NO: 280 - DNA, 25 bp, synthetic construct
  misc_feature 1..25: note = >Female | NM_015690 | 16891029
  source 1..25: mol_type = other DNA
ccatgggctggaggtcaaaggtcac

SEQ ID NO: 281 - DNA, 25 bp, synthetic construct
  misc_feature 1..25: note = >Female | NM_015690 | 16891029
  source 1..25: mol_type = other DNA
caactacctggtggagtcaaccgaa

SEQ ID NO: 282 - DNA, 25 bp, synthetic construct
  misc_feature 1..25: note = >Female | NM_015690 | 16891029
  source 1..25: mol_type = other DNA
gttccaaggctatcacaggcacagt

SEQ ID NO: 283 - DNA, 25 bp, synthetic construct
  misc_feature 1..25: note = >Female | NM_015690 | 16891029
  source 1..25: mol_type = other DNA
tgtatgtctgctgaaacccagggaa

SEQ ID NO: 284 - DNA, 25 bp, synthetic construct
  misc_feature 1..25: note = >Female | NM_015690 | 16891029
  source 1..25: mol_type = other DNA
gaaagctccccttcctgtgcagatg

SEQ ID NO: 285 - DNA, 25 bp, synthetic construct
  misc_feature 1..25: note = >Female | NM_015690 | 16891029
  source 1..25: mol_type = other DNA
gatacttccatgctgggacaggcag

SEQ ID NO: 286 - DNA, 25 bp, synthetic construct
  misc_feature 1..25: note = >Female | NM_015690 | 16891029
  source 1..25: mol_type = other DNA
tataaggaggtcagcatccatgtcc

SEQ ID NO: 287 - DNA, 25 bp, synthetic construct
  misc_feature 1..25: note = >Female | NM_015690 | 16891029
  source 1..25: mol_type = other DNA
cagatgggctgcaacttctgagtcc

SEQ ID NO: 288 - DNA, 25 bp, synthetic construct
  misc_feature 1..25: note = >Female | NM_015690 | 16891029
  source 1..25: mol_type = other DNA
tcaacggaagatggtagcagcagac

SEQ ID NO: 289 - DNA, 25 bp, synthetic construct
  misc_feature 1..25: note = >Female | NM_015690 | 16891029
  source 1..25: mol_type = other DNA
gatagtttctcactggcacccaggg

SEQ ID NO: 290 - DNA, 25 bp, synthetic construct
  misc_feature 1..25: note = >Female | NM_016328 | 17047164
  source 1..25: mol_type = other DNA
tgtaatcccagtaagattgccagcc

SEQ ID NO: 291 - DNA, 25 bp, synthetic construct
  misc_feature 1..25: note = >Female | NM_016328 | 17047164
  source 1..25: mol_type = other DNA
tgatttaaacaactggccggtgggc

SEQ ID NO: 292 - DNA, 25 bp, synthetic construct
  misc_feature 1..25: note = >Female | NM_016328 | 17047164
  source 1..25: mol_type = other DNA
gatctcgtctttgcgggtgaacgcg

SEQ ID NO: 293 - DNA, 25 bp, synthetic construct
  misc_feature 1..25: note = >Female | NM_016328 | 17047164
  source 1..25: mol_type = other DNA
cacctcggcgttcagtttggacagc

SEQ ID NO: 294 - DNA, 25 bp, synthetic construct
  misc_feature 1..25: note = >Female | NM_016328 | 17047164
  source 1..25: mol_type = other DNA
tccgcagaaggtacacatctgagcc

SEQ ID NO: 295 - DNA, 25 bp, synthetic construct
  misc_feature 1..25: note = >Female | NM_016328 | 17047164
  source 1..25: mol_type = other DNA
tcatactcggctggccttcggaagg

SEQ ID NO: 296 - DNA, 25 bp, synthetic construct
  misc_feature 1..25: note = >Female | NM_016328 | 17047164
  source 1..25: mol_type = other DNA
caacagtcggagaagtcttgggcac

SEQ ID NO: 297 - DNA, 25 bp, synthetic construct
  misc_feature 1..25: note = >Female | NM_016328 | 17047164
  source 1..25: mol_type = other DNA
catggacgttctggatgagaggccc

SEQ ID NO: 298 - DNA, 25 bp, synthetic construct
  misc_feature 1..25: note = >Female | NM_016328 | 17047164
  source 1..25: mol_type = other DNA
gttaaacaggatctgcacacactcc

SEQ ID NO: 299 - DNA, 25 bp, synthetic construct
  misc_feature 1..25: note = >Female | NM_016328 | 17047164
  source 1..25: mol_type = other DNA
cacaggcaatcttccggtagggcac

SEQ ID NO: 300 - DNA, 25 bp, synthetic construct
  misc_feature 1..25: note = >Female | NM_016328 | 17047164
  source 1..25: mol_type = other DNA
tgtgaaaattcgctcatcaaacatc

SEQ ID NO: 301 - DNA, 25 bp, synthetic construct
  misc_feature 1..25: note = >Female | NM_016328 | 17047164
  source 1..25: mol_type = other DNA
agcaaggtcaaggacggtggcccta

SEQ ID NO: 302 - DNA, 25 bp, synthetic construct
  misc_feature 1..25: note = >Female | NM_016328 | 17047164
  source 1..25: mol_type = other DNA
aaacttggagtacggcaccttgacg

SEQ ID NO: 303 - DNA, 25 bp, synthetic construct
  misc_feature 1..25: note = >Female | NM_016328 | 17047164
  source 1..25: mol_type = other DNA
gacctgctcccttagtgtgttggcg

SEQ ID NO: 304 - DNA, 25 bp, synthetic construct
  misc_feature 1..25: note = >Female | NM_016328 | 17047164
  source 1..25: mol_type = other DNA
ccaggttctgggagccgaaggtaca

SEQ ID NO: 305 - DNA, 25 bp, synthetic construct
  misc_feature 1..25: note = >Female | NM_016328 | 17047164
  source 1..25: mol_type = other DNA
cccgagtctgttggcgtcatcttca

SEQ ID NO: 306 - DNA, 25 bp, synthetic construct
  misc_feature 1..25: note = >Female | NM_016328 | 17047164
  source 1..25: mol_type = other DNA
tagtttataagggaccagcaccggc

SEQ ID NO: 307 - DNA, 25 bp, synthetic construct
  misc_feature 1..25: note = >Female | NM_016328 | 17047164
  source 1..25: mol_type = other DNA
ccttggcatcattgcagatttctgc

SEQ ID NO: 308 - DNA, 25 bp, synthetic construct
  misc_feature 1..25: note = >Female | NM_016328 | 17047164
  source 1..25: mol_type = other DNA
gagtgccggagtccaaatcggtgca

SEQ ID NO: 309 - DNA, 25 bp, synthetic construct
  misc_feature 1..25: note = >Female | NM_016328 | 17047164
  source 1..25: mol_type = other DNA
gatgtgaattcctgcctgagtgcct

SEQ ID NO: 310 - DNA, 25 bp, synthetic construct
  misc_feature 1..25: note = >Female | NM_016328 | 17047164
  source 1..25: mol_type = other DNA
taattaagaggtcccgggagctgca

SEQ ID NO: 311 - DNA, 25 bp, synthetic construct
  misc_feature 1..25: note = >Female | NM_016328 | 17047164
  source 1..25: mol_type = other DNA
actaaaaggcatcgatacatatccg

SEQ ID NO: 312 - DNA, 25 bp, synthetic construct
  misc_feature 1..25: note = >Female | NM_144639 | 16958674
  source 1..25: mol_type = other DNA
gaatgagattcatgagccctggccc

SEQ ID NO: 313 - DNA, 25 bp, synthetic construct
  misc_feature 1..25: note = >Female | NM_144639 | 16958674
  source 1..25: mol_type = other DNA
taaaatgaggctgtggtggcaccct

SEQ ID NO: 314 - DNA, 25 bp, synthetic construct
  misc_feature 1..25: note = >Female | NM_144639 | 16958674
  source 1..25: mol_type = other DNA
tgaggcagtgtcaccaccaaggtgc

SEQ ID NO: 315 - DNA, 25 bp, synthetic construct
  misc_feature 1..25: note = >Female | NM_144639 | 16958674
  source 1..25: mol_type = other DNA
tggtctggcagatgatctcataggc

SEQ ID NO: 316 - DNA, 25 bp, synthetic construct
  misc_feature 1..25: note = >Female | NM_144639 | 16958674
  source 1..25: mol_type = other DNA
cattggagacatcccagctgagcat

SEQ ID NO: 317 - DNA, 25 bp, synthetic construct
  misc_feature 1..25: note = >Female | NM_144639 | 16958674
  source 1..25: mol_type = other DNA
gacatcccagctgagcatcagcctg

SEQ ID NO: 318 - DNA, 25 bp, synthetic construct
  misc_feature 1..25: note = >Female | NM_144639 | 16958674
  source 1..25: mol_type = other DNA
ctccgttgtgaagggcgacccaggt

SEQ ID NO: 319 - DNA, 25 bp, synthetic construct
  misc_feature 1..25: note = >Female | NM_144639 | 16958674
  source 1..25: mol_type = other DNA
caggcatctcccacgaagttctgca

SEQ ID NO: 320 - DNA, 25 bp, synthetic construct
  misc_feature 1..25: note = >Female | NM_144639 | 16958674
  source 1..25: mol_type = other DNA
cgtcgtaaatgttggaggtctccct

SEQ ID NO: 321 - DNA, 25 bp, synthetic construct
  misc_feature 1..25: note = >Female | NM_144639 | 16958674
  source 1..25: mol_type = other DNA
cacgtcatggtgatctcggctcagg

SEQ ID NO: 322 - DNA, 25 bp, synthetic construct
  misc_feature 1..25: note = >Female | NM_144639 | 16958674
  source 1..25: mol_type = other DNA
tacaggatccttgcctgggagccca

SEQ ID NO: 323 - DNA, 25 bp, synthetic construct
  misc_feature 1..25: note = >Female | NM_144639 | 16958674
  source 1..25: mol_type = other DNA
gatccagcggatgttgtccatgtac

SEQ ID NO: 324 - DNA, 25 bp, synthetic construct
  misc_feature 1..25: note = >Female | NM_144639 | 16958674
  source 1..25: mol_type = other DNA
tactgcagcttcacagacaccttca

SEQ ID NO: 325 - DNA, 25 bp, synthetic construct
  misc_feature 1..25: note = >Female | NM_144639 | 16958674
  source 1..25: mol_type = other DNA
gatgtggccagttcgtctgtgaccg

SEQ ID NO: 326 - DNA, 25 bp, synthetic construct
  misc_feature 1..25: note = >Female | NM_144639 | 16958674
  source 1..25: mol_type = other DNA
gcacacccagcggaaaggcccaaat

SEQ ID NO: 327 - DNA, 25 bp, synthetic construct
  misc_feature 1..25: note = >Female | NM_144639 | 16958674
  source 1..25: mol_type = other DNA
cacataggaagggtagcggaactct

SEQ ID NO: 328 - DNA, 25 bp, synthetic construct
  misc_feature 1..25: note = >Female | NM_144639 | 16958674
  source 1..25: mol_type = other DNA
gggtagcggaactctgtcctgccag

SEQ ID NO: 329 - DNA, 25 bp, synthetic construct
  misc_feature 1..25: note = >Female | NM_144639 | 16958674
  source 1..25: mol_type = other DNA
gcattgccgtagtcccagaagaaga

SEQ ID NO: 330 - DNA, 25 bp, synthetic construct
  misc_feature 1..25: note = >Female | NM_144639 | 16958674
  source 1..25: mol_type = other DNA
tcctcggccaacctgttgatggctg

SEQ ID NO: 331 - DNA, 25 bp, synthetic construct
  misc_feature 1..25: note = >Female | NM_144639 | 16958674
  source 1..25: mol_type = other DNA
caccacgttgccatggtaaccaagg

SEQ ID NO: 332 - DNA, 25 bp, synthetic construct
  misc_feature 1..25: note = >Female | NM_144639 | 16958674
  source 1..25: mol_type = other DNA
ttgccatggtaaccaaggctgagca

SEQ ID NO: 333 - DNA, 25 bp, synthetic construct
  misc_feature 1..25: note = >Female | NM_144639 | 16958674
  source 1..25: mol_type = other DNA
ggaaaggacacacagtcccagggca

SEQ ID NO: 334 - DNA, 25 bp, synthetic construct
  misc_feature 1..25: note = >Female | NM_144639 | 16958674
  source 1..25: mol_type = other DNA
gcagcggtccaagctgtcagtcact

SEQ ID NO: 335 - DNA, 25 bp, synthetic construct
  misc_feature 1..25: note = >Female | NM_144639 | 16958674
  source 1..25: mol_type = other DNA
cctgtggcgtttctcaagggctgct

SEQ ID NO: 336 - DNA, 25 bp, synthetic construct
  misc_feature 1..25: note = >Female | NM_144639 | 16958674
  source 1..25: mol_type = other DNA
caggtaccgacgtgcagcattcaac

SEQ ID NO: 337 - DNA, 25 bp, synthetic construct
  misc_feature 1..25: note = >Female | NM_144639 | 16958674
  source 1..25: mol_type = other DNA
acagtgccatgaacgattccctggg

SEQ ID NO: 338 - DNA, 25 bp, synthetic construct
  misc_feature 1..25: note = >Female | NM_144639 | 16958674
  source 1..25: mol_type = other DNA
gaccgatgtagcagtagctacctgc

SEQ ID NO: 339 - DNA, 25 bp, synthetic construct
  misc_feature 1..25: note = >Female | NM_144639 | 16958674
  source 1..25: mol_type = other DNA
aaggcaaagagcttctcatactccg

SEQ ID NO: 340 - DNA, 25 bp, synthetic construct
  misc_feature 1..25: note = >Female | NM_144639 | 16958674
  source 1..25: mol_type = other DNA
gagcttctcatactccgtccgggag

SEQ ID NO: 341 - DNA, 25 bp, synthetic construct
  misc_feature 1..25: note = >Female | NM_144639 | 16958674
  source 1..25: mol_type = other DNA
gaaagaggccaagtgggtgcccact

SEQ ID NO: 342 - DNA, 25 bp, synthetic construct
  misc_feature 1..25: note = >Female | NM_144639 | 16958674
  source 1..25: mol_type = other DNA
catcttcgacaagtagaacatggtc

SEQ ID NO: 343 - DNA, 25 bp, synthetic construct
  misc_feature 1..25: note = >Female | NM_144639 | 16958674
  source 1..25: mol_type = other DNA
taatcatgtgcatgatggcggcagc

SEQ ID NO: 344 - DNA, 25 bp, synthetic construct
  misc_feature 1..25: note = >Female | NM_144639 | 16958674
  source 1..25: mol_type = other DNA
gggcaaaaccggtacatgtagatgt

SEQ ID NO: 345 - DNA, 25 bp, synthetic construct
  misc_feature 1..25: note = >Female | NM_144639 | 16958674
  source 1..25: mol_type = other DNA
tgtgtccgtacagttgcagctcctg

SEQ ID NO: 346 - DNA, 25 bp, synthetic construct
  misc_feature 1..25: note = >Female | NM_144973 | 16762944
  source 1..25: mol_type = other DNA
gataccctggatcccttcgaatgag

SEQ ID NO: 347 - DNA, 25 bp, synthetic construct
  misc_feature 1..25: note = >Female | NM_144973 | 16762944
  source 1..25: mol_type = other DNA
catacattcgagtgatggcaggaca

SEQ ID NO: 348 - DNA, 25 bp, synthetic construct
  misc_feature 1..25: note = >Female | NM_144973 | 16762944
  source 1..25: mol_type = other DNA
taatagcatttacgtagtggcagaa

SEQ ID NO: 349 - DNA, 25 bp, synthetic construct
  misc_feature 1..25: note = >Female | NM_144973 | 16762944
  source 1..25: mol_type = other DNA
gaacccatggtggaaaacttgctca

SEQ ID NO: 350 - DNA, 25 bp, synthetic construct
  misc_feature 1..25: note = >Female | NM_144973 | 16762944
  source 1..25: mol_type = other DNA
gatgctcaatctcctagccgtggtg

SEQ ID NO: 351 - DNA, 25 bp, synthetic construct
  misc_feature 1..25: note = >Female | NM_144973 | 16762944
  source 1..25: mol_type = other DNA
cctgagttatcgtgaccaatctgaa

SEQ ID NO: 352 - DNA, 25 bp, synthetic construct
  misc_feature 1..25: note = >Female | NM_144973 | 16762944
  source 1..25: mol_type = other DNA
tgggatgatcactgacctatacgga

SEQ ID NO: 353 - DNA, 25 bp, synthetic construct
  misc_feature 1..25: note = >Female | NM_144973 | 16762944
  source 1..25: mol_type = other DNA
cagaaatagtccacagcattgagag

SEQ ID NO: 354 - DNA, 25 bp, synthetic construct
  misc_feature 1..25: note = >Female | NM_144973 | 16762944
  source 1..25: mol_type = other DNA
gagaccctgagcgttggcaacataa

SEQ ID NO: 355 - DNA, 25 bp, synthetic construct
  misc_feature 1..25: note = >Female | NM_144973 | 16762944
  source 1..25: mol_type = other DNA
taaatgtgaccacaaagccgacttc

SEQ ID NO: 356 - DNA, 25 bp, synthetic construct
  misc_feature 1..25: note = >Female | NM_144973 | 16762944
  source 1..25: mol_type = other DNA
ggtctgacagtttgggttgcctcag

SEQ ID NO: 357 - DNA, 25 bp, synthetic construct
  misc_feature 1..25: note = >Female | NM_144973 | 16762944
  source 1..25: mol_type = other DNA
gaagtaaagtgacagaaaccccatc

SEQ ID NO: 358 - DNA, 25 bp, synthetic construct
  misc_feature 1..25: note = >Female | NM_144973 | 16762944
  source 1..25: mol_type = other DNA
gaaacgaaacaatcaggctgggcat

SEQ ID NO: 359 - DNA, 25 bp, synthetic construct
  misc_feature 1..25: note = >Female | NM_144973 | 16762944
  source 1..25: mol_type = other DNA
gcaccccaggaaggtagaccacata

SEQ ID NO: 360 - DNA, 25 bp, synthetic construct
  misc_feature 1..25: note = >Female | NM_144973 | 16762944
  source 1..25: mol_type = other DNA
gaatgctggcgaaggcgttctttcc

SEQ ID NO: 361 - DNA, 25 bp, synthetic construct
  misc_feature 1..25: note = >Female | NM_144973 | 16762944
  source 1..25: mol_type = other DNA
tatcaatccgagtgtcaaagacccg

SEQ ID NO: 362 - DNA, 25 bp, synthetic construct
  misc_feature 1..25: note = >Female | NM_144973 | 16762944
  source 1..25: mol_type = other DNA
caatgagctggtttatttgcttgta

SEQ ID NO: 363 - DNA, 25 bp, synthetic construct
  misc_feature 1..25: note = >Female | NM_144973 | 16762944
  source 1..25: mol_type = other DNA
tgggtaagcataggcacagaagaat

SEQ ID NO: 364 - DNA, 25 bp, synthetic construct
  misc_feature 1..25: note = >Female | NM_144973 | 16762944
  source 1..25: mol_type = other DNA
ggttagcaaagacctctcggagctg

SEQ ID NO: 365 - DNA, 25 bp, synthetic construct
  misc_feature 1..25: note = >Female | NM_144973 | 16762944
  source 1..25: mol_type = other DNA
caaaagtgtggtgatgccttctgcc

SEQ ID NO: 366 - DNA, 25 bp, synthetic construct
  misc_feature 1..25: note = >Female | NM_144973 | 16762944
  source 1..25: mol_type = other DNA
taaaatttcagtgacctccctggag

SEQ ID NO: 367 - DNA, 25 bp, synthetic construct
  misc_feature 1..25: note = >Female | NM_144973 | 16762944
  source 1..25: mol_type = other DNA
tattctgaggatagtgggcgagaac

SEQ ID NO: 368 - DNA, 25 bp, synthetic construct
  misc_feature 1..25: note = >Female | NM_144973 | 16762944
  source 1..25: mol_type = other DNA
gacgatctgcttctagccattggta

SEQ ID NO: 369 - DNA, 25 bp, synthetic construct
  misc_feature 1..25: note = >Female | NM_144973 | 16762944
  source 1..25: mol_type = other DNA
tcgatcccgcacagcacgaagtagt

SEQ ID NO: 370 - DNA, 25 bp, synthetic construct
  misc_feature 1..25: note = >Female | NM_198082 | 16850156
  source 1..25: mol_type = other DNA
caacacgtaggtgaaagcacaggca

SEQ ID NO: 371 - DNA, 25 bp, synthetic construct
  misc_feature 1..25: note = >Female | NM_198082 | 16850156
  source 1..25: mol_type = other DNA
cagtccataatgttgtagttacgga

SEQ ID NO: 372 - DNA, 25 bp, synthetic construct
  misc_feature 1..25: note = >Female | NM_198082 | 16850156
  source 1..25: mol_type = other DNA
ccaggtaggtgcgtgcttctggacc

SEQ ID NO: 373 - DNA, 25 bp, synthetic construct
  misc_feature 1..25: note = >Female | NM_198082 | 16850156
  source 1..25: mol_type = other DNA
caatgggactagaacgacaggagca

SEQ ID NO: 374 - DNA, 25 bp, synthetic construct
  misc_feature 1..25: note = >Female | NM_198082 | 16850156
  source 1..25: mol_type = other DNA
gattggcgaccatgctcctcagggt

SEQ ID NO: 375 - DNA, 25 bp, synthetic construct
  misc_feature 1..25: note = >Female | NM_198082 | 16850156
  source 1..25: mol_type = other DNA
ccaaaggtgggctgcgatccaaagg

SEQ ID NO: 376 - DNA, 25 bp, synthetic construct
  misc_feature 1..25: note = >Female | NM_198082 | 16850156
  source 1..25: mol_type = other DNA
gtgcatcacttctggattcctcttg

SEQ ID NO: 377 - DNA, 25 bp, synthetic construct
  misc_feature 1..25: note = >Female | NM_198082 | 16850156
  source 1..25: mol_type = other DNA
ggcccagctctgcacggagcctgtt

SEQ ID NO: 378 - DNA, 25 bp, synthetic construct
  misc_feature 1..25: note = >Female | NM_198082 | 16850156
  source 1..25: mol_type = other DNA
ctgaccggaggtgcaggaaaagctc

SEQ ID NO: 379 - DNA, 25 bp, synthetic construct
  misc_feature 1..25: note = >Female | NM_198082 | 16850156
  source 1..25: mol_type = other DNA
gaaagacaaaaagagtcgctgctcc

SEQ ID NO: 380 - DNA, 25 bp, synthetic construct
  misc_feature 1..25: note = >Female | NM_198082 | 16850156
  source 1..25: mol_type = other DNA
gaactagcgcctggaactgtggaac

SEQ ID NO: 381 - DNA, 25 bp, synthetic construct
  misc_feature 1..25: note = >Female | NM_198082 | 16850156
  source 1..25: mol_type = other DNA
caacagtctagggtacgaagtcagg

SEQ ID NO: 382 - DNA, 25 bp, synthetic construct
  misc_feature 1..25: note = >Female | NM_198082 | 16850156
  source 1..25: mol_type = other DNA
cctgagtgccagtccagaggatact

SEQ ID NO: 383 - DNA, 25 bp, synthetic construct
  misc_feature 1..25: note = >Female | NM_198082 | 16850156
  source 1..25: mol_type = other DNA
cagtcgtctctgtcgaggtgcggac

SEQ ID NO: 384 - DNA, 25 bp, synthetic construct
  misc_feature 1..25: note = >Female | NM_198082 | 16850156
  source 1..25: mol_type = other DNA
tcatctgggcaatcgcgtttcgcaa

SEQ ID NO: 385 - DNA, 25 bp, synthetic construct
  misc_feature 1..25: note = >Female | NM_198082 | 16850156
  source 1..25: mol_type = other DNA
tggtctcgttccagggtcacggcct

SEQ ID NO: 386 - DNA, 25 bp, synthetic construct
  misc_feature 1..25: note = >Female | NM_198082 | 16850156
  source 1..25: mol_type = other DNA
cagggcctctgacttttggatctgg

SEQ ID NO: 387 - DNA, 25 bp, synthetic construct
  misc_feature 1..25: note = >Female | NM_198082 | 16850156
  source 1..25: mol_type = other DNA
tgagcaatttgagcatcccaggcag

SEQ ID NO: 388 - DNA, 25 bp, synthetic construct
  misc_feature 1..25: note = >Female | NM_198082 | 16850156
  source 1..25: mol_type = other DNA
tctccttggcggtgtcagcctgtct

SEQ ID NO: 389 - DNA, 25 bp, synthetic construct
  misc_feature 1..25: note = >Female | NM_198082 | 16850156
  source 1..25: mol_type = other DNA
tcctggttagctgcacagagtgaag

SEQ ID NO: 390 - DNA, 25 bp, synthetic construct
  misc_feature 1..25: note = >Female | NM_198082 | 16850156
  source 1..25: mol_type = other DNA
tcctctccagctcggcgttggtggc

SEQ ID NO: 391 - DNA, 25 bp, synthetic construct
  misc_feature 1..25: note = >Female | NM_198082 | 16850156
  source 1..25: mol_type = other DNA
tgtcagcctgcagacggaattcgtg

SEQ ID NO: 392 - DNA, 25 bp, synthetic construct
  misc_feature 1..25: note = >Female | NM_213596 | 16769889
  source 1..25: mol_type = other DNA
tgaattcaagggctttcaccccagg

SEQ ID NO: 393 - DNA, 25 bp, synthetic construct
  misc_feature 1..25: note = >Female | NM_213596 | 16769889
  source 1..25: mol_type = other DNA
gagaagtctcactcaacacgggcag

SEQ ID NO: 394 - DNA, 25 bp, synthetic construct
  misc_feature 1..25: note = >Female | NM_213596 | 16769889
  source 1..25: mol_type = other DNA
gacccgtcacctgcaagtctgggaa

SEQ ID NO: 395 - DNA, 25 bp, synthetic construct
  misc_feature 1..25: note = >Female | NM_213596 | 16769889
  source 1..25: mol_type = other DNA
tgttgatgaagtctacaggagccct

SEQ ID NO: 396 - DNA, 25 bp, synthetic construct
  misc_feature 1..25: note = >Female | NM_213596 | 16769889
  source 1..25: mol_type = other DNA
gccggtcggagatcagcttgtccaa

SEQ ID NO: 397 - DNA, 25 bp, synthetic construct
  misc_feature 1..25: note = >Female | NM_213596 | 16769889
  source 1..25: mol_type = other DNA
gttggccatactccggtggatggca

SEQ ID NO: 398 - DNA, 25 bp, synthetic construct
  misc_feature 1..25: note = >Female | NM_213596 | 16769889
  source 1..25: mol_type = other DNA
ccttgcgggaggagccgctcatctt

SEQ ID NO: 399 - DNA, 25 bp, synthetic construct
  misc_feature 1..25: note = >Female | NM_213596 | 16769889
  source 1..25: mol_type = other DNA
gagacaggttgtgccgcaccgagtt

SEQ ID NO: 400 - DNA, 25 bp, synthetic construct
  misc_feature 1..25: note = >Female | NM_213596 | 16769889
  source 1..25: mol_type = other DNA
tcttcagggccatggcgatcagaca

SEQ ID NO: 401 - DNA, 25 bp, synthetic construct
  misc_feature 1..25: note = >Female | NM_213596 | 16769889
  source 1..25: mol_type = other DNA
gaaagggtctcaggctgacagccca

SEQ ID NO: 402 - DNA, 25 bp, synthetic construct
  misc_feature 1..25: note = >Female | NM_213596 | 16769889
  source 1..25: mol_type = other DNA
gggtgcagttcttgagatgaatgca

SEQ ID NO: 403 - DNA, 25 bp, synthetic construct
  misc_feature 1..25: note = >Female | NM_213596 | 16769889
  source 1..25: mol_type = other DNA
caggtgagtacagatgcggcgggtc

SEQ ID NO: 404 - DNA, 25 bp, synthetic construct
  misc_feature 1..25: note = >Female | NM_213596 | 16769889
  source 1..25: mol_type = other DNA
ggtcctgcaggccagatgagggctg

SEQ ID NO: 405 - DNA, 25 bp, synthetic construct
  misc_feature 1..25: note = >Female | NM_213596 | 16769889
  source 1..25: mol_type = other DNA
gtggctcccacatgcaggtcggcca

SEQ ID NO: 406 - DNA, 25 bp, synthetic construct
  misc_feature 1..25: note = >Female | NM_213596 | 16769889
  source 1..25: mol_type = other DNA
gtgagccacgacagcgactgcaggt

SEQ ID NO: 407 - DNA, 25 bp, synthetic construct
  misc_feature 1..25: note = >Female | NM_213596 | 16769889
  source 1..25: mol_type = other DNA
ggtgattttgccctgagtttcgaat

SEQ ID NO: 408 - DNA, 25 bp, synthetic construct
  misc_feature 1..25: note = >Female | NM_213596 | 16769889
  source 1..25: mol_type = other DNA
cattatggatgaggtgtcactttct

SEQ ID NO: 409 - DNA, 25 bp, synthetic construct
  misc_feature 1..25: note = >Female | TCONS_00015170-XLOC_007214 | 17081037
  source 1..25: mol_type = other DNA
tgaaatacagttggctgctcccgct

SEQ ID NO: 410 - DNA, 25 bp, synthetic construct
  misc_feature 1..25: note = >Female | TCONS_00015170-XLOC_007214 | 17081037
  source 1..25: mol_type = other DNA
catgctggctgctgctgtgtaataa

SEQ ID NO: 411 - DNA, 25 bp, synthetic construct
  misc_feature 1..25: note = >Female | TCONS_00015170-XLOC_007214 | 17081037
  source 1..25: mol_type = other DNA
tggtcaagcacattcaggacatcgc

SEQ ID NO: 412 - DNA, 25 bp, synthetic construct
  misc_feature 1..25: note = >Female | TCONS_00015170-XLOC_007214 | 17081037
  source 1..25: mol_type = other DNA
gaataatttccaacgcaaccatcca

SEQ ID NO: 413 - DNA, 25 bp, synthetic construct
  misc_feature 1..25: note = >Female | TCONS_00015170-XLOC_007214 | 17081037
  source 1..25: mol_type = other DNA
tccaaattttgacccacagcgtgtt

SEQ ID NO: 414 - DNA, 25 bp, synthetic construct
  misc_feature 1..25: note = >Female | TCONS_00015170-XLOC_007214 | 17081037
  source 1..25: mol_type = other DNA
ggcattgcggtgactcagttcctca

SEQ ID NO: 415 - DNA, 25 bp, synthetic construct
  misc_feature 1..25: note = >Female | TCONS_00015170-XLOC_007214 | 17081037
  source 1..25: mol_type = other DNA
tagattaacttgtgtgaagcactga

SEQ ID NO: 416 - DNA, 25 bp, synthetic construct
  misc_feature 1..25: note = >Female | TCONS_00015170-XLOC_007214 | 17081037
  source 1..25: mol_type = other DNA
tatggtgtctttaggtccaacctca

SEQ ID NO: 417 - DNA, 25 bp, synthetic construct
  misc_feature 1..25: note = >Female | TCONS_00015170-XLOC_007214 | 17081037
  source 1..25: mol_type = other DNA
tctccctacatgttgtcagaaaaag

SEQ ID NO: 418 - DNA, 25 bp, synthetic construct
  misc_feature 1..25: note = >Female | TCONS_00015170-XLOC_007214 | 17081037
  source 1..25: mol_type = other DNA
caaggtttctaacagcccagttctg

SEQ ID NO: 419 - DNA, 25 bp, synthetic construct
  misc_feature 1..25: note = >Female | TCONS_00015170-XLOC_007214 | 17081037
  source 1..25: mol_type = other DNA
actgttttcagcttgggtgagtcac

SEQ ID NO: 420 - DNA, 25 bp, synthetic construct
  misc_feature 1..25: note = >Female | TCONS_00015170-XLOC_007214 | 17081037
  source 1..25: mol_type = other DNA
ggcccgattacccacagaaagtaca

SEQ ID NO: 421 - DNA, 25 bp, synthetic construct
  misc_feature 1..25: note = >Female | TCONS_00015170-XLOC_007214 | 17081037
  source 1..25: mol_type = other DNA
catcatccacatagggtctccaaaa

SEQ ID NO: 422 - DNA, 25 bp, synthetic construct
  misc_feature 1..25: note = >Female | TCONS_00015170-XLOC_007214 | 17081037
  source 1..25: mol_type = other DNA
ggctttcctggaaccattcacaagg

SEQ ID NO: 423 - DNA, 25 bp, synthetic construct
  misc_feature 1..25: note = >Female | TCONS_00015170-XLOC_007214 | 17081037
  source 1..25: mol_type = other DNA
ctctggccaaaaaagggtctggcac

SEQ ID NO: 424 - DNA, 25 bp, synthetic construct
  misc_feature 1..25: note = >Female | TCONS_00015170-XLOC_007214 | 17081037
  source 1..25: mol_type = other DNA
aaaatatctccggactatggctagg

SEQ ID NO: 425 - DNA, 25 bp, synthetic construct
  misc_feature 1..25: note = >Female | TCONS_00015170-XLOC_007214 | 17081037
  source 1..25: mol_type = other DNA
cattatgtaacagtccagggaggct

SEQ ID NO: 426 - DNA, 25 bp, synthetic construct
  misc_feature 1..25: note = >Female | TCONS_00015170-XLOC_007214 | 17081037
  source 1..25: mol_type = other DNA
catcgctgtctgtctggagggtgcc

SEQ ID NO: 427 - DNA, 25 bp, synthetic construct
  misc_feature 1..25: note = >Female | TCONS_00015170-XLOC_007214 | 17081037
  source 1..25: mol_type = other DNA
caccttgaatgctatcagggaagct

SEQ ID NO: 428 - DNA, 25 bp, synthetic construct
  misc_feature 1..25: note = >Female | TCONS_00015170-XLOC_007214 | 17081037
  source 1..25: mol_type = other DNA
tgttctttgctgtccttgccagtgt

SEQ ID NO: 429 - DNA, 25 bp, synthetic construct
  misc_feature 1..25: note = >Female | TCONS_00015170-XLOC_007214 | 17081037
  source 1..25: mol_type = other DNA
tcctgaactcacatcagaagcatgc

SEQ ID NO: 430 - DNA, 25 bp, synthetic construct
  misc_feature 1..25: note = >Female | TCONS_00015170-XLOC_007214 | 17081037
  source 1..25: mol_type = other DNA
taagcaggtggtaccagcagggttc

SEQ ID NO: 431 - DNA, 25 bp, synthetic construct
  misc_feature 1..25: note = >Female | TCONS_00015170-XLOC_007214 | 17081037
  source 1..25: mol_type = other DNA
ggatagcagatatttcttaaacagg

SEQ ID NO: 432 - DNA, 25 bp, synthetic construct
  misc_feature 1..25: note = >Female | TCONS_00015170-XLOC_007214 | 17081037
  source 1..25: mol_type = other DNA
gatagcagatatttcttaaacagga

SEQ ID NO: 433 - DNA, 25 bp, synthetic construct
  misc_feature 1..25: note = >Female | TCONS_00015170-XLOC_007214 | 17081037
  source 1..25: mol_type = other DNA
atagcagatatttcttaaacaggac

SEQ ID NO: 434 - DNA, 25 bp, synthetic construct
  misc_feature 1..25: note = >Female | AK094845 | 16770033
  source 1..25: mol_type = other DNA
tctttgctgggaactgtggaacacc

SEQ ID NO: 435 - DNA, 25 bp, synthetic construct
  misc_feature 1..25: note = >Female | AK094845 | 16770033
  source 1..25: mol_type = other DNA
tgaacagcccactctatgtaaaggc

SEQ ID NO: 436 - DNA, 25 bp, synthetic construct
  misc_feature 1..25: note = >Female | AK094845 | 16770033
  source 1..25: mol_type = other DNA
cagaccgtgggctagaaagactcaa

SEQ ID NO: 437 - DNA, 25 bp, synthetic construct
  misc_feature 1..25: note = >Female | AK094845 | 16770033
  source 1..25: mol_type = other DNA
cgccgaatgggcttgtagacacaaa

SEQ ID NO: 438 - DNA, 25 bp, synthetic construct
  misc_feature 1..25: note = >Female | AK094845 | 16770033
  source 1..25: mol_type = other DNA
aaatgccataaggatgacagtcacc

SEQ ID NO: 439 - DNA, 25 bp, synthetic construct
  misc_feature 1..25: note = >Female | AK094845 | 16770033
  source 1..25: mol_type = other DNA
aaacaggttacttcctaatggcagc

SEQ ID NO: 440 - DNA, 25 bp, synthetic construct
  misc_feature 1..25: note = >Female | AK094845 | 16770033
  source 1..25: mol_type = other DNA
ggttaggcaattcatccactcactg

SEQ ID NO: 441 - DNA, 25 bp, synthetic construct
  misc_feature 1..25: note = >Female | AK094845 | 16770033
  source 1..25: mol_type = other DNA
tgtgcaaacagctgtaaacagatcc

SEQ ID NO: 442 - DNA, 25 bp, synthetic construct
  misc_feature 1..25: note = >Female | AK094845 | 16770033
  source 1..25: mol_type = other DNA
tgtaaacagatcctctctccagttg

SEQ ID NO: 443 - DNA, 25 bp, synthetic construct
  misc_feature 1..25: note = >Female | AK094845 | 16770033
  source 1..25: mol_type = other DNA
tctcaagatgagtcttgacagggcc

SEQ ID NO: 444 - DNA, 25 bp, synthetic construct
  misc_feature 1..25: note = >Female | AK094845 | 16770033
  source 1..25: mol_type = other DNA
gacagggccttttgagcaaagaagc

SEQ ID NO: 445 - DNA, 25 bp, synthetic construct
  misc_feature 1..25: note = >Female | AK094845 | 16770033
  source 1..25: mol_type = other DNA
aaatctgctatgtttggctgggtgc

SEQ ID NO: 446 - DNA, 25 bp, synthetic construct
  misc_feature 1..25: note = >Female | AK094845 | 16770033
  source 1..25: mol_type = other DNA
ggtgagaaatgggaactacaactga

SEQ ID NO: 447 - DNA, 25 bp, synthetic construct
  misc_feature 1..25: note = >Female | AK094845 | 16770033
  source 1..25: mol_type = other DNA
gcgtaaccagcaacctgtcagaggt

SEQ ID NO: 448 - DNA, 25 bp, synthetic construct
  misc_feature 1..25: note = >Female | AK094845 | 16770033
  source 1..25: mol_type = other DNA
catggttgttcttcagtctcattac

SEQ ID NO: 449 - DNA, 25 bp, synthetic construct
  misc_feature 1..25: note = >Female | AK094845 | 16770033
  source 1..25: mol_type = other DNA
aggccaaggtacagccacggtaacg

SEQ ID NO: 450 - DNA, 25 bp, synthetic construct
  misc_feature 1..25: note = >Female | AK094845 | 16770033
  source 1..25: mol_type = other DNA
aaggtacagccacggtaacgctgga

SEQ ID NO: 451 - DNA, 25 bp, synthetic construct
  misc_feature 1..25: note = >Female | AK094845 | 16770033
  source 1..25: mol_type = other DNA
tacagccacggtaacgctggacgca

SEQ ID NO: 452 - DNA, 25 bp, synthetic construct
  misc_feature 1..25: note = >Female | AK094845 | 16770033
  source 1..25: mol_type = other DNA
catggtaactgtgcgtcctggtcta

SEQ ID NO: 453 - DNA, 25 bp, synthetic construct
  misc_feature 1..25: note = >Female | AK094845 | 16770033
  source 1..25: mol_type = other DNA
gatcaactttccgtctgtgccaacg

SEQ ID NO: 454 - DNA, 25 bp, synthetic construct
  misc_feature 1..25: note = >Female | AK094845 | 16770033
  source 1..25: mol_type = other DNA
tagcatgtaaagtccacgagcacag

SEQ ID NO: 455 - DNA, 25 bp, synthetic construct
  misc_feature 1..25: note = >Female | AK094845 | 16770033
  source 1..25: mol_type = other DNA
ggaagtgaaaccaaagacgggcagc

SEQ ID NO: 456 - DNA, 25 bp, synthetic construct
  misc_feature 1..25: note = >Female | AK094845 | 16770033
  source 1..25: mol_type = other DNA
tgtaacgtatttctggcaaccacag

SEQ ID NO: 457 - DNA, 25 bp, synthetic construct
  misc_feature 1..25: note = >Female | AK094845 | 16770033
  source 1..25: mol_type = other DNA
tagtgcagaaacctgacccaatggc

SEQ ID NO: 458 - DNA, 25 bp, synthetic construct
  misc_feature 1..25: note = >Female | AK094845 | 16770033
  source 1..25: mol_type = other DNA
taatctgccatgttaaagacactgt

SEQ ID NO: 459 - DNA, 25 bp, synthetic construct
  misc_feature 1..25: note = >Female | AK094845 | 16770033
  source 1..25: mol_type = other DNA
cacaggaaagcatcaatgggaggca

SEQ ID NO: 460 - DNA, 25 bp, synthetic construct
  misc_feature 1..25: note = >Female | AK094845 | 16770033
  source 1..25: mol_type = other DNA
gcatgacttgcttccgtgtgactcg

SEQ ID NO: 461 - DNA, 25 bp, synthetic construct
  misc_feature 1..25: note = >Female | AK094845 | 16770033
  source 1..25: mol_type = other DNA
gcattgcaggtaggatgggccccat

SEQ ID NO: 462 - DNA, 25 bp, synthetic construct
  misc_feature 1..25: note = >Female | AK094845 | 16770033
  source 1..25: mol_type = other DNA
cagggaggatgtgcctttcagcaaa

SEQ ID NO: 463 - DNA, 25 bp, synthetic construct
  misc_feature 1..25: note = >Female | AK094845 | 16770033
  source 1..25: mol_type = other DNA
tatgaatgaagcgagaagcttgtcc

SEQ ID NO: 464 - DNA, 25 bp, synthetic construct
  misc_feature 1..25: note = >Female | AK094845 | 16770033
  source 1..25: mol_type = other DNA
aaggtcacatggtaaacagcagcgc

SEQ ID NO: 465 - DNA, 25 bp, synthetic construct
  misc_feature 1..25: note = >Female | AK094845 | 16770033
  source 1..25: mol_type = other DNA
tgcatttactgttctcttggtcccg

SEQ ID NO: 466 - DNA, 25 bp, synthetic construct
  misc_feature 1..25: note = >Female | AK094845 | 16770033
  source 1..25: mol_type = other DNA
gggtcacctccagaactgggtcatc

SEQ ID NO: 467 - DNA, 25 bp, synthetic construct
  misc_feature 1..25: note = >Female | AK094845 | 16770033
  source 1..25: mol_type = other DNA
tcccattttgtctggtaacaggatc

SEQ ID NO: 468 - DNA, 25 bp, synthetic construct
  misc_feature 1..25: note = >Female | AK094845 | 16770033
  source 1..25: mol_type = other DNA
tgtggttcagtagggctcacactgc

SEQ ID NO: 469 - DNA, 25 bp, synthetic construct
  misc_feature 1..25: note = >Female | ENST00000257359 | 16746216
  source 1..25: mol_type = other DNA
ttgaaatggatccacccctgcaggt

SEQ ID NO: 470 - DNA, 25 bp, synthetic construct
  misc_feature 1..25: note = >Female | ENST00000257359 | 16746216
  source 1..25: mol_type = other DNA
cagttgatgatagggtctgccgccc

SEQ ID NO: 471 - DNA, 25 bp, synthetic construct
  misc_feature 1..25: note = >Female | ENST00000257359 | 16746216
  source 1..25: mol_type = other DNA
ttcctgcaggagttgcctttgcccc

SEQ ID NO: 472 - DNA, 25 bp, synthetic construct
  misc_feature 1..25: note = >Female | ENST00000257359 | 16746216
  source 1..25: mol_type = other DNA
gcatttgtccagcttccgaggcgag

SEQ ID NO: 473 - DNA, 25 bp, synthetic construct
  misc_feature 1..25: note = >Female | ENST00000257359 | 16746216
  source 1..25: mol_type = other DNA
gagattcccgtccatgtcagtgtaa

SEQ ID NO: 474 - DNA, 25 bp, synthetic construct
  misc_feature 1..25: note = >Female | ENST00000257359 | 16746216
  source 1..25: mol_type = other DNA
gcatgactggtacttggctctccga

SEQ ID NO: 475 - DNA, 25 bp, synthetic construct
  misc_feature 1..25: note = >Female | ENST00000257359 | 16746216
  source 1..25: mol_type = other DNA
tcacggtgtgaaaactgtactcctc

SEQ ID NO: 476 - DNA, 25 bp, synthetic construct
  misc_feature 1..25: note = >Female | ENST00000257359 | 16746216
  source 1..25: mol_type = other DNA
cagtatctgtgtcactgggtggcca

SEQ ID NO: 477 - DNA, 25 bp, synthetic construct
  misc_feature 1..25: note = >Female | ENST00000257359 | 16746216
  source 1..25: mol_type = other DNA
taatgactgtgtggcctgcatggct

SEQ ID NO: 478 - DNA, 25 bp, synthetic construct
  misc_feature 1..25: note = >Female | ENST00000257359 | 16746216
  source 1..25: mol_type = other DNA
gaaatccggcccaaagatctgcctg

SEQ ID NO: 479 - DNA, 25 bp, synthetic construct
  misc_feature 1..25: note = >Female | ENST00000257359 | 16746216
  source 1..25: mol_type = other DNA
gctctgtgagatacatggcgctgca

SEQ ID NO: 480 - DNA, 25 bp, synthetic construct
  misc_feature 1..25: note = >Female | ENST00000257359 | 16746216
  source 1..25: mol_type = other DNA
catcacgtggtgcttgcccatgggc

SEQ ID NO: 481 - DNA, 25 bp, synthetic construct
  misc_feature 1..25: note = >Female | ENST00000257359 | 16746216
  source 1..25: mol_type = other DNA
acaaatggtcccgatgtctgccaca

SEQ ID NO: 482 - DNA, 25 bp, synthetic construct
  misc_feature 1..25: note = >Female | ENST00000257359 | 16746216
  source 1..25: mol_type = other DNA
gttgaaacgccgctgccagttgcag

SEQ ID NO: 483 - DNA, 25 bp, synthetic construct
  misc_feature 1..25: note = >Female | ENST00000257359 | 16746216
  source 1..25: mol_type = other DNA
tgcttgtagattcgggctgccacag

SEQ ID NO: 484 - DNA, 25 bp, synthetic construct
  misc_feature 1..25: note = >Female | ENST00000257359 | 16746216
  source 1..25: mol_type = other DNA
caaaccgcttggtcctactcgtggc

SEQ ID NO: 485 - DNA, 25 bp, synthetic construct
  misc_feature 1..25: note = >Female | ENST00000257359 | 16746216
  source 1..25: mol_type = other DNA
ggatggtgaactcctcgccgtccag

SEQ ID NO: 486 - DNA, 25 bp, synthetic construct
  misc_feature 1..25: note = >Female | ENST00000257359 | 16746216
  source 1..25: mol_type = other DNA
cgatcttgaactcgggcgctaggaa

SEQ ID NO: 487 - DNA, 25 bp, synthetic construct
  misc_feature 1..25: note = >Female | ENST00000257359 | 16746216
  source 1..25: mol_type = other DNA
aagcccttgccgaaggcggacaggt

SEQ ID NO: 488 - DNA, 25 bp, synthetic construct
  misc_feature 1..25: note = >Female | ENST00000257359 | 16746216
  source 1..25: mol_type = other DNA
gaatcaatcgggtgcaaggccgact

SEQ ID NO: 489 - DNA, 25 bp, synthetic construct
  misc_feature 1..25: note = >Female | ENST00000257359 | 16746216
  source 1..25: mol_type = other DNA
aagaggctcgtgcactggtctcagc

SEQ ID NO: 490 - DNA, 25 bp, synthetic construct
  misc_feature 1..25: note = >Female | ENST00000504429 | 16972455
  source 1..25: mol_type = other DNA
gatagagctatgtccacgaagggcc

SEQ ID NO: 491 - DNA, 25 bp, synthetic construct
  misc_feature 1..25: note = >Female | ENST00000504429 | 16972455
  source 1..25: mol_type = other DNA
tcttatggttatgaggaggtagcca

SEQ ID NO: 492 - DNA, 25 bp, synthetic construct
  misc_feature 1..25: note = >Female | ENST00000504429 | 16972455
  source 1..25: mol_type = other DNA
gtgaagcatctgttaaacacttgca

SEQ ID NO: 493 - DNA, 25 bp, synthetic construct
  misc_feature 1..25: note = >Female | ENST00000504429 | 16972455
  source 1..25: mol_type = other DNA
ttcaaccagaaaagagagcagagtg

SEQ ID NO: 494 - DNA, 25 bp, synthetic construct
  misc_feature 1..25: note = >Female | ENST00000504429 | 16972455
  source 1..25: mol_type = other DNA
tagccactgagaccggcgtttcgag

SEQ ID NO: 495 - DNA, 25 bp, synthetic construct
  misc_feature 1..25: note = >Female | ENST00000504429 | 16972455
  source 1..25: mol_type = other DNA
aagtttgccgcaataacgtgagcca

SEQ ID NO: 496 - DNA, 25 bp, synthetic construct
  misc_feature 1..25: note = >Female | ENST00000504429 | 16972455
  source 1..25: mol_type = other DNA
caaggtcttcgtagggactggtgtc

SEQ ID NO: 497 - DNA, 25 bp, synthetic construct
  misc_feature 1..25: note = >Female | ENST00000504429 | 16972455
  source 1..25: mol_type = other DNA
caaaatcgcccaaggtcttcgtagg

SEQ ID NO: 498 - DNA, 25 bp, synthetic construct
  misc_feature 1..25: note = >Female | ENST00000504429 | 16972455
  source 1..25: mol_type = other DNA
gaggtgcccgcacttcaaaatcgcc

SEQ ID NO: 499 - DNA, 25 bp, synthetic construct
  misc_feature 1..25: note = >Female | ENST00000504429 | 16972455
  source 1..25: mol_type = other DNA
taaacctgctttccacagatgcccg

SEQ ID NO: 500 - DNA, 25 bp, synthetic construct
  misc_feature 1..25: note = >Female | ENST00000504429 | 16972455
  source 1..25: mol_type = other DNA
caaccactttgtcggcggtgagcaa

SEQ ID NO: 501 - DNA, 25 bp, synthetic construct
  misc_feature 1..25: note = >Female | ENST00000504429 | 16972455
  source 1..25: mol_type = other DNA
cagagctatcagagtgtcgcaagaa

SEQ ID NO: 502 - DNA, 25 bp, synthetic construct
  misc_feature 1..25: note = >Female | ENST00000504429 | 16972455
  source 1..25: mol_type = other DNA
ggagaggactggtttcggttataca

SEQ ID NO: 503 - DNA, 25 bp, synthetic construct
  misc_feature 1..25: note = >Female | ENST00000504429 | 16972455
  source 1..25: mol_type = other DNA
tagactgcgaaagtgaagatcccca

SEQ ID NO: 504 - DNA, 25 bp, synthetic construct
  misc_feature 1..25: note = >Female | ENST00000504429 | 16972455
  source 1..25: mol_type = other DNA
tggagtcacaggcagtcgtagactg

SEQ ID NO: 505 - DNA, 25 bp, synthetic construct
  misc_feature 1..25: note = >Female | ENST00000504429 | 16972455
  source 1..25: mol_type = other DNA
aacctatggcttgagagtggacacc

SEQ ID NO: 506 - DNA, 25 bp, synthetic construct
  misc_feature 1..25: note = >Female | ENST00000504429 | 16972455
  source 1..25: mol_type = other DNA
gaagcagtatctggaaaggctccct

SEQ ID NO: 507 - DNA, 25 bp, synthetic construct
  misc_feature 1..25: note = >Female | ENST00000504429 | 16972455
  source 1..25: mol_type = other DNA
tggaaagctggcgacctaaatcggg

SEQ ID NO: 508 - DNA, 25 bp, synthetic construct
  misc_feature 1..25: note = >Female | ENST00000504429 | 16972455
  source 1..25: mol_type = other DNA
gctggtaaggacacgacactggttt

SEQ ID NO: 509 - DNA, 25 bp, synthetic construct
  misc_feature 1..25: note = >Female | ENST00000504429 | 16972455
  source 1..25: mol_type = other DNA
gagaagggaaatccagcaaaccgct

SEQ ID NO: 510 - DNA, 25 bp, synthetic construct
  misc_feature 1..25: note = >Female | ENST00000504429 | 16972455
  source 1..25: mol_type = other DNA
tctgctgatacaaactaggctggcc

SEQ ID NO: 511 - DNA, 25 bp, synthetic construct
  misc_feature 1..25: note = >Female | ENST00000504429 | 16972455
  source 1..25: mol_type = other DNA
aatcacagcagctaatattgtccca

SEQ ID NO: 512 - DNA, 25 bp, synthetic construct
  misc_feature 1..25: note = >Female | ENST00000504429 | 16972455
  source 1..25: mol_type = other DNA
tttgacaccaattgttacactatgc

SEQ ID NO: 513 - DNA, 25 bp, synthetic construct
  misc_feature 1..25: note = >Female | ENST00000504429 | 16972455
  source 1..25: mol_type = other DNA
gattaataacggttaagttgccatg

SEQ ID NO: 514 - DNA, 25 bp, synthetic construct
  misc_feature 1..25: note = >Female | ENST00000504429 | 16972455
  source 1..25: mol_type = other DNA
gcagtttcctgttttatttagtcca

SEQ ID NO: 515 - DNA, 25 bp, synthetic construct
  misc_feature 1..25: note = >Female | ENST00000504429 | 16972455
  source 1..25: mol_type = other DNA
gaatgttctatgagcacttgggtct

SEQ ID NO: 516 - DNA, 25 bp, synthetic construct
  misc_feature 1..25: note = >Female | ENST00000504429 | 16972455
  source 1..25: mol_type = other DNA
tctaagggttcaagaacatgggctc

SEQ ID NO: 517 - DNA, 25 bp, synthetic construct
  misc_feature 1..25: note = >Female | ENST00000504429 | 16972455
  source 1..25: mol_type = other DNA
tgcaggcagtatcatggcagccact

SEQ ID NO: 518 - DNA, 25 bp, synthetic construct
  misc_feature 1..25: note = >Female | ENST00000504429 | 16972455
  source 1..25: mol_type = other DNA
ccagaacgggtgtatgaggcgctaa

SEQ ID NO: 519 - DNA, 25 bp, synthetic construct
  misc_feature 1..25: note = >Female | NM_001142553 | 16980805
  source 1..25: mol_type = other DNA
aatgagcccaatctcgagttgctgg

SEQ ID NO: 520 - DNA, 25 bp, synthetic construct
  misc_feature 1..25: note = >Female | NM_001142553 | 16980805
  source 1..25: mol_type = other DNA
catcagtttccccagataggcagga

SEQ ID NO: 521 - DNA, 25 bp, synthetic construct
  misc_feature 1..25: note = >Female | NM_001142553 | 16980805
  source 1..25: mol_type = other DNA
gagtcagcactgaataccccacagg

SEQ ID NO: 522 - DNA, 25 bp, synthetic construct
  misc_feature 1..25: note = >Female | NM_001142553 | 16980805
  source 1..25: mol_type = other DNA
tgagtcacaattacaccaggcaaag

SEQ ID NO: 523 - DNA, 25 bp, synthetic construct
  misc_feature 1..25: note = >Female | NM_001142553 | 16980805
  source 1..25: mol_type = other DNA
tacagtcgtattagaaagcctgggc

SEQ ID NO: 524 - DNA, 25 bp, synthetic construct
  misc_feature 1..25: note = >Female | NM_001142553 | 16980805
  source 1..25: mol_type = other DNA
tggtgagctcgtaatctagaatcgc

SEQ ID NO: 525 - DNA, 25 bp, synthetic construct
  misc_feature 1..25: note = >Female | NM_001142553 | 16980805
  source 1..25: mol_type = other DNA
tagagcacagggaaagaactccatc

SEQ ID NO: 526 - DNA, 25 bp, synthetic construct
  misc_feature 1..25: note = >Female | NM_001142553 | 16980805
  source 1..25: mol_type = other DNA
catgtgaatgaccaagagacccgtg

SEQ ID NO: 527 - DNA, 25 bp, synthetic construct
  misc_feature 1..25: note = >Female | NM_001142553 | 16980805
  source 1..25: mol_type = other DNA
tattgccagcatccatatcagaggc

SEQ ID NO: 528 - DNA, 25 bp, synthetic construct
  misc_feature 1..25: note = >Female | NM_001142553 | 16980805
  source 1..25: mol_type = other DNA
aattcaaatgaagcacctggcatga

SEQ ID NO: 529 - DNA, 25 bp, synthetic construct
  misc_feature 1..25: note = >Female | NM_001142553 | 16980805
  source 1..25: mol_type = other DNA
ggtgtgccatcatccagtgccagaa

SEQ ID NO: 530 - DNA, 25 bp, synthetic construct
  misc_feature 1..25: note = >Female | NM_001142553 | 16980805
  source 1..25: mol_type = other DNA
cattgaagacatacaccagggttcc

SEQ ID NO: 531 - DNA, 25 bp, synthetic construct
  misc_feature 1..25: note = >Female | NM_001142553 | 16980805
  source 1..25: mol_type = other DNA
aaattgggaggcagatctagagaac

SEQ ID NO: 532 - DNA, 25 bp, synthetic construct
  misc_feature 1..25: note = >Female | NM_001142553 | 16980805
  source 1..25: mol_type = other DNA
tttcccttcagaggcataggtctag

SEQ ID NO: 533 - DNA, 25 bp, synthetic construct
  misc_feature 1..25: note = >Female | NM_001142553 | 16980805
  source 1..25: mol_type = other DNA
tcagtaactgagacgtaaaccgcca

SEQ ID NO: 534 - DNA, 25 bp, synthetic construct
  misc_feature 1..25: note = >Female | NM_001142553 | 16980805
  source 1..25: mol_type = other DNA
aagtctgtcccactaaaggcagcag

SEQ ID NO: 535 - DNA, 25 bp, synthetic construct
  misc_feature 1..25: note = >Female | NM_001142553 | 16980805
  source 1..25: mol_type = other DNA
cattttgcaagaatccgtcctcggg

SEQ ID NO: 536 - DNA, 25 bp, synthetic construct
  misc_feature 1..25: note = >Female | NM_001142553 | 16980805
  source 1..25: mol_type = other DNA
gagcagagaccatcaacgaaagtgt

SEQ ID NO: 537 - DNA, 25 bp, synthetic construct
  misc_feature 1..25: note = >Female | NM_001142553 | 16980805
  source 1..25: mol_type = other DNA
gccttagcagaggtaaaaacatctc

SEQ ID NO: 538 - DNA, 25 bp, synthetic construct
  misc_feature 1..25: note = >Female | NM_001142553 | 16980805
  source 1..25: mol_type = other DNA
tccaggtccacacgaacaaaggctt

SEQ ID NO: 539 - DNA, 25 bp, synthetic construct
  misc_feature 1..25: note = >Female | NM_001142553 | 16980805
  source 1..25: mol_type = other DNA
tcaccagggctaaatgtgtcattct

SEQ ID NO: 540 - DNA, 25 bp, synthetic construct
  misc_feature 1..25: note = >Female | NM_001142553 | 16980805
  source 1..25: mol_type = other DNA
tcatgagggtcgatccggaatgcct

SEQ ID NO: 541 - DNA, 25 bp, synthetic construct
  misc_feature 1..25: note = >Female | NM_001142553 | 16980805
  source 1..25: mol_type = other DNA
tgaacactatagcaccccagagctc

SEQ ID NO: 542 - DNA, 25 bp, synthetic construct
  misc_feature 1..25: note = >Female | NM_001142553 | 16980805
  source 1..25: mol_type = other DNA
tacacctcatatacacaggcatcag

SEQ ID NO: 543 - DNA, 25 bp, synthetic construct
  misc_feature 1..25: note = >Female | NM_001142553 | 16980805
  source 1..25: mol_type = other DNA
gattttgacaaaggcaacttgagca

SEQ ID NO: 544 - DNA, 25 bp, synthetic construct
  misc_feature 1..25: note = >Female | NM_001142553 | 16980805
  source 1..25: mol_type = other DNA
caaaagatggtccacactctgcggt

SEQ ID NO: 545 - DNA, 25 bp, synthetic construct
  misc_feature 1..25: note = >Female | NM_001142553 | 16980805
  source 1..25: mol_type = other DNA
gacccacatgactacagtgccaggg

SEQ ID NO: 546 - DNA, 25 bp, synthetic construct
  misc_feature 1..25: note = >Female | NM_001142553 | 16980805
  source 1..25: mol_type = other DNA
tcattgaggtcagcgacccggagta

SEQ ID NO: 547 - DNA, 25 bp, synthetic construct
  misc_feature 1..25: note = >Female | NM_001142553 | 16980805
  source 1..25: mol_type = other DNA
tccgtggccaccagtagtaactcat

SEQ ID NO: 548 - DNA, 25 bp, synthetic construct
  misc_feature 1..25: note = >Female | NM_001142553 | 16980805
  source 1..25: mol_type = other DNA
cacgtgaattgctggccggttgtca

SEQ ID NO: 549 - DNA, 25 bp, synthetic construct
  misc_feature 1..25: note = >Female | NM_001142553 | 16980805
  source 1..25: mol_type = other DNA
ctgtagcgcacgaagccattgggcc

SEQ ID NO: 550 - DNA, 25 bp, synthetic construct
  misc_feature 1..25: note = >Female | NM_001142553 | 16980805
  source 1..25: mol_type = other DNA
gtcctgctcaaagaccggcgggttg

SEQ ID NO: 551 - DNA, 25 bp, synthetic construct
  misc_feature 1..25: note = >Female | NM_001142553 | 16980805
  source 1..25: mol_type = other DNA
tccggtagcgcaactggaagaacgg

SEQ ID NO: 552 - DNA, 25 bp, synthetic construct
  misc_feature 1..25: note = >Female | NM_001142553 | 16980805
  source 1..25: mol_type = other DNA
tgcacgtggaagtcgtccagcagcg

SEQ ID NO: 553 - DNA, 25 bp, synthetic construct
  misc_feature 1..25: note = >Female | NM_001142553 | 16980805
  source 1..25: mol_type = other DNA
ggagtcctccgacagaaagaagccg

SEQ ID NO: 554 - DNA, 25 bp, synthetic construct
  misc_feature 1..25: note = >Female | NM_001142553 | 16980805
  source 1..25: mol_type = other DNA
ggatgtcacctaccagcgtgtccgg

SEQ ID NO: 555 - DNA, 25 bp, synthetic construct
  misc_feature 1..25: note = >Female | NM_001142553 | 16980805
  source 1..25: mol_type = other DNA
cagcttgtggcaggatcgcagaaaa

SEQ ID NO: 556 - DNA, 25 bp, synthetic construct
  misc_feature 1..25: note = >Female | NM_001142553 | 16980805
  source 1..25: mol_type = other DNA
catctgcaactggtgaaagcgtcct

SEQ ID NO: 557 - DNA, 25 bp, synthetic construct
  misc_feature 1..25: note = >Female | NM_001142553 | 16980805
  source 1..25: mol_type = other DNA
ggagattatatgaagcgcgcacaca

SEQ ID NO: 558 - DNA, 25 bp, synthetic construct
  misc_feature 1..25: note = >Female | NM_003211 | 16756127
  source 1..25: mol_type = other DNA
cctgggcccgatggctggcagtacc

SEQ ID NO: 559 - DNA, 25 bp, synthetic construct
  misc_feature 1..25: note = >Female | NM_003211 | 16756127
  source 1..25: mol_type = other DNA
tggaaacgtataaaaagcttgagct

SEQ ID NO: 560 - DNA, 25 bp, synthetic construct
  misc_feature 1..25: note = >Female | NM_003211 | 16756127
  source 1..25: mol_type = other DNA
actgccatattaggagcttcagcca

SEQ ID NO: 561 - DNA, 25 bp, synthetic construct
  misc_feature 1..25: note = >Female | NM_003211 | 16756127
  source 1..25: mol_type = other DNA
cagacagtctaaatttggagcccac

SEQ ID NO: 562 - DNA, 25 bp, synthetic construct
  misc_feature 1..25: note = >Female | NM_003211 | 16756127
  source 1..25: mol_type = other DNA
tcaggtctccacaggcatcattcag

SEQ ID NO: 563 - DNA, 25 bp, synthetic construct
  misc_feature 1..25: note = >Female | NM_003211 | 16756127
  source 1..25: mol_type = other DNA
ccatatggttcagctggacctcact

SEQ ID NO: 564 - DNA, 25 bp, synthetic construct
  misc_feature 1..25: note = >Female | NM_003211 | 16756127
  source 1..25: mol_type = other DNA
agagatctttgctgccgggcgtggt

SEQ ID NO: 565 - DNA, 25 bp, synthetic construct
  misc_feature 1..25: note = >Female | NM_003211 | 16756127
  source 1..25: mol_type = other DNA
tggagagatctttgctgccgggcgt

SEQ ID NO: 566 - DNA, 25 bp, synthetic construct
  misc_feature 1..25: note = >Female | NM_003211 | 16756127
  source 1..25: mol_type = other DNA
gctcagcttgaactagatacatatg

SEQ ID NO: 567 - DNA, 25 bp, synthetic construct
  misc_feature 1..25: note = >Female | NM_003211 | 16756127
  source 1..25: mol_type = other DNA
gtaatttctgtactagaatacgtcc

SEQ ID NO: 568 - DNA, 25 bp, synthetic construct
  misc_feature 1..25: note = >Female | NM_003211 | 16756127
  source 1..25: mol_type = other DNA
tgtaatttctgtactagaatacgtc

SEQ ID NO: 569 - DNA, 25 bp, synthetic construct
  misc_feature 1..25: note = >Female | NM_003211 | 16756127
  source 1..25: mol_type = other DNA
tacattgcccataatgttagcaaag

SEQ ID NO: 570 - DNA, 25 bp, synthetic construct
  misc_feature 1..25: note = >Female | NM_003211 | 16756127
  source 1..25: mol_type = other DNA
cgaggaaactgagcacatcttgcac

SEQ ID NO: 571 - DNA, 25 bp, synthetic construct
  misc_feature 1..25: note = >Female | NM_003211 | 16756127
  source 1..25: mol_type = other DNA
gtatattgcacctcttgaacgtcca

SEQ ID NO: 572 - DNA, 25 bp, synthetic construct
  misc_feature 1..25: note = >Female | NM_003211 | 16756127
  source 1..25: mol_type = other DNA
gctgcatggattttctccgtaagca

SEQ ID NO: 573 - DNA, 25 bp, synthetic construct
  misc_feature 1..25: note = >Female | NM_003211 | 16756127
  source 1..25: mol_type = other DNA
ttagcccatttgaagagaagccaca

SEQ ID NO: 574 - DNA, 25 bp, synthetic construct
  misc_feature 1..25: note = >Female | NM_003211 | 16756127
  source 1..25: mol_type = other DNA
ggaggaaaatgcaactaggatggcc

SEQ ID NO: 575 - DNA, 25 bp, synthetic construct
  misc_feature 1..25: note = >Female | NM_003211 | 16756127
  source 1..25: mol_type = other DNA
taaaatagttaagcaagcttccgga

SEQ ID NO: 576 - DNA, 25 bp, synthetic construct
  misc_feature 1..25: note = >Female | NM_003211 | 16756127
  source 1..25: mol_type = other DNA
cccgggatcgaggagaggcatatat

SEQ ID NO: 577 - DNA, 25 bp, synthetic construct
  misc_feature 1..25: note = >Female | NM_003211 | 16756127
  source 1..25: mol_type = other DNA
gagccaccgtatcagcttatttgaa

SEQ ID NO: 578 - DNA, 25 bp, synthetic construct
  misc_feature 1..25: note = >Female | NM_003211 | 16756127
  source 1..25: mol_type = other DNA
tgcccattaggaatgccactgaaag

SEQ ID NO: 579 - DNA, 25 bp, synthetic construct
  misc_feature 1..25: note = >Female | NM_003211 | 16756127
  source 1..25: mol_type = other DNA
taaatgactgggtcatccactgccc

SEQ ID NO: 580 - DNA, 25 bp, synthetic construct
  misc_feature 1..25: note = >Female | NM_003211 | 16756127
  source 1..25: mol_type = other DNA
taagcagagctgagaagcaccattc

SEQ ID NO: 581 - DNA, 25 bp, synthetic construct
  misc_feature 1..25: note = >Female | NM_003211 | 16756127
  source 1..25: mol_type = other DNA
ctgccaccccagagtagttaattgg

SEQ ID NO: 582 - DNA, 25 bp, synthetic construct
  misc_feature 1..25: note = >Female | NM_024638 | 16944179
  source 1..25: mol_type = other DNA
ccagtgcagagccaatcagggagta

SEQ ID NO: 583 - DNA, 25 bp, synthetic construct
  misc_feature 1..25: note = >Female | NM_024638 | 16944179
  source 1..25: mol_type = other DNA
cactaccacgtgttgcaaaattctc

SEQ ID NO: 584 - DNA, 25 bp, synthetic construct
  misc_feature 1..25: note = >Female | NM_024638 | 16944179
  source 1..25: mol_type = other DNA
ttttatctcagaggtgtcggcgacg

SEQ ID NO: 585 - DNA, 25 bp, synthetic construct
  misc_feature 1..25: note = >Female | NM_024638 | 16944179
  source 1..25: mol_type = other DNA
tttcagcagaggctagtcgaaaggg

SEQ ID NO: 586 - DNA, 25 bp, synthetic construct
  misc_feature 1..25: note = >Female | NM_024638 | 16944179
  source 1..25: mol_type = other DNA
gatttatctttgctccatgggccat

SEQ ID NO: 587 - DNA, 25 bp, synthetic construct
  misc_feature 1..25: note = >Female | NM_024638 | 16944179
  source 1..25: mol_type = other DNA
ggaaccccgtggatattatgcagcg

SEQ ID NO: 588 - DNA, 25 bp, synthetic construct
  misc_feature 1..25: note = >Female | NM_024638 | 16944179
  source 1..25: mol_type = other DNA
ctggatcgtgcagggagcagtacaa

SEQ ID NO: 589 - DNA, 25 bp, synthetic construct
  misc_feature 1..25: note = >Female | NM_024638 | 16944179
  source 1..25: mol_type = other DNA
tctgagggatacaccataggctgac

SEQ ID NO: 590 - DNA, 25 bp, synthetic construct
  misc_feature 1..25: note = >Female | NM_024638 | 16944179
  source 1..25: mol_type = other DNA
tctccatcggagaggcactggaacc

SEQ ID NO: 591 - DNA, 25 bp, synthetic construct
  misc_feature 1..25: note = >Female | NM_024638 | 16944179
  source 1..25: mol_type = other DNA
tgcagccgcagacagttatccaaga

SEQ ID NO: 592 - DNA, 25 bp, synthetic construct
  misc_feature 1..25: note = >Female | NM_024638 | 16944179
  source 1..25: mol_type = other DNA
gccgcttggctgtctctcgtgctga

SEQ ID NO: 593 - DNA, 25 bp, synthetic construct
  misc_feature 1..25: note = >Female | NM_024638 | 16944179
  source 1..25: mol_type = other DNA
caagcgtagtctagcctccagggtt

SEQ ID NO: 594 - DNA, 25 bp, synthetic construct
  misc_feature 1..25: note = >Female | NM_024638 | 16944179
  source 1..25: mol_type = other DNA
gaataagaacagtgacatgaggaca

SEQ ID NO: 595 - DNA, 25 bp, synthetic construct
  misc_feature 1..25: note = >Female | NM_024638 | 16944179
  source 1..25: mol_type = other DNA
tggccgactaacaccagatatgagc

SEQ ID NO: 596 - DNA, 25 bp, synthetic construct
  misc_feature 1..25: note = >Female | NM_024638 | 16944179
  source 1..25: mol_type = other DNA
taatcaaaactgaaagtcagggcac

SEQ ID NO: 597 - DNA, 25 bp, synthetic construct
  misc_feature 1..25: note = >Female | NM_024638 | 16944179
  source 1..25: mol_type = other DNA
tgatcagataagaagtggccattgg

SEQ ID NO: 598 - DNA, 25 bp, synthetic construct
  misc_feature 1..25: note = >Female | NM_024638 | 16944179
  source 1..25: mol_type = other DNA
tcttggttgcaaccagttgtttcaa

SEQ ID NO: 599 - DNA, 25 bp, synthetic construct
  misc_feature 1..25: note = >Female | NM_024638 | 16944179
  source 1..25: mol_type = other DNA
ccttcagattaatttcaaatgatgt

SEQ ID NO: 600 - DNA, 25 bp, synthetic construct
  misc_feature 1..25: note = >Female | NM_024638 | 16944179
  source 1..25: mol_type = other DNA
tcttctgacaagacgaagtctacaa

SEQ ID NO: 601 - DNA, 25 bp, synthetic construct
  misc_feature 1..25: note = >Female | NM_024638 | 16944179
  source 1..25: mol_type = other DNA
tggctccagtcttctgacaagacga

SEQ ID NO: 602 - DNA, 25 bp, synthetic construct
  misc_feature 1..25: note = >Female | NM_024638 | 16944179
  source 1..25: mol_type = other DNA
cagcagatggtggatgtatgcccga

SEQ ID NO: 603 - DNA, 25 bp, synthetic construct
  misc_feature 1..25: note = >Female | NM_024638 | 16944179
  source 1..25: mol_type = other DNA
cactttttagtgcttcccggatgta

SEQ ID NO: 604 - DNA, 25 bp, synthetic construct
  misc_feature 1..25: note = >Female | NM_024638 | 16944179
  source 1..25: mol_type = other DNA
ctctaatcgttaggccaggcagggc

SEQ ID NO: 605 - DNA, 25 bp, synthetic construct
  misc_feature 1..25: note = >Female | NM_033637 | 16708482
  source 1..25: mol_type = other DNA
tgagtgccttctcttgcagcaccgc

SEQ ID NO: 606 - DNA, 25 bp, synthetic construct
  misc_feature 1..25: note = >Female | NM_033637 | 16708482
  source 1..25: mol_type = other DNA
gtttctccagagagcttcctccgct

SEQ ID NO: 607 - DNA, 25 bp, synthetic construct
  misc_feature 1..25: note = >Female | NM_033637 | 16708482
  source 1..25: mol_type = other DNA
ggttatacaggcatcgcagcgaagg

SEQ ID NO: 608 - DNA, 25 bp, synthetic construct
  misc_feature 1..25: note = >Female | NM_033637 | 16708482
  source 1..25: mol_type = other DNA
tggaaagctgtgagtgcgccagtcc

SEQ ID NO: 609 - DNA, 25 bp, synthetic construct
  misc_feature 1..25: note = >Female | NM_033637 | 16708482
  source 1..25: mol_type = other DNA
caaggttggatggtgcaacagagcc

SEQ ID NO: 610 - DNA, 25 bp, synthetic construct
  misc_feature 1..25: note = >Female | NM_033637 | 16708482
  source 1..25: mol_type = other DNA
ggttcgccattattacagtcttctc

SEQ ID NO: 611 - DNA, 25 bp, synthetic construct
  misc_feature 1..25: note = >Female | NM_033637 | 16708482
  source 1..25: mol_type = other DNA
gggttcgccattattacagtcttct

SEQ ID NO: 612 - DNA, 25 bp, synthetic construct
  misc_feature 1..25: note = >Female | NM_033637 | 16708482
  source 1..25: mol_type = other DNA
tggtttaagcagagtctggcacagc

SEQ ID NO: 613 - DNA, 25 bp, synthetic construct
  misc_feature 1..25: note = >Female | NM_033637 | 16708482
  source 1..25: mol_type = other DNA
tctcagtcttcatagcagtgcttgc

SEQ ID NO: 614 - DNA, 25 bp, synthetic construct
  misc_feature 1..25: note = >Female | NM_033637 | 16708482
  source 1..25: mol_type = other DNA
tgagagtttccgttgcttgggcaca

SEQ ID NO: 615 - DNA, 25 bp, synthetic construct
  misc_feature 1..25: note = >Female | NM_033637 | 16708482
  source 1..25: mol_type = other DNA
taagatacgagtttatgtgcccatg

SEQ ID NO: 616 - DNA, 25 bp, synthetic construct
  misc_feature 1..25: note = >Female | NM_033637 | 16708482
  source 1..25: mol_type = other DNA
tcagcaatatgatccaatccccgag

SEQ ID NO: 617 - DNA, 25 bp, synthetic construct
  misc_feature 1..25: note = >Female | NM_033637 | 16708482
  source 1..25: mol_type = other DNA
tcttccacagcatgccatcagaggt

SEQ ID NO: 618 - DNA, 25 bp, synthetic construct
  misc_feature 1..25: note = >Female | NM_033637 | 16708482
  source 1..25: mol_type = other DNA
cacttcggcagtgaattctctgtaa

SEQ ID NO: 619 - DNA, 25 bp, synthetic construct
  misc_feature 1..25: note = >Female | NM_033637 | 16708482
  source 1..25: mol_type = other DNA
tgttgtctcgaaggccgcttactat

SEQ ID NO: 620 - DNA, 25 bp, synthetic construct
  misc_feature 1..25: note = >Female | NM_033637 | 16708482
  source 1..25: mol_type = other DNA
tatggcctgtgagaattcgcttgca

SEQ ID NO: 621 - DNA, 25 bp, synthetic construct
  misc_feature 1..25: note = >Female | NM_033637 | 16708482
  source 1..25: mol_type = other DNA
tgaccgtggaatccgatgatcctgt

SEQ ID NO: 622 - DNA, 25 bp, synthetic construct
  misc_feature 1..25: note = >Female | NM_033637 | 16708482
  source 1..25: mol_type = other DNA
gaacgatctttggagcaggtcacca

SEQ ID NO: 623 - DNA, 25 bp, synthetic construct
  misc_feature 1..25: note = >Female | NM_033637 | 16708482
  source 1..25: mol_type = other DNA
tagtgttgtcagatgagccactcac

SEQ ID NO: 624 - DNA, 25 bp, synthetic construct
  misc_feature 1..25: note = >Female | NM_033637 | 16708482
  source 1..25: mol_type = other DNA
tggccttctaacactcgtaaacatg

SEQ ID NO: 625 - DNA, 25 bp, synthetic construct
  misc_feature 1..25: note = >Female | NM_033637 | 16708482
  source 1..25: mol_type = other DNA
gaatacaacgcaccaattcctcatg

SEQ ID NO: 626 - DNA, 25 bp, synthetic construct
  misc_feature 1..25: note = >Female | NM_033637 | 16708482
  source 1..25: mol_type = other DNA
gcagccacaagatcccacactttaa

SEQ ID NO: 627 - DNA, 25 bp, synthetic construct
  misc_feature 1..25: note = >Female | NM_033637 | 16708482
  source 1..25: mol_type = other DNA
caagggtccgtagacagagtgtccc

SEQ ID NO: 628 - DNA, 25 bp, synthetic construct
  misc_feature 1..25: note = >Female | NM_033637 | 16708482
  source 1..25: mol_type = other DNA
tagtcgaaaaactcttccggaatgc

SEQ ID NO: 629 - DNA, 25 bp, synthetic construct
  misc_feature 1..25: note = >Female | NM_033637 | 16708482
  source 1..25: mol_type = other DNA
cagctggatcatttaggaagtccca

SEQ ID NO: 630 - DNA, 25 bp, synthetic construct
  misc_feature 1..25: note = >Female | NM_033637 | 16708482
  source 1..25: mol_type = other DNA
caagtatgaggtcagtgtatggtta

SEQ ID NO: 631 - DNA, 25 bp, synthetic construct
  misc_feature 1..25: note = >Female | NM_033637 | 16708482
  source 1..25: mol_type = other DNA
ctgggcaagtatgaggtcagtgtat

SEQ ID NO: 632 - DNA, 25 bp, synthetic construct
  misc_feature 1..25: note = >Female | NM_033637 | 16708482
  source 1..25: mol_type = other DNA
cccaaacggcaggagaaagttccat

SEQ ID NO: 633 - DNA, 25 bp, synthetic construct
  misc_feature 1..25: note = >Female | NM_033637 | 16708482
  source 1..25: mol_type = other DNA
gactaggcttaccacacaagtagga

SEQ ID NO: 634 - DNA, 25 bp, synthetic construct
  misc_feature 1..25: note = >Female | NM_033637 | 16708482
  source 1..25: mol_type = other DNA
tcttggcaaagatacaggcacctgg

SEQ ID NO: 635 - DNA, 25 bp, synthetic construct
  misc_feature 1..25: note = >Female | NM_033637 | 16708482
  source 1..25: mol_type = other DNA
tagattggagagcaaaaccctgggc

SEQ ID NO: 636 - DNA, 25 bp, synthetic construct
  misc_feature 1..25: note = >Female | NM_033655 | 17094394
  source 1..25: mol_type = other DNA
gaagtgccaggagttcagtccacgc

SEQ ID NO: 637 - DNA, 25 bp, synthetic construct
  misc_feature 1..25: note = >Female | NM_033655 | 17094394
  source 1..25: mol_type = other DNA
tcacgatgatagagacagccacagc

SEQ ID NO: 638 - DNA, 25 bp, synthetic construct
  misc_feature 1..25: note = >Female | NM_033655 | 17094394
  source 1..25: mol_type = other DNA
gtaactttctctgttgatagatgcg

SEQ ID NO: 639 - DNA, 25 bp, synthetic construct
  misc_feature 1..25: note = >Female | NM_033655 | 17094394
  source 1..25: mol_type = other DNA
tatggctatggcagtgatgcaaagc

SEQ ID NO: 640 - DNA, 25 bp, synthetic construct
  misc_feature 1..25: note = >Female | NM_033655 | 17094394
  source 1..25: mol_type = other DNA
taaagcgataatgacaagcaccagg

SEQ ID NO: 641 - DNA, 25 bp, synthetic construct
  misc_feature 1..25: note = >Female | NM_033655 | 17094394
  source 1..25: mol_type = other DNA
caccaggtcattcagggccttgtta

SEQ ID NO: 642 - DNA, 25 bp, synthetic construct
  misc_feature 1..25: note = >Female | NM_033655 | 17094394
  source 1..25: mol_type = other DNA
ctccgatgacagcagagtctcttct

SEQ ID NO: 643 - DNA, 25 bp, synthetic construct
  misc_feature 1..25: note = >Female | NM_033655 | 17094394
  source 1..25: mol_type = other DNA
tctgtctgcattaaccaagggctct

SEQ ID NO: 644 - DNA, 25 bp, synthetic construct
  misc_feature 1..25: note = >Female | NM_033655 | 17094394
  source 1..25: mol_type = other DNA
cgagaggcagccagtgaagccacta

SEQ ID NO: 645 - DNA, 25 bp, synthetic construct
  misc_feature 1..25: note = >Female | NM_033655 | 17094394
  source 1..25: mol_type = other DNA
agccagtgaagccactagtcgccgc

SEQ ID NO: 646 - DNA, 25 bp, synthetic construct
  misc_feature 1..25: note = >Female | NM_033655 | 17094394
  source 1..25: mol_type = other DNA
gatggctggctctctgcaagtaaca

SEQ ID NO: 647 - DNA, 25 bp, synthetic construct
  misc_feature 1..25: note = >Female | NM_033655 | 17094394
  source 1..25: mol_type = other DNA
tccggaggattacagaaaagcgact

SEQ ID NO: 648 - DNA, 25 bp, synthetic construct
  misc_feature 1..25: note = >Female | NM_033655 | 17094394
  source 1..25: mol_type = other DNA
tctgagtatatacttcgttgctgaa

SEQ ID NO: 649 - DNA, 25 bp, synthetic construct
  misc_feature 1..25: note = >Female | NM_033655 | 17094394
  source 1..25: mol_type = other DNA
tgcctttgaatacaccatccttgaa

SEQ ID NO: 650 - DNA, 25 bp, synthetic construct
  misc_feature 1..25: note = >Female | NM_033655 | 17094394
  source 1..25: mol_type = other DNA
gaaacaacaggcaaagcagggcttg

SEQ ID NO: 651 - DNA, 25 bp, synthetic construct
  misc_feature 1..25: note = >Female | NM_033655 | 17094394
  source 1..25: mol_type = other DNA
gattatatgtacactcaggcaagag

SEQ ID NO: 652 - DNA, 25 bp, synthetic construct
  misc_feature 1..25: note = >Female | NM_033655 | 17094394
  source 1..25: mol_type = other DNA
tcccaatatgagagatttgacggcg

SEQ ID NO: 653 - DNA, 25 bp, synthetic construct
  misc_feature 1..25: note = >Female | NM_033655 | 17094394
  source 1..25: mol_type = other DNA
ggcgttgaattctgtccctgaggac

SEQ ID NO: 654 - DNA, 25 bp, synthetic construct
  misc_feature 1..25: note = >Female | NM_033655 | 17094394
  source 1..25: mol_type = other DNA
ttagatgtcccatgagaagcaaaca

SEQ ID NO: 655 - DNA, 25 bp, synthetic construct
  misc_feature 1..25: note = >Female | NM_033655 | 17094394
  source 1..25: mol_type = other DNA
taagcaattcctatgatgccatcag

SEQ ID NO: 656 - DNA, 25 bp, synthetic construct
  misc_feature 1..25: note = >Female | NM_033655 | 17094394
  source 1..25: mol_type = other DNA
cataatggaaggatctataattggc

SEQ ID NO: 657 - DNA, 25 bp, synthetic construct
  misc_feature 1..25: note = >Female | NM_033655 | 17094394
  source 1..25: mol_type = other DNA
catgaaaaagatggaactggctgtt

SEQ ID NO: 658 - DNA, 25 bp, synthetic construct
  misc_feature 1..25: note = >Female | NM_033655 | 17094394
  source 1..25: mol_type = other DNA
tgaaacataaaaccaagcactgctg

SEQ ID NO: 659 - DNA, 25 bp, synthetic construct
  misc_feature 1..25: note = >Female | NM_033655 | 17094394
  source 1..25: mol_type = other DNA
tgacttatggaagagcagcactgaa

SEQ ID NO: 660 - DNA, 25 bp, synthetic construct
  misc_feature 1..25: note = >Female | NM_033655 | 17094394
  source 1..25: mol_type = other DNA
ttgacttttgcatttgtgggattac

SEQ ID NO: 661 - DNA, 25 bp, synthetic construct
  misc_feature 1..25: note = >Female | NM_033655 | 17094394
  source 1..25: mol_type = other DNA
catgaccacggcttcttctctgtta

SEQ ID NO: 662 - DNA, 25 bp, synthetic construct
  misc_feature 1..25: note = >Female | NM_033655 | 17094394
  source 1..25: mol_type = other DNA
ggtgaagttgcccatcagccatgtt

SEQ ID NO: 663 - DNA, 25 bp, synthetic construct
  misc_feature 1..25: note = >Female | NM_033655 | 17094394
  source 1..25: mol_type = other DNA
tacagcaataagctcggagttcgtg

SEQ ID NO: 664 - DNA, 25 bp, synthetic construct
  misc_feature 1..25: note = >Female | NM_033655 | 17094394
  source 1..25: mol_type = other DNA
tatgtcattgaggagccagttgcga

SEQ ID NO: 665 - DNA, 25 bp, synthetic construct
  misc_feature 1..25: note = >Female | NM_152891 | 16823203
  source 1..25: mol_type = other DNA
tcagggtgctgccagaatccagtgc

SEQ ID NO: 666 - DNA, 25 bp, synthetic construct
  misc_feature 1..25: note = >Female | NM_152891 | 16823203
  source 1..25: mol_type = other DNA
aattggtttgcccatcactgcgtgc

SEQ ID NO: 667 - DNA, 25 bp, synthetic construct
  misc_feature 1..25: note = >Female | NM_152891 | 16823203
  source 1..25: mol_type = other DNA
gagtcagagctggtgatccccaaag

SEQ ID NO: 668 - DNA, 25 bp, synthetic construct
  misc_feature 1..25: note = >Female | NM_152891 | 16823203
  source 1..25: mol_type = other DNA
agaagctgacgcgagcctgaatcca

SEQ ID NO: 669 - DNA, 25 bp, synthetic construct
  misc_feature 1..25: note = >Female | NM_152891 | 16823203
  source 1..25: mol_type = other DNA
tatatgtggccacactggtgtagac

SEQ ID NO: 670 - DNA, 25 bp, synthetic construct
  misc_feature 1..25: note = >Female | NM_152891 | 16823203
  source 1..25: mol_type = other DNA
cactggtgtagacccctggacggtt

SEQ ID NO: 671 - DNA, 25 bp, synthetic construct
  misc_feature 1..25: note = >Female | NM_152891 | 16823203
  source 1..25: mol_type = other DNA
agactgcaggcaggtcagaggtccc

SEQ ID NO: 672 - DNA, 25 bp, synthetic construct
  misc_feature 1..25: note = >Female | NM_152891 | 16823203
  source 1..25: mol_type = other DNA
aggcagcacaatgcgctcagcctgg

SEQ ID NO: 673 - DNA, 25 bp, synthetic construct
  misc_feature 1..25: note = >Female | NM_152891 | 16823203
  source 1..25: mol_type = other DNA
agcggcacccttactccttgtagcg

SEQ ID NO: 674 - DNA, 25 bp, synthetic construct
  misc_feature 1..25: note = >Female | NM_152891 | 16823203
  source 1..25: mol_type = other DNA
gcggcacccttactccttgtagcgg

SEQ ID NO: 675 - DNA, 25 bp, synthetic construct
  misc_feature 1..25: note = >Female | NM_152891 | 16823203
  source 1..25: mol_type = other DNA
gcacccttactccttgtagcggtcg

SEQ ID NO: 676 - DNA, 25 bp, synthetic construct
  misc_feature 1..25: note = >Female | NM_152891 | 16823203
  source 1..25: mol_type = other DNA
ccttgtagcggtcgccactctggga

SEQ ID NO: 677 - DNA, 25 bp, synthetic construct
  misc_feature 1..25: note = >Female | NM_152891 | 16823203
  source 1..25: mol_type = other DNA
cccagccggtgacccggcatggtgt

SEQ ID NO: 678 - DNA, 25 bp, synthetic construct
  misc_feature 1..25: note = >Female | NM_152891 | 16823203
  source 1..25: mol_type = other DNA
aggcgcacgcggtactcagctggca

SEQ ID NO: 679 - DNA, 25 bp, synthetic construct
  misc_feature 1..25: note = >Female | NM_152891 | 16823203
  source 1..25: mol_type = other DNA
gcgcacgcggtactcagctggcagt

SEQ ID NO: 680 - DNA, 25 bp, synthetic construct
  misc_feature 1..25: note = >Female | NM_152891 | 16823203
  source 1..25: mol_type = other DNA
cgcacgcggtactcagctggcagtg

SEQ ID NO: 681 - DNA, 25 bp, synthetic construct
  misc_feature 1..25: note = >Female | NM_152891 | 16823203
  source 1..25: mol_type = other DNA
gcggtactcagctggcagtgccctc

SEQ ID NO: 682 - DNA, 25 bp, synthetic construct
  misc_feature 1..25: note = >Female | NM_152891 | 16823203
  source 1..25: mol_type = other DNA
cacgtgtgccccacgatgctggatg

SEQ ID NO: 683 - DNA, 25 bp, synthetic construct
  misc_feature 1..25: note = >Female | NM_152891 | 16823203
  source 1..25: mol_type = other DNA
tgtgccccacgatgctggatgctcg

SEQ ID NO: 684 - DNA, 25 bp, synthetic construct
  misc_feature 1..25: note = >Female | NM_152891 | 16823203
  source 1..25: mol_type = other DNA
acgatgctggatgctcgcctgccac

SEQ ID NO: 685 - DNA, 25 bp, synthetic construct
  misc_feature 1..25: note = >Female | NM_152891 | 16823203
  source 1..25: mol_type = other DNA
ccaacgatccgactggacatgcggg

SEQ ID NO: 686 - DNA, 25 bp, synthetic construct
  misc_feature 1..25: note = >Male | ENST00000440123 | 16708531
  source 1..25: mol_type = other DNA
ttttttaaaagctgcgtcacatgca

SEQ ID NO: 687 - DNA, 25 bp, synthetic construct
  misc_feature 1..25: note = >Male | ENST00000440123 | 16708531
  source 1..25: mol_type = other DNA
cttttttaaaagctgcgtcacatgc

SEQ ID NO: 688 - DNA, 25 bp, synthetic construct
  misc_feature 1..25: note = >Male | ENST00000440123 | 16708531
  source 1..25: mol_type = other DNA
ccttttttaaaagctgcgtcacatg

SEQ ID NO: 689 - DNA, 25 bp, synthetic construct
  misc_feature 1..25: note = >Male | ENST00000440123 | 16708531
  source 1..25: mol_type = other DNA
ggagagagatacaccacactgccca

SEQ ID NO: 690 - DNA, 25 bp, synthetic construct
  misc_feature 1..25: note = >Male | ENST00000440123 | 16708531
  source 1..25: mol_type = other DNA
tggagagagatacaccacactgccc

SEQ ID NO: 691 - DNA, 25 bp, synthetic construct
  misc_feature 1..25: note = >Male | ENST00000440123 | 16708531
  source 1..25: mol_type = other DNA
gtggagagagatacaccacactgcc

SEQ ID NO: 692 - DNA, 25 bp, synthetic construct
  misc_feature 1..25: note = >Male | ENST00000440123 | 16708531
  source 1..25: mol_type = other DNA
ggtggagagagatacaccacactgc

SEQ ID NO: 693 - DNA, 25 bp, synthetic construct
  misc_feature 1..25: note = >Male | ENST00000440123 | 16708531
  source 1..25: mol_type = other DNA
gggtggagagagatacaccacactg

SEQ ID NO: 694 - DNA, 25 bp, synthetic construct
  misc_feature 1..25: note = >Male | ENST00000440123 | 16708531
  source 1..25: mol_type = other DNA
cagcctagggtagggacgcgaaaca

SEQ ID NO: 695 - DNA, 25 bp, synthetic construct
  misc_feature 1..25: note = >Male | ENST00000440123 | 16708531
  source 1..25: mol_type = other DNA
atccagcctagggtagggacgcgaa

SEQ ID NO: 696 - DNA, 25 bp, synthetic construct
  misc_feature 1..25: note = >Male | ENST00000440123 | 16708531
  source 1..25: mol_type = other DNA
catccagcctagggtagggacgcga

SEQ ID NO: 697 - DNA, 25 bp, synthetic construct
  misc_feature 1..25: note = >Male | ENST00000440123 | 16708531
  source 1..25: mol_type = other DNA
ccatccagcctagggtagggacgcg

SEQ ID NO: 698 - DNA, 25 bp, synthetic construct
  misc_feature 1..25: note = >Male | ENST00000440123 | 16708531
  source 1..25: mol_type = other DNA
tctccgaagccctcaaaggacagca

SEQ ID NO: 699 - DNA, 25 bp, synthetic construct
  misc_feature 1..25: note = >Male | ENST00000440123 | 16708531
  source 1..25: mol_type = other DNA
atctccgaagccctcaaaggacagc

SEQ ID NO: 700 - DNA, 25 bp, synthetic construct
  misc_feature 1..25: note = >Male | ENST00000440123 | 16708531
  source 1..25: mol_type = other DNA
catctccgaagccctcaaaggacag

SEQ ID NO: 701 - DNA, 25 bp, synthetic construct
  misc_feature 1..25: note = >Male | ENST00000440123 | 16708531
  source 1..25: mol_type = other DNA
tcatctccgaagccctcaaaggaca

SEQ ID NO: 702 - DNA, 25 bp, synthetic construct
  misc_feature 1..25: note = >Male | ENST00000440123 | 16708531
  source 1..25: mol_type = other DNA
tctcatctccgaagccctcaaagga

SEQ ID NO: 703 - DNA, 25 bp, synthetic construct
  misc_feature 1..25: note = >Male | ENST00000440123 | 16708531
  source 1..25: mol_type = other DNA
ttctcatctccgaagccctcaaagg

SEQ ID NO: 704 - DNA, 25 bp, synthetic construct
  misc_feature 1..25: note = >Male | TCONS_00025321-XLOC_012127 | 16831561
  source 1..25: mol_type = other DNA
atgccctccttattaactcatcaca

SEQ ID NO: 705 - DNA, 25 bp, synthetic construct
  misc_feature 1..25: note = >Male | TCONS_00025321-XLOC_012127 | 16831561
  source 1..25: mol_type = other DNA
ggccaggacccgagctcattctgca

SEQ ID NO: 706 - DNA, 25 bp, synthetic construct
  misc_feature 1..25: note = >Male | TCONS_00025321-XLOC_012127 | 16831561
  source 1..25: mol_type = other DNA
acagtgctggccagatgtcccatgg

SEQ ID NO: 707 - DNA, 25 bp, synthetic construct
  misc_feature 1..25: note = >Male | TCONS_00025321-XLOC_012127 | 16831561
  source 1..25: mol_type = other DNA
atgatcccatggaaagatgggctcc

SEQ ID NO: 708 - DNA, 25 bp, synthetic construct
  misc_feature 1..25: note = >Male | TCONS_00025321-XLOC_012127 | 16831561
  source 1..25: mol_type = other DNA
gaaggactctaatgatcccatggaa

SEQ ID NO: 709 - DNA, 25 bp, synthetic construct
  misc_feature 1..25: note = >Male | TCONS_00025321-XLOC_012127 | 16831561
  source 1..25: mol_type = other DNA
caacagctgcaagagaaggactcta

SEQ ID NO: 710 - DNA, 25 bp, synthetic construct
  misc_feature 1..25: note = >Male | TCONS_00025321-XLOC_012127 | 16831561
  source 1..25: mol_type = other DNA
ccaacagctgcaagagaaggactct

SEQ ID NO: 711 - DNA, 25 bp, synthetic construct
  misc_feature 1..25: note = >Male | TCONS_00025321-XLOC_012127 | 16831561
  source 1..25: mol_type = other DNA
gtccacacatcattcctgtctgcga

SEQ ID NO: 712 - DNA, 25 bp, synthetic construct
  misc_feature 1..25: note = >Male | TCONS_00025321-XLOC_012127 | 16831561
  source 1..25: mol_type = other DNA
agtccacacatcattcctgtctgcg

SEQ ID NO: 713 - DNA, 25 bp, synthetic construct
  misc_feature 1..25: note = >Male | TCONS_00025321-XLOC_012127 | 16831561
  source 1..25: mol_type = other DNA
gcaagtccacacatcattcctgtct

SEQ ID NO: 714 - DNA, 25 bp, synthetic construct
  misc_feature 1..25: note = >Male | TCONS_00025321-XLOC_012127 | 16831561
  source 1..25: mol_type = other DNA
ggcaagtccacacatcattcctgtc

SEQ ID NO: 715 - DNA, 25 bp, synthetic construct
  misc_feature 1..25: note = >Male | TCONS_00025321-XLOC_012127 | 16831561
  source 1..25: mol_type = other DNA
tggcaagtccacacatcattcctgt

SEQ ID NO: 716 - DNA, 25 bp, synthetic construct
  misc_feature 1..25: note = >Male | TCONS_00025321-XLOC_012127 | 16831561
  source 1..25: mol_type = other DNA
atggcaagtccacacatcattcctg

SEQ ID NO: 717 - DNA, 25 bp, synthetic construct
  misc_feature 1..25: note = >Male | TCONS_00025321-XLOC_012127 | 16831561
  source 1..25: mol_type = other DNA
catggcaagtccacacatcattcct

SEQ ID NO: 718 - DNA, 25 bp, synthetic construct
  misc_feature 1..25: note = >Male | TCONS_00025321-XLOC_012127 | 16831561
  source 1..25: mol_type = other DNA
caccatggcaagtccacacatcatt

SEQ ID NO: 719 - DNA, 25 bp, synthetic construct
  misc_feature 1..25: note = >Male | TCONS_00025321-XLOC_012127 | 16831561
  source 1..25: mol_type = other DNA
acaccatggcaagtccacacatcat

SEQ ID NO: 720 - DNA, 25 bp, synthetic construct
  misc_feature 1..25: note = >Male | TCONS_00025321-XLOC_012127 | 16831561
  source 1..25: mol_type = other DNA
tgcacaccatggcaagtccacacat

SEQ ID NO: 721 - DNA, 25 bp, synthetic construct
  misc_feature 1..25: note = >Male | TCONS_00025321-XLOC_012127 | 16831561
  source 1..25: mol_type = other DNA
gctcatgcacaccatggcaagtcca

SEQ ID NO: 722 - DNA, 25 bp, synthetic construct
  misc_feature 1..25: note = >Male | TCONS_00025321-XLOC_012127 | 16831561
  source 1..25: mol_type = other DNA
ggctcatgcacaccatggcaagtcc

SEQ ID NO: 723 - DNA, 25 bp, synthetic construct
  misc_feature 1..25: note = >Male | ENST00000488290 | 16712992
  source 1..25: mol_type = other DNA
ccagcccttcttggatgggcagagg

SEQ ID NO: 724 - DNA, 25 bp, synthetic construct
  misc_feature 1..25: note = >Male | ENST00000488290 | 16712992
  source 1..25: mol_type = other DNA
cagcccttcttggatgggcagaggt

SEQ ID NO: 725 - DNA, 25 bp, synthetic construct
  misc_feature 1..25: note = >Male | ENST00000488290 | 16712992
  source 1..25: mol_type = other DNA
cccttcttggatgggcagaggttac

SEQ ID NO: 726 - DNA, 25 bp, synthetic construct
  misc_feature 1..25: note = >Male | ENST00000488290 | 16712992
  source 1..25: mol_type = other DNA
tcttggatgggcagaggttacagtc

SEQ ID NO: 727 - DNA, 25 bp, synthetic construct
  misc_feature 1..25: note = >Male | ENST00000488290 | 16712992
  source 1..25: mol_type = other DNA
tggatgggcagaggttacagtctcc

SEQ ID NO: 728 - DNA, 25 bp, synthetic construct
  misc_feature 1..25: note = >Male | ENST00000488290 | 16712992
  source 1..25: mol_type = other DNA
ggatgggcagaggttacagtctcct

SEQ ID NO: 729 - DNA, 25 bp, synthetic construct
  misc_feature 1..25: note = >Male | ENST00000488290 | 16712992
  source 1..25: mol_type = other DNA
gatgggcagaggttacagtctcctt

SEQ ID NO: 730 - DNA, 25 bp, synthetic construct
  misc_feature 1..25: note = >Male | ENST00000488290 | 16712992
  source 1..25: mol_type = other DNA
atgggcagaggttacagtctccttc

SEQ ID NO: 731 - DNA, 25 bp, synthetic construct
  misc_feature 1..25: note = >Male | ENST00000488290 | 16712992
  source 1..25: mol_type = other DNA
ggcagaggttacagtctccttcagc

SEQ ID NO: 732 - DNA, 25 bp, synthetic construct
  misc_feature 1..25: note = >Male | ENST00000488290 | 16712992
  source 1..25: mol_type = other DNA
gaggttacagtctccttcagcttgc

SEQ ID NO: 733 - DNA, 25 bp, synthetic construct
  misc_feature 1..25: note = >Male | ENST00000430988 | 16895114
  source 1..25: mol_type = other DNA
taatcaaagtttgaaggcagccgca

SEQ ID NO: 734 - DNA, 25 bp, synthetic construct
  misc_feature 1..25: note = >Male | ENST00000430988 | 16895114
  source 1..25: mol_type = other DNA
tgaaggcagccgcatttcctcaggc

SEQ ID NO: 735 - DNA, 25 bp, synthetic construct
  misc_feature 1..25: note = >Male | ENST00000430988 | 16895114
  source 1..25: mol_type = other DNA
gctcaacatcgctgcctgggtggac

SEQ ID NO: 736 - DNA, 25 bp, synthetic construct
  misc_feature 1..25: note = >Male | ENST00000430988 | 16895114
  source 1..25: mol_type = other DNA
tgtgcccagctgttggtggcgctca

SEQ ID NO: 737 - DNA, 25 bp, synthetic construct
  misc_feature 1..25: note = >Male | ENST00000430988 | 16895114
  source 1..25: mol_type = other DNA
gtgcatgccatcatagccttcctgg

SEQ ID NO: 738 - DNA, 25 bp, synthetic construct
  misc_feature 1..25: note = >Male | ENST00000430988 | 16895114
  source 1..25: mol_type = other DNA
catcatagccttcctggtgcaggga

SEQ ID NO: 739 - DNA, 25 bp, synthetic construct
  misc_feature 1..25: note = >Male | ENST00000430988 | 16895114
  source 1..25: mol_type = other DNA
cctgtaagcaccatcaacatccaga

SEQ ID NO: 740 - DNA, 25 bp, synthetic construct
  misc_feature 1..25: note = >Male | ENST00000430988 | 16895114
  source 1..25: mol_type = other DNA
gagaagattccatgcgtctttcggg

SEQ ID NO: 741 - DNA, 25 bp, synthetic construct
  misc_feature 1..25: note = >Male | ENST00000430988 | 16895114
  source 1..25: mol_type = other DNA
aagattccatgcgtctttcgggagc

SEQ ID NO: 742 - DNA, 25 bp, synthetic construct
  misc_feature 1..25: note = >Male | ENST00000430988 | 16895114
  source 1..25: mol_type = other DNA
tgcgtctttcgggagcttgcctaaa

SEQ ID NO: 743 - DNA, 25 bp, synthetic construct
  misc_feature 1..25: note = >Male | ENST00000430988 | 16895114
  source 1..25: mol_type = other DNA
gcgtctttcgggagcttgcctaaac

SEQ ID NO: 744 - DNA, 25 bp, synthetic construct
  misc_feature 1..25: note = >Male | ENST00000430988 | 16895114
  source 1..25: mol_type = other DNA
cgtctttcgggagcttgcctaaaca

SEQ ID NO: 745 - DNA, 25 bp, synthetic construct
  misc_feature 1..25: note = >Male | ENST00000430988 | 16895114
  source 1..25: mol_type = other DNA
tctttcgggagcttgcctaaacacc

SEQ ID NO: 746 - DNA, 25 bp, synthetic construct
  misc_feature 1..25: note = >Male | ENST00000430988 | 16895114
  source 1..25: mol_type = other DNA
ctttcgggagcttgcctaaacacca

SEQ ID NO: 747 - DNA, 25 bp, synthetic construct
  misc_feature 1..25: note = >Male | ENST00000430988 | 16895114
  source 1..25: mol_type = other DNA
tttcgggagcttgcctaaacaccac

SEQ ID NO: 748 - DNA, 25 bp, synthetic construct
  misc_feature 1..25: note = >Male | ENST00000430988 | 16895114
  source 1..25: mol_type = other DNA
caccagcgttgctctgtcatgtgaa

SEQ ID NO: 749 - DNA, 25 bp, synthetic construct
  misc_feature 1..25: note = >Male | ENST00000430988 | 16895114
  source 1..25: mol_type = other DNA
gacacctcagtgtcgtaggtagcgg

SEQ ID NO: 750 - DNA, 25 bp, synthetic construct
  misc_feature 1..25: note = >Male | ENST00000430988 | 16895114
  source 1..25: mol_type = other DNA
taggtagcggagatgcaagtgttcc

SEQ ID NO: 751 - DNA, 25 bp, synthetic construct
  misc_feature 1..25: note = >Male | ENST00000430988 | 16895114
  source 1..25: mol_type = other DNA
tttctcggaaaaggaagccgatgct

SEQ ID NO: 752 - DNA, 25 bp, synthetic construct
  misc_feature 1..25: note = >Male | ENST00000430988 | 16895114
  source 1..25: mol_type = other DNA
ttctcggaaaaggaagccgatgctg

SEQ ID NO: 753 - DNA, 25 bp, synthetic construct
  misc_feature 1..25: note = >Male | ENST00000430988 | 16895114
  source 1..25: mol_type = other DNA
ttctattacaccaaacccagagcgg

SEQ ID NO: 754 - DNA, 25 bp, synthetic construct
  misc_feature 1..25: note = >Male | ENST00000430988 | 16895114
  source 1..25: mol_type = other DNA
tctattacaccaaacccagagcggg

SEQ ID NO: 755 - DNA, 25 bp, synthetic construct
  misc_feature 1..25: note = >Male | ENST00000430988 | 16895114
  source 1..25: mol_type = other DNA
tattacaccaaacccagagcgggca

SEQ ID NO: 756 - DNA, 25 bp, synthetic construct
  misc_feature 1..25: note = >Male | ENST00000430988 | 16895114
  source 1..25: mol_type = other DNA
aagagactacagaagcacaggcgcc

SEQ ID NO: 757 - DNA, 25 bp, synthetic construct
  misc_feature 1..25: note = >Male | ENST00000430988 | 16895114
  source 1..25: mol_type = other DNA
tacagaagcacaggcgcctgctggg

SEQ ID NO: 758 - DNA, 25 bp, synthetic construct
  misc_feature 1..25: note = >Male | ENST00000440657 | 16773468
  source 1..25: mol_type = other DNA
gcagacatttaaacgttaccattca

SEQ ID NO: 759 - DNA, 25 bp, synthetic construct
  misc_feature 1..25: note = >Male | ENST00000440657 | 16773468
  source 1..25: mol_type = other DNA
gcagcagacatttaaacgttaccat

SEQ ID NO: 760 - DNA, 25 bp, synthetic construct
  misc_feature 1..25: note = >Male | ENST00000440657 | 16773468
  source 1..25: mol_type = other DNA
tagcagcagacatttaaacgttacc

SEQ ID NO: 761 - DNA, 25 bp, synthetic construct
  misc_feature 1..25: note = >Male | ENST00000440657 | 16773468
  source 1..25: mol_type = other DNA
ttccgtagcagcagacatttaaacg

SEQ ID NO: 762 - DNA, 25 bp, synthetic construct
  misc_feature 1..25: note = >Male | ENST00000440657 | 16773468
  source 1..25: mol_type = other DNA
gcttccgtagcagcagacatttaaa

SEQ ID NO: 763 - DNA, 25 bp, synthetic construct
  misc_feature 1..25: note = >Male | ENST00000440657 | 16773468
  source 1..25: mol_type = other DNA
tgcttccgtagcagcagacatttaa

SEQ ID NO: 764 - DNA, 25 bp, synthetic construct
  misc_feature 1..25: note = >Male | ENST00000440657 | 16773468
  source 1..25: mol_type = other DNA
tcttgcttccgtagcagcagacatt

SEQ ID NO: 765 - DNA, 25 bp, synthetic construct
  misc_feature 1..25: note = >Male | ENST00000440657 | 16773468
  source 1..25: mol_type = other DNA
gaattttcttgcttccgtagcagca

SEQ ID NO: 766 - DNA, 25 bp, synthetic construct
  misc_feature 1..25: note = >Male | ENST00000440657 | 16773468
  source 1..25: mol_type = other DNA
ccacacagggagtgaattttcttgc

SEQ ID NO: 767 - DNA, 25 bp, synthetic construct
  misc_feature 1..25: note = >Male | ENST00000440657 | 16773468
  source 1..25: mol_type = other DNA
aacagaacacttctcagaatcagca

SEQ ID NO: 768 - DNA, 25 bp, synthetic construct
  misc_feature 1..25: note = >Male | ENST00000440657 | 16773468
  source 1..25: mol_type = other DNA
tgggctggacttaaacagaacactt

SEQ ID NO: 769 - DNA, 25 bp, synthetic construct
  misc_feature 1..25: note = >Male | ENST00000440657 | 16773468
  source 1..25: mol_type = other DNA
gatgggctggacttaaacagaacac

SEQ ID NO: 770 - DNA, 25 bp, synthetic construct
  misc_feature 1..25: note = >Male | ENST00000440657 | 16773468
  source 1..25: mol_type = other DNA
gagaagatgggctggacttaaacag

SEQ ID NO: 771 - DNA, 25 bp, synthetic construct
  misc_feature 1..25: note = >Male | ENST00000440657 | 16773468
  source 1..25: mol_type = other DNA
ggagtaccaagttctgcattcagta

SEQ ID NO: 772 - DNA, 25 bp, synthetic construct
  misc_feature 1..25: note = >Male | ENST00000440657 | 16773468
  source 1..25: mol_type = other DNA
tggagtaccaagttctgcattcagt

SEQ ID NO: 773 - DNA, 25 bp, synthetic construct
  misc_feature 1..25: note = >Male | ENST00000440657 | 16773468
  source 1..25: mol_type = other DNA
gatctggagtaccaagttctgcatt

SEQ ID NO: 774 - DNA, 25 bp, synthetic construct
  misc_feature 1..25: note = >Male | ENST00000440657 | 16773468
  source 1..25: mol_type = other DNA
catttacttctgcagtttgatctgg

SEQ ID NO: 775 - DNA, 25 bp, synthetic construct
  misc_feature 1..25: note = >Male | ProbSetID_17118935 | 17118935
  source 1..25: mol_type = other DNA
aggattacacacctcgcaaaccgct

SEQ ID NO: 776 - DNA, 25 bp, synthetic construct
  misc_feature 1..25: note = >Male | ProbSetID_17118935 | 17118935
  source 1..25: mol_type = other DNA
taggattacacacctcgcaaaccgc

SEQ ID NO: 777 - DNA, 25 bp, synthetic construct
  misc_feature 1..25: note = >Male | ProbSetID_17118935 | 17118935
  source 1..25: mol_type = other DNA
ctaggattacacacctcgcaaaccg

SEQ ID NO: 778 - DNA, 25 bp, synthetic construct
  misc_feature 1..25: note = >Male | ProbSetID_17118935 | 17118935
  source 1..25: mol_type = other DNA
actaggattacacacctcgcaaacc

SEQ ID NO: 779 - DNA, 25 bp, synthetic construct
  misc_feature 1..25: note = >Male | ProbSetID_17118935 | 17118935
  source 1..25: mol_type = other DNA
aactaggattacacacctcgcaaac

SEQ ID NO: 780 - DNA, 25 bp, synthetic construct
  misc_feature 1..25: note = >Male | ProbSetID_17118935 | 17118935
  source 1..25: mol_type = other DNA
taactaggattacacacctcgcaaa

SEQ ID NO: 781 - DNA, 25 bp, synthetic construct
  misc_feature 1..25: note = >Male | ProbSetID_17118935 | 17118935
  source 1..25: mol_type = other DNA
gtaactaggattacacacctcgcaa

SEQ ID NO: 782 - DNA, 25 bp, synthetic construct
  misc_feature 1..25: note = >Male | NM_007117 | 16945454
  source 1..25: mol_type = other DNA
aaccaatgtagaacgcagacgggcg

SEQ ID NO: 783 - DNA, 25 bp, synthetic construct
  misc_feature 1..25: note = >Male | NM_007117 | 16945454
  source 1..25: mol_type = other DNA
acacggagacgacggagaaacacag

SEQ ID NO: 784 - DNA, 25 bp, synthetic construct
  misc_feature 1..25: note = >Male | NM_007117 | 16945454
  source 1..25: mol_type = other DNA
tagagggcgagacaaagaccgagat

SEQ ID NO: 785 - DNA, 25 bp, synthetic construct
  misc_feature 1..25: note = >Male | NM_007117 | 16945454
  source 1..25: mol_type = other DNA
cgattaaacacaggcgcacgagaga

SEQ ID NO: 786 - DNA, 25 bp, synthetic construct
  misc_feature 1..25: note = >Male | NM_007117 | 16945454
  source 1..25: mol_type = other DNA
gatctgttgacgagaacacgcgtgt

SEQ ID NO: 787 - DNA, 25 bp, synthetic construct
  misc_feature 1..25: note = >Male | NM_007117 | 16945454
  source 1..25: mol_type = other DNA
tccgcagtcggcaggtcaggagtct

SEQ ID NO: 788 - DNA, 25 bp, synthetic construct
  misc_feature 1..25: note = >Male | NM_007117 | 16945454
  source 1..25: mol_type = other DNA
cggtcaggttcagggtcaaagccag

SEQ ID NO: 789 - DNA, 25 bp, synthetic construct
  misc_feature 1..25: note = >Male | NM_007117 | 16945454
  source 1..25: mol_type = other DNA
caccggtcaggttcagggtcaaagc

SEQ ID NO: 790 - DNA, 25 bp, synthetic construct
  misc_feature 1..25: note = >Male | NM_007117 | 16945454
  source 1..25: mol_type = other DNA
cctggatgctgacgtttggagagcc

SEQ ID NO: 791 - DNA, 25 bp, synthetic construct
  misc_feature 1..25: note = >Male | NM_007117 | 16945454
  source 1..25: mol_type = other DNA
tgtgctgggttacatcgactgacca

SEQ ID NO: 792 - DNA, 25 bp, synthetic construct
  misc_feature 1..25: note = >Male | NM_007117 | 16945454
  source 1..25: mol_type = other DNA
ctgccgcttcgggacagcatatgca

SEQ ID NO: 793 - DNA, 25 bp, synthetic construct
  misc_feature 1..25: note = >Male | NM_007117 | 16945454
  source 1..25: mol_type = other DNA
tttgagccttgggatctgccagcct

SEQ ID NO: 794 - DNA, 25 bp, synthetic construct
  misc_feature 1..25: note = >Male | NM_007117 | 16945454
  source 1..25: mol_type = other DNA
gcgtttttcaggcatcaggtcttcc

SEQ ID NO: 795 - DNA, 25 bp, synthetic construct
  misc_feature 1..25: note = >Male | NM_007117 | 16945454
  source 1..25: mol_type = other DNA
cagaaggcccgcttgaccataggct

SEQ ID NO: 796 - DNA, 25 bp, synthetic construct
  misc_feature 1..25: note = >Male | NM_007117 | 16945454
  source 1..25: mol_type = other DNA
cgaccagggtgctgccgcttttcct

SEQ ID NO: 797 - DNA, 25 bp, synthetic construct
  misc_feature 1..25: note = >Male | NM_007117 | 16945454
  source 1..25: mol_type = other DNA
ttagaccacaaactcgacttcaggg

SEQ ID NO: 798 - DNA, 25 bp, synthetic construct
  misc_feature 1..25: note = >Male | NM_007117 | 16945454
  source 1..25: mol_type = other DNA
gctcaggcttatggtcagggatcta

SEQ ID NO: 799 - DNA, 25 bp, synthetic construct
  misc_feature 1..25: note = >Male | NM_007117 | 16945454
  source 1..25: mol_type = other DNA
cagaaggaccatcttagccaaggct

SEQ ID NO: 800 - DNA, 25 bp, synthetic construct
  misc_feature 1..25: note = >Male | NM_007117 | 16945454
  source 1..25: mol_type = other DNA
tggaagctggatttgcacacaacgc

SEQ ID NO: 801 - DNA, 25 bp, synthetic construct
  misc_feature 1..25: note = >Male | NM_007117 | 16945454
  source 1..25: mol_type = other DNA
gtttacacatggagaatcctgccag

SEQ ID NO: 802 - DNA, 25 bp, synthetic construct
  misc_feature 1..25: note = >Male | NM_007117 | 16945454
  source 1..25: mol_type = other DNA
tccaagcagtaacacagctgtgggt

SEQ ID NO: 803 - DNA, 25 bp, synthetic construct
  misc_feature 1..25: note = >Male | NM_007117 | 16945454
  source 1..25: mol_type = other DNA
tacaaaccacaattcctaggcgatg

SEQ ID NO: 804 - DNA, 25 bp, synthetic construct
  misc_feature 1..25: note = >Male | XR_109422 | 16848054
  source 1..25: mol_type = other DNA
ggtgtgtgagcactataggacctat

SEQ ID NO: 805 - DNA, 25 bp, synthetic construct
  misc_feature 1..25: note = >Male | XR_109422 | 16848054
  source 1..25: mol_type = other DNA
taggcagcctgtaattgtcatcacc

SEQ ID NO: 806 - DNA, 25 bp, synthetic construct
  misc_feature 1..25: note = >Male | XR_109422 | 16848054
  source 1..25: mol_type = other DNA
cctatggacagagggattaagaact

SEQ ID NO: 807 - DNA, 25 bp, synthetic construct
  misc_feature 1..25: note = >Male | XR_109422 | 16848054
  source 1..25: mol_type = other DNA
taatcaccaagatccccagaattgt

SEQ ID NO: 808 - DNA, 25 bp, synthetic construct
  misc_feature 1..25: note = >Male | XR_109422 | 16848054
  source 1..25: mol_type = other DNA
taggtggtccgagacattctgctga

SEQ ID NO: 809 - DNA, 25 bp, synthetic construct
  misc_feature 1..25: note = >Male | XR_109422 | 16848054
  source 1..25: mol_type = other DNA
tctgctgattggacatagacaggct

SEQ ID NO: 810 - DNA, 25 bp, synthetic construct
  misc_feature 1..25: note = >Male | XR_109422 | 16848054
  source 1..25: mol_type = other DNA
agtggtatggtaagtcaatttctcc

SEQ ID NO: 811 - DNA, 25 bp, synthetic construct
  misc_feature 1..25: note = >Male | XR_109422 | 16848054
  source 1..25: mol_type = other DNA
tgtctcgggctttctgtccatttgg

SEQ ID NO: 812 - DNA, 25 bp, synthetic construct
  misc_feature 1..25: note = >Male | XR_109422 | 16848054
  source 1..25: mol_type = other DNA
tgataacgtagagtggttcttccca

SEQ ID NO: 813 - DNA, 25 bp, synthetic construct
  misc_feature 1..25: note = >Male | XR_109422 | 16848054
  source 1..25: mol_type = other DNA
taacgtagagtggttcttcccatgg

SEQ ID NO: 814 - DNA, 25 bp, synthetic construct
  misc_feature 1..25: note = >Male | XR_109422 | 16848054
  source 1..25: mol_type = other DNA
aacgtagagtggttcttcccatgga

SEQ ID NO: 815 - DNA, 25 bp, synthetic construct
  misc_feature 1..25: note = >Male | XR_109422 | 16848054
  source 1..25: mol_type = other DNA
gaaaagcccacataatctctggctg

SEQ ID NO: 816 - DNA, 25 bp, synthetic construct
  misc_feature 1..25: note = >Male | XR_109422 | 16848054
  source 1..25: mol_type = other DNA
cctcagcgttgttcccgtgacttca

SEQ ID NO: 817 - DNA, 25 bp, synthetic construct
  misc_feature 1..25: note = >Male | XR_109422 | 16848054
  source 1..25: mol_type = other DNA
tttttctttggcagatcgttccaca

SEQ ID NO: 818 - DNA, 25 bp, synthetic construct
  misc_feature 1..25: note = >Male | XR_109422 | 16848054
  source 1..25: mol_type = other DNA
ttctttggcagatcgttccacaagg

SEQ ID NO: 819 - DNA, 25 bp, synthetic construct
  misc_feature 1..25: note = >Male | XR_109422 | 16848054
  source 1..25: mol_type = other DNA
tctttggcagatcgttccacaaggc

SEQ ID NO: 820 - DNA, 25 bp, synthetic construct
  misc_feature 1..25: note = >Male | XR_109422 | 16848054
  source 1..25: mol_type = other DNA
ctttggcagatcgttccacaaggcc

SEQ ID NO: 821 - DNA, 25 bp, synthetic construct
  misc_feature 1..25: note = >Male | XR_109422 | 16848054
  source 1..25: mol_type = other DNA
tttggcagatcgttccacaaggcca

SEQ ID NO: 822 - DNA, 25 bp, synthetic construct
  misc_feature 1..25: note = >Male | XR_109422 | 16848054
  source 1..25: mol_type = other DNA
ttggcagatcgttccacaaggccat

SEQ ID NO: 823 - DNA, 25 bp, synthetic construct
  misc_feature 1..25: note = >Male | XR_109422 | 16848054
  source 1..25: mol_type = other DNA
tttatattcgaccactccctagacc

SEQ ID NO: 824 - DNA, 25 bp, synthetic construct
  misc_feature 1..25: note = >Male | ENST00000456944 | 17095583
  source 1..25: mol_type = other DNA
tcagggaaagaggtacaatgcaatc

SEQ ID NO: 825 - DNA, 25 bp, synthetic construct
  misc_feature 1..25: note = >Male | ENST00000456944 | 17095583
  source 1..25: mol_type = other DNA
tgcaatcagtagaaaggtcaccaga

SEQ ID NO: 826 - DNA, 25 bp, synthetic construct
  misc_feature 1..25: note = >Male | ENST00000456944 | 17095583
  source 1..25: mol_type = other DNA
tacggtcattaccacgacaaaggga

SEQ ID NO: 827 - DNA, 25 bp, synthetic construct
  misc_feature 1..25: note = >Male | ENST00000456944 | 17095583
  source 1..25: mol_type = other DNA
gagcacagaatttccagcaggcagg

SEQ ID NO: 828 - DNA, 25 bp, synthetic construct
  misc_feature 1..25: note = >Male | ENST00000456944 | 17095583
  source 1..25: mol_type = other DNA
tccagcaggcaggatgcatccacag

SEQ ID NO: 829 - DNA, 25 bp, synthetic construct
  misc_feature 1..25: note = >Male | ENST00000456944 | 17095583
  source 1..25: mol_type = other DNA
ccagcagaggccgtgagaaaagcct

SEQ ID NO: 830 - DNA, 25 bp, synthetic construct
  misc_feature 1..25: note = >Male | ENST00000456944 | 17095583
  source 1..25: mol_type = other DNA
tcttgttagtaaatgcagcctgtct

SEQ ID NO: 831 - DNA, 25 bp, synthetic construct
  misc_feature 1..25: note = >Male | ENST00000456944 | 17095583
  source 1..25: mol_type = other DNA
taaatgcagcctgtcttaatactgc

SEQ ID NO: 832 - DNA, 25 bp, synthetic construct
  misc_feature 1..25: note = >Male | ENST00000456944 | 17095583
  source 1..25: mol_type = other DNA
tatgcattgtttccatccccaaatg

SEQ ID NO: 833 - DNA, 25 bp, synthetic construct
  misc_feature 1..25: note = >Male | ENST00000456944 | 17095583
  source 1..25: mol_type = other DNA
gaacttgtgattaaaggtccatgca

SEQ ID NO: 834 - DNA, 25 bp, synthetic construct
  misc_feature 1..25: note = >Male | ENST00000456944 | 17095583
  source 1..25: mol_type = other DNA
tgattaaaggtccatgcaacccaga

SEQ ID NO: 835 - DNA, 25 bp, synthetic construct
  misc_feature 1..25: note = >Male | ENST00000456944 | 17095583
  source 1..25: mol_type = other DNA
gattaaaggtccatgcaacccagaa

SEQ ID NO: 836 - DNA, 25 bp, synthetic construct
  misc_feature 1..25: note = >Male | ENST00000456944 | 17095583
  source 1..25: mol_type = other DNA
taaaggtccatgcaacccagaaatg

SEQ ID NO: 837 - DNA, 25 bp, synthetic construct
  misc_feature 1..25: note = >Male | ENST00000456944 | 17095583
  source 1..25: mol_type = other DNA
tgttggaagccagaagtgacctgag

SEQ ID NO: 838 - DNA, 25 bp, synthetic construct
  misc_feature 1..25: note = >Male | ENST00000456944 | 17095583
  source 1..25: mol_type = other DNA
ctggcctgctctccttcaaggtgaa

SEQ ID NO: 839 - DNA, 25 bp, synthetic construct
  misc_feature 1..25: note = >Male | ENST00000456944 | 17095583
  source 1..25: mol_type = other DNA
tgaaacccactgtgccaagggttgg

SEQ ID NO: 840 - DNA, 25 bp, synthetic construct
  misc_feature 1..25: note = >Male | ENST00000456944 | 17095583
  source 1..25: mol_type = other DNA
tgtgattctcaccaacatcttcagg

SEQ ID NO: 841 - DNA, 25 bp, synthetic construct
  misc_feature 1..25: note = >Male | ENST00000456944 | 17095583
  source 1..25: mol_type = other DNA
taagcaaagtctctcccatgaaaca

SEQ ID NO: 842 - DNA, 25 bp, synthetic construct
  misc_feature 1..25: note = >Male | NM_080717 | 16757527
  source 1..25: mol_type = other DNA
ctgtggctgcgcagacccggcactg

SEQ ID NO: 843 - DNA, 25 bp, synthetic construct
  misc_feature 1..25: note = >Male | NM_080717 | 16757527
  source 1..25: mol_type = other DNA
acgtccggaacctgtggctgcgcag

SEQ ID NO: 844 - DNA, 25 bp, synthetic construct
  misc_feature 1..25: note = >Male | NM_080717 | 16757527
  source 1..25: mol_type = other DNA
aagacgtccggaacctgtggctgcg

SEQ ID NO: 845 - DNA, 25 bp, synthetic construct
  misc_feature 1..25: note = >Male | NM_080717 | 16757527
  source 1..25: mol_type = other DNA
ccaagacgtccggaacctgtggctg

SEQ ID NO: 846 - DNA, 25 bp, synthetic construct
  misc_feature 1..25: note = >Male | NM_080717 | 16757527
  source 1..25: mol_type = other DNA
cattcctccggagaaagccgggttg

SEQ ID NO: 847 - DNA, 25 bp, synthetic construct
  misc_feature 1..25: note = >Male | NM_080717 | 16757527
  source 1..25: mol_type = other DNA
ctcattcctccggagaaagccgggt

SEQ ID NO: 848 - DNA, 25 bp, synthetic construct
  misc_feature 1..25: note = >Male | NM_080717 | 16757527
  source 1..25: mol_type = other DNA
tagagcggcgcctcggtattcattt

SEQ ID NO: 849 - DNA, 25 bp, synthetic construct
  misc_feature 1..25: note = >Male | NM_080717 | 16757527
  source 1..25: mol_type = other DNA
tatgcaagtctgcaagagggcactg

SEQ ID NO: 850 - DNA, 25 bp, synthetic construct
  misc_feature 1..25: note = >Male | NM_080717 | 16757527
  source 1..25: mol_type = other DNA
gcaaggcacattacggagaactccc

SEQ ID NO: 851 - DNA, 25 bp, synthetic construct
  misc_feature 1..25: note = >Male | NM_080717 | 16757527
  source 1..25: mol_type = other DNA
gccactctgggacgaattgcattct

SEQ ID NO: 852 - DNA, 25 bp, synthetic construct
  misc_feature 1..25: note = >Male | NM_080717 | 16757527
  source 1..25: mol_type = other DNA
tcggcgggccctggtacaataatta

SEQ ID NO: 853 - DNA, 25 bp, synthetic construct
  misc_feature 1..25: note = >Male | NM_080717 | 16757527
  source 1..25: mol_type = other DNA
aaaatagagtgcctcgtgcctcggc

SEQ ID NO: 854 - DNA, 25 bp, synthetic construct
  misc_feature 1..25: note = >Male | NM_080717 | 16757527
  source 1..25: mol_type = other DNA
cttagaaattgtgcccattgatcag

SEQ ID NO: 855 - DNA, 25 bp, synthetic construct
  misc_feature 1..25: note = >Male | NM_080717 | 16757527
  source 1..25: mol_type = other DNA
gcttagaaattgtgcccattgatca

SEQ ID NO: 856 - DNA, 25 bp, synthetic construct
  misc_feature 1..25: note = >Male | NM_080717 | 16757527
  source 1..25: mol_type = other DNA
tgcttagaaattgtgcccattgatc

SEQ ID NO: 857 - DNA, 25 bp, synthetic construct
  misc_feature 1..25: note = >Male | NM_080717 | 16757527
  source 1..25: mol_type = other DNA
ctgcttagaaattgtgcccattgat

SEQ ID NO: 858 - DNA, 25 bp, synthetic construct
  misc_feature 1..25: note = >Male | NM_080717 | 16757527
  source 1..25: mol_type = other DNA
aagtttgcgagcatccaccactgcg

SEQ ID NO: 859 - DNA, 25 bp, synthetic construct
  misc_feature 1..25: note = >Male | NM_080717 | 16757527
  source 1..25: mol_type = other DNA
tagtgggtttccagcggtgcgcaaa

SEQ ID NO: 860 - DNA, 25 bp, synthetic construct
  misc_feature 1..25: note = >Male | NM_080717 | 16757527
  source 1..25: mol_type = other DNA
tacgcggtacgttttgcaactcaac

SEQ ID NO: 861 - DNA, 25 bp, synthetic construct
  misc_feature 1..25: note = >Male | NM_080717 | 16757527
  source 1..25: mol_type = other DNA
ggcgtctacgcggtacgttttgcaa

SEQ ID NO: 862 - DNA, 25 bp, synthetic construct
  misc_feature 1..25: note = >Male | NM_080717 | 16757527
  source 1..25: mol_type = other DNA
tgggcaggcctagctcttctctcgg

SEQ ID NO: 863 - DNA, 25 bp, synthetic construct
  misc_feature 1..25: note = >Male | NM_080717 | 16757527
  source 1..25: mol_type = other DNA
cgctctccggctctgtgctgggcag

SEQ ID NO: 864 - DNA, 25 bp, synthetic construct
  misc_feature 1..25: note = >Male | NM_080717 | 16757527
  source 1..25: mol_type = other DNA
cagaatcctttgaatgccggtccag

SEQ ID NO: 865 - DNA, 25 bp, synthetic construct
  misc_feature 1..25: note = >Male | NM_080717 | 16757527
  source 1..25: mol_type = other DNA
gaaagcgacagttctccgaaaggag

SEQ ID NO: 866 - DNA, 25 bp, synthetic construct
  misc_feature 1..25: note = >Male | NM_080717 | 16757527
  source 1..25: mol_type = other DNA
tatcaacagcaatgggcccctttgg

SEQ ID NO: 867 - DNA, 25 bp, synthetic construct
  misc_feature 1..25: note = >Male | NM_080717 | 16757527
  source 1..25: mol_type = other DNA
cattgggatccttctcagctgggcc

SEQ ID NO: 868 - DNA, 25 bp, synthetic construct
  misc_feature 1..25: note = >Male | TCONS_l2_00026656-XLOC_l2_013873 | 17058221
  source 1..25: mol_type = other DNA
cccgcagagcctggaaaggcactga

SEQ ID NO: 869 - DNA, 25 bp, synthetic construct
  misc_feature 1..25: note = >Male | TCONS_l2_00026656-XLOC_l2_013873 | 17058221
  source 1..25: mol_type = other DNA
ccgcagagcctggaaaggcactgac

SEQ ID NO: 870 - DNA, 25 bp, synthetic construct
  misc_feature 1..25: note = >Male | TCONS_l2_00026656-XLOC_l2_013873 | 17058221
  source 1..25: mol_type = other DNA
cgcagagcctggaaaggcactgacc

SEQ ID NO: 871 - DNA, 25 bp, synthetic construct
  misc_feature 1..25: note = >Male | TCONS_l2_00026656-XLOC_l2_013873 | 17058221
  source 1..25: mol_type = other DNA
gcagagcctggaaaggcactgaccc

SEQ ID NO: 872 - DNA, 25 bp, synthetic construct
  misc_feature 1..25: note = >Male | TCONS_l2_00026656-XLOC_l2_013873 | 17058221
  source 1..25: mol_type = other DNA
ggaatacctctatgctgttttaagc

SEQ ID NO: 873 - DNA, 25 bp, synthetic construct
  misc_feature 1..25: note = >Male | TCONS_l2_00026656-XLOC_l2_013873 | 17058221
  source 1..25: mol_type = other DNA
cccaactgggtatggttcagatctg

SEQ ID NO: 874 - DNA, 25 bp, synthetic construct
  misc_feature 1..25: note = >Male | TCONS_l2_00026656-XLOC_l2_013873 | 17058221
  source 1..25: mol_type = other DNA
ccaactgggtatggttcagatctgc

SEQ ID NO: 875 - DNA, 25 bp, synthetic construct
  misc_feature 1..25: note = >Male | TCONS_l2_00026656-XLOC_l2_013873 | 17058221
  source 1..25: mol_type = other DNA
caactgggtatggttcagatctgca

SEQ ID NO: 876 - DNA, 25 bp, synthetic construct
  misc_feature 1..25: note = >Male | TCONS_l2_00026656-XLOC_l2_013873 | 17058221
  source 1..25: mol_type = other DNA
tgtgctgggtcggagtttgtcaacc

SEQ ID NO: 877 - DNA, 25 bp, synthetic construct
  misc_feature 1..25: note = >Male | TCONS_l2_00026656-XLOC_l2_013873 | 17058221
  source 1..25: mol_type = other DNA
aggctccactccgaggaaaaaccag

SEQ ID NO: 878 - DNA, 25 bp, synthetic construct
  misc_feature 1..25: note = >Male | TCONS_l2_00026656-XLOC_l2_013873 | 17058221
  source 1..25: mol_type = other DNA
gaggaaaaaccagcgcagctccagg

SEQ ID NO: 879 - DNA, 25 bp, synthetic construct
  misc_feature 1..25: note = >Male | TCONS_l2_00026656-XLOC_l2_013873 | 17058221
  source 1..25: mol_type = other DNA
gcactgtagcgccactgtgacgtcg

SEQ ID NO: 880 - DNA, 25 bp, synthetic construct
  misc_feature 1..25: note = >Male | ExonID_5262586 | 17025653
  source 1..25: mol_type = other DNA
gactttaagcaaacagacaggcctg

SEQ ID NO: 881 - DNA, 25 bp, synthetic construct
  misc_feature 1..25: note = >Male | ExonID_5262586 | 17025653
  source 1..25: mol_type = other DNA
gagtctcttcatgcctctgcgctga

SEQ ID NO: 882 - DNA, 25 bp, synthetic construct
  misc_feature 1..25: note = >Male | ExonID_5262586 | 17025653
  source 1..25: mol_type = other DNA
tttgagcacttgcatcgccgtcagg

SEQ ID NO: 883 - DNA, 25 bp, synthetic construct
  misc_feature 1..25: note = >Male | ExonID_5262586 | 17025653
  source 1..25: mol_type = other DNA
tagctgcgtcagatatcgagtccca

SEQ ID NO: 884 - DNA, 25 bp, synthetic construct
  misc_feature 1..25: note = >Male | ExonID_5262586 | 17025653
  source 1..25: mol_type = other DNA
tgcaatcatgctctgaggcctccca

SEQ ID NO: 885 - DNA, 25 bp, synthetic construct
  misc_feature 1..25: note = >Male | ExonID_5262586 | 17025653
  source 1..25: mol_type = other DNA
tcttattgctgatgaccgtcctccg

SEQ ID NO: 886 - DNA, 25 bp, synthetic construct
  misc_feature 1..25: note = >Male | ExonID_5262586 | 17025653
  source 1..25: mol_type = other DNA
ttagactcagaagcctaggagcctg

SEQ ID NO: 887 - DNA, 25 bp, synthetic construct
  misc_feature 1..25: note = >Male | ExonID_5262586 | 17025653
  source 1..25: mol_type = other DNA
tccgtttgaagagagctcgcagcaa

SEQ ID NO: 888 - DNA, 25 bp, synthetic construct
  misc_feature 1..25: note = >Male | ExonID_5262586 | 17025653
  source 1..25: mol_type = other DNA
gatgagagccatggtctccttcctg

SEQ ID NO: 889 - DNA, 25 bp, synthetic construct
  misc_feature 1..25: note = >Male | ExonID_5262586 | 17025653
  source 1..25: mol_type = other DNA
tgtactcgatcgtcccgcagaagga

SEQ ID NO: 890 - DNA, 25 bp, synthetic construct
  misc_feature 1..25: note = >Male | ExonID_5262586 | 17025653
  source 1..25: mol_type = other DNA
tggagatggtctaaagccaaggcca

SEQ ID NO: 891 - DNA, 25 bp, synthetic construct
  misc_feature 1..25: note = >Male | ExonID_5262586 | 17025653
  source 1..25: mol_type = other DNA
gatccttgtcctaaaaccttcagca

SEQ ID NO: 892 - DNA, 25 bp, synthetic construct
  misc_feature 1..25: note = >Male | ExonID_5262586 | 17025653
  source 1..25: mol_type = other DNA
caagtctgttcctgaatcggctgca

SEQ ID NO: 893 - DNA, 25 bp, synthetic construct
  misc_feature 1..25: note = >Male | ExonID_5262586 | 17025653
  source 1..25: mol_type = other DNA
tcaagggacgagaattcagatcatc

SEQ ID NO: 894 - DNA, 25 bp, synthetic construct
  misc_feature 1..25: note = >Male | ExonID_5262586 | 17025653
  source 1..25: mol_type = other DNA
gacttcctgcgcaggtacacagaga

SEQ ID NO: 895 - DNA, 25 bp, synthetic construct
  misc_feature 1..25: note = >Male | ExonID_5262586 | 17025653
  source 1..25: mol_type = other DNA
cgagctgcggtcacaaagggcaggc

SEQ ID NO: 896 - DNA, 25 bp, synthetic construct
  misc_feature 1..25: note = >Male | ExonID_5262586 | 17025653
  source 1..25: mol_type = other DNA
taattgttgatcagcaaacgcagcc

SEQ ID NO: 897 - DNA, 25 bp, synthetic construct
  misc_feature 1..25: note = >Male | ExonID_5262586 | 17025653
  source 1..25: mol_type = other DNA
cagcttcttgtgtggcagcctgatc

SEQ ID NO: 898 - DNA, 25 bp, synthetic construct
  misc_feature 1..25: note = >Male | ExonID_5262586 | 17025653
  source 1..25: mol_type = other DNA
ttatcaccttgtgggctgctgcgcc

SEQ ID NO: 899 - DNA, 25 bp, synthetic construct
  misc_feature 1..25: note = >Male | ExonID_5262586 | 17025653
  source 1..25: mol_type = other DNA
tacctttgtcttgcaggcagctgag

SEQ ID NO: 900 - DNA, 25 bp, synthetic construct
  misc_feature 1..25: note = >Male | ExonID_5262586 | 17025653
  source 1..25: mol_type = other DNA
aactgctcacagatatttaatttca

SEQ ID NO: 901 - DNA, 25 bp, synthetic construct
  misc_feature 1..25: note = >Male | ExonID_5262586 | 17025653
  source 1..25: mol_type = other DNA
caatctccccaaacaaagcagagtg

SEQ ID NO: 902 - DNA, 25 bp, synthetic construct
  misc_feature 1..25: note = >Male | ExonID_5262586 | 17025653
  source 1..25: mol_type = other DNA
gatctttttccatagttccagcaac

SEQ ID NO: 903 - DNA, 25 bp, synthetic construct
  misc_feature 1..25: note = >Male | ExonID_5262586 | 17025653
  source 1..25: mol_type = other DNA
gttgagtctgaggatgccgctgcca

SEQ ID NO: 904 - DNA, 25 bp, synthetic construct
  misc_feature 1..25: note = >Male | ExonID_5262586 | 17025653
  source 1..25: mol_type = other DNA
taggccatataggacgctggtcaaa

SEQ ID NO: 905 - DNA, 25 bp, synthetic construct
  misc_feature 1..25: note = >Male | AK293404 | 16868954
  source 1..25: mol_type = other DNA
tgacttaccttacctgagcttcaga

SEQ ID NO: 906 - DNA, 25 bp, synthetic construct
  misc_feature 1..25: note = >Male | AK293404 | 16868954
  source 1..25: mol_type = other DNA
acagacagagtgttccgggtcccct

SEQ ID NO: 907 - DNA, 25 bp, synthetic construct
  misc_feature 1..25: note = >Male | AK293404 | 16868954
  source 1..25: mol_type = other DNA
ttgtgcttctgcaaggctcgccgga

SEQ ID NO: 908 - DNA, 25 bp, synthetic construct
  misc_feature 1..25: note = >Male | AK293404 | 16868954
  source 1..25: mol_type = other DNA
gatgcttcgatacaggttcccgtgg

SEQ ID NO: 909 - DNA, 25 bp, synthetic construct
  misc_feature 1..25: note = >Male | AK293404 | 16868954
  source 1..25: mol_type = other DNA
tgatgagcttttctctcgggcaagc

SEQ ID NO: 910 - DNA, 25 bp, synthetic construct
  misc_feature 1..25: note = >Male | AK293404 | 16868954
  source 1..25: mol_type = other DNA
actgagacgcttggtgaggctgatc

SEQ ID NO: 911 - DNA, 25 bp, synthetic construct
  misc_feature 1..25: note = >Male | AK293404 | 16868954
  source 1..25: mol_type = other DNA
tgtcaggagccccactgtagtaagg

SEQ ID NO: 912 - DNA, 25 bp, synthetic construct
  misc_feature 1..25: note = >Male | AK293404 | 16868954
  source 1..25: mol_type = other DNA
ttgtccccatcccaaactgtaatca

SEQ ID NO: 913 - DNA, 25 bp, synthetic construct
  misc_feature 1..25: note = >Male | AK293404 | 16868954
  source 1..25: mol_type = other DNA
tcttcttgggatccctcagtggcct

SEQ ID NO: 914 - DNA, 25 bp, synthetic construct
  misc_feature 1..25: note = >Male | AK293404 | 16868954
  source 1..25: mol_type = other DNA
tttcctgaaagtagatagcggttcc

SEQ ID NO: 915 - DNA, 25 bp, synthetic construct
  misc_feature 1..25: note = >Male | AK293404 | 16868954
  source 1..25: mol_type = other DNA
agaagttccgcagttctcggcagcg

SEQ ID NO: 916 - DNA, 25 bp, synthetic construct
  misc_feature 1..25: note = >Male | AK293404 | 16868954
  source 1..25: mol_type = other DNA
ttcctccgcgcaggcctctgaagag

SEQ ID NO: 917 - DNA, 25 bp, synthetic construct
  misc_feature 1..25: note = >Male | AK293404 | 16868954
  source 1..25: mol_type = other DNA
aaatcctgagggtggtcctgcagcc

SEQ ID NO: 918 - DNA, 25 bp, synthetic construct
  misc_feature 1..25: note = >Male | AK293404 | 16868954
  source 1..25: mol_type = other DNA
tgttgaagctcagatcttgtaccgc

SEQ ID NO: 919 - DNA, 25 bp, synthetic construct
  misc_feature 1..25: note = >Male | AK293404 | 16868954
  source 1..25: mol_type = other DNA
tgccgccgcagggagacactgtaca

SEQ ID NO: 920 - DNA, 25 bp, synthetic construct
  misc_feature 1..25: note = >Male | AK293404 | 16868954
  source 1..25: mol_type = other DNA
agctctttgcctgctgtgcgctcca

SEQ ID NO: 921 - DNA, 25 bp, synthetic construct
  misc_feature 1..25: note = >Male | ENST00000338315 | 16877665
  source 1..25: mol_type = other DNA
gcacggccgtgtaagctgcaggtca

SEQ ID NO: 922 - DNA, 25 bp, synthetic construct
  misc_feature 1..25: note = >Male | ENST00000338315 | 16877665
  source 1..25: mol_type = other DNA
catttactgagtcagcacggccgtg

SEQ ID NO: 923 - DNA, 25 bp, synthetic construct
  misc_feature 1..25: note = >Male | ENST00000338315 | 16877665
  source 1..25: mol_type = other DNA
ttgtacagaatgagaggcgaccggc

SEQ ID NO: 924 - DNA, 25 bp, synthetic construct
  misc_feature 1..25: note = >Male | ENST00000338315 | 16877665
  source 1..25: mol_type = other DNA
aaggcatgagccgtccagaccctgt

SEQ ID NO: 925 - DNA, 25 bp, synthetic construct
  misc_feature 1..25: note = >Male | ENST00000338315 | 16877665
  source 1..25: mol_type = other DNA
tagaaagtcgtgtaccagaggcctc

SEQ ID NO: 926 - DNA, 25 bp, synthetic construct
  misc_feature 1..25: note = >Male | ENST00000338315 | 16877665
  source 1..25: mol_type = other DNA
gtgagcgctgtgtagggcacctgga

SEQ ID NO: 927 - DNA, 25 bp, synthetic construct
  misc_feature 1..25: note = >Male | ENST00000338315 | 16877665
  source 1..25: mol_type = other DNA
gccggacacgatgagcccgtggaca

SEQ ID NO: 928 - DNA, 25 bp, synthetic construct
  misc_feature 1..25: note = >Male | ENST00000338315 | 16877665
  source 1..25: mol_type = other DNA
cagggtggctggatgcatcccagaa

SEQ ID NO: 929 - DNA, 25 bp, synthetic construct
  misc_feature 1..25: note = >Male | ENST00000338315 | 16877665
  source 1..25: mol_type = other DNA
actcctttcaaagggcacctgaccg

SEQ ID NO: 930 - DNA, 25 bp, synthetic construct
  misc_feature 1..25: note = >Male | ENST00000338315 | 16877665
  source 1..25: mol_type = other DNA
ggtaagtcactacaaccacggcagc

SEQ ID NO: 931 - DNA, 25 bp, synthetic construct
  misc_feature 1..25: note = >Male | ENST00000338315 | 16877665
  source 1..25: mol_type = other DNA
tcacccctaggcacagtaaactgat

SEQ ID NO: 932 - DNA, 25 bp, synthetic construct
  misc_feature 1..25: note = >Male | ENST00000338315 | 16877665
  source 1..25: mol_type = other DNA
gatgaacaggaaggagatcaccagc

SEQ ID NO: 933 - DNA, 25 bp, synthetic construct
  misc_feature 1..25: note = >Male | ENST00000338315 | 16877665
  source 1..25: mol_type = other DNA
ctgaacagcagcagagatgaacagg

SEQ ID NO: 934 - DNA, 25 bp, synthetic construct
  misc_feature 1..25: note = >Male | ENST00000338315 | 16877665
  source 1..25: mol_type = other DNA
ggacagttagtaccaggccctggac

SEQ ID NO: 935 - DNA, 25 bp, synthetic construct
  misc_feature 1..25: note = >Male | ENST00000338315 | 16877665
  source 1..25: mol_type = other DNA
agatcccaaaggctgacgtcttctt

SEQ ID NO: 936 - DNA, 25 bp, synthetic construct
  misc_feature 1..25: note = >Male | ENST00000338315 | 16877665
  source 1..25: mol_type = other DNA
gatacaaaggccacgacatatgcca

SEQ ID NO: 937 - DNA, 25 bp, synthetic construct
  misc_feature 1..25: note = >Male | ENST00000338315 | 16877665
  source 1..25: mol_type = other DNA
caggtttacatgaaggaggtgacac

SEQ ID NO: 938 - DNA, 25 bp, synthetic construct
  misc_feature 1..25: note = >Male | ENST00000338315 | 16877665
  source 1..25: mol_type = other DNA
cacatgcgccagacagcttggtgaa

SEQ ID NO: 939 - DNA, 25 bp, synthetic construct
  misc_feature 1..25: note = >Male | ENST00000338315 | 16877665
  source 1..25: mol_type = other DNA
gcttgcttgcagacccctgccttat

SEQ ID NO: 940 - DNA, 25 bp, synthetic construct
  misc_feature 1..25: note = >Male | ENST00000338315 | 16877665
  source 1..25: mol_type = other DNA
ttaggccaggctgtagctggtcctc

SEQ ID NO: 941 - DNA, 25 bp, synthetic construct
  misc_feature 1..25: note = >Male | ENST00000338315 | 16877665
  source 1..25: mol_type = other DNA
gaaaggtcacatactaaactgccag

SEQ ID NO: 942 - DNA, 25 bp, synthetic construct
  misc_feature 1..25: note = >Male | ENST00000338315 | 16877665
  source 1..25: mol_type = other DNA
agggtcccctacacaagagtttctg

SEQ ID NO: 943 - DNA, 25 bp, synthetic construct
  misc_feature 1..25: note = >Male | ENST00000432646 | 17051908
  source 1..25: mol_type = other DNA
tagacccagtcttgcctcctgagaa

SEQ ID NO: 944 - DNA, 25 bp, synthetic construct
  misc_feature 1..25: note = >Male | ENST00000432646 | 17051908
  source 1..25: mol_type = other DNA
cctgaatacaatgcaagggagggag

SEQ ID NO: 945 - DNA, 25 bp, synthetic construct
  misc_feature 1..25: note = >Male | ENST00000432646 | 17051908
  source 1..25: mol_type = other DNA
tgtcattccctaggcatgctcagga

SEQ ID NO: 946 - DNA, 25 bp, synthetic construct
  misc_feature 1..25: note = >Male | ENST00000432646 | 17051908
  source 1..25: mol_type = other DNA
gcacattttgttctagatggagttc

SEQ ID NO: 947 - DNA, 25 bp, synthetic construct
  misc_feature 1..25: note = >Male | ENST00000432646 | 17051908
  source 1..25: mol_type = other DNA
caacaagagaggtcggtgtgccaca

SEQ ID NO: 948 - DNA, 25 bp, synthetic construct
  misc_feature 1..25: note = >Male | ENST00000432646 | 17051908
  source 1..25: mol_type = other DNA
ggttggtcaccaatccctgtggaat

SEQ ID NO: 949 - DNA, 25 bp, synthetic construct
  misc_feature 1..25: note = >Male | ENST00000432646 | 17051908
  source 1..25: mol_type = other DNA
cttggaaggctgactcatgttgtct

SEQ ID NO: 950 - DNA, 25 bp, synthetic construct
  misc_feature 1..25: note = >Male | ENST00000432646 | 17051908
  source 1..25: mol_type = other DNA
cctgcgtttctgtgccatggagtca

SEQ ID NO: 951 - DNA, 25 bp, synthetic construct
  misc_feature 1..25: note = >Male | ENST00000432646 | 17051908
  source 1..25: mol_type = other DNA
ccttctaagttctctgcgacgctca

SEQ ID NO: 952 - DNA, 25 bp, synthetic construct
  misc_feature 1..25: note = >Male | ENST00000432646 | 17051908
  source 1..25: mol_type = other DNA
aaatgcacggtgcaggatgctcccg

SEQ ID NO: 953 - DNA, 25 bp, synthetic construct
  misc_feature 1..25: note = >Male | ENST00000432646 | 17051908
  source 1..25: mol_type = other DNA
gctgcgtcttccatgcgatccggat

SEQ ID NO: 954 - DNA, 25 bp, synthetic construct
  misc_feature 1..25: note = >Male | ENST00000432646 | 17051908
  source 1..25: mol_type = other DNA
tagagtgatttggagaactgactcc

SEQ ID NO: 955 - DNA, 25 bp, synthetic construct
  misc_feature 1..25: note = >Male | ENST00000432646 | 17051908
  source 1..25: mol_type = other DNA
acagttgaccggtccccaagatcag

SEQ ID NO: 956 - DNA, 25 bp, synthetic construct
  misc_feature 1..25: note = >Male | ENST00000432646 | 17051908
  source 1..25: mol_type = other DNA
caaaagatacagcatgttgcatgag

SEQ ID NO: 957 - DNA, 25 bp, synthetic construct
  misc_feature 1..25: note = >Male | ENST00000432646 | 17051908
  source 1..25: mol_type = other DNA
tcatcgtcattcctctggtaggcga

SEQ ID NO: 958 - DNA, 25 bp, synthetic construct
  misc_feature 1..25: note = >Male | ENST00000432646 | 17051908
  source 1..25: mol_type = other DNA
tgcattcttctgcttgggagcgaca

SEQ ID NO: 959 - DNA, 25 bp, synthetic construct
  misc_feature 1..25: note = >Male | ENST00000432646 | 17051908
  source 1..25: mol_type = other DNA
ctatctcgtatctttcttggcgact

SEQ ID NO: 960 - DNA, 25 bp, synthetic construct
  misc_feature 1..25: note = >Male | ENST00000432646 | 17051908
  source 1..25: mol_type = other DNA
gattttctccaatgctccctcgctt

SEQ ID NO: 961 - DNA, 25 bp, synthetic construct
  misc_feature 1..25: note = >Male | ENST00000432646 | 17051908
  source 1..25: mol_type = other DNA
tcttttagcttgcactttagttccc

SEQ ID NO: 962 - DNA, 25 bp, synthetic construct
  misc_feature 1..25: note = >Male | ENST00000432646 | 17051908
  source 1..25: mol_type = other DNA
taagtttgtgggtcatgaattctcc

SEQ ID NO: 963 - DNA, 25 bp, synthetic construct
  misc_feature 1..25: note = >Male | ENST00000432646 | 17051908
  source 1..25: mol_type = other DNA
gaatatcctgtgtcctccagaacaa

SEQ ID NO: 964 - DNA, 25 bp, synthetic construct
  misc_feature 1..25: note = >Male | ENST00000432646 | 17051908
  source 1..25: mol_type = other DNA
tgagttttcgatcggcttcctcctg

SEQ ID NO: 965 - DNA, 25 bp, synthetic construct
  misc_feature 1..25: note = >Male | ENST00000432646 | 17051908
  source 1..25: mol_type = other DNA
tgtcaggcagactcaagggagacct

SEQ ID NO: 966 - DNA, 25 bp, synthetic construct
  misc_feature 1..25: note = >Male | ENST00000432646 | 17051908
  source 1..25: mol_type = other DNA
tctggcgtttctcagcagcttctgc

SEQ ID NO: 967 - DNA, 25 bp, synthetic construct
  misc_feature 1..25: note = >Male | ENST00000432646 | 17051908
  source 1..25: mol_type = other DNA
tgtcatctgacaagccatcttctgg

SEQ ID NO: 968 - DNA, 25 bp, synthetic construct
  misc_feature 1..25: note = >Male | ENST00000432646 | 17051908
  source 1..25: mol_type = other DNA
ggtatactgctccagtctgctgtca

SEQ ID NO: 969 - DNA, 25 bp, synthetic construct
  misc_feature 1..25: note = >Male | ENST00000432646 | 17051908
  source 1..25: mol_type = other DNA
tatttggagtaagatggcccgggct

SEQ ID NO: 970 - DNA, 25 bp, synthetic construct
  misc_feature 1..25: note = >Male | ENST00000432646 | 17051908
  source 1..25: mol_type = other DNA
cattgatgcggctagaaacccctac

SEQ ID NO: 971 - DNA, 25 bp, synthetic construct
  misc_feature 1..25: note = >Male | ENST00000432646 | 17051908
  source 1..25: mol_type = other DNA
gagaaagacccactccggaagaaga

SEQ ID NO: 972 - DNA, 25 bp, synthetic construct
  misc_feature 1..25: note = >Male | ENST00000432646 | 17051908
  source 1..25: mol_type = other DNA
gcagaaaaacatcgataggtggcca

SEQ ID NO: 973 - DNA, 25 bp, synthetic construct
  misc_feature 1..25: note = >Male | ENST00000432646 | 17051908
  source 1..25: mol_type = other DNA
cagattgcttttcccagaggttccg

SEQ ID NO: 974 - DNA, 25 bp, synthetic construct
  misc_feature 1..25: note = >Male | ENST00000432646 | 17051908
  source 1..25: mol_type = other DNA
tctgaaatactagtcttcccaatag

SEQ ID NO: 975 - DNA, 25 bp, synthetic construct
  misc_feature 1..25: note = >Male | ENST00000432646 | 17051908
  source 1..25: mol_type = other DNA
aactgagctcgtcctgcgtcttgag

SEQ ID NO: 976 - DNA, 25 bp, synthetic construct
  misc_feature 1..25: note = >Male | ENST00000432646 | 17051908
  source 1..25: mol_type = other DNA
tgacgggagtcaatgctacctggga

SEQ ID NO: 977 - DNA, 25 bp, synthetic construct
  misc_feature 1..25: note = >Male | ENST00000518932 | 17080053
  source 1..25: mol_type = other DNA
catgcccatgcaagaggaatgtgat

SEQ ID NO: 978 - DNA, 25 bp, synthetic construct
  misc_feature 1..25: note = >Male | ENST00000518932 | 17080053
  source 1..25: mol_type = other DNA
tcagttgcaagatgacgctcagtcg

SEQ ID NO: 979 - DNA, 25 bp, synthetic construct
  misc_feature 1..25: note = >Male | ENST00000518932 | 17080053
  source 1..25: mol_type = other DNA
caataaagtgctcttcatagttcat

SEQ ID NO: 980 - DNA, 25 bp, synthetic construct
  misc_feature 1..25: note = >Male | ENST00000518932 | 17080053
  source 1..25: mol_type = other DNA
tttctgtgatttcaggtgccacctg

SEQ ID NO: 981 - DNA, 25 bp, synthetic construct
  misc_feature 1..25: note = >Male | ENST00000518932 | 17080053
  source 1..25: mol_type = other DNA
ggtgccacctgttcttcactattag

SEQ ID NO: 982 - DNA, 25 bp, synthetic construct
  misc_feature 1..25: note = >Male | ENST00000518932 | 17080053
  source 1..25: mol_type = other DNA
tcctaaatgttgagataccccaagt

SEQ ID NO: 983 - DNA, 25 bp, synthetic construct
  misc_feature 1..25: note = >Male | ENST00000518932 | 17080053
  source 1..25: mol_type = other DNA
ggttaccacatgaatgcagagagca

SEQ ID NO: 984 - DNA, 25 bp, synthetic construct
  misc_feature 1..25: note = >Male | ENST00000518932 | 17080053
  source 1..25: mol_type = other DNA
gcctgtatgtcttctccaagttcca

SEQ ID NO: 985 - DNA, 25 bp, synthetic construct
  misc_feature 1..25: note = >Male | ENST00000518932 | 17080053
  source 1..25: mol_type = other DNA
aagctaatcgcagaagccttcaggg

SEQ ID NO: 986 - DNA, 25 bp, synthetic construct
  misc_feature 1..25: note = >Male | ENST00000518932 | 17080053
  source 1..25: mol_type = other DNA
aatcagctggcttaattaattgccc

SEQ ID NO: 987 - DNA, 25 bp, synthetic construct
  misc_feature 1..25: note = >Male | ENST00000518932 | 17080053
  source 1..25: mol_type = other DNA
tccaccagatttttaccacaagttg

SEQ ID NO: 988 - DNA, 25 bp, synthetic construct
  misc_feature 1..25: note = >Male | ENST00000518932 | 17080053
  source 1..25: mol_type = other DNA
ttccctgtgtagatgaatgaagcca

SEQ ID NO: 989 - DNA, 25 bp, synthetic construct
  misc_feature 1..25: note = >Male | ENST00000518932 | 17080053
  source 1..25: mol_type = other DNA
tactgacacagaagcttgggagcaa

SEQ ID NO: 990 - DNA, 25 bp, synthetic construct
  misc_feature 1..25: note = >Male | ENST00000518932 | 17080053
  source 1..25: mol_type = other DNA
catggatgggcgagttacttaacac

SEQ ID NO: 991 - DNA, 25 bp, synthetic construct
  misc_feature 1..25: note = >Male | ENST00000518932 | 17080053
  source 1..25: mol_type = other DNA
taatcaatcagataatttacatcac

SEQ ID NO: 992 - DNA, 25 bp, synthetic construct
  misc_feature 1..25: note = >Male | ENST00000518932 | 17080053
  source 1..25: mol_type = other DNA
caaaagagaaacaggcacctcttca

SEQ ID NO: 993 - DNA, 25 bp, synthetic construct
  misc_feature 1..25: note = >Male | ENST00000518932 | 17080053
  source 1..25: mol_type = other DNA
caataaaagtgttgaaggtgcctcc

SEQ ID NO: 994 - DNA, 25 bp, synthetic construct
  misc_feature 1..25: note = >Male | ENST00000518932 | 17080053
  source 1..25: mol_type = other DNA
cttaagctccactatgagagaagtg

SEQ ID NO: 995 - DNA, 25 bp, synthetic construct
  misc_feature 1..25: note = >Male | ENST00000518932 | 17080053
  source 1..25: mol_type = other DNA
taagctccactatgagagaagtgtg

SEQ ID NO: 996 - DNA, 25 bp, synthetic construct
  misc_feature 1..25: note = >Male | ENST00000518932 | 17080053
  source 1..25: mol_type = other DNA
tgtgaagaacttaataatcaccagc

SEQ ID NO: 997 - DNA, 25 bp, synthetic construct
  misc_feature 1..25: note = >Male | ENST00000518932 | 17080053
  source 1..25: mol_type = other DNA
tggtatccggtgagcattaaactct

SEQ ID NO: 998 - DNA, 25 bp, synthetic construct
  misc_feature 1..25: note = >Male | ENST00000518932 | 17080053
  source 1..25: mol_type = other DNA
aactctgttgtctcagtgtaactga

SEQ ID NO: 999 - DNA, 25 bp, synthetic construct
  misc_feature 1..25: note = >Male | ENST00000518932 | 17080053
  source 1..25: mol_type = other DNA
cagtggtcctgtgtacttgctagtc

SEQ ID NO: 1000 - DNA, 25 bp, synthetic construct
  misc_feature 1..25: note = >Male | ENST00000518932 | 17080053
  source 1..25: mol_type = other DNA
tctgctggtggattcattgtaggaa

SEQ ID NO: 1001 - DNA, 25 bp, synthetic construct
  misc_feature 1..25: note = >Male | ENST00000518932 | 17080053
  source 1..25: mol_type = other DNA
gaatgcgtactacatactgtaagga

SEQ ID NO: 1002 - DNA, 25 bp, synthetic construct
  misc_feature 1..25: note = >Male | ENST00000518932 | 17080053
  source 1..25: mol_type = other DNA
tgtgctgcggtcaagaataggtcga

SEQ ID NO: 1003 - DNA, 25 bp, synthetic construct
  misc_feature 1..25: note = >Male | NM_001033551 | 16842003
  source 1..25: mol_type = other DNA
taaacttctaggacgagtggacagc

SEQ ID NO: 1004 - DNA, 25 bp, synthetic construct
  misc_feature 1..25: note = >Male | NM_001033551 | 16842003
  source 1..25: mol_type = other DNA
tattggtagtgtcgtctcagcagcg

SEQ ID NO: 1005 - DNA, 25 bp, synthetic construct
  misc_feature 1..25: note = >Male | NM_001033551 | 16842003
  source 1..25: mol_type = other DNA
tcacagggcgaagagggcatcctct

SEQ ID NO: 1006 - DNA, 25 bp, synthetic construct
  misc_feature 1..25: note = >Male | NM_001033551 | 16842003
  source 1..25: mol_type = other DNA
gcagctttggctctttcttcaagga

SEQ ID NO: 1007 - DNA, 25 bp, synthetic construct
  misc_feature 1..25: note = >Male | NM_001033551 | 16842003
  source 1..25: mol_type = other DNA
tcggtcctgagccacacctcaatgt

SEQ ID NO: 1008 - DNA, 25 bp, synthetic construct
  misc_feature 1..25: note = >Male | NM_001033551 | 16842003
  source 1..25: mol_type = other DNA
gcaaatgataagaagcaggctcccg

SEQ ID NO: 1009 - DNA, 25 bp, synthetic construct
  misc_feature 1..25: note = >Male | NM_001033551 | 16842003
  source 1..25: mol_type = other DNA
tgagtgtccactctcacattgcggg

SEQ ID NO: 1010 - DNA, 25 bp, synthetic construct
  misc_feature 1..25: note = >Male | NM_001033551 | 16842003
  source 1..25: mol_type = other DNA
cattccttgggttataagccagctg

SEQ ID NO: 1011 - DNA, 25 bp, synthetic construct
  misc_feature 1..25: note = >Male | NM_001033551 | 16842003
  source 1..25: mol_type = other DNA
gggttataagccagctgtctatggg

SEQ ID NO: 1012 - DNA, 25 bp, synthetic construct
  misc_feature 1..25: note = >Male | NM_001033551 | 16842003
  source 1..25: mol_type = other DNA
tgtctagtgcagaagcaagtcctcc

SEQ ID NO: 1013 - DNA, 25 bp, synthetic construct
  misc_feature 1..25: note = >Male | NM_001033551 | 16842003
  source 1..25: mol_type = other DNA
tctcgctgtaaggaatgggttcacc

SEQ ID NO: 1014 - DNA, 25 bp, synthetic construct
  misc_feature 1..25: note = >Male | NM_001033551 | 16842003
  source 1..25: mol_type = other DNA
gcaaacatgtcaaagccgtcacggg

SEQ ID NO: 1015 - DNA, 25 bp, synthetic construct
  misc_feature 1..25: note = >Male | NM_001033551 | 16842003
  source 1..25: mol_type = other DNA
tgagggtgccactgacgctctctgt

SEQ ID NO: 1016 - DNA, 25 bp, synthetic construct
  misc_feature 1..25: note = >Male | NM_001033551 | 16842003
  source 1..25: mol_type = other DNA
gctgtgttccccaccattgggctca

SEQ ID NO: 1017 - DNA, 25 bp, synthetic construct
  misc_feature 1..25: note = >Male | NM_001033551 | 16842003
  source 1..25: mol_type = other DNA
gcattttgaacggatcggccagacc

SEQ ID NO: 1018 - DNA, 25 bp, synthetic construct
  misc_feature 1..25: note = >Male | NM_001033551 | 16842003
  source 1..25: mol_type = other DNA
gatcggccagacctgtatcgttcga

SEQ ID NO: 1019 - DNA, 25 bp, synthetic construct
  misc_feature 1..25: note = >Male | NM_001033551 | 16842003
  source 1..25: mol_type = other DNA
aagaaatccctctgttatggcaaca

SEQ ID NO: 1020 - DNA, 25 bp, synthetic construct
  misc_feature 1..25: note = >Male | NM_001033551 | 16842003
  source 1..25: mol_type = other DNA
tggcaacaatcttttcaagggaaaa

SEQ ID NO: 1021 - DNA, 25 bp, synthetic construct
  misc_feature 1..25: note = >Male | NM_001033551 | 16842003
  source 1..25: mol_type = other DNA
tgaggtcatcgttcacatgcagcag

SEQ ID NO: 1022 - DNA, 25 bp, synthetic construct
  misc_feature 1..25: note = >Male | NM_001033551 | 16842003
  source 1..25: mol_type = other DNA
tcctcattggacacgcgggagatga

SEQ ID NO: 1023 - DNA, 25 bp, synthetic construct
  misc_feature 1..25: note = >Male | NM_001033551 | 16842003
  source 1..25: mol_type = other DNA
tagggaacacatgcacctctgggta

SEQ ID NO: 1024 - DNA, 25 bp, synthetic construct
  misc_feature 1..25: note = >Male | NM_001033551 | 16842003
  source 1..25: mol_type = other DNA
ccatcacttggtacagactgcctca

SEQ ID NO: 1025 - DNA, 25 bp, synthetic construct
  misc_feature 1..25: note = >Male | NM_001033551 | 16842003
  source 1..25: mol_type = other DNA
ttgtgaatggctcatgtccccaggc

SEQ ID NO: 1026 - DNA, 25 bp, synthetic construct
  misc_feature 1..25: note = >Male | NM_001033551 | 16842003
  source 1..25: mol_type = other DNA
ctctcaaaaagctgcctgggccttg

SEQ ID NO: 1027 - DNA, 25 bp, synthetic construct
  misc_feature 1..25: note = >Male | NM_001033551 | 16842003
  source 1..25: mol_type = other DNA
tgtgtttcctcgaacgacgtccagt

SEQ ID NO: 1028 - DNA, 25 bp, synthetic construct
  misc_feature 1..25: note = >Male | NM_001033551 | 16842003
  source 1..25: mol_type = other DNA
cgaggaataggaaccagcacttgtc

SEQ ID NO: 1029 - DNA, 25 bp, synthetic construct
  misc_feature 1..25: note = >Male | NM_001033551 | 16842003
  source 1..25: mol_type = other DNA
ggcatggtcgcagctggatccactt

SEQ ID NO: 1030 - DNA, 25 bp, synthetic construct
  misc_feature 1..25: note = >Male | NM_001033551 | 16842003
  source 1..25: mol_type = other DNA
catatatgtgcacaacgccggtgag

SEQ ID NO: 1031 - DNA, 25 bp, synthetic construct
  misc_feature 1..25: note = >Male | NM_001033551 | 16842003
  source 1..25: mol_type = other DNA
gactgcttcgaaaggcatcagccca

SEQ ID NO: 1032 - DNA, 25 bp, synthetic construct
  misc_feature 1..25: note = >Male | NM_001033551 | 16842003
  source 1..25: mol_type = other DNA
gatgaaatctcggttggccacaagg

SEQ ID NO: 1033 - DNA, 25 bp, synthetic construct
  misc_feature 1..25: note = >Male | NM_001033551 | 16842003
  source 1..25: mol_type = other DNA
tctgtagttccggttcccgttgagc

SEQ ID NO: 1034 - DNA, 25 bp, synthetic construct
  misc_feature 1..25: note = >Male | NM_001033551 | 16842003
  source 1..25: mol_type = other DNA
tcttcagggctcgaatggcatcctt

SEQ ID NO: 1035 - DNA, 25 bp, synthetic construct
  misc_feature 1..25: note = >Male | NM_001033551 | 16842003
  source 1..25: mol_type = other DNA
tgtcacagatctccatattcaacgt

SEQ ID NO: 1036 - DNA, 25 bp, synthetic construct
  misc_feature 1..25: note = >Male | NM_001033551 | 16842003
  source 1..25: mol_type = other DNA
gaggcactgccccactggtgtgctg

SEQ ID NO: 1037 - DNA, 25 bp, synthetic construct
  misc_feature 1..25: note = >Male | NM_001033551 | 16842003
  source 1..25: mol_type = other DNA
ggactcccgtcctgactgacacttg

SEQ ID NO: 1038 - DNA, 25 bp, synthetic construct
  misc_feature 1..25: note = >Male | NM_001033551 | 16842003
  source 1..25: mol_type = other DNA
tctggaacgccgagcttagggcctt

SEQ ID NO: 1039 - DNA, 25 bp, synthetic construct
  misc_feature 1..25: note = >Male | NM_001105206 | 17022799
  source 1..25: mol_type = other DNA
ccagtaggaattgcgtgtgctctca

SEQ ID NO: 1040 - DNA, 25 bp, synthetic construct
  misc_feature 1..25: note = >Male | NM_001105206 | 17022799
  source 1..25: mol_type = other DNA
acaggagttgatgcttacggcgccg

SEQ ID NO: 1041 - DNA, 25 bp, synthetic construct
  misc_feature 1..25: note = >Male | NM_001105206 | 17022799
  source 1..25: mol_type = other DNA
cacatggttcacttcagagtccaca

SEQ ID NO: 1042 - DNA, 25 bp, synthetic construct
  misc_feature 1..25: note = >Male | NM_001105206 | 17022799
  source 1..25: mol_type = other DNA
cacagagactctgcttgggtgtaac

SEQ ID NO: 1043 - DNA, 25 bp, synthetic construct
  misc_feature 1..25: note = >Male | NM_001105206 | 17022799
  source 1..25: mol_type = other DNA
aactgcttctgggacggacttcaaa

SEQ ID NO: 1044 - DNA, 25 bp, synthetic construct
  misc_feature 1..25: note = >Male | NM_001105206 | 17022799
  source 1..25: mol_type = other DNA
gaagactttcttctaggactcggag

SEQ ID NO: 1045 - DNA, 25 bp, synthetic construct
  misc_feature 1..25: note = >Male | NM_001105206 | 17022799
  source 1..25: mol_type = other DNA
catgccatgggaggaacgagttctc

SEQ ID NO: 1046 - DNA, 25 bp, synthetic construct
  misc_feature 1..25: note = >Male | NM_001105206 | 17022799
  source 1..25: mol_type = other DNA
gagtttcagagcaacaggatcccat

SEQ ID NO: 1047 - DNA, 25 bp, synthetic construct
  misc_feature 1..25: note = >Male | NM_001105206 | 17022799
  source 1..25: mol_type = other DNA
tgtggaccttttcagtataccgttg

SEQ ID NO: 1048 - DNA, 25 bp, synthetic construct
  misc_feature 1..25: note = >Male | NM_001105206 | 17022799
  source 1..25: mol_type = other DNA
tactgagcactgattggtgagccac

SEQ ID NO: 1049 - DNA, 25 bp, synthetic construct
  misc_feature 1..25: note = >Male | NM_001105206 | 17022799
  source 1..25: mol_type = other DNA
gacccactctaggttggataagcag

SEQ ID NO: 1050 - DNA, 25 bp, synthetic construct
  misc_feature 1..25: note = >Male | NM_001105206 | 17022799
  source 1..25: mol_type = other DNA
gctaatgacgaagtgggacagccca

SEQ ID NO: 1051 - DNA, 25 bp, synthetic construct
  misc_feature 1..25: note = >Male | NM_001105206 | 17022799
  source 1..25: mol_type = other DNA
tgtccattgaaatatgctctgcgag

SEQ ID NO: 1052 - DNA, 25 bp, synthetic construct
  misc_feature 1..25: note = >Male | NM_001105206 | 17022799
  source 1..25: mol_type = other DNA
tcttttggaactggaagcccttcat

SEQ ID NO: 1053 - DNA, 25 bp, synthetic construct
  misc_feature 1..25: note = >Male | NM_001105206 | 17022799
  source 1..25: mol_type = other DNA
tgtaagatttctggaggagctcctc

SEQ ID NO: 1054 - DNA, 25 bp, synthetic construct
  misc_feature 1..25: note = >Male | NM_001105206 | 17022799
  source 1..25: mol_type = other DNA
gggtccttcagttgctctttagata

SEQ ID NO: 1055 - DNA, 25 bp, synthetic construct
  misc_feature 1..25: note = >Male | NM_001105206 | 17022799
  source 1..25: mol_type = other DNA
gggccaccgctgaatccaaaatcat

SEQ ID NO: 1056 - DNA, 25 bp, synthetic construct
  misc_feature 1..25: note = >Male | NM_001105206 | 17022799
  source 1..25: mol_type = other DNA
catcgaagaagtaactggcagcccg

SEQ ID NO: 1057 - DNA, 25 bp, synthetic construct
  misc_feature 1..25: note = >Male | NM_001105206 | 17022799
  source 1..25: mol_type = other DNA
ggcagccaacaaagccaggcaggtt

SEQ ID NO: 1058 - DNA, 25 bp, synthetic construct
  misc_feature 1..25: note = >Male | NM_001105206 | 17022799
  source 1..25: mol_type = other DNA
tgtgctacttagactcgggactgtt

SEQ ID NO: 1059 - DNA, 25 bp, synthetic construct
  misc_feature 1..25: note = >Male | NM_001105206 | 17022799
  source 1..25: mol_type = other DNA
taagcaggccaggaactgacgggct

SEQ ID NO: 1060 - DNA, 25 bp, synthetic construct
  misc_feature 1..25: note = >Male | NM_001105206 | 17022799
  source 1..25: mol_type = other DNA
ggtttcatgtacaggctcagagacg

SEQ ID NO: 1061 - DNA, 25 bp, synthetic construct
  misc_feature 1..25: note = >Male | NM_001105206 | 17022799
  source 1..25: mol_type = other DNA
tcaaccgtacgaagctgatccagga

SEQ ID NO: 1062 - DNA, 25 bp, synthetic construct
  misc_feature 1..25: note = >Male | NM_001105206 | 17022799
  source 1..25: mol_type = other DNA
tgatcagtctagactgccccaggcg

SEQ ID NO: 1063 - DNA, 25 bp, synthetic construct
  misc_feature 1..25: note = >Male | NM_001105206 | 17022799
  source 1..25: mol_type = other DNA
tcactgagtctggttttaagggcac

SEQ ID NO: 1064 - DNA, 25 bp, synthetic construct
  misc_feature 1..25: note = >Male | NM_001105206 | 17022799
  source 1..25: mol_type = other DNA
ctctggcttgattgaggaggttctc

SEQ ID NO: 1065 - DNA, 25 bp, synthetic construct
  misc_feature 1..25: note = >Male | NM_001105206 | 17022799
  source 1..25: mol_type = other DNA
gaaggtcctgtgcatggtcaatagc

SEQ ID NO: 1066 - DNA, 25 bp, synthetic construct
  misc_feature 1..25: note = >Male | NM_001105206 | 17022799
  source 1..25: mol_type = other DNA
tcagagagtccgcagatgtgctcag

SEQ ID NO: 1067 - DNA, 25 bp, synthetic construct
  misc_feature 1..25: note = >Male | NM_001105206 | 17022799
  source 1..25: mol_type = other DNA
tgcttgaagcggacacatgaccact

SEQ ID NO: 1068 - DNA, 25 bp, synthetic construct
  misc_feature 1..25: note = >Male | NM_001105206 | 17022799
  source 1..25: mol_type = other DNA
tccaggacgacaggaaacagagtgc

SEQ ID NO: 1069 - DNA, 25 bp, synthetic construct
  misc_feature 1..25: note = >Male | NM_001105206 | 17022799
  source 1..25: mol_type = other DNA
catccacgagctcccgttgggtgaa

SEQ ID NO: 1070 - DNA, 25 bp, synthetic construct
  misc_feature 1..25: note = >Male | NM_001105206 | 17022799
  source 1..25: mol_type = other DNA
tatcatgggcttgctctaccagctg

SEQ ID NO: 1071 - DNA, 25 bp, synthetic construct
  misc_feature 1..25: note = >Male | NM_001105206 | 17022799
  source 1..25: mol_type = other DNA
taaacgtaaatcttcctggcatgtg

SEQ ID NO: 1072 - DNA, 25 bp, synthetic construct
  misc_feature 1..25: note = >Male | NM_001105206 | 17022799
  source 1..25: mol_type = other DNA
taaactgtgggctctctgaatggac

SEQ ID NO: 1073 - DNA, 25 bp, synthetic construct
  misc_feature 1..25: note = >Male | NM_001105206 | 17022799
  source 1..25: mol_type = other DNA
ttatactccttcctgcaattccagt

SEQ ID NO: 1074 - DNA, 25 bp, synthetic construct
  misc_feature 1..25: note = >Male | NM_001105206 | 17022799
  source 1..25: mol_type = other DNA
tctacgtcagacagaaggcttttca

SEQ ID NO: 1075 - DNA, 25 bp, synthetic construct
  misc_feature 1..25: note = >Male | NM_001105206 | 17022799
  source 1..25: mol_type = other DNA
tcattcacgtgcctatgagcggcgg

SEQ ID NO: 1076 - DNA, 25 bp, synthetic construct
  misc_feature 1..25: note = >Male | NM_001105206 | 17022799
  source 1..25: mol_type = other DNA
tgccctagggaatcaagtggaccac

SEQ ID NO: 1077 - DNA, 25 bp, synthetic construct
  misc_feature 1..25: note = >Male | NM_001105206 | 17022799
  source 1..25: mol_type = other DNA
ccatagtagccaggagcgcaacgtt

SEQ ID NO: 1078 - DNA, 25 bp, synthetic construct
  misc_feature 1..25: note = >Male | NM_001105206 | 17022799
  source 1..25: mol_type = other DNA
gaaagcctctcgtgctctgtggaga

SEQ ID NO: 1079 - DNA, 25 bp, synthetic construct
  misc_feature 1..25: note = >Male | NM_001105206 | 17022799
  source 1..25: mol_type = other DNA
tcgttacaaatgcaccgaacagctc

SEQ ID NO: 1080 - DNA, 25 bp, synthetic construct
  misc_feature 1..25: note = >Male | NM_001105206 | 17022799
  source 1..25: mol_type = other DNA
ggtgctcccctgatggaatctccga

SEQ ID NO: 1081 - DNA, 25 bp, synthetic construct
  misc_feature 1..25: note = >Male | NM_001105206 | 17022799
  source 1..25: mol_type = other DNA
ggcaacctgcaagaactgactgaaa

SEQ ID NO: 1082 - DNA, 25 bp, synthetic construct
  misc_feature 1..25: note = >Male | NM_001105206 | 17022799
  source 1..25: mol_type = other DNA
ggaattgccattacagtcgcagggc

SEQ ID NO: 1083 - DNA, 25 bp, synthetic construct
  misc_feature 1..25: note = >Male | NM_001105206 | 17022799
  source 1..25: mol_type = other DNA
aaatgcctttaacactgtcttatca

SEQ ID NO: 1084 - DNA, 25 bp, synthetic construct
  misc_feature 1..25: note = >Male | NM_001105206 | 17022799
  source 1..25: mol_type = other DNA
gacaaaatgactactgggacacgca

SEQ ID NO: 1085 - DNA, 25 bp, synthetic construct
  misc_feature 1..25: note = >Male | NM_001105206 | 17022799
  source 1..25: mol_type = other DNA
aagtttccgaactgacctagcccag

SEQ ID NO: 1086 - DNA, 25 bp, synthetic construct
  misc_feature 1..25: note = >Male | NM_001105206 | 17022799
  source 1..25: mol_type = other DNA
cgaactgacctagcccaggtgaaac

SEQ ID NO: 1087 - DNA, 25 bp, synthetic construct
  misc_feature 1..25: note = >Male | NM_001105206 | 17022799
  source 1..25: mol_type = other DNA
caggtgaaactctcaaggcactggg

SEQ ID NO: 1088 - DNA, 25 bp, synthetic construct
  misc_feature 1..25: note = >Male | NM_001105206 | 17022799
  source 1..25: mol_type = other DNA
ggttcgctcgtctcaggcgggtctt

SEQ ID NO: 1089 - DNA, 25 bp, synthetic construct
  misc_feature 1..25: note = >Male | NM_001105206 | 17022799
  source 1..25: mol_type = other DNA
ggtcttgcctgccaaccgctgagct

SEQ ID NO: 1090 - DNA, 25 bp, synthetic construct
  misc_feature 1..25: note = >Male | NM_001105206 | 17022799
  source 1..25: mol_type = other DNA
aaccgctgagctcccttcaatgtca

SEQ ID NO: 1091 - DNA, 25 bp, synthetic construct
  misc_feature 1..25: note = >Male | NM_001105206 | 17022799
  source 1..25: mol_type = other DNA
ttctccgctgacatccagtagtgct

SEQ ID NO: 1092 - DNA, 25 bp, synthetic construct
  misc_feature 1..25: note = >Male | NM_001105206 | 17022799
  source 1..25: mol_type = other DNA
tctccgctgacatccagtagtgctc

SEQ ID NO: 1093 - DNA, 25 bp, synthetic construct
  misc_feature 1..25: note = >Male | NM_001105206 | 17022799
  source 1..25: mol_type = other DNA
cagtagtgctcttccagggctcggg

SEQ ID NO: 1094 - DNA, 25 bp, synthetic construct
  misc_feature 1..25: note = >Male | NM_001105206 | 17022799
  source 1..25: mol_type = other DNA
tgtgggtctccgtaggtctcccgcg

SEQ ID NO: 1095 - DNA, 25 bp, synthetic construct
  misc_feature 1..25: note = >Male | NM_001105206 | 17022799
  source 1..25: mol_type = other DNA
tccgtgtgcagtatcccgaggtggc

SEQ ID NO: 1096 - DNA, 25 bp, synthetic construct
  misc_feature 1..25: note = >Male | NM_001105206 | 17022799
  source 1..25: mol_type = other DNA
gaaataaagggaaagtgcgagagta

SEQ ID NO: 1097 - DNA, 25 bp, synthetic construct
  misc_feature 1..25: note = >Male | NM_001105206 | 17022799
  source 1..25: mol_type = other DNA
tcccgcagaggttctggctcaagaa

SEQ ID NO: 1098 - DNA, 25 bp, synthetic construct
  misc_feature 1..25: note = >Male | NM_001105206 | 17022799
  source 1..25: mol_type = other DNA
tacggcatcaaaaggcagacggatt

SEQ ID NO: 1099 - DNA, 25 bp, synthetic construct
  misc_feature 1..25: note = >Male | NM_001105206 | 17022799
  source 1..25: mol_type = other DNA
gctgtcctggcttccttctcacatt

SEQ ID NO: 1100 - DNA, 25 bp, synthetic construct
  misc_feature 1..25: note = >Male | NM_001105206 | 17022799
  source 1..25: mol_type = other DNA
tggcttttcagacactgaggtccgc

SEQ ID NO: 1101 - DNA, 25 bp, synthetic construct
  misc_feature 1..25: note = >Male | NM_001105206 | 17022799
  source 1..25: mol_type = other DNA
cagggtgcccactttcttgagcaag

SEQ ID NO: 1102 - DNA, 25 bp, synthetic construct
  misc_feature 1..25: note = >Male | NM_182480 | 16786369
  source 1..25: mol_type = other DNA
tctgtccagctgaaatgaccgtggg

SEQ ID NO: 1103 - DNA, 25 bp, synthetic construct
  misc_feature 1..25: note = >Male | NM_182480 | 16786369
  source 1..25: mol_type = other DNA
aaccagacgccaaagctagaaaggg

SEQ ID NO: 1104 - DNA, 25 bp, synthetic construct
  misc_feature 1..25: note = >Male | NM_182480 | 16786369
  source 1..25: mol_type = other DNA
ctacgtagtgcgtccggagcagagc

SEQ ID NO: 1105 - DNA, 25 bp, synthetic construct
  misc_feature 1..25: note = >Male | NM_182480 | 16786369
  source 1..25: mol_type = other DNA
cacggtgtctgttgaggcgccggac

SEQ ID NO: 1106 - DNA, 25 bp, synthetic construct
  misc_feature 1..25: note = >Male | NM_182480 | 16786369
  source 1..25: mol_type = other DNA
tctttgggcaagtcgtgctagggct

SEQ ID NO: 1107 - DNA, 25 bp, synthetic construct
  misc_feature 1..25: note = >Male | NM_182480 | 16786369
  source 1..25: mol_type = other DNA
gatggacccacatgcctcaaggatg

SEQ ID NO: 1108 - DNA, 25 bp, synthetic construct
  misc_feature 1..25: note = >Male | NM_182480 | 16786369
  source 1..25: mol_type = other DNA
agagtgacttcacaaggacctgggc

SEQ ID NO: 1109 - DNA, 25 bp, synthetic construct
  misc_feature 1..25: note = >Male | NM_182480 | 16786369
  source 1..25: mol_type = other DNA
tcatgttgcagatatggtcccaggc

SEQ ID NO: 1110 - DNA, 25 bp, synthetic construct
  misc_feature 1..25: note = >Male | NM_182480 | 16786369
  source 1..25: mol_type = other DNA
tgcattcgccgaaaggctctgtatc

SEQ ID NO: 1111 - DNA, 25 bp, synthetic construct
  misc_feature 1..25: note = >Male | NM_182480 | 16786369
  source 1..25: mol_type = other DNA
gtggactccgatatcttaagacaga

SEQ ID NO: 1112 - DNA, 25 bp, synthetic construct
  misc_feature 1..25: note = >Male | NM_182480 | 16786369
  source 1..25: mol_type = other DNA
tcaaacattatcagggcctctgagc

SEQ ID NO: 1113 - DNA, 25 bp, synthetic construct
  misc_feature 1..25: note = >Male | NM_182480 | 16786369
  source 1..25: mol_type = other DNA
tcccaatgaatgcaaggtggaagat

SEQ ID NO: 1114 - DNA, 25 bp, synthetic construct
  misc_feature 1..25: note = >Male | NM_182480 | 16786369
  source 1..25: mol_type = other DNA
cagaggatttctttatggtggacac

SEQ ID NO: 1115 - DNA, 25 bp, synthetic construct
  misc_feature 1..25: note = >Male | NM_182480 | 16786369
  source 1..25: mol_type = other DNA
gagaggattgttaagccactgcacc

SEQ ID NO: 1116 - DNA, 25 bp, synthetic construct
  misc_feature 1..25: note = >Male | NM_182480 | 16786369
  source 1..25: mol_type = other DNA
tgctcctgtagagaaccgtcactcg

SEQ ID NO: 1117 - DNA, 25 bp, synthetic construct
  misc_feature 1..25: note = >Male | NM_182480 | 16786369
  source 1..25: mol_type = other DNA
taaatcgaaaaccagagtccctaga

SEQ ID NO: 1118 - DNA, 25 bp, synthetic construct
  misc_feature 1..25: note = >Male | NM_182480 | 16786369
  source 1..25: mol_type = other DNA
ctaagacggcccatgagattgctcc

SEQ ID NO: 1119 - DNA, 25 bp, synthetic construct
  misc_feature 1..25: note = >Male | NM_182480 | 16786369
  source 1..25: mol_type = other DNA
tactccggagttgtgaccatctgca

SEQ ID NO: 1120 - DNA, 25 bp, synthetic construct
  misc_feature 1..25: note = >Male | NM_182480 | 16786369
  source 1..25: mol_type = other DNA
tatgaataaagtccccagatggcag

SEQ ID NO: 1121 - DNA, 25 bp, synthetic construct
  misc_feature 1..25: note = >Male | NM_182480 | 16786369
  source 1..25: mol_type = other DNA
ggaagaaatctctgccaggctacgt

SEQ ID NO: 1122 - DNA, 25 bp, synthetic construct
  misc_feature 1..25: note = >Male | NM_182480 | 16786369
  source 1..25: mol_type = other DNA
gggaacttacaaacagcctgggcag

SEQ ID NO: 1123 - DNA, 25 bp, synthetic construct
  misc_feature 1..25: note = >Male | NM_182480 | 16786369
  source 1..25: mol_type = other DNA
tcatgggacgtggaccaaaccaagg

SEQ ID NO: 1124 - DNA, 25 bp, synthetic construct
  misc_feature 1..25: note = >Male | NM_182480 | 16786369
  source 1..25: mol_type = other DNA
aaaggcagagttaacggcatccaca

SEQ ID NO: 1125 - DNA, 25 bp, synthetic construct
  misc_feature 1..25: note = >Male | NM_182480 | 16786369
  source 1..25: mol_type = other DNA
taagccccatgctgaaattcgctcg

SEQ ID NO: 1126 - DNA, 25 bp, synthetic construct
  misc_feature 1..25: note = >Male | NM_182480 | 16786369
  source 1..25: mol_type = other DNA
caccagctgtgtcgatgaagtccgt

SEQ ID NO: 1127 - DNA, 25 bp, synthetic construct
  misc_feature 1..25: note = >Male | NM_182480 | 16786369
  source 1..25: mol_type = other DNA
aacagaactcggcttttggcatcca

SEQ ID NO: 1128 - DNA, 25 bp, synthetic construct
  misc_feature 1..25: note = >Male | NM_182480 | 16786369
  source 1..25: mol_type = other DNA
ccgtactgaggtgatgggccaagct

SEQ ID NO: 1129 - DNA, 25 bp, synthetic construct
  misc_feature 1..25: note = >Male | NM_182480 | 16786369
  source 1..25: mol_type = other DNA
gagtggagacactgcatttgtggcc

SEQ ID NO: 1130 - DNA, 25 bp, synthetic construct
  misc_feature 1..25: note = >Male | NM_182480 | 16786369
  source 1..25: mol_type = other DNA
aaatatgtatgatgggtcctgggca

SEQ ID NO: 1131 - DNA, 25 bp, synthetic construct
  misc_feature 1..25: note = >Male | ENST00000481363 | 16920377
  source 1..25: mol_type = other DNA
gcaaagcagcatagcaacaatcgtg

SEQ ID NO: 1132 - DNA, 25 bp, synthetic construct
  misc_feature 1..25: note = >Male | ENST00000481363 | 16920377
  source 1..25: mol_type = other DNA
gttgtggtcacaaccaacgtagtaa

SEQ ID NO: 1133 - DNA, 25 bp, synthetic construct
  misc_feature 1..25: note = >Male | ENST00000481363 | 16920377
  source 1..25: mol_type = other DNA
gatttcactgtgtctgcattgctcc

SEQ ID NO: 1134 - DNA, 25 bp, synthetic construct
  misc_feature 1..25: note = >Male | ENST00000481363 | 16920377
  source 1..25: mol_type = other DNA
tgggaacgccgtcagtagcacttgg

SEQ ID NO: 1135 - DNA, 25 bp, synthetic construct
  misc_feature 1..25: note = >Male | ENST00000481363 | 16920377
  source 1..25: mol_type = other DNA
catctgcactgatacccgactggga

SEQ ID NO: 1136 - DNA, 25 bp, synthetic construct
  misc_feature 1..25: note = >Male | ENST00000481363 | 16920377
  source 1..25: mol_type = other DNA
gcgttattcacaacggaggtggccc

SEQ ID NO: 1137 - DNA, 25 bp, synthetic construct
  misc_feature 1..25: note = >Male | ENST00000481363 | 16920377
  source 1..25: mol_type = other DNA
taaaagctcgcccacaaatgttgca

SEQ ID NO: 1138 - DNA, 25 bp, synthetic construct
  misc_feature 1..25: note = >Male | ENST00000481363 | 16920377
  source 1..25: mol_type = other DNA
tgtcgagggtcccacaaatgtgcca

SEQ ID NO: 1139 - DNA, 25 bp, synthetic construct
  misc_feature 1..25: note = >Male | ENST00000481363 | 16920377
  source 1..25: mol_type = other DNA
gataaacgtggaagggagactgctc

SEQ ID NO: 1140 - DNA, 25 bp, synthetic construct
  misc_feature 1..25: note = >Male | ENST00000481363 | 16920377
  source 1..25: mol_type = other DNA
tcatcatggcagatagcgccggtgc

SEQ ID NO: 1141 - DNA, 25 bp, synthetic construct
  misc_feature 1..25: note = >Male | ENST00000481363 | 16920377
  source 1..25: mol_type = other DNA
cattcgttgggatcagtggtggcct

SEQ ID NO: 1142 - DNA, 25 bp, synthetic construct
  misc_feature 1..25: note = >Male | ENST00000481363 | 16920377
  source 1..25: mol_type = other DNA
tgtcctggaactcggcaaacagctg

SEQ ID NO: 1143 - DNA, 25 bp, synthetic construct
  misc_feature 1..25: note = >Male | ENST00000481363 | 16920377
  source 1..25: mol_type = other DNA
aacctgctgagacatgtggctgccc

SEQ ID NO: 1144 - DNA, 25 bp, synthetic construct
  misc_feature 1..25: note = >Male | ENST00000481363 | 16920377
  source 1..25: mol_type = other DNA
tttaggtacaccacagactccgcat

SEQ ID NO: 1145 - DNA, 25 bp, synthetic construct
  misc_feature 1..25: note = >Male | ENST00000481363 | 16920377
  source 1..25: mol_type = other DNA
gaagtcttctgaaggcacaggcccc

SEQ ID NO: 1146 - DNA, 25 bp, synthetic construct
  misc_feature 1..25: note = >Male | ENST00000481363 | 16920377
  source 1..25: mol_type = other DNA
tacacattgcaactccgaacttggg

SEQ ID NO: 1147 - DNA, 25 bp, synthetic construct
  misc_feature 1..25: note = >Male | ENST00000481363 | 16920377
  source 1..25: mol_type = other DNA
gatgcctggaagtctctcgaagcta

SEQ ID NO: 1148 - DNA, 25 bp, synthetic construct
  misc_feature 1..25: note = >Male | ENST00000481363 | 16920377
  source 1..25: mol_type = other DNA
tcaggtataccccatcatcaagata

SEQ ID NO: 1149 - DNA, 25 bp, synthetic construct
  misc_feature 1..25: note = >Male | ENST00000481363 | 16920377
  source 1..25: mol_type = other DNA
gcatatctcagacccagcagctaag

SEQ ID NO: 1150 - DNA, 25 bp, synthetic construct
  misc_feature 1..25: note = >Male | ENST00000481363 | 16920377
  source 1..25: mol_type = other DNA
cacaagacattcacacgagcataca

SEQ ID NO: 1151 - DNA, 25 bp, synthetic construct
  misc_feature 1..25: note = >Male | ENST00000481363 | 16920377
  source 1..25: mol_type = other DNA
tcacacgagcatacaatgatctagg

SEQ ID NO: 1152 - DNA, 25 bp, synthetic construct
  misc_feature 1..25: note = >Male | ENST00000481363 | 16920377
  source 1..25: mol_type = other DNA
tgactcttaaaacagcagcagctct

SEQ ID NO: 1153 - DNA, 25 bp, synthetic construct
  misc_feature 1..25: note = >Male | ENST00000481363 | 16920377
  source 1..25: mol_type = other DNA
aggccaccactggtaaataccgcaa

SEQ ID NO: 1154 - DNA, 25 bp, synthetic construct
  misc_feature 1..25: note = >Male | ENST00000481363 | 16920377
  source 1..25: mol_type = other DNA
ttaggaagtcggaaatcaaagatgg

SEQ ID NO: 1155 - DNA, 25 bp, synthetic construct
  misc_feature 1..25: note = >Male | ENST00000481363 | 16920377
  source 1..25: mol_type = other DNA
taggaagtcggaaatcaaagatggc

SEQ ID NO: 1156 - DNA, 25 bp, synthetic construct
  misc_feature 1..25: note = >Male | ENST00000481363 | 16920377
  source 1..25: mol_type = other DNA
aaggttagggcgaagatcggcaggc

SEQ ID NO: 1157 - DNA, 25 bp, synthetic construct
  misc_feature 1..25: note = >Male | ENST00000481363 | 16920377
  source 1..25: mol_type = other DNA
tggtcctcctccgagttgatgtgct

SEQ ID NO: 1158 - DNA, 25 bp, synthetic construct
  misc_feature 1..25: note = >Male | ENST00000481363 | 16920377
  source 1..25: mol_type = other DNA
cgggagagccgcagttatttgccct

SEQ ID NO: 1159 - DNA, 25 bp, synthetic construct
  misc_feature 1..25: note = >Male | NM_138375 | 16851430
  source 1..25: mol_type = other DNA
taccctgtcgtttatctcataagca

SEQ ID NO: 1160 - DNA, 25 bp, synthetic construct
  misc_feature 1..25: note = >Male | NM_138375 | 16851430
  source 1..25: mol_type = other DNA
cataaactgggaccaatgtggacag

SEQ ID NO: 1161 - DNA, 25 bp, synthetic construct
  misc_feature 1..25: note = >Male | NM_138375 | 16851430
  source 1..25: mol_type = other DNA
tgagtgaacgagttgaggcgtcccc

SEQ ID NO: 1162 - DNA, 25 bp, synthetic construct
  misc_feature 1..25: note = >Male | NM_138375 | 16851430
  source 1..25: mol_type = other DNA
tgagtcattcaaacaccaggaagaa

SEQ ID NO: 1163 - DNA, 25 bp, synthetic construct
  misc_feature 1..25: note = >Male | NM_138375 | 16851430
  source 1..25: mol_type = other DNA
gaggcttcagaccactccaaactca

SEQ ID NO: 1164 - DNA, 25 bp, synthetic construct
  misc_feature 1..25: note = >Male | NM_138375 | 16851430
  source 1..25: mol_type = other DNA
tatattcttgcatggcagcccctct

SEQ ID NO: 1165 - DNA, 25 bp, synthetic construct
  misc_feature 1..25: note = >Male | NM_138375 | 16851430
  source 1..25: mol_type = other DNA
tccaaggaagatctctgggagatga

SEQ ID NO: 1166 - DNA, 25 bp, synthetic construct
  misc_feature 1..25: note = >Male | NM_138375 | 16851430
  source 1..25: mol_type = other DNA
gggcgttctcctcaatatcttccag

SEQ ID NO: 1167 - DNA, 25 bp, synthetic construct
  misc_feature 1..25: note = >Male | NM_138375 | 16851430
  source 1..25: mol_type = other DNA
gggtgaacctgagagagttcgacat

SEQ ID NO: 1168 - DNA, 25 bp, synthetic construct
  misc_feature 1..25: note = >Male | NM_138375 | 16851430
  source 1..25: mol_type = other DNA
aaattctttggtctgggtgaacctg

SEQ ID NO: 1169 - DNA, 25 bp, synthetic construct
  misc_feature 1..25: note = >Male | NM_138375 | 16851430
  source 1..25: mol_type = other DNA
cattcctggtatcgtgttgccgcat

SEQ ID NO: 1170 - DNA, 25 bp, synthetic construct
  misc_feature 1..25: note = >Male | NM_138375 | 16851430
  source 1..25: mol_type = other DNA
ccattcctggtatcgtgttgccgca

SEQ ID NO: 1171 - DNA, 25 bp, synthetic construct
  misc_feature 1..25: note = >Male | NM_138375 | 16851430
  source 1..25: mol_type = other DNA
cagaaggatcttctgccactgataa

SEQ ID NO: 1172 - DNA, 25 bp, synthetic construct
  misc_feature 1..25: note = >Male | NM_138375 | 16851430
  source 1..25: mol_type = other DNA
acttgggtactgtcgcgatacggca

SEQ ID NO: 1173 - DNA, 25 bp, synthetic construct
  misc_feature 1..25: note = >Male | NM_138375 | 16851430
  source 1..25: mol_type = other DNA
cagttgattgtcttcctccgtccaa

SEQ ID NO: 1174 - DNA, 25 bp, synthetic construct
  misc_feature 1..25: note = >Male | NM_138375 | 16851430
  source 1..25: mol_type = other DNA
tctttcaaactcactgcaccagttg

SEQ ID NO: 1175 - DNA, 25 bp, synthetic construct
  misc_feature 1..25: note = >Male | NM_138375 | 16851430
  source 1..25: mol_type = other DNA
tatttctccgggctccaaaggcatt

SEQ ID NO: 1176 - DNA, 25 bp, synthetic construct
  misc_feature 1..25: note = >Male | NM_138375 | 16851430
  source 1..25: mol_type = other DNA
ctgcggtggctcaggttacggaact

SEQ ID NO: 1177 - DNA, 25 bp, synthetic construct
  misc_feature 1..25: note = >Male | NM_138375 | 16851430
  source 1..25: mol_type = other DNA
ggtcatagtccataaagtctcccag

SEQ ID NO: 1178 - DNA, 25 bp, synthetic construct
  misc_feature 1..25: note = >Male | NM_138375 | 16851430
  source 1..25: mol_type = other DNA
tcagaacgcgtttgtgtttgccaca

SEQ ID NO: 1179 - DNA, 25 bp, synthetic construct
  misc_feature 1..25: note = >Male | NM_138375 | 16851430
  source 1..25: mol_type = other DNA
tgtcagcttaatgtgaggaaacttc

SEQ ID NO: 1180 - DNA, 25 bp, synthetic construct
  misc_feature 1..25: note = >Male | NM_138375 | 16851430
  source 1..25: mol_type = other DNA
aagcttccgcatctctcgtttcaga

SEQ ID NO: 1181 - DNA, 25 bp, synthetic construct
  misc_feature 1..25: note = >Male | NM_138375 | 16851430
  source 1..25: mol_type = other DNA
taacaccgggaattcaaaggcaatc

SEQ ID NO: 1182 - DNA, 25 bp, synthetic construct
  misc_feature 1..25: note = >Male | NM_138375 | 16851430
  source 1..25: mol_type = other DNA
caagtggagggcgaattccaaggcc

SEQ ID NO: 1183 - DNA, 25 bp, synthetic construct
  misc_feature 1..25: note = >Male | NM_138375 | 16851430
  source 1..25: mol_type = other DNA
tcatggacgtgcaaaaggagacctc

SEQ ID NO: 1184 - DNA, 25 bp, synthetic construct
  misc_feature 1..25: note = >Male | NM_005035 | 16866470
  source 1..25: mol_type = other DNA
aaaagagctttatttacacactgac

SEQ ID NO: 1185 - DNA, 25 bp, synthetic construct
  misc_feature 1..25: note = >Male | NM_005035 | 16866470
  source 1..25: mol_type = other DNA
ctggcttgggcaccgcctgcagtgt

SEQ ID NO: 1186 - DNA, 25 bp, synthetic construct
  misc_feature 1..25: note = >Male | NM_005035 | 16866470
  source 1..25: mol_type = other DNA
gagcagaaccgcttgaccaggaatc

SEQ ID NO: 1187 - DNA, 25 bp, synthetic construct
  misc_feature 1..25: note = >Male | NM_005035 | 16866470
  source 1..25: mol_type = other DNA
agaaccgcttgaccaggaatctgga

SEQ ID NO: 1188 - DNA, 25 bp, synthetic construct
  misc_feature 1..25: note = >Male | NM_005035 | 16866470
  source 1..25: mol_type = other DNA
tggttcatgacggagacatcagctg

SEQ ID NO: 1189 - DNA, 25 bp, synthetic construct
  misc_feature 1..25: note = >Male | NM_005035 | 16866470
  source 1..25: mol_type = other DNA
tgcgtgagtccagtaacagtcgtgc

SEQ ID NO: 1190 - DNA, 25 bp, synthetic construct
  misc_feature 1..25: note = >Male | NM_005035 | 16866470
  source 1..25: mol_type = other DNA
gaagccgttcttctgcttacgtgtg

SEQ ID NO: 1191 - DNA, 25 bp, synthetic construct
  misc_feature 1..25: note = >Male | NM_005035 | 16866470
  source 1..25: mol_type = other DNA
tcttctgcttacgtgtgttgggctt

SEQ ID NO: 1192 - DNA, 25 bp, synthetic construct
  misc_feature 1..25: note = >Male | NM_005035 | 16866470
  source 1..25: mol_type = other DNA
accttggagtccaggcgatagggct

SEQ ID NO: 1193 - DNA, 25 bp, synthetic construct
  misc_feature 1..25: note = >Male | NM_005035 | 16866470
  source 1..25: mol_type = other DNA
ccacagagcccatgtgggagatgag

SEQ ID NO: 1194 - DNA, 25 bp, synthetic construct
  misc_feature 1..25: note = >Male | NM_005035 | 16866470
  source 1..25: mol_type = other DNA
gaagacctggcgtacgagatagtga

SEQ ID NO: 1195 - DNA, 25 bp, synthetic construct
  misc_feature 1..25: note = >Male | NM_005035 | 16866470
  source 1..25: mol_type = other DNA
cgtacgagatagtgagaggcctccc

SEQ ID NO: 1196 - DNA, 25 bp, synthetic construct
  misc_feature 1..25: note = >Male | NM_005035 | 16866470
  source 1..25: mol_type = other DNA
caccttgcgggtgatgaaaccttcc

SEQ ID NO: 1197 - DNA, 25 bp, synthetic construct
  misc_feature 1..25: note = >Male | NM_005035 | 16866470
  source 1..25: mol_type = other DNA
cataatgctgcaggccgttgcaaga

SEQ ID NO: 1198 - DNA, 25 bp, synthetic construct
  misc_feature 1..25: note = >Male | NM_005035 | 16866470
  source 1..25: mol_type = other DNA
gattgaccaggtggatcttgagcca

SEQ ID NO: 1199 - DNA, 25 bp, synthetic construct
  misc_feature 1..25: note = >Male | NM_005035 | 16866470
  source 1..25: mol_type = other DNA
gctgagcaggaaagcaccagagtgc

SEQ ID NO: 1200 - DNA, 25 bp, synthetic construct
  misc_feature 1..25: note = >Male | NM_005035 | 16866470
  source 1..25: mol_type = other DNA
cggaaggaatacacgtggtagagca

SEQ ID NO: 1201 - DNA, 25 bp, synthetic construct
  misc_feature 1..25: note = >Male | NM_005035 | 16866470
  source 1..25: mol_type = other DNA
aacgatgcggcttgtccaggctgca

SEQ ID NO: 1202 - DNA, 25 bp, synthetic construct
  misc_feature 1..25: note = >Male | NM_005035 | 16866470
  source 1..25: mol_type = other DNA
gaggtacttcctgtagtggttctgc

SEQ ID NO: 1203 - DNA, 25 bp, synthetic construct
  misc_feature 1..25: note = >Male | NM_005035 | 16866470
  source 1..25: mol_type = other DNA
tacacctcgcgctctaggcggttct

SEQ ID NO: 1204 - DNA, 25 bp, synthetic construct
  misc_feature 1..25: note = >Male | NM_005035 | 16866470
  source 1..25: mol_type = other DNA
catgtggagctgcttctcaaagagg

SEQ ID NO: 1205 - DNA, 25 bp, synthetic construct
  misc_feature 1..25: note = >Male | NM_005035 | 16866470
  source 1..25: mol_type = other DNA
taggacagcaggtccggagtcaagc

SEQ ID NO: 1206 - DNA, 25 bp, synthetic construct
  misc_feature 1..25: note = >Male | NM_005035 | 16866470
  source 1..25: mol_type = other DNA
taacacatataccagctccttgaag

SEQ ID NO: 1207 - DNA, 25 bp, synthetic construct
  misc_feature 1..25: note = >Male | NM_005035 | 16866470
  source 1..25: mol_type = other DNA
gggtccgcttatccttctccagtat

SEQ ID NO: 1208 - DNA, 25 bp, synthetic construct
  misc_feature 1..25: note = >Male | NM_005035 | 16866470
  source 1..25: mol_type = other DNA
tgggtggctggaggctaccatctcc

SEQ ID NO: 1209 - DNA, 25 bp, synthetic construct
  misc_feature 1..25: note = >Male | NM_005035 | 16866470
  source 1..25: mol_type = other DNA
ccgcaaggccgtagggctcgtttga

SEQ ID NO: 1210 - DNA, 25 bp, synthetic construct
  misc_feature 1..25: note = >Male | NM_005035 | 16866470
  source 1..25: mol_type = other DNA
tagggctcgtttgagccccgccgct

SEQ ID NO: 1211 - DNA, 25 bp, synthetic construct
  misc_feature 1..25: note = >Male | ENST00000503919 | 17000583
  source 1..25: mol_type = other DNA
tgttcttcctagatgctgacaagtc

SEQ ID NO: 1212 - DNA, 25 bp, synthetic construct
  misc_feature 1..25: note = >Male | ENST00000503919 | 17000583
  source 1..25: mol_type = other DNA
tgtatcagaccacgctcctctgggc

SEQ ID NO: 1213 - DNA, 25 bp, synthetic construct
  misc_feature 1..25: note = >Male | ENST00000503919 | 17000583
  source 1..25: mol_type = other DNA
tcaagtatgaaagcaaggcacccgc

SEQ ID NO: 1214 - DNA, 25 bp, synthetic construct
  misc_feature 1..25: note = >Male | ENST00000503919 | 17000583
  source 1..25: mol_type = other DNA
acaaacatctccgtggtcagcagca

SEQ ID NO: 1215 - DNA, 25 bp, synthetic construct
  misc_feature 1..25: note = >Male | ENST00000503919 | 17000583
  source 1..25: mol_type = other DNA
tattgatggcgccagggttggactc

SEQ ID NO: 1216 - DNA, 25 bp, synthetic construct
  misc_feature 1..25: note = >Male | ENST00000503919 | 17000583
  source 1..25: mol_type = other DNA
agaagagggccgtgaacttgccgat

SEQ ID NO: 1217 - DNA, 25 bp, synthetic construct
  misc_feature 1..25: note = >Male | ENST00000503919 | 17000583
  source 1..25: mol_type = other DNA
aggaccagcatgatagccgcaccat

SEQ ID NO: 1218 - DNA, 25 bp, synthetic construct
  misc_feature 1..25: note = >Male | ENST00000503919 | 17000583
  source 1..25: mol_type = other DNA
taattcccaggacgccaatgttggg

SEQ ID NO: 1219 - DNA, 25 bp, synthetic construct
  misc_feature 1..25: note = >Male | ENST00000503919 | 17000583
  source 1..25: mol_type = other DNA
tagccccgcgatgatgcagcaaatg

SEQ ID NO: 1220 - DNA, 25 bp, synthetic construct
  misc_feature 1..25: note = >Male | ENST00000503919 | 17000583
  source 1..25: mol_type = other DNA
ctcaatgatgcctgccagagtggcg

SEQ ID NO: 1221 - DNA, 25 bp, synthetic construct
  misc_feature 1..25: note = >Male | ENST00000503919 | 17000583
  source 1..25: mol_type = other DNA
caaggtcaggacatagcagagcagc

SEQ ID NO: 1222 - DNA, 25 bp, synthetic construct
  misc_feature 1..25: note = >Male | ENST00000503919 | 17000583
  source 1..25: mol_type = other DNA
ggaggtctctgtccaggctaacctg

SEQ ID NO: 1223 - DNA, 25 bp, synthetic construct
  misc_feature 1..25: note = >Male | ENST00000503919 | 17000583
  source 1..25: mol_type = other DNA
agtgaggcccttgccccagcggtag

SEQ ID NO: 1224 - DNA, 25 bp, synthetic construct
  misc_feature 1..25: note = >Male | ENST00000503919 | 17000583
  source 1..25: mol_type = other DNA
gaaggtgaggttgcgcaggtactgg

SEQ ID NO: 1225 - DNA, 25 bp, synthetic construct
  misc_feature 1..25: note = >Male | ENST00000503919 | 17000583
  source 1..25: mol_type = other DNA
ccgggaagaaggaccaaggacatga

SEQ ID NO: 1226 - DNA, 25 bp, synthetic construct
  misc_feature 1..25: note = >Male | ENST00000503919 | 17000583
  source 1..25: mol_type = other DNA
aaggaccaaggacatgaagagggta

SEQ ID NO: 1227 - DNA, 25 bp, synthetic construct
  misc_feature 1..25: note = >Male | ENST00000503919 | 17000583
  source 1..25: mol_type = other DNA
gctcggtcgccagcagcttggaaga

SEQ ID NO: 1228 - DNA, 25 bp, synthetic construct
  misc_feature 1..25: note = >Male | ENST00000503919 | 17000583
  source 1..25: mol_type = other DNA
ccgtatccgtgggtgccaaatatga

SEQ ID NO: 1229 - DNA, 25 bp, synthetic construct
  misc_feature 1..25: note = >Male | ENST00000503919 | 17000583
  source 1..25: mol_type = other DNA
gggcagactccagttaccgtagatc

SEQ ID NO: 1230 - DNA, 25 bp, synthetic construct
  misc_feature 1..25: note = >Male | ENST00000503919 | 17000583
  source 1..25: mol_type = other DNA
tagagtcagggatacacacacggac

SEQ ID NO: 1231 - DNA, 25 bp, synthetic construct
  misc_feature 1..25: note = >Male | ENST00000503919 | 17000583
  source 1..25: mol_type = other DNA
acggaccaggtacagagacagagag

SEQ ID NO: 1232 - DNA, 25 bp, synthetic construct
  misc_feature 1..25: note = >Male | ENST00000503919 | 17000583
  source 1..25: mol_type = other DNA
taaccatgtgctggtcgtggcccac

SEQ ID NO: 1233 - DNA, 25 bp, synthetic construct
  misc_feature 1..25: note = >Male | ENST00000503919 | 17000583
  source 1..25: mol_type = other DNA
ggcacggcgatggtaccactgaagc

SEQ ID NO: 1234 - DNA, 25 bp, synthetic construct
  misc_feature 1..25: note = >Male | ENST00000503919 | 17000583
  source 1..25: mol_type = other DNA
caagggattcccagagagctactgc

SEQ ID NO: 1235 - DNA, 25 bp, synthetic construct
  misc_feature 1..25: note = >Male | ENST00000503919 | 17000583
  source 1..25: mol_type = other DNA
caagatattcctgcggcttccgtgg

SEQ ID NO: 1236 - DNA, 25 bp, synthetic construct
  misc_feature 1..25: note = >Male | ENST00000503919 | 17000583
  source 1..25: mol_type = other DNA
cacaggtaccaaggtggcacgtcct

SEQ ID NO: 1237 - DNA, 25 bp, synthetic construct
  misc_feature 1..25: note = >Male | ENST00000503919 | 17000583
  source 1..25: mol_type = other DNA
caaacttaggctctgtgggtagcgg

SEQ ID NO: 1238 - DNA, 25 bp, synthetic construct
  misc_feature 1..25: note = >Male | ENST00000503919 | 17000583
  source 1..25: mol_type = other DNA
gatgacttgacaaaggccaaggagg

SEQ ID NO: 1239 - DNA, 25 bp, synthetic construct
  misc_feature 1..25: note = >Male | ENST00000503919 | 17000583
  source 1..25: mol_type = other DNA
ccaaggaggaggactttactccaca

SEQ ID NO: 1240 - DNA, 25 bp, synthetic construct
  misc_feature 1..25: note = >Male | ENST00000503919 | 17000583
  source 1..25: mol_type = other DNA
tactgctcccaatttccaaagttgc

SEQ ID NO: 1241 - DNA, 25 bp, synthetic construct
  misc_feature 1..25: note = >Male | NM_024807 | 17019044
  source 1..25: mol_type = other DNA
tatacaacaggatggtggctacacc

SEQ ID NO: 1242 - DNA, 25 bp, synthetic construct
  misc_feature 1..25: note = >Male | NM_024807 | 17019044
  source 1..25: mol_type = other DNA
agctttctaaggaggcagcggccag

SEQ ID NO: 1243 - DNA, 25 bp, synthetic construct
  misc_feature 1..25: note = >Male | NM_024807 | 17019044
  source 1..25: mol_type = other DNA
tcttccaggtgggtcacgtgtagaa

SEQ ID NO: 1244 - DNA, 25 bp, synthetic construct
  misc_feature 1..25: note = >Male | NM_024807 | 17019044
  source 1..25: mol_type = other DNA
ggtcacgtgtagaaggatcgctgca

SEQ ID NO: 1245 - DNA, 25 bp, synthetic construct
  misc_feature 1..25: note = >Male | NM_024807 | 17019044
  source 1..25: mol_type = other DNA
gaaggatcgctgcacatgctgtagc

SEQ ID NO: 1246 - DNA, 25 bp, synthetic construct
  misc_feature 1..25: note = >Male | NM_024807 | 17019044
  source 1..25: mol_type = other DNA
acccatagaccatgatcagcatcag

SEQ ID NO: 1247 - DNA, 25 bp, synthetic construct
  misc_feature 1..25: note = >Male | NM_024807 | 17019044
  source 1..25: mol_type = other DNA
caagcacagtggagtaaacatcctg

SEQ ID NO: 1248 - DNA, 25 bp, synthetic construct
  misc_feature 1..25: note = >Male | NM_024807 | 17019044
  source 1..25: mol_type = other DNA
cacagtggagtaaacatcctggtgc

SEQ ID NO: 1249 - DNA, 25 bp, synthetic construct
  misc_feature 1..25: note = >Male | NM_024807 | 17019044
  source 1..25: mol_type = other DNA
tgctgaggtccccagacttagtgga

SEQ ID NO: 1250 - DNA, 25 bp, synthetic construct
  misc_feature 1..25: note = >Male | NM_024807 | 17019044
  source 1..25: mol_type = other DNA
gagcctaggcaaggtgatgagtcct

SEQ ID NO: 1251 - DNA, 25 bp, synthetic construct
  misc_feature 1..25: note = >Male | NM_024807 | 17019044
  source 1..25: mol_type = other DNA
tttcacccagactcgggcaaagcca

SEQ ID NO: 1252 - DNA, 25 bp, synthetic construct
  misc_feature 1..25: note = >Male | NM_024807 | 17019044
  source 1..25: mol_type = other DNA
tccccttcaaggagcctcacttttg

SEQ ID NO: 1253 - DNA, 25 bp, synthetic construct
  misc_feature 1..25: note = >Male | NM_024807 | 17019044
  source 1..25: mol_type = other DNA
tttgtgtatacactgtcagcagagg

SEQ ID NO: 1254 - DNA, 25 bp, synthetic construct
  misc_feature 1..25: note = >Male | NM_024807 | 17019044
  source 1..25: mol_type = other DNA
ttgtgtatacactgtcagcagaggg

SEQ ID NO: 1255 - DNA, 25 bp, synthetic construct
  misc_feature 1..25: note = >Male | NM_024807 | 17019044
  source 1..25: mol_type = other DNA
tgagacgcaaccctgtggccacagc

SEQ ID NO: 1256 - DNA, 25 bp, synthetic construct
  misc_feature 1..25: note = >Male | NM_024807 | 17019044
  source 1..25: mol_type = other DNA
gacacaggatgtgccacctgggcct

SEQ ID NO: 1257 - DNA, 25 bp, synthetic construct
  misc_feature 1..25: note = >Male | NR_003697 | 17057414
  source 1..25: mol_type = other DNA
tagggccagaggtctgtctgccaga

SEQ ID NO: 1258 - DNA, 25 bp, synthetic construct
  misc_feature 1..25: note = >Male | NR_003697 | 17057414
  source 1..25: mol_type = other DNA
cttccagaagggcaacaagcgaggt

SEQ ID NO: 1259 - DNA, 25 bp, synthetic construct
  misc_feature 1..25: note = >Male | NR_003697 | 17057414
  source 1..25: mol_type = other DNA
ggttccaaggaacagcctggcagcc

SEQ ID NO: 1260 - DNA, 25 bp, synthetic construct
  misc_feature 1..25: note = >Male | NR_003697 | 17057414
  source 1..25: mol_type = other DNA
ctggcagccactgaaggtatcttct

SEQ ID NO: 1261 - DNA, 25 bp, synthetic construct
  misc_feature 1..25: note = >Male | NR_003697 | 17057414
  source 1..25: mol_type = other DNA
gaaacactgacggatggcaggcact

SEQ ID NO: 1262 - DNA, 25 bp, synthetic construct
  misc_feature 1..25: note = >Male | NR_003697 | 17057414
  source 1..25: mol_type = other DNA
cggtcgcctcccagtttcatggaca

SEQ ID NO: 1263 - DNA, 25 bp, synthetic construct
  misc_feature 1..25: note = >Male | NR_003697 | 17057414
  source 1..25: mol_type = other DNA
ctggtagaaaatacaaaattcctcg

SEQ ID NO: 1264 - DNA, 25 bp, synthetic construct
  misc_feature 1..25: note = >Male | NR_003697 | 17057414
  source 1..25: mol_type = other DNA
ctcgattagggacaagggaccactg

SEQ ID NO: 1265 - DNA, 25 bp, synthetic construct
  misc_feature 1..25: note = >Male | NR_003697 | 17057414
  source 1..25: mol_type = other DNA
tcaaaacaatagcaatagccagagc

SEQ ID NO: 1266 - DNA, 25 bp, synthetic construct
  misc_feature 1..25: note = >Male | NR_003697 | 17057414
  source 1..25: mol_type = other DNA
gcaatagccagagcatcagcatttt

SEQ ID NO: 1267 - DNA, 25 bp, synthetic construct
  misc_feature 1..25: note = >Male | NR_003697 | 17057414
  source 1..25: mol_type = other DNA
gagccattggactactacaagaaag

SEQ ID NO: 1268 - DNA, 25 bp, synthetic construct
  misc_feature 1..25: note = >Male | NR_003697 | 17057414
  source 1..25: mol_type = other DNA
gactactacaagaaaggagcgcagg

SEQ ID NO: 1269 - DNA, 25 bp, synthetic construct
  misc_feature 1..25: note = >Male | NR_003697 | 17057414
  source 1..25: mol_type = other DNA
gagcgcaggactctaccctttcaca

SEQ ID NO: 1270 - DNA, 25 bp, synthetic construct
  misc_feature 1..25: note = >Male | NR_003697 | 17057414
  source 1..25: mol_type = other DNA
aacgcacagcaagaatgcctgccct

SEQ ID NO: 1271 - DNA, 25 bp, synthetic construct
  misc_feature 1..25: note = >Male | NR_003697 | 17057414
  source 1..25: mol_type = other DNA
gagttgctgtgacagtaagctcttc

SEQ ID NO: 1272 - DNA, 25 bp, synthetic construct
  misc_feature 1..25: note = >Male | NR_003697 | 17057414
  source 1..25: mol_type = other DNA
ttccactttgagaccgtcacctcag

SEQ ID NO: 1273 - DNA, 25 bp, synthetic construct
  misc_feature 1..25: note = >Male | NR_003697 | 17057414
  source 1..25: mol_type = other DNA
tttgagaccgtcacctcagccacgg

SEQ ID NO: 1274 - DNA, 25 bp, synthetic construct
  misc_feature 1..25: note = >Male | NR_003697 | 17057414
  source 1..25: mol_type = other DNA
ggtggatgactagactgccgaagac

SEQ ID NO: 1275 - DNA, 25 bp, synthetic construct
  misc_feature 1..25: note = >Male | NR_003697 | 17057414
  source 1..25: mol_type = other DNA
tggatgactagactgccgaagaccg

SEQ ID NO: 1276 - DNA, 25 bp, synthetic construct
  misc_feature 1..25: note = >Male | NR_003697 | 17057414
  source 1..25: mol_type = other DNA
cgctgcgactacgaagtacggagta

SEQ ID NO: 1277 - DNA, 25 bp, synthetic construct
  misc_feature 1..25: note = >Male | NM_000786 | 17059640
  source 1..25: mol_type = other DNA
ttaggccccgagttacaatttgaga

SEQ ID NO: 1278 - DNA, 25 bp, synthetic construct
  misc_feature 1..25: note = >Male | NM_000786 | 17059640
  source 1..25: mol_type = other DNA
tatttggcaagagcgatgagtactc

SEQ ID NO: 1279 - DNA, 25 bp, synthetic construct
  misc_feature 1..25: note = >Male | NM_000786 | 17059640
  source 1..25: mol_type = other DNA
cattttgatcttcgtttgtaacgga

SEQ ID NO: 1280 - DNA, 25 bp, synthetic construct
  misc_feature 1..25: note = >Male | NM_000786 | 17059640
  source 1..25: mol_type = other DNA
ttttccccaatacaacgatgacgcc

SEQ ID NO: 1281 - DNA, 25 bp, synthetic construct
  misc_feature 1..25: note = >Male | NM_000786 | 17059640
  source 1..25: mol_type = other DNA
gctccaaatggcacataggcaaact

SEQ ID NO: 1282 - DNA, 25 bp, synthetic construct
  misc_feature 1..25: note = >Male | NM_000786 | 17059640
  source 1..25: mol_type = other DNA
taagtctttgattgacagtgggaga

SEQ ID NO: 1283 - DNA, 25 bp, synthetic construct
  misc_feature 1..25: note = >Male | NM_000786 | 17059640
  source 1..25: mol_type = other DNA
taaaaactggctagataataggcaa

SEQ ID NO: 1284 - DNA, 25 bp, synthetic construct
  misc_feature 1..25: note = >Male | NM_000786 | 17059640
  source 1..25: mol_type = other DNA
ggagttctggccattctcatcatga

SEQ ID NO: 1285 - DNA, 25 bp, synthetic construct
  misc_feature 1..25: note = >Male | NM_000786 | 17059640
  source 1..25: mol_type = other DNA
tctctggccaaaaagaagcccatcc

SEQ ID NO: 1286 - DNA, 25 bp, synthetic construct
  misc_feature 1..25: note = >Male | NM_000786 | 17059640
  source 1..25: mol_type = other DNA
gaggatgtatgctgccctgccaaga

SEQ ID NO: 1287 - DNA, 25 bp, synthetic construct
  misc_feature 1..25: note = >Male | NM_000786 | 17059640
  source 1..25: mol_type = other DNA
tatccttgatttcccgatgagctct

SEQ ID NO: 1288 - DNA, 25 bp, synthetic construct
  misc_feature 1..25: note = >Male | NM_000786 | 17059640
  source 1..25: mol_type = other DNA
caatggctagctgttaaaattatga

SEQ ID NO: 1289 - DNA, 25 bp, synthetic construct
  misc_feature 1..25: note = >Male | NM_000786 | 17059640
  source 1..25: mol_type = other DNA
ttaaagtgggctatgttaaggccac

SEQ ID NO: 1290 - DNA, 25 bp, synthetic construct
  misc_feature 1..25: note = >Male | NM_000786 | 17059640
  source 1..25: mol_type = other DNA
agtgcagcagcatcactccccagaa

SEQ ID NO: 1291 - DNA, 25 bp, synthetic construct
  misc_feature 1..25: note = >Male | NM_000786 | 17059640
  source 1..25: mol_type = other DNA
gtgcagcagcatcactccccagaag

SEQ ID NO: 1292 - DNA, 25 bp, synthetic construct
  misc_feature 1..25: note = >Male | NM_000786 | 17059640
  source 1..25: mol_type = other DNA
tctagaaattcaattggacttttcc

SEQ ID NO: 1293 - DNA, 25 bp, synthetic construct
  misc_feature 1..25: note = >Male | NM_000786 | 17059640
  source 1..25: mol_type = other DNA
aatgctatggcatgcccaaggaatg

SEQ ID NO: 1294 - DNA, 25 bp, synthetic construct
  misc_feature 1..25: note = >Male | NM_000786 | 17059640
  source 1..25: mol_type = other DNA
gcatggacaagaggttgccgcctgt

SEQ ID NO: 1295 - DNA, 25 bp, synthetic construct
  misc_feature 1..25: note = >Male | NM_000786 | 17059640
  source 1..25: mol_type = other DNA
catggacaagaggttgccgcctgtc

SEQ ID NO: 1296 - DNA, 25 bp, synthetic construct
  misc_feature 1..25: note = >Male | NM_000786 | 17059640
  source 1..25: mol_type = other DNA
ggaaacactgaaggccgaggtcgcc

SEQ ID NO: 1297 - DNA, 25 bp, synthetic construct
  misc_feature 1..25: note = >Male | NM_000786 | 17059640
  source 1..25: mol_type = other DNA
aatcgacggaacgagagaagctggc

SEQ ID NO: 1298 - DNA, 25 bp, synthetic construct
  misc_feature 1..25: note = >Male | NM_000786 | 17059640
  source 1..25: mol_type = other DNA
tgaagaggcgatcaatccctgagaa

SEQ ID NO: 1299 - DNA, 25 bp, synthetic construct
  misc_feature 1..25: note = >Male | NM_000786 | 17059640
  source 1..25: mol_type = other DNA
gatcaatccctgagaatcgcgggcg

SEQ ID NO: 1300 - DNA, 25 bp, synthetic construct
  misc_feature 1..25: note = >Male | ExonID_5299845 | 17076434
  source 1..25: mol_type = other DNA
cagggacctgaatgcaaaactagcc

SEQ ID NO: 1301 - DNA, 25 bp, synthetic construct
  misc_feature 1..25: note = >Male | ExonID_5299845 | 17076434
  source 1..25: mol_type = other DNA
gaggtcaccttcaatcgaggcacga

SEQ ID NO: 1302 - DNA, 25 bp, synthetic construct
  misc_feature 1..25: note = >Male | ExonID_5299845 | 17076434
  source 1..25: mol_type = other DNA
ggctcatgagagaagacggaatcct

SEQ ID NO: 1303 - DNA, 25 bp, synthetic construct
  misc_feature 1..25: note = >Male | ExonID_5299845 | 17076434
  source 1..25: mol_type = other DNA
ttgaaaagttcctccacaggcacac

SEQ ID NO: 1304 - DNA, 25 bp, synthetic construct
  misc_feature 1..25: note = >Male | ExonID_5299845 | 17076434
  source 1..25: mol_type = other DNA
catactccacgatgacatacaaggg

SEQ ID NO: 1305 - DNA, 25 bp, synthetic construct
  misc_feature 1..25: note = >Male | ExonID_5299845 | 17076434
  source 1..25: mol_type = other DNA
tctgagatcaggtctgacaagtctt

SEQ ID NO: 1306 - DNA, 25 bp, synthetic construct
  misc_feature 1..25: note = >Male | ExonID_5299845 | 17076434
  source 1..25: mol_type = other DNA
tgagactcatttacttgggaaggga

SEQ ID NO: 1307 - DNA, 25 bp, synthetic construct
  misc_feature 1..25: note = >Male | ExonID_5299845 | 17076434
  source 1..25: mol_type = other DNA
tagtcagggcttcccaacaatggca

SEQ ID NO: 1308 - DNA, 25 bp, synthetic construct
  misc_feature 1..25: note = >Male | ExonID_5299845 | 17076434
  source 1..25: mol_type = other DNA
gaaggcccctgtgcaatagatgatg

SEQ ID NO: 1309 - DNA, 25 bp, synthetic construct
  misc_feature 1..25: note = >Male | ExonID_5299845 | 17076434
  source 1..25: mol_type = other DNA
tgcaagggccagctgagaagtcact

SEQ ID NO: 1310 - DNA, 25 bp, synthetic construct
  misc_feature 1..25: note = >Male | ExonID_5299845 | 17076434
  source 1..25: mol_type = other DNA
caggacatcgagaggagaagttaca

SEQ ID NO: 1311 - DNA, 25 bp, synthetic construct
  misc_feature 1..25: note = >Male | ExonID_5299845 | 17076434
  source 1..25: mol_type = other DNA
gagtccgatagagttacccgccaag

SEQ ID NO: 1312 - DNA, 25 bp, synthetic construct
  misc_feature 1..25: note = >Male | ExonID_5299845 | 17076434
  source 1..25: mol_type = other DNA
tctaagagacgcagagcaagggaag

SEQ ID NO: 1313 - DNA, 25 bp, synthetic construct
  misc_feature 1..25: note = >Male | ExonID_5299845 | 17076434
  source 1..25: mol_type = other DNA
catggatccacacaaagccaaggaa

SEQ ID NO: 1314 - DNA, 25 bp, synthetic construct
  misc_feature 1..25: note = >Male | ExonID_5299845 | 17076434
  source 1..25: mol_type = other DNA
cgcatccgagctattaatccccgaa

SEQ ID NO: 1315 - DNA, 25 bp, synthetic construct
  misc_feature 1..25: note = >Male | ExonID_5299845 | 17076434
  source 1..25: mol_type = other DNA
gatcgtacgtaactcaggacacagg

SEQ ID NO: 1316 - DNA, 25 bp, synthetic construct
  misc_feature 1..25: note = >Male | ExonID_5299845 | 17076434
  source 1..25: mol_type = other DNA
atgaactccacgttgctacccaggg

SEQ ID NO: 1317 - DNA, 25 bp, synthetic construct
  misc_feature 1..25: note = >Male | ExonID_5299845 | 17076434
  source 1..25: mol_type = other DNA
catttttcaaccagcgcagtgtggg

SEQ ID NO: 1318 - DNA, 25 bp, synthetic construct
  misc_feature 1..25: note = >Male | ExonID_5299845 | 17076434
  source 1..25: mol_type = other DNA
acttagcagaacaggcaccgtgacc

SEQ ID NO: 1319 - DNA, 25 bp, synthetic construct
  misc_feature 1..25: note = >Male | ExonID_5299845 | 17076434
  source 1..25: mol_type = other DNA
ttaatcagagcagatagggcaccca

SEQ ID NO: 1320 - DNA, 25 bp, synthetic construct
  misc_feature 1..25: note = >Male | ExonID_5299845 | 17076434
  source 1..25: mol_type = other DNA
gagcagggataccaccacctgttca

SEQ ID NO: 1321 - DNA, 25 bp, synthetic construct
  misc_feature 1..25: note = >Male | ExonID_5299845 | 17076434
  source 1..25: mol_type = other DNA
taagccgagtaccaagtccaaatgg

SEQ ID NO: 1322 - DNA, 25 bp, synthetic construct
  misc_feature 1..25: note = >Male | ExonID_5299845 | 17076434
  source 1..25: mol_type = other DNA
cacctccgaggtgtgtcctggaagc

SEQ ID NO: 1323 - DNA, 25 bp, synthetic construct
  misc_feature 1..25: note = >Male | ExonID_5299845 | 17076434
  source 1..25: mol_type = other DNA
tgctggttacgcaagcatagaggcc

SEQ ID NO: 1324 - DNA, 25 bp, synthetic construct
  misc_feature 1..25: note = >Male | ExonID_5299845 | 17076434
  source 1..25: mol_type = other DNA
gactatattaggacgggaggccaca

SEQ ID NO: 1325 - DNA, 25 bp, synthetic construct
  misc_feature 1..25: note = >Male | ExonID_5299845 | 17076434
  source 1..25: mol_type = other DNA
agaagcatctcaccgaaatcccggg

SEQ ID NO: 1326 - DNA, 25 bp, synthetic construct
  misc_feature 1..25: note = >Male | ExonID_5299845 | 17076434
  source 1..25: mol_type = other DNA
gacggcctagcggtgcagagtgtgg

SEQ ID NO: 1327 - DNA, 25 bp, synthetic construct
  misc_feature 1..25: note = >Male | ExonID_5299845 | 17076434
  source 1..25: mol_type = other DNA
tagaggttggtgacaaggctccaca

SEQ ID NO: 1328 - DNA, 25 bp, synthetic construct
  misc_feature 1..25: note = >Male | ExonID_5299845 | 17076434
  source 1..25: mol_type = other DNA
aaagagcctaagagtgtggagccag

SEQ ID NO: 1329 - DNA, 25 bp, synthetic construct
  misc_feature 1..25: note = >Male | ExonID_5299845 | 17076434
  source 1..25: mol_type = other DNA
tactgatccaacatacagggtggac

SEQ ID NO: 1330 - DNA, 25 bp, synthetic construct
  misc_feature 1..25: note = >Male | ExonID_5299845 | 17076434
  source 1..25: mol_type = other DNA
tctatttctatagggcgccacagct

SEQ ID NO: 1331 - DNA, 25 bp, synthetic construct
  misc_feature 1..25: note = >Male | ExonID_5299845 | 17076434
  source 1..25: mol_type = other DNA
taaacaagtcattagggaggcaggc

SEQ ID NO: 1332 - DNA, 25 bp, synthetic construct
  misc_feature 1..25: note = >Male | ExonID_5299845 | 17076434
  source 1..25: mol_type = other DNA
gatgcggcccaaatcagtccttaaa

SEQ ID NO: 1333 - DNA, 25 bp, synthetic construct
  misc_feature 1..25: note = >Male | ExonID_5299845 | 17076434
  source 1..25: mol_type = other DNA
cggcggcgaataacttggtgagatc

SEQ ID NO: 1334 - DNA, 25 bp, synthetic construct
  misc_feature 1..25: note = >Male | ExonID_5299845 | 17076434
  source 1..25: mol_type = other DNA
ctcccgagcgcgagttggaggaaaa

SEQ ID NO: 1335 - DNA, 25 bp, synthetic construct
  misc_feature 1..25: note = >Male | ExonID_5299845 | 17076434
  source 1..25: mol_type = other DNA
agctcggaccggagaaaagtccttg

SEQ ID NO: 1336 - DNA, 25 bp, synthetic construct
  misc_feature 1..25: note = >Male | TCONS_00017554-XLOC_008249 | 17114699
  source 1..25: mol_type = other DNA
cattccatacacacaaacattccat

SEQ ID NO: 1337 - DNA, 25 bp, synthetic construct
  misc_feature 1..25: note = >Male | TCONS_00017554-XLOC_008249 | 17114699
  source 1..25: mol_type = other DNA
attccatacacacaaacattccata

SEQ ID NO: 1338 - DNA, 25 bp, synthetic construct
  misc_feature 1..25: note = >Male | TCONS_00017554-XLOC_008249 | 17114699
  source 1..25: mol_type = other DNA
ttccatacacacaaacattccatac

SEQ ID NO: 1339 - DNA, 25 bp, synthetic construct
  misc_feature 1..25: note = >Male | TCONS_00017554-XLOC_008249 | 17114699
  source 1..25: mol_type = other DNA
tccatacacacaaacattccataca

SEQ ID NO: 1340 - DNA, 25 bp, synthetic construct
  misc_feature 1..25: note = >Male | TCONS_00017554-XLOC_008249 | 17114699
  source 1..25: mol_type = other DNA
tacacacaaacattccatacacaca

SEQ ID NO: 1341 - DNA, 25 bp, synthetic construct
  misc_feature 1..25: note = >Male | TCONS_00017554-XLOC_008249 | 17114699
  source 1..25: mol_type = other DNA
tacacacacatattccatacacaca

SEQ ID NO: 1342 - DNA, 25 bp, synthetic construct
  misc_feature 1..25: note = >Male | TCONS_00017554-XLOC_008249 | 17114699
  source 1..25: mol_type = other DNA
atattccatacacacacattccata

SEQ ID NO: 1343 - DNA, 25 bp, synthetic construct
  misc_feature 1..25: note = >Male | TCONS_00017554-XLOC_008249 | 17114699
  source 1..25: mol_type = other DNA
catacacagacacattctgtacaca

SEQ ID NO: 1344 - DNA, 25 bp, synthetic construct
  misc_feature 1..25: note = >Male | TCONS_00017554-XLOC_008249 | 17114699
  source 1..25: mol_type = other DNA
agacacattctgtacacacagacac

SEQ ID NO: 1345 - DNA, 25 bp, synthetic construct
  misc_feature 1..25: note = >Male | TCONS_00017554-XLOC_008249 | 17114699
  source 1..25: mol_type = other DNA
gacacattctgtacacacagacaca

SEQ ID NO: 1346 - DNA, 25 bp, synthetic construct
  misc_feature 1..25: note = >Male | TCONS_00017554-XLOC_008249 | 17114699
  source 1..25: mol_type = other DNA
acagacacacattccatacacagac

SEQ ID NO: 1347 - DNA, 25 bp, synthetic construct
  misc_feature 1..25: note = >Male | TCONS_00017554-XLOC_008249 | 17114699
  source 1..25: mol_type = other DNA
cacacattccatacacagacacatt

SEQ ID NO: 1348 - DNA, 25 bp, synthetic construct
  misc_feature 1..25: note = >Male | TCONS_00017554-XLOC_008249 | 17114699
  source 1..25: mol_type = other DNA
acacattccatacacagacacattc